SEQUENCE LISTING (855 sequences)

SEQ ID NO: 1 - protein, 1273 aa, Severe acute respiratory syndrome-related coronavirus
  source 1..1273: note = Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 12 aa, Severe acute respiratory syndrome-related coronavirus
  source 1..12: note = Severe acute respiratory syndrome coronavirus 2
MFVFLVLLPLVS

SEQ ID NO: 3 - protein, 1201 aa, Severe acute respiratory syndrome-related coronavirus
  source 1..1201: note = Severe acute respiratory syndrome coronavirus 2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 4 - protein, 21 aa, Severe acute respiratory syndrome-related coronavirus
  source 1..21: note = Severe acute respiratory syndrome coronavirus 2
WYIWLGFIAGLIAIVMVTIML

SEQ ID NO: 5 - protein, 39 aa, Severe acute respiratory syndrome-related coronavirus
  source 1..39: note = Severe acute respiratory syndrome coronavirus 2
CCMTSCCSCLKGCCSCGSCCKFDEDDSEPVLKGVKLHYT

SEQ ID NO: 6 - protein, 673 aa, Severe acute respiratory syndrome-related coronavirus
  source 1..673: note = Severe acute respiratory syndrome coronavirus 2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 7 - protein, 223 aa, Severe acute respiratory syndrome-related coronavirus
  source 1..223: note = Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 72 aa, Severe acute respiratory syndrome-related coronavirus
  source 1..72: note = Severe acute respiratory syndrome coronavirus 2
NSNNLDSKVGGNYNYLYRLFRKSNLKPFERDISTEIYQAGSTPCNGVEGFNCYFPLQSYGFQPTNGVGYQPY

SEQ ID NO: 9 - protein, 588 aa, Severe acute respiratory syndrome-related coronavirus
  source 1..588: note = Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 458 aa, Severe acute respiratory syndrome-related coronavirus
  source 1..458: note = Severe acute respiratory syndrome coronavirus 2
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSLMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLVGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1273 aa, synthetic construct
MFVFLVLLPLVSSQCVNLITRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1273 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKSGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1269 aa, synthetic construct
MFVFLVLLPLVSSQCVNLITRTQSYTNSFTRGVYYPDKVFRSSVLHSTHDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKSGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 805 aa, Homo sapiens [sequence omitted]

SEQ ID NO: 29 - protein, 17 aa, Homo sapiens
MSSSSWLLLSLVAVTAA

SEQ ID NO: 30 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 31 - protein, 21 aa, Homo sapiens
IWLIVFGVVMGVIVVGIVILI

SEQ ID NO: 32 - protein, 44 aa, Homo sapiens
FTGIRDRKKKNKARSGENPYASIDISKGENNPGFQNTDDVQTSF

SEQ ID NO: 33 - protein, 788 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 34 - protein, 691 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSR

SEQ ID NO: 35 - protein, 555 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLL

SEQ ID NO: 36 - protein, 118 aa, synthetic construct
EVQLVESGGGLVQPGGSLRLSCAASEIIVSRNYMTWVRQAPGKGLEWLAVLYAGGSSFYADSVKGRFTISRDNSKNTLFLQMNSLRAEDTAVYYCARDLSLSGGFDYWGQGALVTVSS

SEQ ID NO: 37 - protein, 8 aa, synthetic construct
EIIVSRNY

SEQ ID NO: 38 - protein, 7 aa, synthetic construct
LYAGGSS

SEQ ID NO: 39 - protein, 12 aa, synthetic construct
ARDLSLSGGFDY

SEQ ID NO: 40 - protein, 25 aa, synthetic construct [sequence omitted]

SEQ ID NO: 41 - protein, 17 aa, synthetic construct
MTWVRQAPGKGLEWLAV

SEQ ID NO: 42 - protein, 38 aa, synthetic construct
FYADSVKGRFTISRDNSKNTLFLQMNSLRAEDTAVYYC

SEQ ID NO: 43 - protein, 11 aa, synthetic construct
WGQGALVTVSS

SEQ ID NO: 44 - protein, 107 aa, synthetic construct
DIQLTQSPSFLSASVGDRVTITCRASQGISNYLAWYQQNPGKAPKLLIYAVSTLHSGVPSRFSGSGSGTEFTLTISSLQPEDFATYYCQHLNTDSCTFGQGTKLEIK

SEQ ID NO: 45 - protein, 6 aa, synthetic construct
QGISNY

SEQ ID NO: 46 - skipped

SEQ ID NO: 47 - protein, 9 aa, synthetic construct
QHLNTDSCT

SEQ ID NO: 48 - protein, 26 aa, synthetic construct
DIQLTQSPSFLSASVGDRVTITCRAS

SEQ ID NO: 49 - protein, 17 aa, synthetic construct
LAWYQQNPGKAPKLLIY

SEQ ID NO: 50 - protein, 36 aa, synthetic construct
TLHSGVPSRFSGSGSGTEFTLTISSLQPEDFATYYC

SEQ ID NO: 51 - protein, 10 aa, synthetic construct
FGQGTKLEIK

SEQ ID NO: 52 - protein, 119 aa, synthetic construct
EFQLVESGGRLVRPGGSLRLSCVASEIIVSRNYMSWIRQAPGKGLEWVSILYAGGTTYYADSVKGRFTISRDNSKNTLYLQLNSLRVEDTAIYYCVRPIVGGRGGMDVWGQGTTVTVSS

SEQ ID NO: 53 - protein, 7 aa, synthetic construct
LYAGGTT

SEQ ID NO: 54 - protein, 13 aa, synthetic construct
VRPIVGGRGGMDV

SEQ ID NO: 55 - protein, 25 aa, synthetic construct
EFQLVESGGRLVRPGGSLRLSCVAS

SEQ ID NO: 56 - protein, 17 aa, synthetic construct
MSWIRQAPGKGLEWVSI

SEQ ID NO: 57 - protein, 38 aa, synthetic construct
YYADSVKGRFTISRDNSKNTLYLQLNSLRVEDTAIYYC

SEQ ID NO: 58 - protein, 11 aa, synthetic construct
WGQGTTVTVSS

SEQ ID NO: 59 - protein, 107 aa, synthetic construct
DIQMTQSPSSLSASVGDRVTITCQASQDINKYLNWYQQKPGKAPKLLIYDASNLEPGVPSRFSGSGSGTDFVFTITSLQPEDIATYYCHYYDDVPYTFGQGTQLEIK

SEQ ID NO: 60 - protein, 6 aa, synthetic construct
QDINKY

SEQ ID NO: 61 - skipped

SEQ ID NO: 62 - protein, 9 aa, synthetic construct
HYYDDVPYT

SEQ ID NO: 63 - protein, 26 aa, synthetic construct
DIQMTQSPSSLSASVGDRVTITCQAS

SEQ ID NO: 64 - protein, 17 aa, synthetic construct
LNWYQQKPGKAPKLLIY

SEQ ID NO: 65 - protein, 36 aa, synthetic construct
NLEPGVPSRFSGSGSGTDFVFTITSLQPEDIATYYC

SEQ ID NO: 66 - protein, 10 aa, synthetic construct
FGQGTQLEIK

SEQ ID NO: 67 - protein, 123 aa, synthetic construct
QVQLVQSGAEVKKPGSSVRVSCEASGDTFSRYAISWVRQAPGQGLEWMGRIIPMFGMANSAQKFQARVTISADKSTSTAYMEVSSLRSEDTAVYYCATDTFYPNDGVHRMEYWGQGALVIVSS

SEQ ID NO: 68 - protein, 8 aa, synthetic construct
GDTFSRYA

SEQ ID NO: 69 - protein, 8 aa, synthetic construct
IIPMFGMA

SEQ ID NO: 70 - protein, 16 aa, synthetic construct
ATDTFYPNDGVHRMEY

SEQ ID NO: 71 - protein, 25 aa, synthetic construct
QVQLVQSGAEVKKPGSSVRVSCEAS

SEQ ID NO: 72 - protein, 17 aa, synthetic construct
ISWVRQAPGQGLEWMGR

SEQ ID NO: 73 - protein, 38 aa, synthetic construct
NSAQKFQARVTISADKSTSTAYMEVSSLRSEDTAVYYC

SEQ ID NO: 74 - protein, 11 aa, synthetic construct
WGQGALVIVSS

SEQ ID NO: 75 - protein, 107 aa, synthetic construct
DIQMTQSPSSLSASVGDRVTITCRASQTISNYLNWYQQKPGKAPNLLIYGTSILQSGVPSRFSGSGSGTDFTLTISSLQPEDFATYYCQQSHSAPRTFGPGTKVDIK

SEQ ID NO: 76 - protein, 6 aa, synthetic construct
QTISNY

SEQ ID NO: 77 - skipped

SEQ ID NO: 78 - protein, 9 aa, synthetic construct
QQSHSAPRT

SEQ ID NO: 79 - protein, 26 aa, synthetic construct [sequence omitted]

SEQ ID NO: 80 - protein, 17 aa, synthetic construct
LNWYQQKPGKAPNLLIY

SEQ ID NO: 81 - protein, 36 aa, synthetic construct
ILQSGVPSRFSGSGSGTDFTLTISSLQPEDFATYYC

SEQ ID NO: 82 - protein, 10 aa, synthetic construct
FGPGTKVDIK

SEQ ID NO: 83 - protein, 125 aa, synthetic construct
QVQLVQSGAEVKKPGSSVKVSCEASGGTFSTYAISWVRQAPGQGLEWMGRIIPIFGIANYAQKFQGRVTITADKSTSTAYMEVSSLRSEDTAVYYCATTFYDHSSTYRTHSMDVWGQGTTVTVSS

SEQ ID NO: 84 - protein, 8 aa, synthetic construct
GGTFSTYA

SEQ ID NO: 85 - protein, 8 aa, synthetic construct
IIPIFGIA

SEQ ID NO: 86 - protein, 18 aa, synthetic construct
ATTFYDHSSTYRTHSMDV

SEQ ID NO: 87 - protein, 25 aa, synthetic construct
QVQLVQSGAEVKKPGSSVKVSCEAS

SEQ ID NO: 88 - protein, 38 aa, synthetic construct
NYAQKFQGRVTITADKSTSTAYMEVSSLRSEDTAVYYC

SEQ ID NO: 89 - protein, 107 aa, synthetic construct
DIQMTQSPSSLSASVGDRVTITCRASQSISNYLNWYQQRPGKAPKLLIYAASGLQSGVPSRFSGSGSGTNFTLTISTLHPEDFATYYCQQTHSTPRAFGGGTKVEIK

SEQ ID NO: 90 - protein, 6 aa, synthetic construct
QSISNY

SEQ ID NO: 91 - skipped

SEQ ID NO: 92 - protein, 9 aa, synthetic construct
QQTHSTPRA

SEQ ID NO: 93 - protein, 17 aa, synthetic construct
LNWYQQRPGKAPKLLIY

SEQ ID NO: 94 - protein, 36 aa, synthetic construct
GLQSGVPSRFSGSGSGTNFTLTISTLHPEDFATYYC

SEQ ID NO: 95 - protein, 10 aa, synthetic construct
FGGGTKVEIK

SEQ ID NO: 96 - protein, 119 aa, synthetic construct
DFQLVESGGGLVRPGGSLRLSCVASEITVSRNYMSWIRQAPGKGLEWVSIIYPGGTTYYADSVKGRFTISRDNSKNTMYLQLNSLRPEDTAIYYCVRPIVRGGGGMDVWGQGTTVTVSS

SEQ ID NO: 97 - protein, 8 aa, synthetic construct
EITVSRNY

SEQ ID NO: 98 - protein, 7 aa, synthetic construct
IYPGGTT

SEQ ID NO: 99 - protein, 13 aa, synthetic construct
VRPIVRGGGGMDV

SEQ ID NO: 100 - protein, 25 aa, synthetic construct
DFQLVESGGGLVRPGGSLRLSCVAS

SEQ ID NO: 101 - protein, 38 aa, synthetic construct
YYADSVKGRFTISRDNSKNTMYLQLNSLRPEDTAIYYC

SEQ ID NO: 102 - protein, 107 aa, synthetic construct
DIQMTQSPSSLSASVGDRVTITCQASQDINKYLNWYQQKPGKAPKLLIYDATNLEPGVPSRFSGSGSGTDFVFTITSLQPEDIATYYCQYYDDVPYTFGQGTQLEIK

SEQ ID NO: 103 - skipped

SEQ ID NO: 104 - protein, 9 aa, synthetic construct
QYYDDVPYT

SEQ ID NO: 105 - protein, 121 aa, synthetic construct
EVQLVESGGGLVQPGGSLRLSCSASGFTFNNYVMHWVRQAPGKGLEYVSAINSNGGSTYYAGSVKGRFTISRDNSNNTLYLQMSSLRAEDTAVYYCIKDAGYYSSLGVDSWGQGTLVTVSS

SEQ ID NO: 106 - protein, 8 aa, synthetic construct
GFTFNNYV

SEQ ID NO: 107 - protein, 8 aa, synthetic construct
INSNGGST

SEQ ID NO: 108 - protein, 14 aa, synthetic construct
IKDAGYYSSLGVDS

SEQ ID NO: 109 - protein, 25 aa, synthetic construct
EVQLVESGGGLVQPGGSLRLSCSAS

SEQ ID NO: 110 - protein, 17 aa, synthetic construct
MHWVRQAPGKGLEYVSA

SEQ ID NO: 111 - protein, 38 aa, synthetic construct
YYAGSVKGRFTISRDNSNNTLYLQMSSLRAEDTAVYYC

SEQ ID NO: 112 - protein, 11 aa, synthetic construct
WGQGTLVTVSS

SEQ ID NO: 113 - protein, 107 aa, synthetic construct
EIVLTQSPATLSLSPGERATLSCRASQSVGSYLAWFQQKPGQAPRLLIYDASYRATGIPARFSGSGSGTDFTLTISSLEPEDVGIYYCQQRSNWPQTFGGGTKVDIK

SEQ ID NO: 114 - protein, 6 aa, synthetic construct
QSVGSY

SEQ ID NO: 115 - protein, 9 aa, synthetic construct
QQRSNWPQT

SEQ ID NO: 116 - protein, 26 aa, synthetic construct [sequence omitted]

SEQ ID NO: 117 - protein, 17 aa, synthetic construct
LAWFQQKPGQAPRLLIY

SEQ ID NO: 118 - protein, 36 aa, synthetic construct
YRATGIPARFSGSGSGTDFTLTISSLEPEDVGIYYC

SEQ ID NO: 119 - protein, 10 aa, synthetic construct
FGGGTKVDIK

SEQ ID NO: 120 - protein, 128 aa, synthetic construct
EVQLVESGGGLVKPGGSLRLSCAASGFTVSTYIINWVRQAPGKGLEWVSSITSESDYMFDADSVRGRFTISRDNAKNLVYLQMNSLRAEDTAVYYCARDQGAYSGYDLSPGGDAFDVWGQGTMVTVSS

SEQ ID NO: 121 - protein, 8 aa, synthetic construct
GFTVSTYI

SEQ ID NO: 122 - protein, 8 aa, synthetic construct
ITSESDYM

SEQ ID NO: 123 - protein, 21 aa, synthetic construct
ARDQGAYSGYDLSPGGDAFDV

SEQ ID NO: 124 - protein, 25 aa, synthetic construct
EVQLVESGGGLVKPGGSLRLSCAAS

SEQ ID NO: 125 - protein, 17 aa, synthetic construct
INWVRQAPGKGLEWVSS

SEQ ID NO: 126 - protein, 38 aa, synthetic construct
FDADSVRGRFTISRDNAKNLVYLQMNSLRAEDTAVYYC

SEQ ID NO: 127 - protein, 11 aa, synthetic construct
WGQGTMVTVSS

SEQ ID NO: 128 - protein, 107 aa, synthetic construct
DIQMTQSPSTLSASVGDRVTITCRASQSISTWLAWYQQKPGKAPKLLIYKASNLESGVPSRFSGSGSGTEFTLTISRLQPDDSATYCCQHYNSYPYTFGQGTKVEIK

SEQ ID NO: 129 - protein, 6 aa, synthetic construct
QSISTW

SEQ ID NO: 130 - skipped

SEQ ID NO: 131 - protein, 9 aa, synthetic construct
QHYNSYPYT

SEQ ID NO: 132 - protein, 26 aa, synthetic construct
DIQMTQSPSTLSASVGDRVTITCRAS

SEQ ID NO: 133 - protein, 17 aa, synthetic construct
LAWYQQKPGKAPKLLIY

SEQ ID NO: 134 - protein, 36 aa, synthetic construct
NLESGVPSRFSGSGSGTEFTLTISRLQPDDSATYCC

SEQ ID NO: 135 - protein, 10 aa, synthetic construct
FGQGTKVEIK

SEQ ID NO: 136 - protein, 118 aa, synthetic construct
EVQLVQSRAEVKKPGESLKISCKGSGYSFTTYWIGWVRQMPGKGLEWMGIIRPGDSDTRYSPSFQGQVTISADKSITTAYLQWSSLKASDTAMYYCAMTYSGDQYDFWGQGTVVTVSS

SEQ ID NO: 137 - protein, 8 aa, synthetic construct
GYSFTTYW

SEQ ID NO: 138 - protein, 8 aa, synthetic construct
IRPGDSDT

SEQ ID NO: 139 - protein, 11 aa, synthetic construct
AMTYSGDQYDF

SEQ ID NO: 140 - protein, 25 aa, synthetic construct
EVQLVQSRAEVKKPGESLKISCKGS

SEQ ID NO: 141 - protein, 17 aa, synthetic construct
IGWVRQMPGKGLEWMGI

SEQ ID NO: 142 - protein, 38 aa, synthetic construct
RYSPSFQGQVTISADKSITTAYLQWSSLKASDTAMYYC

SEQ ID NO: 143 - protein, 11 aa, synthetic construct
WGQGTVVTVSS

SEQ ID NO: 144 - protein, 107 aa, synthetic construct
DIQMTQSPSTLSASVGDRVTITCRASQSIHTWLAWYQQTPGKAPKLLIYKASLLESGVPSRFSGSGSGTEFTLTISSLQPGDFATYYCQHYNSYSHTFGQGTKVEIK

SEQ ID NO: 145 - protein, 6 aa, synthetic construct
QSIHTW

SEQ ID NO: 146 - protein, 9 aa, synthetic construct
QHYNSYSHT

SEQ ID NO: 147 - protein, 17 aa, synthetic construct
LAWYQQTPGKAPKLLIY

SEQ ID NO: 148 - protein, 36 aa, synthetic construct
LLESGVPSRFSGSGSGTEFTLTISSLQPGDFATYYC

SEQ ID NO: 149 - protein, 121 aa, synthetic construct
QVQLVQSGAEVKKPGASVKVSCKISGYTLTDFSIHWVRQAPGKGLEWMAGFDPEHRETIFAQKFQGRVAMTEATSTDTAYMELSSLRSDDTAVYYCATTGDFDSWRGYYLWGQGTLVTVSA

SEQ ID NO: 150 - protein, 8 aa, synthetic construct
GYTLTDFS

SEQ ID NO: 151 - protein, 8 aa, synthetic construct
FDPEHRET

SEQ ID NO: 152 - protein, 14 aa, synthetic construct
ATTGDFDSWRGYYL

SEQ ID NO: 153 - protein, 25 aa, synthetic construct
QVQLVQSGAEVKKPGASVKVSCKIS

SEQ ID NO: 154 - protein, 17 aa, synthetic construct
IHWVRQAPGKGLEWMAG

SEQ ID NO: 155 - protein, 38 aa, synthetic construct
IFAQKFQGRVAMTEATSTDTAYMELSSLRSDDTAVYYC

SEQ ID NO: 156 - protein, 11 aa, synthetic construct
WGQGTLVTVSA

SEQ ID NO: 157 - protein, 107 aa, synthetic construct
DIQLTQSPSSVSASVGDSVTITCRASQGISRWLAWYQQKPGKAPRLLIYSASTLQSGVPSRFSGSGSGTDFTLTISSLQPDDFATYYCQHTNNFPFSFGPGTKVDIK

SEQ ID NO: 158 - protein, 6 aa, synthetic construct
QGISRW

SEQ ID NO: 159 - skipped

SEQ ID NO: 160 - protein, 9 aa, synthetic construct
QHTNNFPFS

SEQ ID NO: 161 - protein, 26 aa, synthetic construct
DIQLTQSPSSVSASVGDSVTITCRAS

SEQ ID NO: 162 - protein, 17 aa, synthetic construct
LAWYQQKPGKAPRLLIY

SEQ ID NO: 163 - protein, 36 aa, synthetic construct
TLQSGVPSRFSGSGSGTDFTLTISSLQPDDFATYYC

SEQ ID NO: 164 - protein, 128 aa, synthetic construct
QVQLVQSGAEVKKPGSSVKVSCRASGDTFSTYTITWVRQAPGQGLEWMGRIIPILDTADYAQKFQGRVTITADKSTSTAYMELSSLRSEDTAMYYCAKNYPNGYSGYDYFSWDGFDPWGQGTLVTVSS

SEQ ID NO: 165 - protein, 8 aa, synthetic construct
GDTFSTYT

SEQ ID NO: 166 - protein, 8 aa, synthetic construct
IIPILDTA

SEQ ID NO: 167 - protein, 21 aa, synthetic construct
AKNYPNGYSGYDYFSWDGFDP

SEQ ID NO: 168 - protein, 25 aa, synthetic construct
QVQLVQSGAEVKKPGSSVKVSCRAS

SEQ ID NO: 169 - protein, 17 aa, synthetic construct
ITWVRQAPGQGLEWMGR

SEQ ID NO: 170 - protein, 38 aa, synthetic construct
DYAQKFQGRVTITADKSTSTAYMELSSLRSEDTAMYYC

SEQ ID NO: 171 - protein, 111 aa, synthetic construct
QSVLTQPPSVSGAPGQRVTISCTGSSSNIGANYEVHWYQQLPGTAPKLLIFGHSNRPSAVPDRFSGSKSGTSASLAITGLQTEDEADYYCQSYDSSLSGVVFGGGTKLTVL

SEQ ID NO: 172 - protein, 9 aa, synthetic construct
SSNIGANYE

SEQ ID NO: 173 - skipped

SEQ ID NO: 174 - protein, 11 aa, synthetic construct
QSYDSSLSGVV

SEQ ID NO: 175 - protein, 25 aa, synthetic construct
QSVLTQPPSVSGAPGQRVTISCTGS

SEQ ID NO: 176 - protein, 17 aa, synthetic construct
VHWYQQLPGTAPKLLIF

SEQ ID NO: 177 - protein, 36 aa, synthetic construct
NRPSAVPDRFSGSKSGTSASLAITGLQTEDEADYYC

SEQ ID NO: 178 - protein, 10 aa, synthetic construct
FGGGTKLTVL

SEQ ID NO: 179 - protein, 116 aa, synthetic construct
QVQLQESGPRLVKPSGTLSLSCAVSGGPFSNTNWWSWIRQTPGKGLEWIGEINDSGNTVYNPALKSRVTMSVDKSKKQFSLNLHSLTAADTAVYFCARVWGHFDYWGQGVRVTVSS

SEQ ID NO: 180 - protein, 9 aa, synthetic construct
GGPFSNTNW

SEQ ID NO: 181 - protein, 7 aa, synthetic construct
INDSGNT

SEQ ID NO: 182 - protein, 9 aa, synthetic construct
ARVWGHFDY

SEQ ID NO: 183 - protein, 25 aa, synthetic construct
QVQLQESGPRLVKPSGTLSLSCAVS

SEQ ID NO: 184 - protein, 17 aa, synthetic construct
WSWIRQTPGKGLEWIGE

SEQ ID NO: 185 - protein, 38 aa, synthetic construct
VYNPALKSRVTMSVDKSKKQFSLNLHSLTAADTAVYFC

SEQ ID NO: 186 - protein, 11 aa, synthetic construct
WGQGVRVTVSS

SEQ ID NO: 187 - protein, 106 aa, synthetic construct
DIQMTQSPSSLYASVADRVTITCRASQGISNSLAWYQQQPGKAPQLLLYAASTLESGVPSRFSGSGSGTDFTLTISSLQPEDFATYYCQQYYSLRTFGGGTKVEIK

SEQ ID NO: 188 - protein, 6 aa, synthetic construct
QGISNS

SEQ ID NO: 189 - protein, 8 aa, synthetic construct
QQYYSLRT

SEQ ID NO: 190 - protein, 26 aa, synthetic construct
DIQMTQSPSSLYASVADRVTITCRAS

SEQ ID NO: 191 - protein, 17 aa, synthetic construct
LAWYQQQPGKAPQLLLY

SEQ ID NO: 192 - protein, 36 aa, synthetic construct
TLESGVPSRFSGSGSGTDFTLTISSLQPEDFATYYC

SEQ ID NO: 193 - protein, 122 aa, synthetic construct
EVQLLESGGGLVQPGGSLRLSCAASGFTFSGSDMSWVRQAPGKGLEWVSVIGGSGTYAYYSDSVKGRFTISRDNSKNMLFLQMNSLRAEDTAIYYCAKETGFLWFGELLDSWGQGALVTVSS

SEQ ID NO: 194 - protein, 8 aa, synthetic construct
GFTFSGSD

SEQ ID NO: 195 - protein, 8 aa, synthetic construct
IGGSGTYA

SEQ ID NO: 196 - protein, 15 aa, synthetic construct
AKETGFLWFGELLDS

SEQ ID NO: 197 - protein, 25 aa, synthetic construct
EVQLLESGGGLVQPGGSLRLSCAAS

SEQ ID NO: 198 - protein, 17 aa, synthetic construct
MSWVRQAPGKGLEWVSV

SEQ ID NO: 199 - protein, 38 aa, synthetic construct
YYSDSVKGRFTISRDNSKNMLFLQMNSLRAEDTAIYYC

SEQ ID NO: 200 - protein, 108 aa, synthetic construct
DIQMTQSPSSLSASVGDRVTITCRASEAISNSLAWYQQRPGKAPRLLLYAAATLESGVPPRFSGSGSGTDFTLTISTLQPEDFATYYCQQYYSPPPRTFGQGTKVEIK

SEQ ID NO: 201 - protein, 6 aa, synthetic construct
EAISNS

SEQ ID NO: 202 - skipped

SEQ ID NO: 203 - protein, 10 aa, synthetic construct
QQYYSPPPRT

SEQ ID NO: 204 - protein, 17 aa, synthetic construct
LAWYQQRPGKAPRLLLY

SEQ ID NO: 205 - protein, 36 aa, synthetic construct
TLESGVPPRFSGSGSGTDFTLTISTLQPEDFATYYC

SEQ ID NO: 206 - protein, 122 aa, synthetic construct
EVQLVESGGGLVQPGRSLRLSCAASGFTFDEYAMHWVRQAPGKGLEWVSGISFNSGSVGYAGAAKGRFTISRDNAKKSLYLEMNSLRDEDTAFYYCAKDRGEHWLVRLFDSWGQGTLVTVSS

SEQ ID NO: 207 - protein, 8 aa, synthetic construct
GFTFDEYA

SEQ ID NO: 208 - protein, 8 aa, synthetic construct
ISFNSGSV

SEQ ID NO: 209 - protein, 15 aa, synthetic construct
AKDRGEHWLVRLFDS

SEQ ID NO: 210 - protein, 25 aa, synthetic construct [sequence omitted]

SEQ ID NO: 211 - protein, 17 aa, synthetic construct
MHWVRQAPGKGLEWVSG

SEQ ID NO: 212 - protein, 38 aa, synthetic construct
GYAGAAKGRFTISRDNAKKSLYLEMNSLRDEDTAFYYC

SEQ ID NO: 213 - protein, 106 aa, synthetic construct
DIQMTQSPSTLSASVGDRVTITCRPSQSIDRWLAWYQQKPGKAPTLLISAASSLETGVPSRFSGSGSGTEFTLTISSLQPDDFATYYCQQYNGYSMFGPGTKVEIK

SEQ ID NO: 214 - protein, 6 aa, synthetic construct
QSIDRW

SEQ ID NO: 215 - protein, 8 aa, synthetic construct
QQYNGYSM

SEQ ID NO: 216 - protein, 26 aa, synthetic construct
DIQMTQSPSTLSASVGDRVTITCRPS

SEQ ID NO: 217 - protein, 17 aa, synthetic construct
LAWYQQKPGKAPTLLIS

SEQ ID NO: 218 - protein, 36 aa, synthetic construct
SLETGVPSRFSGSGSGTEFTLTISSLQPDDFATYYC

SEQ ID NO: 219 - protein, 10 aa, synthetic construct
FGPGTKVEIK

SEQ ID NO: 220 - protein, 123 aa, synthetic construct
EVQLVESGGGLVQPGGSLRLSCATSGFTFDDFAMHWVRQGPGKGLEWVSGISWNSGKIAYTDSVKGRFSISRDNAKNSLYLQMNSLRPEDTALYYCTKDHAPSAILGDILTGFDPWGQGTLVT

SEQ ID NO: 221 - protein, 8 aa, synthetic construct
GFTFDDFA

SEQ ID NO: 222 - protein, 8 aa, synthetic construct
ISWNSGKI

SEQ ID NO: 223 - protein, 19 aa, synthetic construct
TKDHAPSAILGDILTGFDP

SEQ ID NO: 224 - protein, 25 aa, synthetic construct
EVQLVESGGGLVQPGGSLRLSCATS

SEQ ID NO: 225 - protein, 17 aa, synthetic construct
MHWVRQGPGKGLEWVSG

SEQ ID NO: 226 - protein, 38 aa, synthetic construct
AYTDSVKGRFSISRDNAKNSLYLQMNSLRPEDTALYYC

SEQ ID NO: 227 - protein, 8 aa, synthetic construct
WGQGTLVT

SEQ ID NO: 228 - protein, 107 aa, synthetic construct
DIQMTQSPVSLSASVGDRVTITCRASQSISVYLNWYQQKPGKAPKLLMYAASILQSGVPSRFSGSGSATDFTLTITSLQPEDFATYFCQQSFTMPPTFGQGTNLEIK

SEQ ID NO: 229 - protein, 6 aa, synthetic construct
QSISVY

SEQ ID NO: 230 - protein, 9 aa, synthetic construct
QQSFTMPPT

SEQ ID NO: 231 - protein, 26 aa, synthetic construct
DIQMTQSPVSLSASVGDRVTITCRAS

SEQ ID NO: 232 - protein, 17 aa, synthetic construct
LNWYQQKPGKAPKLLMY

SEQ ID NO: 233 - protein, 36 aa, synthetic construct
ILQSGVPSRFSGSGSATDFTLTITSLQPEDFATYFC

SEQ ID NO: 234 - protein, 10 aa, synthetic construct
FGQGTNLEIK

SEQ ID NO: 235 - protein, 122 aa, synthetic construct
QVQLVQSGAEVKKPGASVKVSCKASGYIFNNYAIQWVRQAPGQRLEWMAWIHTGNGDTKYSQKFQGRVTITRDTSASTAYMELSSLRSEDTAIYYCARVRPNWNTLGWFDPWGQGTLVTVSS

SEQ ID NO: 236 - protein, 8 aa, synthetic construct
GYIFNNYA

SEQ ID NO: 237 - protein, 8 aa, synthetic construct
IHTGNGDT

SEQ ID NO: 238 - protein, 15 aa, synthetic construct
ARVRPNWNTLGWFDP

SEQ ID NO: 239 - protein, 25 aa, synthetic construct [sequence omitted]

SEQ ID NO: 240 - protein, 17 aa, synthetic construct
IQWVRQAPGQRLEWMAW

SEQ ID NO: 241 - protein, 38 aa, synthetic construct
KYSQKFQGRVTITRDTSASTAYMELSSLRSEDTAIYYC

SEQ ID NO: 242 - protein, 111 aa, synthetic construct
NFMLTQPHSVSESPGKTVTISCTGSGGSIATNYVQWYQQRPGSAPTTVIYEDNERPSGVPDRFSGSIDSSSNSASLTISGLRTEDEADYYCQSYDISTHWVFGGGTKLTVL

SEQ ID NO: 243 - protein, 8 aa, synthetic construct
GGSIATNY

SEQ ID NO: 244 - skipped

SEQ ID NO: 245 - protein, 10 aa, synthetic construct
QSYDISTHWV

SEQ ID NO: 246 - protein, 25 aa, synthetic construct
NFMLTQPHSVSESPGKTVTISCTGS

SEQ ID NO: 247 - protein, 17 aa, synthetic construct
VQWYQQRPGSAPTTVIY

SEQ ID NO: 248 - protein, 38 aa, synthetic construct
ERPSGVPDRFSGSIDSSSNSASLTISGLRTEDEADYYC

SEQ ID NO: 249 - protein, 118 aa, synthetic construct
EVQLVESGGTSVRPGGSLRLSCSASGFSVRSNFMTWVRQAPGKGLECVSVIYSGSGGSTFYADSVKGRFTISKDDSKNTLYLQMNSLRAEDTAIYYCAREVSHAFDLWGQGTMVTVSS

SEQ ID NO: 250 - protein, 10 aa, synthetic construct
GFSVRSNFMT

SEQ ID NO: 251 - protein, 7 aa, synthetic construct
SGSGGST

SEQ ID NO: 252 - protein, 10 aa, synthetic construct
AREVSHAFDL

SEQ ID NO: 253 - protein, 25 aa, synthetic construct
EVQLVESGGTSVRPGGSLRLSCSAS

SEQ ID NO: 254 - protein, 17 aa, synthetic construct
WVRQAPGKGLECVSVIY

SEQ ID NO: 255 - protein, 38 aa, synthetic construct
FYADSVKGRFTISKDDSKNTLYLQMNSLRAEDTAIYYC

SEQ ID NO: 256 - protein, 108 aa, synthetic construct
DIQMTQSPSTLSASVGDRVTITCRASQTIGRWLAWYQQRPGKAPSLLIYMASILESGVPLRFSGSGSGTEFTLTISGLQPDDFATYYCQQYNSDSPYSFGQGTKLEIK

SEQ ID NO: 257 - protein, 6 aa, synthetic construct
QTIGRW

SEQ ID NO: 258 - skipped

SEQ ID NO: 259 - protein, 10 aa, synthetic construct
QQYNSDSPYS

SEQ ID NO: 260 - protein, 17 aa, synthetic construct
LAWYQQRPGKAPSLLIY

SEQ ID NO: 261 - protein, 36 aa, synthetic construct
ILESGVPLRFSGSGSGTEFTLTISGLQPDDFATYYC

SEQ ID NO: 262 - protein, 118 aa, synthetic construct
QVQLQESGPGLVKPSETLSLTCTVSGGSLTSYYWSWIRQPPGGGLEWIGHIYYTGITDHNPSLKSRVTISLDTSRNQFSLKVRSVTAADTALYYCARAAGSSDYFDFWGQGTPVTVSS

SEQ ID NO: 263 - protein, 8 aa, synthetic construct
GGSLTSYY

SEQ ID NO: 264 - protein, 7 aa, synthetic construct
IYYTGIT

SEQ ID NO: 265 - protein, 12 aa, synthetic construct
ARAAGSSDYFDF

SEQ ID NO: 266 - protein, 25 aa, synthetic construct
QVQLQESGPGLVKPSETLSLTCTVS

SEQ ID NO: 267 - protein, 17 aa, synthetic construct
WSWIRQPPGGGLEWIGH

SEQ ID NO: 268 - protein, 38 aa, synthetic construct
DHNPSLKSRVTISLDTSRNQFSLKVRSVTAADTALYYC

SEQ ID NO: 269 - protein, 11 aa, synthetic construct
WGQGTPVTVSS

SEQ ID NO: 270 - protein, 106 aa, synthetic construct
DIQMTQSPSTLSASVGDRVTITCRASQSISRWLAWYQQKPGKAPKLLIYMASILESGVPSRFSGGGSGTEFTLTISSLQPDDFATYYCQQYNSYRTFGQGTKVEIK

SEQ ID NO: 271 - protein, 6 aa, synthetic construct
QSISRW

SEQ ID NO: 272 - protein, 8 aa, synthetic construct
QQYNSYRT

SEQ ID NO: 273 - protein, 36 aa, synthetic construct
ILESGVPSRFSGGGSGTEFTLTISSLQPDDFATYYC

SEQ ID NO: 274 - protein, 118 aa, synthetic construct
QVQLQESGPGLVKPSETLSLTCTVSGGSITSYYWSWIRQSPGKGLEWIGHIYYSGSTDYNPSLKSRVTISVDTSKNQFSLKLTYVAAADTAVYFCARAGGSSDYFDYWGQGTLVTVSS

SEQ ID NO: 275 - protein, 8 aa, synthetic construct
GGSITSYY

SEQ ID NO: 276 - protein, 7 aa, synthetic construct
IYYSGST

SEQ ID NO: 277 - protein, 12 aa, synthetic construct
ARAGGSSDYFDY

SEQ ID NO: 278 - protein, 17 aa, synthetic construct
WSWIRQSPGKGLEWIGH

SEQ ID NO: 279 - protein, 38 aa, synthetic construct
DYNPSLKSRVTISVDTSKNQFSLKLTYVAAADTAVYFC

SEQ ID NO: 280 - protein, 106 aa, synthetic construct
DIQMTQSPSTLSASVGDRVTITCRASQSIGRWLAWYQQKPGKAPKFLIYMASILEDGVPSRFSGSGSGTEFTLTITSLQPDDFATYYCQQYNDYRTFGQGTKVEIK

SEQ ID NO: 281 - protein, 6 aa, synthetic construct
QSIGRW

SEQ ID NO: 282 - protein, 8 aa, synthetic construct
QQYNDYRT

SEQ ID NO: 283 - protein, 17 aa, synthetic construct
LAWYQQKPGKAPKFLIY

SEQ ID NO: 284 - protein, 36 aa, synthetic construct
ILEDGVPSRFSGSGSGTEFTLTITSLQPDDFATYYC

SEQ ID NO: 285 - protein, 129 aa, synthetic construct
EVQLVESGGGLVKPGGSLRLSCAASGFIFSRNGMHWVRQAPGKGLEWVSSIDNDGTYMYYADSVRGRFTVSRDNAKNSLFLQLNSLRAEDTAVYYCARDRFGYYDALTDSYNAGYFDSWGQGTLVTVSS

SEQ ID NO: 286 - protein, 8 aa, synthetic construct
GFIFSRNG

SEQ ID NO: 287 - protein, 8 aa, synthetic construct
IDNDGTYM

SEQ ID NO: 288 - protein, 22 aa, synthetic construct
ARDRFGYYDALTDSYNAGYFDS

SEQ ID NO: 289 - protein, 17 aa, synthetic construct
MHWVRQAPGKGLEWVSS

SEQ ID NO: 290 - protein, 38 aa, synthetic construct
YYADSVRGRFTVSRDNAKNSLFLQLNSLRAEDTAVYYC

SEQ ID NO: 291 - protein, 107 aa, synthetic construct
DIQMTQSPSSLSASVGDSVTITCRASQDIYNYLAWFQQKPGKAPKSLIYTASKLESGVPSKFSGSGSGTDFTLTISSLQPEDFATYYCQQYHSSPITFGQGTRVEIK

SEQ ID NO: 292 - protein, 6 aa, synthetic construct
QDIYNY

SEQ ID NO: 293 - skipped

SEQ ID NO: 294 - protein, 9 aa, synthetic construct
QQYHSSPIT

SEQ ID NO: 295 - protein, 26 aa, synthetic construct
DIQMTQSPSSLSASVGDSVTITCRAS

SEQ ID NO: 296 - protein, 17 aa, synthetic construct
LAWFQQKPGKAPKSLIY

SEQ ID NO: 297 - protein, 36 aa, synthetic construct
KLESGVPSKFSGSGSGTDFTLTISSLQPEDFATYYC

SEQ ID NO: 298 - protein, 10 aa, synthetic construct
FGQGTRVEIK

SEQ ID NO: 299 - protein, 129 aa, synthetic construct
EVQLVESGGGLVKPGGSLRLSCAASGFAFSTNGMNWVRQVPGKGLEWVSSISSTSEYTYYTESVKGRFTISRDNAKNSLFLQMNSLRAEDTAVYYCVRDRFGYYDVLASSYNVGFFQSWGQGALVTVSS

SEQ ID NO: 300 - protein, 8 aa, synthetic construct
GFAFSTNG

SEQ ID NO: 301 - protein, 8 aa, synthetic construct
ISSTSEYT

SEQ ID NO: 302 - protein, 22 aa, synthetic construct
VRDRFGYYDVLASSYNVGFFQS

SEQ ID NO: 303 - protein, 17 aa, synthetic construct
MNWVRQVPGKGLEWVSS

SEQ ID NO: 304 - protein, 38 aa, synthetic construct
YYTESVKGRFTISRDNAKNSLFLQMNSLRAEDTAVYYC

SEQ ID NO: 305 - protein, 107 aa, synthetic construct
DIQMTQSPSSLSASVGDGVTITCRASQDISNNLAWFQQKPGKAPKCLIYSASSLQSGVPLKFRGSGSGTDFTLSITSLDPEDFATYYCQQYISYPITFGQGTRLDIK

SEQ ID NO: 306 - protein, 6 aa, synthetic construct
QDISNN

SEQ ID NO: 307 - protein, 9 aa, synthetic construct
QQYISYPIT

SEQ ID NO: 308 - protein, 26 aa, synthetic construct
DIQMTQSPSSLSASVGDGVTITCRAS

SEQ ID NO: 309 - protein, 17 aa, synthetic construct
LAWFQQKPGKAPKCLIY

SEQ ID NO: 310 - protein, 36 aa, synthetic construct
SLQSGVPLKFRGSGSGTDFTLSITSLDPEDFATYYC

SEQ ID NO: 311 - protein, 10 aa, synthetic construct
FGQGTRLDIK

SEQ ID NO: 312 - protein, 133 aa, synthetic construct
QVQLVQSGAEVKKPGSSVEVSCKASGGTFSSYAISWVRQAPGQGLEWMGRIIPSLGITNSAENFQGRVTITADKSTSTVYMELSSLRSEDTAVYYCARDFHPLYQSCSSTSCYDEWEPRMDVWGQGTTVTVSS

SEQ ID NO: 313 - protein, 8 aa, synthetic construct
GGTFSSYA

SEQ ID NO: 314 - protein, 8 aa, synthetic construct
IIPSLGIT

SEQ ID NO: 315 - protein, 26 aa, synthetic construct
ARDFHPLYQSCSSTSCYDEWEPRMDV

SEQ ID NO: 316 - protein, 25 aa, synthetic construct
QVQLVQSGAEVKKPGSSVEVSCKAS

SEQ ID NO: 317 - protein, 38 aa, synthetic construct
NSAENFQGRVTITADKSTSTVYMELSSLRSEDTAVYYC

SEQ ID NO: 318 - protein, 110 aa, synthetic construct
QSVLTQPPSASGTPGQRVTISCHGSSSNIGSKTVNWYQQLPGTAPKLLIYSNDQRPSGVPDRFSGSKSGTSASLAISGLQSEDEADYYCAAWDGSLDGPVFGGGTKLTVL

SEQ ID NO: 319 - protein, 8 aa, synthetic construct
SSNIGSKT

SEQ ID NO: 320 - skipped

SEQ ID NO: 321 - protein, 11 aa, synthetic construct
AAWDGSLDGPV

SEQ ID NO: 322 - protein, 25 aa, synthetic construct
QSVLTQPPSASGTPGQRVTISCHGS

SEQ ID NO: 323 - protein, 17 aa, synthetic construct
VNWYQQLPGTAPKLLIY

SEQ ID NO: 324 - protein, 36 aa, synthetic construct
QRPSGVPDRFSGSKSGTSASLAISGLQSEDEADYYC

SEQ ID NO: 325 - protein, 133 aa, synthetic construct
QVQLVQSGAEVKKPGSSVKVSCKASGGTFSSYGISWVRQAPGQGLEWMGRIIPSLSITNSAEKFQGRVTITADKSTSTAYMELSRLRSEDTALYYCARDFHPRYEFCDSTSCYDEWEPRMDVWGQGTTVTVSS

SEQ ID NO: 326 - protein, 8 aa, synthetic construct
GGTFSSYG

SEQ ID NO: 327 - protein, 8 aa, synthetic construct
IIPSLSIT

SEQ ID NO: 328 - protein, 26 aa, synthetic construct
ARDFHPRYEFCDSTSCYDEWEPRMDV

SEQ ID NO: 329 - protein, 25 aa, synthetic construct [sequence omitted]

SEQ ID NO: 330 - protein, 38 aa, synthetic construct
NSAEKFQGRVTITADKSTSTAYMELSRLRSEDTALYYC

SEQ ID NO: 331 - protein, 110 aa, synthetic construct
QSVLTQSPSASGTPGQRVIISCSGSSSNIGRKTVNWYQQLPGTAPKLVIYSNDQRPSGVPDRFSGSKSGTSASLAISGLQSEDEADYYCAAWDDSLDGPVFGGGTKLTVL

SEQ ID NO: 332 - protein, 8 aa, synthetic construct
SSNIGRKT

SEQ ID NO: 333 - protein, 11 aa, synthetic construct
AAWDDSLDGPV

SEQ ID NO: 334 - protein, 25 aa, synthetic construct
QSVLTQSPSASGTPGQRVIISCSGS

SEQ ID NO: 335 - protein, 17 aa, synthetic construct
VNWYQQLPGTAPKLVIY

SEQ ID NO: 336 - protein, 133 aa, synthetic construct
QVQLVQSGAEVKKPGSAVKVSCKASGGTFSSYPITWVRQAPGQGLEWVGRAIPILGITSTAQKFQGRVTIIADKSTSTAYMELSRLRSEDTAVYYCARDFHPRYQDCDSTSCYDQWEPRMDVWGQGTTVTVSS

SEQ ID NO: 337 - protein, 8 aa, synthetic construct
GGTFSSYP

SEQ ID NO: 338 - protein, 8 aa, synthetic construct
AIPILGIT

SEQ ID NO: 339 - protein, 26 aa, synthetic construct
ARDFHPRYQDCDSTSCYDQWEPRMDV

SEQ ID NO: 340 - protein, 25 aa, synthetic construct
QVQLVQSGAEVKKPGSAVKVSCKAS

SEQ ID NO: 341 - protein, 17 aa, synthetic construct
ITWVRQAPGQGLEWVGR

SEQ ID NO: 342 - protein, 38 aa, synthetic construct
STAQKFQGRVTIIADKSTSTAYMELSRLRSEDTAVYYC

SEQ ID NO: 343 - protein, 110 aa, synthetic construct
QSVLTQPPSASGTPGQRVTISCSGSSSNIGSNTVNWYQQLPGTAPKLLIYTNNQRPSGVPNRFSASKSGTSASLAISGLQSEDEADYYCAAWDDRLSGPVFGGGTKVTVL

SEQ ID NO: 344 - protein, 8 aa, synthetic construct
SSNIGSNT

SEQ ID NO: 345 - skipped

SEQ ID NO: 346 - protein, 11 aa, synthetic construct
AAWDDRLSGPV

SEQ ID NO: 347 - protein, 25 aa, synthetic construct
QSVLTQPPSASGTPGQRVTISCSGS

SEQ ID NO: 348 - protein, 36 aa, synthetic construct
QRPSGVPNRFSASKSGTSASLAISGLQSEDEADYYC

SEQ ID NO: 349 - protein, 10 aa, synthetic construct
FGGGTKVTVL

SEQ ID NO: 350 - protein, 123 aa, synthetic construct
QVQLVQSGAEVKKPGSSVKVSCEASGDTFSRYAINWVRQAPGQGLEWMGRIIPMFGNTNYAQKFQGRFTITADKSRGTAYMEVIGLTSADTAVYYCATSPFYYSDGGYPFDFWGQGTLVTVSS

SEQ ID NO: 351 - protein, 8 aa, synthetic construct
IIPMFGNT

SEQ ID NO: 352 - protein, 16 aa, synthetic construct
ATSPFYYSDGGYPFDF

SEQ ID NO: 353 - protein, 17 aa, synthetic construct
INWVRQAPGQGLEWMGR

SEQ ID NO: 354 - protein, 38 aa, synthetic construct
NYAQKFQGRFTITADKSRGTAYMEVIGLTSADTAVYYC

SEQ ID NO: 355 - protein, 108 aa, synthetic construct
EIVLTQSPGTLSLSPGERATLSCRASQSLNSAYLAWYQQRAGLAPRLLIYGASSRATGIPDKFSGSGSGTDFTLTISRLEPEDFAVYYCQQYAYSPRTFGQGTKVEIK

SEQ ID NO: 356 - protein, 7 aa, synthetic construct
QSLNSAY

SEQ ID NO: 357 - skipped

SEQ ID NO: 358 - protein, 9 aa, synthetic construct
QQYAYSPRT

SEQ ID NO: 359 - protein, 26 aa, synthetic construct [sequence omitted]

SEQ ID NO: 360 - protein, 17 aa, synthetic construct
LAWYQQRAGLAPRLLIY

SEQ ID NO: 361 - protein, 36 aa, synthetic construct
SRATGIPDKFSGSGSGTDFTLTISRLEPEDFAVYYC

SEQ ID NO: 362 - protein, 133 aa, synthetic construct
QVQLVQSGAEVKKPGSSVKVSCKASRGTFSNYAISWVRQAPGQGLEWMGRIIPSLSITNSAEKFQGRVTMTADKSTSTVYMELSRLRSEDTAVYYCARDFHPRYEYCSSTSCYDEWEPRMDVWGQGTTVTVSS

SEQ ID NO: 363 - protein, 8 aa, synthetic construct
RGTFSNYA

SEQ ID NO: 364 - protein, 26 aa, synthetic construct
ARDFHPRYEYCSSTSCYDEWEPRMDV

SEQ ID NO: 365 - protein, 38 aa, synthetic construct
NSAEKFQGRVTMTADKSTSTVYMELSRLRSEDTAVYYC

SEQ ID NO: 366 - protein, 110 aa, synthetic construct
QSVLTQSPSASGTPGQRVIISCSGSSSNIGRKTVNWYQQLPGTAPKLLMYSNDQRPSGVPDRFSGSKSGTSASLAISGLQSEDEADYYCAAWDDSLDGPVFGGGTKLTVL

SEQ ID NO: 367 - protein, 17 aa, synthetic construct
VNWYQQLPGTAPKLLMY

SEQ ID NO: 368 - protein, 123 aa, synthetic construct
QVQLVQSGAEVKKPGSSVKVSCKASGGTFSSHTVSWVRQAPGQGLEWMGRIIPPFGIVNYAQKFQGRVTMTADESTSTAYMELSSLRSEDTAVYYCARSNVVVVTEAGWFDPWGQGTLVTVSS

SEQ ID NO: 369 - protein, 8 aa, synthetic construct
GGTFSSHT

SEQ ID NO: 370 - protein, 8 aa, synthetic construct
IIPPFGIV

SEQ ID NO: 371 - protein, 16 aa, synthetic construct
ARSNVVVVTEAGWFDP

SEQ ID NO: 372 - protein, 17 aa, synthetic construct
VSWVRQAPGQGLEWMGR

SEQ ID NO: 373 - protein, 38 aa, synthetic construct
NYAQKFQGRVTMTADESTSTAYMELSSLRSEDTAVYYC

SEQ ID NO: 374 - protein, 107 aa, synthetic construct
DIQMTQSPSSVSASVGDRVSISCRASQDISSSLAWYQQKPGQAPKVLIYDASTLQSGVPSRFSGSGSGTDFTLTISNLRPDDFATYFCQQAHSFPFTFGPGTKVDIR

SEQ ID NO: 375 - protein, 6 aa, synthetic construct
QDISSS

SEQ ID NO: 376 - protein, 9 aa, synthetic construct
QQAHSFPFT

SEQ ID NO: 377 - protein, 26 aa, synthetic construct
DIQMTQSPSSVSASVGDRVSISCRAS

SEQ ID NO: 378 - protein, 17 aa, synthetic construct
LAWYQQKPGQAPKVLIY

SEQ ID NO: 379 - protein, 36 aa, synthetic construct
TLQSGVPSRFSGSGSGTDFTLTISNLRPDDFATYFC

SEQ ID NO: 380 - protein, 10 aa, synthetic construct
FGPGTKVDIR

SEQ ID NO: 381 - protein, 117 aa, synthetic construct
EVQLVESGGGLVQPGGSLRLSCGASGITVSSNYMNWVRQAPGKGLEWVSTLYAGGSTFYADSVKGRFIISRDNSKNTLYLQMNSLRADDTAVYYCARDLVDYGMDVWGQGTTVTVSS

SEQ ID NO: 382 - protein, 8 aa, synthetic construct
GITVSSNY

SEQ ID NO: 383 - protein, 7 aa, synthetic construct
LYAGGST

SEQ ID NO: 384 - protein, 11 aa, synthetic construct
ARDLVDYGMDV

SEQ ID NO: 385 - protein, 25 aa, synthetic construct
EVQLVESGGGLVQPGGSLRLSCGAS

SEQ ID NO: 386 - protein, 17 aa, synthetic construct
MNWVRQAPGKGLEWVST

SEQ ID NO: 387 - protein, 38 aa, synthetic construct
FYADSVKGRFIISRDNSKNTLYLQMNSLRADDTAVYYC

SEQ ID NO: 388 - protein, 108 aa, synthetic construct
DIQLTQSPSSLSASVGDRVTITCRASQGISSYLAWYQQKPGKAPKLLIYAASTLQSGVPSRFSGSGSGTDFTLTISSLQPEDFATYYCQQLNSYSPFTFGPGTKVDIK

SEQ ID NO: 389 - protein, 6 aa, synthetic construct
QGISSY

SEQ ID NO: 390 - protein, 10 aa, synthetic construct
QQLNSYSPFT

SEQ ID NO: 391 - protein, 26 aa, synthetic construct [sequence omitted]

SEQ ID NO: 392 - protein, 36 aa, synthetic construct
TLQSGVPSRFSGSGSGTDFTLTISSLQPEDFATYYC

SEQ ID NO: 393 - protein, 123 aa, synthetic construct
QFQLVQSGAEVKKPGSSVKVSCRASGGSFTSHAISWVRQAPGQGFEWMGRIIPMFGIANYAPKFQGRVTMSADKFKDIVYMEVNSLTSEDTALYYCARSQPMTSVTTLWFDPWGQGTLVTVSS

SEQ ID NO: 394 - protein, 8 aa, synthetic construct
GGSFTSHA

SEQ ID NO: 395 - protein, 8 aa, synthetic construct
IIPMFGIA

SEQ ID NO: 396 - protein, 16 aa, synthetic construct
ARSQPMTSVTTLWFDP

SEQ ID NO: 397 - protein, 25 aa, synthetic construct
QFQLVQSGAEVKKPGSSVKVSCRAS

SEQ ID NO: 398 - protein, 17 aa, synthetic construct
ISWVRQAPGQGFEWMGR

SEQ ID NO: 399 - protein, 38 aa, synthetic construct
NYAPKFQGRVTMSADKFKDIVYMEVNSLTSEDTALYYC

SEQ ID NO: 400 - protein, 108 aa, synthetic construct
EIVLTQSPGTLSLSPGERATLSCRASEPVGGSYLAWYQQKPGQAPRLLIHGASSRATGIPDRFSGSGSGTDFVLTISRLEPEDFAVYHCQQYASSPYTFGQGTKLEIK

SEQ ID NO: 401 - protein, 7 aa, synthetic construct
EPVGGSY

SEQ ID NO: 402 - protein, 9 aa, synthetic construct
QQYASSPYT

SEQ ID NO: 403 - protein, 17 aa, synthetic construct
LAWYQQKPGQAPRLLIH

SEQ ID NO: 404 - protein, 36 aa, synthetic construct
SRATGIPDRFSGSGSGTDFVLTISRLEPEDFAVYHC

SEQ ID NO: 405 - protein, 123 aa, synthetic construct
QVHLVQSGAEVKKPGSSVKVSCKTSGDTFTRYAINWVRQAPGQGLEWMGRIIPMFGIPNYAQKFQGRVTMTADKSTDIAYMELSSLRSEDTAVYYCARSSFYSDSSGYYLDYWGQGTLVTVSS

SEQ ID NO: 406 - protein, 8 aa, synthetic construct
GDTFTRYA

SEQ ID NO: 407 - protein, 8 aa, synthetic construct
IIPMFGIP

SEQ ID NO: 408 - protein, 16 aa, synthetic construct
ARSSFYSDSSGYYLDY

SEQ ID NO: 409 - protein, 25 aa, synthetic construct
QVHLVQSGAEVKKPGSSVKVSCKTS

SEQ ID NO: 410 - protein, 38 aa, synthetic construct
NYAQKFQGRVTMTADKSTDIAYMELSSLRSEDTAVYYC

SEQ ID NO: 411 - protein, 108 aa, synthetic construct
EIVLTQSPGTLSLSPGERATLSCRASQNLDSNYLAWYQQKPGQAPRLLIYGASIRATGIPDRFSGSGSGTDFTLTISRLEPEDFAVYHCQQYHNSPRTFGQGTKVEIK

SEQ ID NO: 412 - protein, 7 aa, synthetic construct
QNLDSNY

SEQ ID NO: 413 - protein, 9 aa, synthetic construct
QQYHNSPRT

SEQ ID NO: 414 - protein, 17 aa, synthetic construct
LAWYQQKPGQAPRLLIY

SEQ ID NO: 415 - protein, 36 aa, synthetic construct
IRATGIPDRFSGSGSGTDFTLTISRLEPEDFAVYHC

SEQ ID NO: 416 - protein, 123 aa, synthetic construct
QVQLVQSGAEVKKPGSSVKVSCKASGDTFTKYAITWVRQAPGEGLEWMGRIIPRFGMANYAQNFQGRVTMTADQSTSTAYMELTSLRSNDTAVYYCATTFYFDSSYYHAMDYWGQGSLVTVSS

SEQ ID NO: 417 - protein, 8 aa, synthetic construct
GDTFTKYA

SEQ ID NO: 418 - protein, 8 aa, synthetic construct
IIPRFGMA

SEQ ID NO: 419 - protein, 16 aa, synthetic construct
ATTFYFDSSYYHAMDY

SEQ ID NO: 420 - protein, 17 aa, synthetic construct
ITWVRQAPGEGLEWMGR

SEQ ID NO: 421 - protein, 38 aa, synthetic construct
NYAQNFQGRVTMTADQSTSTAYMELTSLRSNDTAVYYC

SEQ ID NO: 422 - protein, 11 aa, synthetic construct
WGQGSLVTVSS

SEQ ID NO: 423 - protein, 107 aa, synthetic construct
DIQMTQSPSSLSASVGDRVTITCRASQNIDTYLIWYQQKPGKAPNLLVYGASTLQSGVPSRFSGSGSGTDFTLTISSLQPEDFATYYCQQTYNAPRTFGQGTRLDIK

SEQ ID NO: 424 - protein, 6 aa, synthetic construct
QNIDTY

SEQ ID NO: 425 - protein, 9 aa, synthetic construct
QQTYNAPRT

SEQ ID NO: 426 - protein, 17 aa, synthetic construct
LIWYQQKPGKAPNLLVY

SEQ ID NO: 427 - protein, 125 aa, synthetic construct
QVQLVQSGAEVKKPGSSVKVSCKASGDTFSRYAISWVRQAPGQGLEWMGRIIPMFGTANYAQNFQGRVTITADKSTSTAYMELTSLRSEDTAVYYCATTYFYDSDRDRTHSMDVWGPGSAVTVSS

SEQ ID NO: 428 - protein, 8 aa, synthetic construct
IIPMFGTA

SEQ ID NO: 429 - protein, 18 aa, synthetic construct
ATTYFYDSDRDRTHSMDV

SEQ ID NO: 430 - protein, 38 aa, synthetic construct
NYAQNFQGRVTITADKSTSTAYMELTSLRSEDTAVYYC

SEQ ID NO: 431 - protein, 11 aa, synthetic construct
WGPGSAVTVSS

SEQ ID NO: 432 - protein, 107 aa, synthetic construct
DIQMTQSPSSLSASVGDRVTITCRTSQSVGNYLNWYQQKPGKAPNLLIYAASTLQNGVPSRFSGSGSGTDFTLTISSLQPEDFATYYCQQTHSTPRAFGGGTKVEIK

SEQ ID NO: 433 - protein, 6 aa, synthetic construct
QSVGNY

SEQ ID NO: 434 - protein, 26 aa, synthetic construct
DIQMTQSPSSLSASVGDRVTITCRTS

SEQ ID NO: 435 - protein, 36 aa, synthetic construct
TLQNGVPSRFSGSGSGTDFTLTISSLQPEDFATYYC

SEQ ID NO: 436 - protein, 116 aa, synthetic construct
EVQLVESGGGLVQPGGSLRVTCVASGFAVRTNFMTWVRQAPGKGLQCVSVIYGDGSTYYADSVKGRFSISRDNSKNTVYLQMNSLSAEDTAVYYCAREVSHAFDLWGPGTMVTVSS

SEQ ID NO: 437 - protein, 8 aa, synthetic construct
GFAVRTNF

SEQ ID NO: 438 - protein, 7 aa, synthetic construct
IYGDGST

SEQ ID NO: 439 - protein, 25 aa, synthetic construct
EVQLVESGGGLVQPGGSLRVTCVAS

SEQ ID NO: 440 - protein, 17 aa, synthetic construct
MTWVRQAPGKGLQCVSV

SEQ ID NO: 441 - protein, 38 aa, synthetic construct
YYADSVKGRFSISRDNSKNTVYLQMNSLSAEDTAVYYC

SEQ ID NO: 442 - protein, 11 aa, synthetic construct
WGPGTMVTVSS

SEQ ID NO: 443 - protein, 108 aa, synthetic construct
DIQLTQSPSTLSASLGDRVTITCRASQSISGWLAWYQQKPGRAPQLLIYKASLLETGVPSRFSGSGSGTVFTLTISSLQPDDFATYYCQQYDTYSPYTFGQGTKLEIK

SEQ ID NO: 444 - protein, 6 aa, synthetic construct
QSISGW

SEQ ID NO: 445 - protein, 10 aa, synthetic construct
QQYDTYSPYT

SEQ ID NO: 446 - protein, 26 aa, synthetic construct
DIQLTQSPSTLSASLGDRVTITCRAS

SEQ ID NO: 447 - protein, 17 aa, synthetic construct
LAWYQQKPGRAPQLLIY

SEQ ID NO: 448 - protein, 36 aa, synthetic construct
LLETGVPSRFSGSGSGTVFTLTISSLQPDDFATYYC

SEQ ID NO: 449 - protein, 117 aa, synthetic construct
EVQLVESGGGLVQPGGSLRLSCGASGITVSSNYMNWVRQAPGKGLEWVSTLYAGGSTFYADSVKGRFIISRDNSKNTLYLQMNSLRAEDTAVYYCARDLVDYGMDVWGQGTTVTVSS

SEQ ID NO: 450 - protein, 38 aa, synthetic construct
FYADSVKGRFIISRDNSKNTLYLQMNSLRAEDTAVYYC

SEQ ID NO: 451 - protein, 108 aa, synthetic construct
AIQLTQSPSSLSASVGDRVTITCRASQGISSYLAWYQQKPGKAPKLLIYAASTLQSGVPSRFSGSGSGTDFTLTISSLQPEDFATYYCQQLNSYSPFTFGPGTKVDIK

SEQ ID NO: 452 - protein, 26 aa, synthetic construct
AIQLTQSPSSLSASVGDRVTITCRAS

SEQ ID NO: 453 - protein, 117 aa, synthetic construct
EVQLVESGGGLVQSGGSLRLSCAASGIIVSRNYMSWVRQAPGKGLEWVSVIYPGGSTFYADSVKGRFTISRDNSKNTLYLQMNSLRAEDTAVFYCARDRGEDIFDFWGQGTMVSVSS

SEQ ID NO: 454 - protein, 8 aa, synthetic construct
GIIVSRNY

SEQ ID NO: 455 - protein, 7 aa, synthetic construct
IYPGGST

SEQ ID NO: 456 - protein, 11 aa, synthetic construct
ARDRGEDIFDF

SEQ ID NO: 457 - protein, 25 aa, synthetic construct
EVQLVESGGGLVQSGGSLRLSCAAS

SEQ ID NO: 458 - protein, 38 aa, synthetic construct
FYADSVKGRFTISRDNSKNTLYLQMNSLRAEDTAVFYC

SEQ ID NO: 459 - protein, 11 aa, synthetic construct
WGQGTMVSVSS

SEQ ID NO: 460 - protein, 108 aa, synthetic construct
DIQLTQSPTFLSASVGDRVTITCRASQGISSYLAWYQQKPGKAPKLLIYAASTLQGGVPSRFSGSGSGTEFTLTISSLQPEDFATYYCQQLNSSPGFTFGPGTKVEIK

SEQ ID NO: 461 - protein, 10 aa, synthetic construct
QQLNSSPGFT

SEQ ID NO: 462 - protein, 26 aa, synthetic construct
DIQLTQSPTFLSASVGDRVTITCRAS

SEQ ID NO: 463 - protein, 36 aa, synthetic construct
TLQGGVPSRFSGSGSGTEFTLTISSLQPEDFATYYC

SEQ ID NO: 464 - protein, 118 aa, synthetic construct
QVQLQESGPGLVKPSETLSLTCTVSGDSIYSYSWTWIRQPPGKGLEWIGQLYYNGSTSYNASLKSRVSISLDTSKNQFSLKLRFVTAADTAMYYCARVEYYSNYFDPWGQGTQVTVSS

SEQ ID NO: 465 - protein, 8 aa, synthetic construct
GDSIYSYS

SEQ ID NO: 466 - protein, 7 aa, synthetic construct
LYYNGST

SEQ ID NO: 467 - protein, 12 aa, synthetic construct
ARVEYYSNYFDP

SEQ ID NO: 468 - protein, 17 aa, synthetic construct
WTWIRQPPGKGLEWIGQ

SEQ ID NO: 469 - protein, 38 aa, synthetic construct
SYNASLKSRVSISLDTSKNQFSLKLRFVTAADTAMYYC

SEQ ID NO: 470 - protein, 11 aa, synthetic construct
WGQGTQVTVSS

SEQ ID NO: 471 - protein, 107 aa, synthetic construct
EIVLTQSPATLSLSPGERATLSCRASQSVGSYLAWYQQKPGQAPRLLIYDASNRVPGIPARFSGSGSGTDFTLTISSLPPEDFAVYYCQQRSDWPPTFGGGTKVEIR

SEQ ID NO: 472 - protein, 9 aa, synthetic construct
QQRSDWPPT

SEQ ID NO: 473 - protein, 36 aa, synthetic construct
NRVPGIPARFSGSGSGTDFTLTISSLPPEDFAVYYC

SEQ ID NO: 474 - protein, 10 aa, synthetic construct
FGGGTKVEIR

SEQ ID NO: 475 - protein, 118 aa, synthetic construct
EVQLVESGGGLVQPGGSLRVSCAASGIIVSRNYMTWVRQAPGKGLEWVSVIYAGGSTFYADSVKGRFTISRDNSKNTLYLQMNSLRVDDTAVYYCARDLEIAGGMDVWGQGTTVTVSS

SEQ ID NO: 476 - protein, 7 aa, synthetic construct
IYAGGST

SEQ ID NO: 477 - protein, 12 aa, synthetic construct
ARDLEIAGGMDV

SEQ ID NO: 478 - protein, 25 aa, synthetic construct
EVQLVESGGGLVQPGGSLRVSCAAS

SEQ ID NO: 479 - protein, 17 aa, synthetic construct
MTWVRQAPGKGLEWVSV

SEQ ID NO: 480 - protein, 38 aa, synthetic construct
FYADSVKGRFTISRDNSKNTLYLQMNSLRVDDTAVYYC

SEQ ID NO: 481 - protein, 107 aa, synthetic construct
AIQLTQSPSSLSASVGDRVTITCRASEGISNYLAWYQQKPGKAPKVLIYAASTLQSGAPPRFSGSGSGTDFTLTISSLQPEDFATYYCQQLNSYPITFGQGTRLEIK

SEQ ID NO: 482 - protein, 6 aa, synthetic construct
EGISNY

SEQ ID NO: 483 - protein, 9 aa, synthetic construct
QQLNSYPIT

SEQ ID NO: 484 - protein, 17 aa, synthetic construct
LAWYQQKPGKAPKVLIY

SEQ ID NO: 485 - protein, 36 aa, synthetic construct
TLQSGAPPRFSGSGSGTDFTLTISSLQPEDFATYYC

SEQ ID NO: 486 - protein, 10 aa, synthetic construct
FGQGTRLEIK

SEQ ID NO: 487 - protein, 123 aa, synthetic construct
QVQLVQSGAEVKKPGSSVKVSCKASGDTFSRDAISWVRQAPGQGLEWMGRIIPMFGIANYAQNFQGRVTMTADKYTSTAYMELSSLRSEDTAVYYCARGGYQYESSGYHLDHWGQGTLVTVSS

SEQ ID NO: 488 - protein, 8 aa, synthetic construct
GDTFSRDA

SEQ ID NO: 489 - protein, 16 aa, synthetic construct
ARGGYQYESSGYHLDH

SEQ ID NO: 490 - protein, 38 aa, synthetic construct
NYAQNFQGRVTMTADKYTSTAYMELSSLRSEDTAVYYC

SEQ ID NO: 491 - protein, 108 aa, synthetic construct
EIVLTQSPGTLSLSPGERATLSCRASQSLSSSYLAWYQQKPGQAPRLLIYGTSSRATGIPDRFSGSGSGTDFTLTISRLEPEDFAVYYCHQYSNSPRTFGQGTKVEVK

SEQ ID NO: 492 - protein, 7 aa, synthetic construct
QSLSSSY

SEQ ID NO: 493 - protein, 9 aa, synthetic construct
HQYSNSPRT

SEQ ID NO: 494 - protein, 36 aa, synthetic construct [sequence omitted]

SEQ ID NO: 495 - protein, 10 aa, synthetic construct
FGQGTKVEVK

SEQ ID NO: 496 - protein, 120 aa, synthetic construct
QVQLVQSGAEVKKPGSSVKISCKASGDTSSSYTINWVRQAPGQGLEWMGRIIPMFNRANYARKFQGRVTMTADRSTDTAYMEVSSLTSDDTAVYYCARTWIEPHNWFDPWGQGTLVTVSS

SEQ ID NO: 497 - protein, 8 aa, synthetic construct
GDTSSSYT

SEQ ID NO: 498 - protein, 8 aa, synthetic construct
IIPMFNRA

SEQ ID NO: 499 - protein, 13 aa, synthetic construct
ARTWIEPHNWFDP

SEQ ID NO: 500 - protein, 25 aa, synthetic construct
QVQLVQSGAEVKKPGSSVKISCKAS

SEQ ID NO: 501 - protein, 38 aa, synthetic construct
NYARKFQGRVTMTADRSTDTAYMEVSSLTSDDTAVYYC

SEQ ID NO: 502 - protein, 108 aa, synthetic construct
EIVLRQSPGTLSLSPGERATLSCRASQSVSGNHLAWYQRKPGQAPRLLIYAASSRATGIPDRFSGSGSGTDFTLTINRLEPEDFAVFYCQQYGTSPPTFGGGTKVEIK

SEQ ID NO: 503 - protein, 7 aa, synthetic construct
QSVSGNH

SEQ ID NO: 504 - protein, 9 aa, synthetic construct
QQYGTSPPT

SEQ ID NO: 505 - protein, 26 aa, synthetic construct
EIVLRQSPGTLSLSPGERATLSCRAS

SEQ ID NO: 506 - protein, 17 aa, synthetic construct
LAWYQRKPGQAPRLLIY

SEQ ID NO: 507 - protein, 36 aa, synthetic construct
SRATGIPDRFSGSGSGTDFTLTINRLEPEDFAVFYC

SEQ ID NO: 508 - protein, 120 aa, synthetic construct
QVQLQESGPRLVKPSETLSLTCTVSGGSISSYYWTWIRQPPGKGLEWIGYISYSGSTNYNPSLRSRVTMSVDTSKNQFSLDLNSVTAADTAVYYCATDGGGFYPGYFPHWGQGTLVTVSS

SEQ ID NO: 509 - protein, 8 aa, synthetic construct
GGSISSYY

SEQ ID NO: 510 - protein, 7 aa, synthetic construct
ISYSGST

SEQ ID NO: 511 - protein, 14 aa, synthetic construct
ATDGGGFYPGYFPH

SEQ ID NO: 512 - protein, 25 aa, synthetic construct
QVQLQESGPRLVKPSETLSLTCTVS

SEQ ID NO: 513 - protein, 17 aa, synthetic construct
WTWIRQPPGKGLEWIGY

SEQ ID NO: 514 - protein, 38 aa, synthetic construct
NYNPSLRSRVTMSVDTSKNQFSLDLNSVTAADTAVYYC

SEQ ID NO: 515 - protein, 108 aa, synthetic construct
DIQMTQSPSSLSASVGDRVTITCRASQSISDYVNWYQQKPGRAPRLLIYAPSTLQSGVPSRFSGSGSGTDFTLTISNLQPEDFGTYFCQQSYSTPPYTFGQGTKVEMK

SEQ ID NO: 516 - protein, 6 aa, synthetic construct
QSISDY

SEQ ID NO: 517 - skipped

SEQ ID NO: 518 - protein, 10 aa, synthetic construct
QQSYSTPPYT

SEQ ID NO: 519 - protein, 17 aa, synthetic construct
VNWYQQKPGRAPRLLIY

SEQ ID NO: 520 - protein, 36 aa, synthetic construct
TLQSGVPSRFSGSGSGTDFTLTISNLQPEDFGTYFC

SEQ ID NO: 521 - protein, 10 aa, synthetic construct
FGQGTKVEMK

SEQ ID NO: 522 - protein, 121 aa, synthetic construct
QVQLVQSGAEVKKPGASVKVSCKGSGYTFTDYGISWVRQAPGQGLEWMGWIRAYNGNTDYAQKFQGRVTMTRDTSTTTAYMELRSLRSDDTAVYFCALNLIAAAATPFDHWGQGTLVTVSS

SEQ ID NO: 523 - protein, 8 aa, synthetic construct
GYTFTDYG

SEQ ID NO: 524 - protein, 8 aa, synthetic construct
IRAYNGNT

SEQ ID NO: 525 - protein, 14 aa, synthetic construct
ALNLIAAAATPFDH

SEQ ID NO: 526 - protein, 25 aa, synthetic construct
QVQLVQSGAEVKKPGASVKVSCKGS

SEQ ID NO: 527 - protein, 17 aa, synthetic construct
ISWVRQAPGQGLEWMGW

SEQ ID NO: 528 - protein, 38 aa, synthetic construct
DYAQKFQGRVTMTRDTSTTTAYMELRSLRSDDTAVYFC

SEQ ID NO: 529 - protein, 108 aa, synthetic construct
EIVLTQSPGTLSLSRGERATLSCRASQSVSSSYLAWYQQKRGQAPRLLIYGATSRATGIPDRFSGSGSGTDFTLTISRLEPEDFAVYYCQHYGSSPPPFGGGTKVEIR

SEQ ID NO: 530 - protein, 7 aa, synthetic construct
QSVSSSY

SEQ ID NO: 531 - skipped

SEQ ID NO: 532 - protein, 9 aa, synthetic construct
QHYGSSPPP

SEQ ID NO: 533 - protein, 26 aa, synthetic construct
EIVLTQSPGTLSLSRGERATLSCRAS

SEQ ID NO: 534 - protein, 17 aa, synthetic construct
LAWYQQKRGQAPRLLIY

SEQ ID NO: 535 - protein, 121 aa, synthetic construct
EVQLLESGGGLVQPGGSLRLSCAGSGFTFSSYAMSWVRQAPGKGLEWVSSISSSGGLTFYADSVRGRSTISRDHSRNTLYLQMNSLRAEDTALYYCAREQGDSYDDYFDYWGQGTLVTVSS

SEQ ID NO: 536 - protein, 8 aa, synthetic construct
GFTFSSYA

SEQ ID NO: 537 - protein, 8 aa, synthetic construct
ISSSGGLT

SEQ ID NO: 538 - protein, 14 aa, synthetic construct
AREQGDSYDDYFDY

SEQ ID NO: 539 - protein, 25 aa, synthetic construct
EVQLLESGGGLVQPGGSLRLSCAGS

SEQ ID NO: 540 - protein, 17 aa, synthetic construct
MSWVRQAPGKGLEWVSS

SEQ ID NO: 541 - protein, 38 aa, synthetic construct
FYADSVRGRSTISRDHSRNTLYLQMNSLRAEDTALYYC

SEQ ID NO: 542 - protein, 108 aa, synthetic construct
DIQMTQSPSSLSASVGDRVTITCRASQGIRSSLAWYQQRPGKAPKLLLSAASILESGVPSRFSGSGSGTDYTLTINGLQPDDFATYYCQQYYVTPGITFGGGTKVEIR

SEQ ID NO: 543 - protein, 6 aa, synthetic construct
QGIRSS

SEQ ID NO: 544 - protein, 10 aa, synthetic construct
QQYYVTPGIT

SEQ ID NO: 545 - protein, 17 aa, synthetic construct
LAWYQQRPGKAPKLLLS

SEQ ID NO: 546 - protein, 36 aa, synthetic construct
ILESGVPSRFSGSGSGTDYTLTINGLQPDDFATYYC

SEQ ID NO: 547 - protein, 121 aa, synthetic construct
EVQLVESGGGLVKPGGSLRLSCAASGFTFSRHTMNWVRQAPGKGLEWVSSIGGSTPLSFYAESVKGRFSISRDNAKSSLFLQMSSLRADDTAVYYCVRDSIASVTTLFDLWGRGTVVTVSS

SEQ ID NO: 548 - protein, 8 aa, synthetic construct
GFTFSRHT

SEQ ID NO: 549 - protein, 8 aa, synthetic construct
IGGSTPLS

SEQ ID NO: 550 - protein, 14 aa, synthetic construct
VRDSIASVTTLFDL

SEQ ID NO: 551 - protein, 17 aa, synthetic construct
MNWVRQAPGKGLEWVSS

SEQ ID NO: 552 - protein, 38 aa, synthetic construct
FYAESVKGRFSISRDNAKSSLFLQMSSLRADDTAVYYC

SEQ ID NO: 553 - protein, 11 aa, synthetic construct
WGRGTVVTVSS

SEQ ID NO: 554 - protein, 109 aa, synthetic construct
DIQMTQSPSSLSASVGDRVTITCRASQTIRNYINWYQQRPGKAPRLLIYDASTLQSGVPSRFSGGGSGTDFTLTISSLEPEDFATYYCQQSYVSPPEDSFGQGTKLEIK

SEQ ID NO: 555 - protein, 6 aa, synthetic construct
QTIRNY

SEQ ID NO: 556 - protein, 11 aa, synthetic construct
QQSYVSPPEDS

SEQ ID NO: 557 - protein, 17 aa, synthetic construct
INWYQQRPGKAPRLLIY

SEQ ID NO: 558 - protein, 36 aa, synthetic construct
TLQSGVPSRFSGGGSGTDFTLTISSLEPEDFATYYC

SEQ ID NO: 559 - protein, 123 aa, synthetic construct
QVQLQQSGAEVKKPGSSVKVSCESSGGTFTSHAINWVRQAPGQRLEWMGRIIPVLGVAAYAQKFQGRVTLTADKFTSTAYMELSSLRSEDTAVYFCARSAYNYDSSGYHFDYWGQGTLVLVSS

SEQ ID NO: 560 - protein, 8 aa, synthetic construct
GGTFTSHA

SEQ ID NO: 561 - protein, 8 aa, synthetic construct
IIPVLGVA

SEQ ID NO: 562 - protein, 16 aa, synthetic construct
ARSAYNYDSSGYHFDY

SEQ ID NO: 563 - protein, 25 aa, synthetic construct
QVQLQQSGAEVKKPGSSVKVSCESS

SEQ ID NO: 564 - protein, 17 aa, synthetic construct
INWVRQAPGQRLEWMGR

SEQ ID NO: 565 - protein, 38 aa, synthetic construct
AYAQKFQGRVTLTADKFTSTAYMELSSLRSEDTAVYFC

SEQ ID NO: 566 - protein, 11 aa, synthetic construct
WGQGTLVLVSS

SEQ ID NO: 567 - protein, 106 aa, synthetic construct
DIQMTQSPSTLSASVGDRVTITCRASQSISNWLAWYQQKPGTAPKLLIFKASTLQSGVPSRFSGSGSGTEFTLTISSLQPDDFATYHCQQYHSSRSFGQGTKVEIK

SEQ ID NO: 568 - protein, 6 aa, synthetic construct
QSISNW

SEQ ID NO: 569 - protein, 8 aa, synthetic construct
QQYHSSRS

SEQ ID NO: 570 - protein, 17 aa, synthetic construct
LAWYQQKPGTAPKLLIF

SEQ ID NO: 571 - protein, 36 aa, synthetic construct
TLQSGVPSRFSGSGSGTEFTLTISSLQPDDFATYHC

SEQ ID NO: 572 - protein, 123 aa, synthetic construct
QVQLVQSGAEVKKPGSSMTVSCEASGDTFSRYSINWVRQAPGQGFEWMGRIIPMFGLATYAHKFQGRVTISADKSTTTAHMELNRLTSEDTAIYYCATAPFYYDNSTYPFDFWGQGTLVTVSS

SEQ ID NO: 573 - protein, 8 aa, synthetic construct
GDTFSRYS

SEQ ID NO: 574 - protein, 8 aa, synthetic construct
IIPMFGLA

SEQ ID NO: 575 - protein, 16 aa, synthetic construct
ATAPFYYDNSTYPFDF

SEQ ID NO: 576 - protein, 25 aa, synthetic construct
QVQLVQSGAEVKKPGSSMTVSCEAS

SEQ ID NO: 577 - protein, 17 aa, synthetic construct
INWVRQAPGQGFEWMGR

SEQ ID NO: 578 - protein, 38 aa, synthetic construct
TYAHKFQGRVTISADKSTTTAHMELNRLTSEDTAIYYC

SEQ ID NO: 579 - protein, 107 aa, synthetic construct
DIQLTQSPSSLSASVGDRVTITCRASQSINNYLNWYQHKPGKAPKLLIYGASTLQSGVPSRFSGSGSGSDFTLTISSLQPEDFATYYCQQSYNAPRTFGQGTRLEIK

SEQ ID NO: 580 - protein, 6 aa, synthetic construct
QSINNY

SEQ ID NO: 581 - protein, 9 aa, synthetic construct
QQSYNAPRT

SEQ ID NO: 582 - protein, 17 aa, synthetic construct
LNWYQHKPGKAPKLLIY

SEQ ID NO: 583 - protein, 36 aa, synthetic construct
TLQSGVPSRFSGSGSGSDFTLTISSLQPEDFATYYC

SEQ ID NO: 584 - protein, 123 aa, synthetic construct
QVHLVQSGAEVKKPGSSVKVSCKASGGTFSSHTVSWVRQAPGQGLEWMGRIIPPFGIVNYAQKFQGRVTITADESTSTAYMELSSLSSEDTAVYYCARSNVVVVTEAGWFDPWGQGTLVTVSS

SEQ ID NO: 585 - protein, 25 aa, synthetic construct
QVHLVQSGAEVKKPGSSVKVSCKAS

SEQ ID NO: 586 - protein, 38 aa, synthetic construct
NYAQKFQGRVTITADESTSTAYMELSSLSSEDTAVYYC

SEQ ID NO: 587 - protein, 107 aa, synthetic construct
DIQMTQSPSSVSASVGDRVTITCRASQGISSSLAWYQQKPGKAPKVLIFDASTLQSGVPSRFSGSGSGTDFTLTISNLRPEDFATYYCQQAHSFPFTFGPGTKVDIK

SEQ ID NO: 588 - protein, 6 aa, synthetic construct
QGISSS

SEQ ID NO: 589 - protein, 26 aa, synthetic construct [sequence omitted]

SEQ ID NO: 590 - protein, 17 aa, synthetic construct
LAWYQQKPGKAPKVLIF

SEQ ID NO: 591 - protein, 36 aa, synthetic construct
TLQSGVPSRFSGSGSGTDFTLTISNLRPEDFATYYC

SEQ ID NO: 592 - protein, 123 aa, synthetic construct
QVQLVQSGAEVKKPGSSVKVSCEASGDTFSRYAITWVRQAPGQGLEWMGRIIPMFGSANYAQKFQGRVSFTADKSTSTASMELSSLRSEDTAVYYCATSPYYSDSGGYYCDYWGQGSLVTVSS

SEQ ID NO: 593 - protein, 8 aa, synthetic construct
IIPMFGSA

SEQ ID NO: 594 - protein, 16 aa, synthetic construct
ATSPYYSDSGGYYCDY

SEQ ID NO: 595 - protein, 38 aa, synthetic construct
NYAQKFQGRVSFTADKSTSTASMELSSLRSEDTAVYYC

SEQ ID NO: 596 - protein, 107 aa, synthetic construct
DIQMTQSPSSLSASVGDRVTITCRASQGISNYLAWYQQKPGKAPKLLLYGASRLQSGVPSRFSGSGSGTDYTLTISSLQPEDFATFYCQQYYSAPRTFGQGTKVEIK

SEQ ID NO: 597 - protein, 9 aa, synthetic construct
QQYYSAPRT

SEQ ID NO: 598 - protein, 17 aa, synthetic construct
LAWYQQKPGKAPKLLLY

SEQ ID NO: 599 - protein, 36 aa, synthetic construct
RLQSGVPSRFSGSGSGTDYTLTISSLQPEDFATFYC

SEQ ID NO: 600 - protein, 118 aa, synthetic construct
QVQLQQWGAGLLKPSETLSLTCTVYGGSISGYYWNWIRQPPGKGLEWIGEIDHSGNTNYNSSLKSRITIALDTSKKEISLKVTSVTAADTAVYYCARLDYYHYSQDAWGQGTTVTVSS

SEQ ID NO: 601 - protein, 8 aa, synthetic construct
GGSISGYY

SEQ ID NO: 602 - protein, 7 aa, synthetic construct
IDHSGNT

SEQ ID NO: 603 - protein, 12 aa, synthetic construct
ARLDYYHYSQDA

SEQ ID NO: 604 - protein, 25 aa, synthetic construct
QVQLQQWGAGLLKPSETLSLTCTVY

SEQ ID NO: 605 - protein, 17 aa, synthetic construct
WNWIRQPPGKGLEWIGE

SEQ ID NO: 606 - protein, 38 aa, synthetic construct
NYNSSLKSRITIALDTSKKEISLKVTSVTAADTAVYYC

SEQ ID NO: 607 - protein, 106 aa, synthetic construct
EIVLTQSPDFQSVTPKERVTITCRASQSIGASLHWYQLKPNQSPKLLIKYASQSISGVPSRFSGSGSGTDFTLTISSLEAEDAATYYCLQSSNLYSFGQGTKLEIK

SEQ ID NO: 608 - protein, 6 aa, synthetic construct
QSIGAS

SEQ ID NO: 609 - skipped

SEQ ID NO: 610 - protein, 8 aa, synthetic construct
LQSSNLYS

SEQ ID NO: 611 - protein, 26 aa, synthetic construct
EIVLTQSPDFQSVTPKERVTITCRAS

SEQ ID NO: 612 - protein, 17 aa, synthetic construct
LHWYQLKPNQSPKLLIK

SEQ ID NO: 613 - protein, 36 aa, synthetic construct
QSISGVPSRFSGSGSGTDFTLTISSLEAEDAATYYC

SEQ ID NO: 614 - protein, 125 aa, synthetic construct
QVQLVQSGAEVKKPGSSVKVSCKASGGTFTTYAIAWVRQAPGQGLEWMGRIIPTLDNPNYAQKFQGRVSITADKSTGTSYMELRSLRSDDTAVYYCAHVSYDSSGPHLSWYFNLWGRGTLITVSS

SEQ ID NO: 615 - protein, 8 aa, synthetic construct
GGTFTTYA

SEQ ID NO: 616 - protein, 8 aa, synthetic construct
IIPTLDNP

SEQ ID NO: 617 - protein, 18 aa, synthetic construct
AHVSYDSSGPHLSWYFNL

SEQ ID NO: 618 - protein, 18 aa, synthetic construct
AIAWVRQAPGQGLEWMGR

SEQ ID NO: 619 - protein, 38 aa, synthetic construct
NYAQKFQGRVSITADKSTGTSYMELRSLRSDDTAVYYC

SEQ ID NO: 620 - protein, 11 aa, synthetic construct
WGRGTLITVSS

SEQ ID NO: 621 - protein, 103 aa, synthetic construct
DIQMTQSPSTLSASVGDRVTITCRASQSIPSRLAWYQHKPGEAPKLLIYEASTLESGVPSRFSGSGSGTEFTLTITSLQPDDFATYFCQGWNAFGGGTKVEIK

SEQ ID NO: 622 - protein, 6 aa, synthetic construct
QSIPSR

SEQ ID NO: 623 - skipped

SEQ ID NO: 624 - protein, 5 aa, synthetic construct
QGWNA

SEQ ID NO: 625 - protein, 17 aa, synthetic construct
LAWYQHKPGEAPKLLIY

SEQ ID NO: 626 - protein, 36 aa, synthetic construct
TLESGVPSRFSGSGSGTEFTLTITSLQPDDFATYFC

SEQ ID NO: 627 - protein, 127 aa, synthetic construct
QVQLVQSGAAVKKPGSSVKVSCEASGGTFSRFAVNWVRQAPGQGLEWMGRIIPLFAIANYPQRFQGRVIMSADRSTNITYMELSRLTSEDTAVYYCAREIDTVIDPAMDVFHGMEVWGQGTTVIVSS

SEQ ID NO: 628 - protein, 8 aa, synthetic construct
GGTFSRFA

SEQ ID NO: 629 - protein, 8 aa, synthetic construct
IIPLFAIA

SEQ ID NO: 630 - protein, 20 aa, synthetic construct
AREIDTVIDPAMDVFHGMEV

SEQ ID NO: 631 - protein, 25 aa, synthetic construct
QVQLVQSGAAVKKPGSSVKVSCEAS

SEQ ID NO: 632 - protein, 17 aa, synthetic construct
VNWVRQAPGQGLEWMGR

SEQ ID NO: 633 - protein, 38 aa, synthetic construct
NYPQRFQGRVIMSADRSTNITYMELSRLTSEDTAVYYC

SEQ ID NO: 634 - protein, 11 aa, synthetic construct
WGQGTTVIVSS

SEQ ID NO: 635 - protein, 110 aa, synthetic construct
QSVLTQPPSASGTPGQRVTISCSGRSSNIGSNYVHWYQQLPGTAPKLLIYRNNQRPSGVPDRFSGSKSGTSASLAISGLRSEDEADYYCAGWDDSLSGAVFGGGTKLSVL

SEQ ID NO: 636 - protein, 8 aa, synthetic construct
SSNIGSNY

SEQ ID NO: 637 - skipped

SEQ ID NO: 638 - protein, 11 aa, synthetic construct
AGWDDSLSGAV

SEQ ID NO: 639 - protein, 25 aa, synthetic construct
QSVLTQPPSASGTPGQRVTISCSGR

SEQ ID NO: 640 - protein, 17 aa, synthetic construct
VHWYQQLPGTAPKLLIY

SEQ ID NO: 641 - protein, 36 aa, synthetic construct
QRPSGVPDRFSGSKSGTSASLAISGLRSEDEADYYC

SEQ ID NO: 642 - protein, 10 aa, synthetic construct
FGGGTKLSVL

SEQ ID NO: 643 - protein, 129 aa, synthetic construct
EVQLVESGGGLVKPGESLRLSCAASGFTFSSSSLNWVRQAPGKGLEWVSSISISGDYTYYADSVKGRFTISRDNGKNSVYLQMDRLRVEDTAMYYCARDRFGFYDMLAHSYNVGFFQSWGQGTLVAVSS

SEQ ID NO: 644 - protein, 8 aa, synthetic construct
GFTFSSSS

SEQ ID NO: 645 - protein, 8 aa, synthetic construct
ISISGDYT

SEQ ID NO: 646 - protein, 22 aa, synthetic construct
ARDRFGFYDMLAHSYNVGFFQS

SEQ ID NO: 647 - protein, 25 aa, synthetic construct
EVQLVESGGGLVKPGESLRLSCAAS

SEQ ID NO: 648 - protein, 17 aa, synthetic construct
LNWVRQAPGKGLEWVSS

SEQ ID NO: 649 - protein, 38 aa, synthetic construct
YYADSVKGRFTISRDNGKNSVYLQMDRLRVEDTAMYYC

SEQ ID NO: 650 - protein, 11 aa, synthetic construct
WGQGTLVAVSS

SEQ ID NO: 651 - protein, 107 aa, synthetic construct
DIRMTQSPSSLSASVGDSVTITCRASQDITNYLAWFQQKPGKAPKSLIYSASILQSGVPSKFSGSGSGTDFTLTISSLQPEDFATYYCQQYLTYPITFGQGTRLEIK

SEQ ID NO: 652 - protein, 6 aa, synthetic construct
QDITNY

SEQ ID NO: 653 - protein, 9 aa, synthetic construct
QQYLTYPIT

SEQ ID NO: 654 - protein, 26 aa, synthetic construct
DIRMTQSPSSLSASVGDSVTITCRAS

SEQ ID NO: 655 - protein, 36 aa, synthetic construct
ILQSGVPSKFSGSGSGTDFTLTISSLQPEDFATYYC

SEQ ID NO: 656 - protein, 130 aa, synthetic construct
QVQLVQSGSEVKKPGSSVKVSCRASGDTFSSHSLSWVRQAPGQGLEWMGRIVPMFGITNYAQNFQGRVTFTADKSTSTAYMELSSLRSEDTAVYYCARAGTMITFGGLTDDGQLNWFDPWGQGTLVTVSS

SEQ ID NO: 657 - protein, 8 aa, synthetic construct
GDTFSSHS

SEQ ID NO: 658 - protein, 8 aa, synthetic construct
IVPMFGIT

SEQ ID NO: 659 - protein, 23 aa, synthetic construct
ARAGTMITFGGLTDDGQLNWFDP

SEQ ID NO: 660 - protein, 25 aa, synthetic construct
QVQLVQSGSEVKKPGSSVKVSCRAS

SEQ ID NO: 661 - protein, 17 aa, synthetic construct
LSWVRQAPGQGLEWMGR

SEQ ID NO: 662 - protein, 38 aa, synthetic construct
NYAQNFQGRVTFTADKSTSTAYMELSSLRSEDTAVYYC

SEQ ID NO: 663 - protein, 112 aa, synthetic construct
DVVLTQSPLSLSVTLGQPASISCRSSQSLEYSDGDTYLNWFHQRPGQSPRRLIYKVSNRDSGVPDRFSGSGAGTDFTLKISKVEAEDVGVYYCMQGTNWPPNFGPGTKVDIK

SEQ ID NO: 664 - protein, 11 aa, synthetic construct
QSLEYSDGDTY

SEQ ID NO: 665 - skipped

SEQ ID NO: 666 - protein, 9 aa, synthetic construct
MQGTNWPPN

SEQ ID NO: 667 - protein, 26 aa, synthetic construct
DVVLTQSPLSLSVTLGQPASISCRSS

SEQ ID NO: 668 - protein, 17 aa, synthetic construct
LNWFHQRPGQSPRRLIY

SEQ ID NO: 669 - protein, 36 aa, synthetic construct
NRDSGVPDRFSGSGAGTDFTLKISKVEAEDVGVYYC

SEQ ID NO: 670 - protein, 330 aa, Homo sapiens [sequence omitted]

SEQ ID NO: 671 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 672 - protein, 12 aa, Homo sapiens
EPKSCDKTHTCP

SEQ ID NO: 673 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 674 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 675 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 676 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 677 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 679 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 680 - protein, 107 aa, Homo sapiens [sequence omitted]

SEQ ID NO: 681 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 682 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 683 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 684 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 685 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 686 - protein, 223 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 687 - protein, 223 aa, synthetic construct
RVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNF

SEQ ID NO: 688 - protein, 223 aa, synthetic construct
RVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNF

SEQ ID NO: 689 - protein, 223 aa, synthetic construct
RVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNF

SEQ ID NO: 690 - protein, 223 aa, synthetic construct
RVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNF

SEQ ID NO: 691 - protein, 223 aa, synthetic construct
RVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNF

SEQ ID NO: 692 - protein, 223 aa, synthetic construct; identical to SEQ ID NO: 691

SEQ ID NO: 693 - protein, 223 aa, synthetic construct
RVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNF

SEQ ID NO: 694 - protein, 223 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNF

SEQ ID NO: 695 - protein, 223 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNF

SEQ ID NO: 696 - protein, 223 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNF

SEQ ID NO: 697 - protein, 223 aa, synthetic construct
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNF

SEQ ID NO: 698 - protein, 223 aa, synthetic construct; identical to SEQ ID NO: 697

SEQ ID NO: 699 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 700 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 701 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 702 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 703 - protein, 72 aa, synthetic construct
NSNKLDSKVSGNYNYLYRLFRKSNLKPFERDISTEIYQAGNKPCNGVAGFNCYFPLRSYSFRPTYGVGHQPY

SEQ ID NO: 704 - protein, 72 aa, synthetic construct
NSNKLDSKVGGNYNYLYRLFRKSNLKPFERDISTEIYQAGNKPCNGVAGFNCYFPLRSYGFRPTYGVGHQPY

SEQ ID NO: 705 - protein, 72 aa, synthetic construct
NSNKLDSKVGGNYNYRYRLFRKSNLKPFERDISTEIYQAGNKPCNGVAGVNCYFPLQSYGFRPTYGVGHQPY

SEQ ID NO: 706 - protein, 72 aa, synthetic construct
NSNKLDSKVSGNYNYLYRLFRKSKLKPFERDISTEIYQAGNKPCNGVAGFNCYFPLQSYGFRPTYGVGHQPY

SEQ ID NO: 707 - protein, 72 aa, synthetic construct
NSNKLDSKVSGNYNYLYRLFRKSKLKPFERDISTEIYQAGNKPCNGVAGSNCYFPLQSYGFRPTYGVGHQPY

SEQ ID NO: 708 - protein, 72 aa, synthetic construct; identical to SEQ ID NO: 705

SEQ ID NO: 709 - protein, 72 aa, synthetic construct; identical to SEQ ID NO: 705

SEQ ID NO: 710 - protein, 72 aa, synthetic construct
NSNKLDSTVGGNYNYRYRLFRKSKLKPFERDISTEIYQAGNKPCNGVAGVNCYFPLQSYGFRPTYGVGHQPY

SEQ ID NO: 711 - protein, 72 aa, synthetic construct
NSNKLDSKPSGNYNYLYRLFRKSKLKPFERDISTEIYQAGNKPCNGVAGSNCYSPLQSYGFRPTYGVGHQPY

SEQ ID NO: 712 - protein, 72 aa, synthetic construct
NSNKLDSKPSGNYNYLYRLFRKSKLKPFERDISTEIYQAGNRPCNGVAGPNCYSPLQSYGFRPTYGVGHQPY

SEQ ID NO: 713 - protein, 72 aa, synthetic construct
NSNKLDSKPSGNYNYLYRLFRKSKLKPFERDISTEIYQAGNKPCNGVAGPNCYSPLQSYGFRPTYGVGHQPY

SEQ ID NO: 714 - protein, 72 aa, synthetic construct
NSNKLDSKPSGNYNYLYRLLRKSKLKPFERDISTEIYQAGNKPCNGVAGPNCYSPLQSYGFRPTYGVGHQPY

SEQ ID NO: 715 - protein, 72 aa, synthetic construct; identical to SEQ ID NO: 714

SEQ ID NO: 716 - protein, 72 aa, synthetic construct
NSNNLDSKVGGNYNYLYRLFRKSNLKPFERDISTEIYQAGSTPCNGVEGFNCYFPLQSYGFQPTYGVGYQPY

SEQ ID NO: 717 - protein, 72 aa, synthetic construct
NSNNLDSKVGGNYNYLYRLFRKSNLKPFERDISTEIYQAGSTPCNGVKGFNCYFPLQSYGFQPTYGVGYQPY

SEQ ID NO: 718 - protein, 72 aa, synthetic construct
NSNNLDSKVGGNYNYRYRLFRKSNLKPFERDISTEIYQAGSKPCNGVEGFNCYFPLQSYGFQPTNGVGYQPY

SEQ ID NO: 719 - protein, 72 aa, synthetic construct; identical to SEQ ID NO: 717

SEQ ID NO: 720 - protein, 448 aa, synthetic construct
EVQLVESGGGLVQPGGSLRLSCAASEIIVSRNYMTWVRQAPGKGLEWLAVLYAGGSSFYADSVKGRFTISRDNSKNTLFLQMNSLRAEDTAVYYCARDLSLSGGFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 721 - protein, 214 aa, synthetic construct
DIQLTQSPSFLSASVGDRVTITCRASQGISNYLAWYQQNPGKAPKLLIYAVSTLHSGVPSRFSGSGSGTEFTLTISSLQPEDFATYYCQHLNTDSCTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - protein, 449 aa, synthetic construct
EFQLVESGGRLVRPGGSLRLSCVASEIIVSRNYMSWIRQAPGKGLEWVSILYAGGTTYYADSVKGRFTISRDNSKNTLYLQLNSLRVEDTAIYYCVRPIVGGRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 723 - protein, 214 aa, synthetic construct
DIQMTQSPSSLSASVGDRVTITCQASQDINKYLNWYQQKPGKAPKLLIYDASNLEPGVPSRFSGSGSGTDFVFTITSLQPEDIATYYCHYYDDVPYTFGQGTQLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 724 - protein, 453 aa, synthetic construct
QVQLVQSGAEVKKPGSSVRVSCEASGDTFSRYAISWVRQAPGQGLEWMGRIIPMFGMANSAQKFQARVTISADKSTSTAYMEVSSLRSEDTAVYYCATDTFYPNDGVHRMEYWGQGALVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 725 - protein, 214 aa, synthetic construct
DIQMTQSPSSLSASVGDRVTITCRASQTISNYLNWYQQKPGKAPNLLIYGTSILQSGVPSRFSGSGSGTDFTLTISSLQPEDFATYYCQQSHSAPRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 726 - protein, 455 aa, synthetic construct
QVQLVQSGAEVKKPGSSVKVSCEASGGTFSTYAISWVRQAPGQGLEWMGRIIPIFGIANYAQKFQGRVTITADKSTSTAYMEVSSLRSEDTAVYYCATTFYDHSSTYRTHSMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 727 - protein, 214 aa, synthetic construct
DIQMTQSPSSLSASVGDRVTITCRASQSISNYLNWYQQRPGKAPKLLIYAASGLQSGVPSRFSGSGSGTNFTLTISTLHPEDFATYYCQQTHSTPRAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 728 - protein, 449 aa, synthetic construct
DFQLVESGGGLVRPGGSLRLSCVASEITVSRNYMSWIRQAPGKGLEWVSIIYPGGTTYYADSVKGRFTISRDNSKNTMYLQLNSLRPEDTAIYYCVRPIVRGGGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 729 - protein, 214 aa, synthetic construct
DIQMTQSPSSLSASVGDRVTITCQASQDINKYLNWYQQKPGKAPKLLIYDATNLEPGVPSRFSGSGSGTDFVFTITSLQPEDIATYYCQYYDDVPYTFGQGTQLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 730 - protein, 451 aa, synthetic construct
EVQLVESGGGLVQPGGSLRLSCSASGFTFNNYVMHWVRQAPGKGLEYVSAINSNGGSTYYAGSVKGRFTISRDNSNNTLYLQMSSLRAEDTAVYYCIKDAGYYSSLGVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 731 - protein, 214 aa, synthetic construct
EIVLTQSPATLSLSPGERATLSCRASQSVGSYLAWFQQKPGQAPRLLIYDASYRATGIPARFSGSGSGTDFTLTISSLEPEDVGIYYCQQRSNWPQTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 732 - protein, 458 aa, synthetic construct
EVQLVESGGGLVKPGGSLRLSCAASGFTVSTYIINWVRQAPGKGLEWVSSITSESDYMFDADSVRGRFTISRDNAKNLVYLQMNSLRAEDTAVYYCARDQGAYSGYDLSPGGDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 733 - protein, 214 aa, synthetic construct
DIQMTQSPSTLSASVGDRVTITCRASQSISTWLAWYQQKPGKAPKLLIYKASNLESGVPSRFSGSGSGTEFTLTISRLQPDDSATYCCQHYNSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - protein, 448 aa, synthetic construct
EVQLVQSRAEVKKPGESLKISCKGSGYSFTTYWIGWVRQMPGKGLEWMGIIRPGDSDTRYSPSFQGQVTISADKSITTAYLQWSSLKASDTAMYYCAMTYSGDQYDFWGQGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 735 - protein, 214 aa, synthetic construct
DIQMTQSPSTLSASVGDRVTITCRASQSIHTWLAWYQQTPGKAPKLLIYKASLLESGVPSRFSGSGSGTEFTLTISSLQPGDFATYYCQHYNSYSHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 736 - protein, 451 aa, synthetic construct
QVQLVQSGAEVKKPGASVKVSCKISGYTLTDFSIHWVRQAPGKGLEWMAGFDPEHRETIFAQKFQGRVAMTEATSTDTAYMELSSLRSDDTAVYYCATTGDFDSWRGYYLWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 737 - protein, 214 aa, synthetic construct
DIQLTQSPSSVSASVGDSVTITCRASQGISRWLAWYQQKPGKAPRLLIYSASTLQSGVPSRFSGSGSGTDFTLTISSLQPDDFATYYCQHTNNFPFSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 738 - protein, 458 aa, synthetic construct
QVQLVQSGAEVKKPGSSVKVSCRASGDTFSTYTITWVRQAPGQGLEWMGRIIPILDTADYAQKFQGRVTITADKSTSTAYMELSSLRSEDTAMYYCAKNYPNGYSGYDYFSWDGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 739 - protein, 218 aa, synthetic construct
QSVLTQPPSVSGAPGQRVTISCTGSSSNIGANYEVHWYQQLPGTAPKLLIFGHSNRPSAVPDRFSGSKSGTSASLAITGLQTEDEADYYCQSYDSSLSGVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 740 - protein, 446 aa, synthetic construct
QVQLQESGPRLVKPSGTLSLSCAVSGGPFSNTNWWSWIRQTPGKGLEWIGEINDSGNTVYNPALKSRVTMSVDKSKKQFSLNLHSLTAADTAVYFCARVWGHFDYWGQGVRVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 741 - protein, 213 aa, synthetic construct
DIQMTQSPSSLYASVADRVTITCRASQGISNSLAWYQQQPGKAPQLLLYAASTLESGVPSRFSGSGSGTDFTLTISSLQPEDFATYYCQQYYSLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 742 - protein, 452 aa, synthetic construct
EVQLLESGGGLVQPGGSLRLSCAASGFTFSGSDMSWVRQAPGKGLEWVSVIGGSGTYAYYSDSVKGRFTISRDNSKNMLFLQMNSLRAEDTAIYYCAKETGFLWFGELLDSWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 743 - protein, 215 aa, synthetic construct
DIQMTQSPSSLSASVGDRVTITCRASEAISNSLAWYQQRPGKAPRLLLYAAATLESGVPPRFSGSGSGTDFTLTISTLQPEDFATYYCQQYYSPPPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 744 - protein, 452 aa, synthetic construct
EVQLVESGGGLVQPGRSLRLSCAASGFTFDEYAMHWVRQAPGKGLEWVSGISFNSGSVGYAGAAKGRFTISRDNAKKSLYLEMNSLRDEDTAFYYCAKDRGEHWLVRLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 745 - protein, 213 aa, synthetic construct
DIQMTQSPSTLSASVGDRVTITCRPSQSIDRWLAWYQQKPGKAPTLLISAASSLETGVPSRFSGSGSGTEFTLTISSLQPDDFATYYCQQYNGYSMFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 746 - protein, 453 aa, synthetic construct
EVQLVESGGGLVQPGGSLRLSCATSGFTFDDFAMHWVRQGPGKGLEWVSGISWNSGKIAYTDSVKGRFSISRDNAKNSLYLQMNSLRPEDTALYYCTKDHAPSAILGDILTGFDPWGQGTLVTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 747 - protein, 214 aa, synthetic construct
DIQMTQSPVSLSASVGDRVTITCRASQSISVYLNWYQQKPGKAPKLLMYAASILQSGVPSRFSGSGSATDFTLTITSLQPEDFATYFCQQSFTMPPTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 748 - protein, 452 aa, synthetic construct
QVQLVQSGAEVKKPGASVKVSCKASGYIFNNYAIQWVRQAPGQRLEWMAWIHTGNGDTKYSQKFQGRVTITRDTSASTAYMELSSLRSEDTAIYYCARVRPNWNTLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 749 - protein, 218 aa, synthetic construct
NFMLTQPHSVSESPGKTVTISCTGSGGSIATNYVQWYQQRPGSAPTTVIYEDNERPSGVPDRFSGSIDSSSNSASLTISGLRTEDEADYYCQSYDISTHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 750 - protein, 448 aa, synthetic construct
EVQLVESGGTSVRPGGSLRLSCSASGFSVRSNFMTWVRQAPGKGLECVSVIYSGSGGSTFYADSVKGRFTISKDDSKNTLYLQMNSLRAEDTAIYYCAREVSHAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 751 - protein, 215 aa, synthetic construct
DIQMTQSPSTLSASVGDRVTITCRASQTIGRWLAWYQQRPGKAPSLLIYMASILESGVPLRFSGSGSGTEFTLTISGLQPDDFATYYCQQYNSDSPYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 752 - protein, 448 aa, synthetic construct
QVQLQESGPGLVKPSETLSLTCTVSGGSLTSYYWSWIRQPPGGGLEWIGHIYYTGITDHNPSLKSRVTISLDTSRNQFSLKVRSVTAADTALYYCARAAGSSDYFDFWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 753 - protein, 213 aa, synthetic construct
DIQMTQSPSTLSASVGDRVTITCRASQSISRWLAWYQQKPGKAPKLLIYMASILESGVPSRFSGGGSGTEFTLTISSLQPDDFATYYCQQYNSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 754 - protein, 448 aa, synthetic construct
QVQLQESGPGLVKPSETLSLTCTVSGGSITSYYWSWIRQSPGKGLEWIGHIYYSGSTDYNPSLKSRVTISVDTSKNQFSLKLTYVAAADTAVYFCARAGGSSDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 755 - protein, 106 aa, synthetic construct; identical to SEQ ID NO: 280

SEQ ID NO: 756 - protein, 459 aa, synthetic construct
EVQLVESGGGLVKPGGSLRLSCAASGFIFSRNGMHWVRQAPGKGLEWVSSIDNDGTYMYYADSVRGRFTVSRDNAKNSLFLQLNSLRAEDTAVYYCARDRFGYYDALTDSYNAGYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 757 - protein, 214 aa, synthetic construct
DIQMTQSPSSLSASVGDSVTITCRASQDIYNYLAWFQQKPGKAPKSLIYTASKLESGVPSKFSGSGSGTDFTLTISSLQPEDFATYYCQQYHSSPITFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 758 - protein, 459 aa, synthetic construct
EVQLVESGGGLVKPGGSLRLSCAASGFAFSTNGMNWVRQVPGKGLEWVSSISSTSEYTYYTESVKGRFTISRDNAKNSLFLQMNSLRAEDTAVYYCVRDRFGYYDVLASSYNVGFFQSWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 759 - protein, 214 aa, synthetic construct
DIQMTQSPSSLSASVGDGVTITCRASQDISNNLAWFQQKPGKAPKCLIYSASSLQSGVPLKFRGSGSGTDFTLSITSLDPEDFATYYCQQYISYPITFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 760 - protein, 463 aa, synthetic construct
QVQLVQSGAEVKKPGSSVEVSCKASGGTFSSYAISWVRQAPGQGLEWMGRIIPSLGITNSAENFQGRVTITADKSTSTVYMELSSLRSEDTAVYYCARDFHPLYQSCSSTSCYDEWEPRMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 761 - protein, 217 aa, synthetic construct
QSVLTQPPSASGTPGQRVTISCHGSSSNIGSKTVNWYQQLPGTAPKLLIYSNDQRPSGVPDRFSGSKSGTSASLAISGLQSEDEADYYCAAWDGSLDGPVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 762 - protein, 463 aa, synthetic construct
QVQLVQSGAEVKKPGSSVKVSCKASGGTFSSYGISWVRQAPGQGLEWMGRIIPSLSITNSAEKFQGRVTITADKSTSTAYMELSRLRSEDTALYYCARDFHPRYEFCDSTSCYDEWEPRMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 763 - protein, 217 aa, synthetic construct
QSVLTQSPSASGTPGQRVIISCSGSSSNIGRKTVNWYQQLPGTAPKLVIYSNDQRPSGVPDRFSGSKSGTSASLAISGLQSEDEADYYCAAWDDSLDGPVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 463 aa, synthetic construct
QVQLVQSGAEVKKPGSAVKVSCKASGGTFSSYPITWVRQAPGQGLEWVGRAIPILGITSTAQKFQGRVTIIADKSTSTAYMELSRLRSEDTAVYYCARDFHPRYQDCDSTSCYDQWEPRMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 217 aa, synthetic construct
QSVLTQPPSASGTPGQRVTISCSGSSSNIGSNTVNWYQQLPGTAPKLLIYTNNQRPSGVPNRFSASKSGTSASLAISGLQSEDEADYYCAAWDDRLSGPVFGGGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 453 aa, synthetic construct
QVQLVQSGAEVKKPGSSVKVSCEASGDTFSRYAINWVRQAPGQGLEWMGRIIPMFGNTNYAQKFQGRFTITADKSRGTAYMEVIGLTSADTAVYYCATSPFYYSDGGYPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 767 - protein, 215 aa, synthetic construct
EIVLTQSPGTLSLSPGERATLSCRASQSLNSAYLAWYQQRAGLAPRLLIYGASSRATGIPDKFSGSGSGTDFTLTISRLEPEDFAVYYCQQYAYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 768 - protein, 463 aa, synthetic construct
QVQLVQSGAEVKKPGSSVKVSCKASRGTFSNYAISWVRQAPGQGLEWMGRIIPSLSITNSAEKFQGRVTMTADKSTSTVYMELSRLRSEDTAVYYCARDFHPRYEYCSSTSCYDEWEPRMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 769 - protein, 217 aa, synthetic construct
QSVLTQSPSASGTPGQRVIISCSGSSSNIGRKTVNWYQQLPGTAPKLLMYSNDQRPSGVPDRFSGSKSGTSASLAISGLQSEDEADYYCAAWDDSLDGPVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 770 - protein, 453 aa, synthetic construct
QVQLVQSGAEVKKPGSSVKVSCKASGGTFSSHTVSWVRQAPGQGLEWMGRIIPPFGIVNYAQKFQGRVTMTADESTSTAYMELSSLRSEDTAVYYCARSNVVVVTEAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 771 - protein, 214 aa, synthetic construct
DIQMTQSPSSVSASVGDRVSISCRASQDISSSLAWYQQKPGQAPKVLIYDASTLQSGVPSRFSGSGSGTDFTLTISNLRPDDFATYFCQQAHSFPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 772 - protein, 447 aa, synthetic construct
EVQLVESGGGLVQPGGSLRLSCGASGITVSSNYMNWVRQAPGKGLEWVSTLYAGGSTFYADSVKGRFIISRDNSKNTLYLQMNSLRADDTAVYYCARDLVDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 773 - protein, 215 aa, synthetic construct
DIQLTQSPSSLSASVGDRVTITCRASQGISSYLAWYQQKPGKAPKLLIYAASTLQSGVPSRFSGSGSGTDFTLTISSLQPEDFATYYCQQLNSYSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 774 - protein, 453 aa, synthetic construct
QFQLVQSGAEVKKPGSSVKVSCRASGGSFTSHAISWVRQAPGQGFEWMGRIIPMFGIANYAPKFQGRVTMSADKFKDIVYMEVNSLTSEDTALYYCARSQPMTSVTTLWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 775 - protein, 215 aa, synthetic construct
EIVLTQSPGTLSLSPGERATLSCRASEPVGGSYLAWYQQKPGQAPRLLIHGASSRATGIPDRFSGSGSGTDFVLTISRLEPEDFAVYHCQQYASSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 776 - protein, 453 aa, synthetic construct
QVHLVQSGAEVKKPGSSVKVSCKTSGDTFTRYAINWVRQAPGQGLEWMGRIIPMFGIPNYAQKFQGRVTMTADKSTDIAYMELSSLRSEDTAVYYCARSSFYSDSSGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 777 - protein, 215 aa, synthetic construct
EIVLTQSPGTLSLSPGERATLSCRASQNLDSNYLAWYQQKPGQAPRLLIYGASIRATGIPDRFSGSGSGTDFTLTISRLEPEDFAVYHCQQYHNSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 778 - protein, 453 aa, synthetic construct
QVQLVQSGAEVKKPGSSVKVSCKASGDTFTKYAITWVRQAPGEGLEWMGRIIPRFGMANYAQNFQGRVTMTADQSTSTAYMELTSLRSNDTAVYYCATTFYFDSSYYHAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 779 - protein, 214 aa, synthetic construct
DIQMTQSPSSLSASVGDRVTITCRASQNIDTYLIWYQQKPGKAPNLLVYGASTLQSGVPSRFSGSGSGTDFTLTISSLQPEDFATYYCQQTYNAPRTFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 780 - protein, 455 aa, synthetic construct
QVQLVQSGAEVKKPGSSVKVSCKASGDTFSRYAISWVRQAPGQGLEWMGRIIPMFGTANYAQNFQGRVTITADKSTSTAYMELTSLRSEDTAVYYCATTYFYDSDRDRTHSMDVWGPGSAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 781 - protein, 214 aa, synthetic construct
DIQMTQSPSSLSASVGDRVTITCRTSQSVGNYLNWYQQKPGKAPNLLIYAASTLQNGVPSRFSGSGSGTDFTLTISSLQPEDFATYYCQQTHSTPRAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 782 - protein, 446 aa, synthetic construct
EVQLVESGGGLVQPGGSLRVTCVASGFAVRTNFMTWVRQAPGKGLQCVSVIYGDGSTYYADSVKGRFSISRDNSKNTVYLQMNSLSAEDTAVYYCAREVSHAFDLWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 783 - protein, 215 aa, synthetic construct
DIQLTQSPSTLSASLGDRVTITCRASQSISGWLAWYQQKPGRAPQLLIYKASLLETGVPSRFSGSGSGTVFTLTISSLQPDDFATYYCQQYDTYSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 784 - protein, 447 aa, synthetic construct
EVQLVESGGGLVQPGGSLRLSCGASGITVSSNYMNWVRQAPGKGLEWVSTLYAGGSTFYADSVKGRFIISRDNSKNTLYLQMNSLRAEDTAVYYCARDLVDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 785 - protein, 215 aa, synthetic construct
AIQLTQSPSSLSASVGDRVTITCRASQGISSYLAWYQQKPGKAPKLLIYAASTLQSGVPSRFSGSGSGTDFTLTISSLQPEDFATYYCQQLNSYSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 786 - protein, 447 aa, synthetic construct
EVQLVESGGGLVQSGGSLRLSCAASGIIVSRNYMSWVRQAPGKGLEWVSVIYPGGSTFYADSVKGRFTISRDNSKNTLYLQMNSLRAEDTAVFYCARDRGEDIFDFWGQGTMVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 787 - protein, 215 aa, synthetic construct
DIQLTQSPTFLSASVGDRVTITCRASQGISSYLAWYQQKPGKAPKLLIYAASTLQGGVPSRFSGSGSGTEFTLTISSLQPEDFATYYCQQLNSSPGFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 788 - protein, 448 aa, synthetic construct
QVQLQESGPGLVKPSETLSLTCTVSGDSIYSYSWTWIRQPPGKGLEWIGQLYYNGSTSYNASLKSRVSISLDTSKNQFSLKLRFVTAADTAMYYCARVEYYSNYFDPWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 789 - protein, 214 aa, synthetic construct
EIVLTQSPATLSLSPGERATLSCRASQSVGSYLAWYQQKPGQAPRLLIYDASNRVPGIPARFSGSGSGTDFTLTISSLPPEDFAVYYCQQRSDWPPTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 790 - protein, 448 aa, synthetic construct
EVQLVESGGGLVQPGGSLRVSCAASGIIVSRNYMTWVRQAPGKGLEWVSVIYAGGSTFYADSVKGRFTISRDNSKNTLYLQMNSLRVDDTAVYYCARDLEIAGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 791 - protein, 214 aa, synthetic construct
AIQLTQSPSSLSASVGDRVTITCRASEGISNYLAWYQQKPGKAPKVLIYAASTLQSGAPPRFSGSGSGTDFTLTISSLQPEDFATYYCQQLNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 792 - protein, 453 aa, synthetic construct
QVQLVQSGAEVKKPGSSVKVSCKASGDTFSRDAISWVRQAPGQGLEWMGRIIPMFGIANYAQNFQGRVTMTADKYTSTAYMELSSLRSEDTAVYYCARGGYQYESSGYHLDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 793 - protein, 215 aa, synthetic construct
EIVLTQSPGTLSLSPGERATLSCRASQSLSSSYLAWYQQKPGQAPRLLIYGTSSRATGIPDRFSGSGSGTDFTLTISRLEPEDFAVYYCHQYSNSPRTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 794 - protein, 450 aa, synthetic construct
QVQLVQSGAEVKKPGSSVKISCKASGDTSSSYTINWVRQAPGQGLEWMGRIIPMFNRANYARKFQGRVTMTADRSTDTAYMEVSSLTSDDTAVYYCARTWIEPHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 795 - protein, 215 aa, synthetic construct
EIVLRQSPGTLSLSPGERATLSCRASQSVSGNHLAWYQRKPGQAPRLLIYAASSRATGIPDRFSGSGSGTDFTLTINRLEPEDFAVFYCQQYGTSPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 796 - protein, 450 aa, synthetic construct
QVQLQESGPRLVKPSETLSLTCTVSGGSISSYYWTWIRQPPGKGLEWIGYISYSGSTNYNPSLRSRVTMSVDTSKNQFSLDLNSVTAADTAVYYCATDGGGFYPGYFPHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 797 - protein, 215 aa, synthetic construct
DIQMTQSPSSLSASVGDRVTITCRASQSISDYVNWYQQKPGRAPRLLIYAPSTLQSGVPSRFSGSGSGTDFTLTISNLQPEDFGTYFCQQSYSTPPYTFGQGTKVEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 798 - protein, 451 aa, synthetic construct
QVQLVQSGAEVKKPGASVKVSCKGSGYTFTDYGISWVRQAPGQGLEWMGWIRAYNGNTDYAQKFQGRVTMTRDTSTTTAYMELRSLRSDDTAVYFCALNLIAAAATPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 799 - protein, 215 aa, synthetic construct
EIVLTQSPGTLSLSRGERATLSCRASQSVSSSYLAWYQQKRGQAPRLLIYGATSRATGIPDRFSGSGSGTDFTLTISRLEPEDFAVYYCQHYGSSPPPFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 800 - protein, 451 aa, synthetic construct
EVQLLESGGGLVQPGGSLRLSCAGSGFTFSSYAMSWVRQAPGKGLEWVSSISSSGGLTFYADSVRGRSTISRDHSRNTLYLQMNSLRAEDTALYYCAREQGDSYDDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 801 - protein, 215 aa, synthetic construct
DIQMTQSPSSLSASVGDRVTITCRASQGIRSSLAWYQQRPGKAPKLLLSAASILESGVPSRFSGSGSGTDYTLTINGLQPDDFATYYCQQYYVTPGITFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 802 - protein, 451 aa, synthetic construct
EVQLVESGGGLVKPGGSLRLSCAASGFTFSRHTMNWVRQAPGKGLEWVSSIGGSTPLSFYAESVKGRFSISRDNAKSSLFLQMSSLRADDTAVYYCVRDSIASVTTLFDLWGRGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 803 - protein, 216 aa, synthetic construct
DIQMTQSPSSLSASVGDRVTITCRASQTIRNYINWYQQRPGKAPRLLIYDASTLQSGVPSRFSGGGSGTDFTLTISSLEPEDFATYYCQQSYVSPPEDSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 804 - protein, 453 aa, synthetic construct
QVQLQQSGAEVKKPGSSVKVSCESSGGTFTSHAINWVRQAPGQRLEWMGRIIPVLGVAAYAQKFQGRVTLTADKFTSTAYMELSSLRSEDTAVYFCARSAYNYDSSGYHFDYWGQGTLVLVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 805 - protein, 213 aa, synthetic construct
DIQMTQSPSTLSASVGDRVTITCRASQSISNWLAWYQQKPGTAPKLLIFKASTLQSGVPSRFSGSGSGTEFTLTISSLQPDDFATYHCQQYHSSRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 806 - protein, 453 aa, synthetic construct
QVQLVQSGAEVKKPGSSMTVSCEASGDTFSRYSINWVRQAPGQGFEWMGRIIPMFGLATYAHKFQGRVTISADKSTTTAHMELNRLTSEDTAIYYCATAPFYYDNSTYPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 807 - protein, 214 aa, synthetic construct
DIQLTQSPSSLSASVGDRVTITCRASQSINNYLNWYQHKPGKAPKLLIYGASTLQSGVPSRFSGSGSGSDFTLTISSLQPEDFATYYCQQSYNAPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 808 - protein, 453 aa, synthetic construct
QVHLVQSGAEVKKPGSSVKVSCKASGGTFSSHTVSWVRQAPGQGLEWMGRIIPPFGIVNYAQKFQGRVTITADESTSTAYMELSSLSSEDTAVYYCARSNVVVVTEAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 809 - protein, 214 aa, synthetic construct
DIQMTQSPSSVSASVGDRVTITCRASQGISSSLAWYQQKPGKAPKVLIFDASTLQSGVPSRFSGSGSGTDFTLTISNLRPEDFATYYCQQAHSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 810 - protein, 453 aa, synthetic construct
QVQLVQSGAEVKKPGSSVKVSCEASGDTFSRYAITWVRQAPGQGLEWMGRIIPMFGSANYAQKFQGRVSFTADKSTSTASMELSSLRSEDTAVYYCATSPYYSDSGGYYCDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 811 - protein, 214 aa, synthetic construct
DIQMTQSPSSLSASVGDRVTITCRASQGISNYLAWYQQKPGKAPKLLLYGASRLQSGVPSRFSGSGSGTDYTLTISSLQPEDFATFYCQQYYSAPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 812 - protein, 448 aa, synthetic construct
QVQLQQWGAGLLKPSETLSLTCTVYGGSISGYYWNWIRQPPGKGLEWIGEIDHSGNTNYNSSLKSRITIALDTSKKEISLKVTSVTAADTAVYYCARLDYYHYSQDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 813 - protein, 213 aa, synthetic construct
EIVLTQSPDFQSVTPKERVTITCRASQSIGASLHWYQLKPNQSPKLLIKYASQSISGVPSRFSGSGSGTDFTLTISSLEAEDAATYYCLQSSNLYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 814 - protein, 455 aa, synthetic construct
QVQLVQSGAEVKKPGSSVKVSCKASGGTFTTYAIAWVRQAPGQGLEWMGRIIPTLDNPNYAQKFQGRVSITADKSTGTSYMELRSLRSDDTAVYYCAHVSYDSSGPHLSWYFNLWGRGTLITVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 815 - protein, 210 aa, synthetic construct
DIQMTQSPSTLSASVGDRVTITCRASQSIPSRLAWYQHKPGEAPKLLIYEASTLESGVPSRFSGSGSGTEFTLTITSLQPDDFATYFCQGWNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 816 - protein, 457 aa, synthetic construct
QVQLVQSGAAVKKPGSSVKVSCEASGGTFSRFAVNWVRQAPGQGLEWMGRIIPLFAIANYPQRFQGRVIMSADRSTNITYMELSRLTSEDTAVYYCAREIDTVIDPAMDVFHGMEVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 817 - protein, 217 aa, synthetic construct
QSVLTQPPSASGTPGQRVTISCSGRSSNIGSNYVHWYQQLPGTAPKLLIYRNNQRPSGVPDRFSGSKSGTSASLAISGLRSEDEADYYCAGWDDSLSGAVFGGGTKLSVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 818 - protein, 459 aa, synthetic construct
EVQLVESGGGLVKPGESLRLSCAASGFTFSSSSLNWVRQAPGKGLEWVSSISISGDYTYYADSVKGRFTISRDNGKNSVYLQMDRLRVEDTAMYYCARDRFGFYDMLAHSYNVGFFQSWGQGTLVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 819 - protein, 214 aa, synthetic construct
DIRMTQSPSSLSASVGDSVTITCRASQDITNYLAWFQQKPGKAPKSLIYSASILQSGVPSKFSGSGSGTDFTLTISSLQPEDFATYYCQQYLTYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 820 - protein, 460 aa, synthetic construct
QVQLVQSGSEVKKPGSSVKVSCRASGDTFSSHSLSWVRQAPGQGLEWMGRIVPMFGITNYAQNFQGRVTFTADKSTSTAYMELSSLRSEDTAVYYCARAGTMITFGGLTDDGQLNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 821 - protein, 219 aa, synthetic construct
DVVLTQSPLSLSVTLGQPASISCRSSQSLEYSDGDTYLNWFHQRPGQSPRRLIYKVSNRDSGVPDRFSGSGAGTDFTLKISKVEAEDVGVYYCMQGTNWPPNFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 822 - protein, 118 aa, synthetic construct
  VARIANT 10: note = X can be any amino acid
  VARIANT 13: note = X can be any amino acid
  VARIANT 23: note = X can be any amino acid
  VARIANT 35: note = X can be any amino acid
  VARIANT 37: note = X can be any amino acid
  VARIANT 48..50: note = X can be any amino acid
  VARIANT 56..58: note = X can be any amino acid
  VARIANT 79: note = X can be any amino acid
  VARIANT 82: note = X can be any amino acid
  VARIANT 87: note = X can be any amino acid
  VARIANT 92: note = X can be any amino acid
  VARIANT 96: note = X can be any amino acid
  VARIANT 98..102: note = X can be any amino acid
  VARIANT 105: note = X can be any amino acid
  VARIANT 107: note = X can be any amino acid
  VARIANT 112..113: note = X can be any amino acid
EVQLVESGGXLVXPGGSLRLSCXASEIIVSRNYMXWXRQAPGKGLEWXXXLYAGGXXXYADSVKGRFTISRDNSKNTLXLQXNSLRXEDTAXYYCXRXXXXXGGXDXWGQGXXVTVSS

SEQ ID NO: 823 - protein, 107 aa, synthetic construct
  VARIANT 4: note = X can be any amino acid
  VARIANT 10: note = X can be any amino acid
  VARIANT 24: note = X can be any amino acid
  VARIANT 28: note = X can be any amino acid
  VARIANT 30..31: note = X can be any amino acid
  VARIANT 34: note = X can be any amino acid
  VARIANT 39: note = X can be any amino acid
  VARIANT 50..51: note = X can be any amino acid
  VARIANT 53: note = X can be any amino acid
  VARIANT 55..56: note = X can be any amino acid
  VARIANT 70: note = X can be any amino acid
  VARIANT 72..73: note = X can be any amino acid
  VARIANT 76: note = X can be any amino acid
  VARIANT 83: note = X can be any amino acid
  VARIANT 89..96: note = X can be any amino acid
  VARIANT 103: note = X can be any amino acid
DIQXTQSPSXLSASVGDRVTITCXASQXIXXYLXWYQQXPGKAPKLLIYXXSXLXXGVPSRFSGSGSGTXFXXTIXSLQPEDXATYYCXXXXXXXXTFGQGTXLEIK

SEQ ID NO: 824 - protein, 120 aa, synthetic construct
QVQLQESGPGLVKPSETLSLTCTVSGGFIGPHYWSWVRQPPGKGLEWIGYIYISGSTNYNPSLKSRLTISVDMSKSQFSLTLSSATAADTAVYYCARGGGYLETGPFEYWGQGTLVTVSS

SEQ ID NO: 825 - protein, 8 aa, synthetic construct
GGFIGPHY

SEQ ID NO: 826 - protein, 7 aa, synthetic construct
IYISGST

SEQ ID NO: 827 - protein, 14 aa, synthetic construct
ARGGGYLETGPFEY

SEQ ID NO: 828 - protein, 17 aa, synthetic construct
WSWVRQPPGKGLEWIGY

SEQ ID NO: 829 - protein, 38 aa, synthetic construct
NYNPSLKSRLTISVDMSKSQFSLTLSSATAADTAVYYC

SEQ ID NO: 830 - protein, 112 aa, synthetic construct
DIVMTQSPLSLPVTPGEPASISCRSSQSLLQNNGYNYLAWYLQKPGQSPQLLIYLSSTRASGVPDRFSGSGSGTDFTLKISRVEAEDVGVYYCMQSLQIPGTFGQGTRLEIK

SEQ ID NO: 831 - protein, 11 aa, synthetic construct
QSLLQNNGYNY

SEQ ID NO: 832 - skipped

SEQ ID NO: 833 - protein, 9 aa, synthetic construct
MQSLQIPGT

SEQ ID NO: 834 - protein, 26 aa, synthetic construct
DIVMTQSPLSLPVTPGEPASISCRSS

SEQ ID NO: 835 - protein, 17 aa, synthetic construct
LAWYLQKPGQSPQLLIY

SEQ ID NO: 836 - protein, 37 aa, synthetic construct
TRASGVPDRFSGSGSGTDFTLKISRVEAEDVGVYYCM

SEQ ID NO: 837 - protein, 450 aa, synthetic construct
QVQLQESGPGLVKPSETLSLTCTVSGGFIGPHYWSWVRQPPGKGLEWIGYIYISGSTNYNPSLKSRLTISVDMSKSQFSLTLSSATAADTAVYYCARGGGYLETGPFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 838 - protein, 219 aa, synthetic construct
DIVMTQSPLSLPVTPGEPASISCRSSQSLLQNNGYNYLAWYLQKPGQSPQLLIYLSSTRASGVPDRFSGSGSGTDFTLKISRVEAEDVGVYYCMQSLQIPGTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 839 - protein, 119 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSS

SEQ ID NO: 840 - protein, 10 aa, synthetic construct
GFSLSISGVG

SEQ ID NO: 841 - protein, 7 aa, synthetic construct
IYWDDDK

SEQ ID NO: 842 - protein, 11 aa, synthetic construct
AHHSISTIFDH

SEQ ID NO: 843 - protein, 25 aa, synthetic construct
QITLKESGPTLVKPTQTLTLTCTFS

SEQ ID NO: 844 - protein, 15 aa, synthetic construct
WLRQPPGKALEWLAL

SEQ ID NO: 845 - protein, 38 aa, synthetic construct
RYSPSLKSRLTISKDTSKNQVVLKMTNIDPVDTATYYC

SEQ ID NO: 846 - protein, 109 aa, synthetic construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVL

SEQ ID NO: 847 - protein, 9 aa, synthetic construct
SSDVGDYNY

SEQ ID NO: 848 - skipped

SEQ ID NO: 849 - protein, 9 aa, synthetic construct
SSYTTSSAV

SEQ ID NO: 850 - protein, 25 aa, synthetic construct
QSALTQPASVSGSPGQSITISCTAT

SEQ ID NO: 851 - protein, 17 aa, synthetic construct
VSWYQQHPGKAPKLMIF

SEQ ID NO: 852 - protein, 36 aa, synthetic construct
DRPSGISNRFSGSKSGNTASLTISGLQAEDEADYYC

SEQ ID NO: 853 - protein, 12 aa, synthetic construct
AVFGGGTKLTVL

SEQ ID NO: 854 - protein, 449 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 855 - protein, 215 aa, synthetic construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0209318 A1
(43) Pub. Date: Jul. 23, 2026

(54) SARS-COV-2 SPIKE PROTEIN-BINDING MOLECULES

(71) Applicant: National University of Singapore, Singapore (SG)

(72) Inventors: Linfa Wang, Singapore (SG); Wan Ni Chia, Singapore (SG); Chee Wah Tan, Singapore (SG); Feng Zhu, Singapore (SG)

(21) Appl. No.: 19/141,758

(22) PCT Filed: Dec. 20, 2023
(86) PCT No.: PCT/EP2023/087111

§ 371 Date: Jun. 20, 2025

(30) Foreign Application Priority Data
Dec. 21, 2022 (SG) 10202260528T

(51) Int. Cl. C07K 16/104; A61K 40/31; A61K 40/46; A61P 31/14; G01N 33/569

(52) U.S. Cl. CPC C07K 16/104; A61K 40/31; A61K 40/46; A61P 31/14; G01N 33/56983; A61K 2239/13; A61K 2239/29; C07K 2317/31; C07K 2317/565; C07K 2317/76; G01N 2333/165

(57) ABSTRACT

SARS-CoV-2 spike protein-binding molecules are disclosed. Also disclosed are nucleic acids and expression vectors encoding, compositions comprising, and methods using, the SARS-CoV-2 spike protein-binding molecules.

This application claims priority from SG 10202260528T filed 21 Dec. 2022, the contents and elements of which are herein incorporated by reference for all purposes.

### TECHNICAL FIELD

The present disclosure relates to the fields of molecular biology, more specifically antibody technology. The present disclosure also relates to methods of medical treatment and prophylaxis.

### BACKGROUND

The human infectious disease pandemic COVID-19 caused by severe acute respiratory syndrome coronavirus 2 (SARS-CoV-2) and its protracted ongoing outbreaks have caused devastating economic and human losses globally. The emergence of SARS-CoV-2 variants has presented a significant challenge to broad-spectrum treatment/prevention of COVID-19.

Antibodies capable of inhibiting interaction between the spike protein of SARS-CoV-2 and SARS-CoV-2 variants and the spike protein receptor ACE2, and thus capable of inhibiting infection of ACE2-expressing cells by such viruses, are described e.g. in WO 2022/245288 A1 and Westendorf et al., Cell Reports (2022) 39(7): 110812. However, there remains an unmet need for antibodies capable of neutralising infection by a broader range of sarbecoviruses, including emerging and future SARS-CoV-2 variants, and other sarbecoviruses of pandemic potential.

### SUMMARY

In a first aspect, the present disclosure provides an antigen-binding molecule, optionally isolated, that binds to a sarbecovirus spike protein, wherein the antigen-binding molecule comprises: (i) a VH region comprising HC-CDR1, HC-CDR2 and HC-CDR3 as indicated in column A of Table A, and (ii) a VL region comprising LC-CDR1, LC-CDR2 and LC-CDR3 as indicated in column B of Table A, wherein the sequences of Columns A and B are selected from the same row of Table A.

In some embodiments, the antigen-binding molecule comprises:
(i) a heavy chain variable (VH) region incorporating the following CDRs:
   HC-CDR1 having the amino acid sequence of SEQ ID NO37
   HC-CDR2 having the amino acid sequence of SEQ ID NO:53
   HC-CDR3 having the amino acid sequence of SEQ ID NO:54; and
(ii) a light chain variable (VL) region incorporating the following CDRs:
   LC-CDR1 having the amino acid sequence of SEQ ID NO:60
   LC-CDR2 having the amino acid sequence of SEQ ID NO:61
   LC-CDR3 having the amino acid sequence of SEQ ID NO:62.

In some embodiments, the antigen-binding molecule comprises: (i) a VH region comprising an amino acid sequence indicated in column A of Table C, and (ii) a VL region comprising an amino acid sequence indicated in column B of Table C, wherein the sequences of columns A and B are selected from the same row of Table C.

In some embodiments, the antigen-binding molecule comprises:
a VH region having an amino acid sequence having at least 70% amino acid sequence identity to SEQ ID NO:52; and
a VL region having an amino acid sequence having at least 70% amino acid sequence identity to SEQ ID NO:59.

In some embodiments, the antigen-binding molecule is a multispecific antigen-binding molecule, and the antigen-binding molecule further comprises an antigen-binding domain which binds to an antigen other than a sarbecovirus spike protein.

The present disclosure also provides a chimeric antigen receptor (CAR) comprising an antigen-binding molecule according to the present disclosure.

The present disclosure also provides a nucleic acid, or a plurality of nucleic acids, optionally isolated, encoding an antigen-binding molecule according to the present disclosure, or a CAR according to the present disclosure.

The present disclosure also provides an expression vector, or a plurality of expression vectors, comprising a nucleic acid or a plurality of nucleic acids according to the present disclosure.

The present disclosure also provides a cell comprising an antigen-binding molecule, CAR, nucleic acid or plurality of nucleic acids, or expression vector or plurality of expression vectors according to the present disclosure.

The present disclosure also provides a method comprising culturing a cell according to the present disclosure under conditions suitable for expression of an antigen-binding molecule or CAR by the cell.

The present disclosure also provides a composition comprising an antigen-binding molecule, CAR, nucleic acid or plurality of nucleic acids, expression vector or plurality of expression vectors, or cell according to the present disclosure, and a pharmaceutically acceptable carrier, diluent, excipient or adjuvant.

In some embodiments, the composition further comprises:
(a) an antigen-binding molecule that binds to a sarbecovirus spike protein, comprising a VH region having an amino acid sequence having at least 70% amino acid sequence identity to SEQ ID NO:824, and a VL region having an amino acid sequence having at least 70% amino acid sequence identity to SEQ ID NO:830; or
(b) an antigen-binding molecule that binds to a sarbecovirus spike protein, comprising a VH region having an amino acid sequence having at least 70% amino acid sequence identity to SEQ ID NO:839, and a VL region having an amino acid sequence having at least 70% amino acid sequence identity to SEQ ID NO:846.

The present disclosure also provides a combination comprising: (i) an antigen-binding molecule according to the present disclosure, and (ii) (a) an antigen-binding molecule that binds to a sarbecovirus spike protein, comprising a VH region having an amino acid sequence having at least 70% amino acid sequence identity to SEQ ID NO:824, and a VL region having an amino acid sequence having at least 70% amino acid sequence identity to SEQ ID NO:830; or (b) an antigen-binding molecule that binds to a sarbecovirus spike protein, comprising a VH region having an amino acid sequence having at least 70% amino acid sequence identity to SEQ ID NO:839, and a VL region having an amino acid sequence having at least 70% amino acid sequence identity to SEQ ID NO:846.

The present disclosure also provides an antigen-binding molecule, CAR, nucleic acid or plurality of nucleic acids, expression vector or plurality of expression vectors, cell, composition, or combination according to the present disclosure, for use in a method of medical treatment or prophylaxis.

The present disclosure also provides an antigen-binding molecule, CAR, nucleic acid or plurality of nucleic acids, expression vector or plurality of expression vectors, cell, composition, or combination according to the present disclosure, for use in treating or preventing a disease or condition characterised by infection with a sarbecovirus, optionally wherein the disease or condition characterised by infection with a sarbecovirus is COVID-19.

The present disclosure also provides the use of an antigen-binding molecule, CAR, nucleic acid or plurality of nucleic acids, expression vector or plurality of expression vectors, cell, composition, or combination according to the present disclosure in the manufacture of a medicament for treating or preventing a disease or condition characterised by infection with a sarbecovirus, optionally wherein the disease or condition characterised by infection with a sarbecovirus is COVID-19.

The present disclosure also provides a method of treating or preventing a disease or condition characterised by infection with a sarbecovirus in a subject, comprising to a subject a therapeutically- or prophylactically-effective amount of an antigen-binding molecule, CAR, nucleic acid or plurality of nucleic acids, expression vector or plurality of expression vectors, cell, composition, or combination according to the present disclosure in the manufacture of a medicament for treating or preventing a disease or condition characterised by infection with a sarbecovirus, optionally wherein the disease or condition characterised by infection with a sarbecovirus is COVID-19.

The present disclosure also provides an in vitro complex, optionally isolated, comprising an antigen-binding molecule according to the present disclosure bound to a sarbecovirus or a sarbecovirus spike protein.

The present disclosure also provides a method for detecting a sarbecovirus or a sarbecovirus spike protein in a sample, comprising contacting a sample containing, or suspected to contain, a sarbecovirus or a sarbecovirus spike protein with an antigen-binding molecule according to the present disclosure, and detecting the formation of a complex of the antigen-binding molecule with a sarbecovirus or a sarbecovirus spike protein.

The present disclosure also provides a method of selecting or stratifying a subject for treatment with a sarbecovirus-targeted agent, the method comprising contacting, in vitro, a sample from the subject with an antigen-binding molecule according to the present disclosure, and detecting the formation of a complex of the antigen-binding molecule with a sarbecovirus or a sarbecovirus spike protein.

The present disclosure also provides the use of an antigen-binding molecule according to the present disclosure as an in vitro or in vivo diagnostic or prognostic agent.

### DESCRIPTION

The present disclosure provides antigen-binding molecules that bind to sarbecovirus spike proteins (e.g. SARS-CoV-2 spike protein and/or SARS-CoV-2 variant spike proteins), having novel biophysical and/or functional properties as compared to antigen-binding molecules disclosed in the prior art.

In particular, the present disclosure provides antigen-binding molecules that bind to sarbecovirus spike proteins and inhibit interaction between the sarbecovirus spike proteins and ACE2. Such antigen-binding molecules are useful for inhibiting infection of ACE2-expressing cells by sarbecoviruses comprising such spike proteins.

The antigen-binding molecules of the present disclosure are capable of interaction between ACE2 and a broad spectrum of different sarbecovirus spike proteins, including a wide range of SARS-CoV-2 variant spike proteins. The antigen-binding molecules of the present disclosure are therefore useful to treat and prevent disease caused by a wide range of sarbecoviruses, including a broad spectrum of SARS-CoV-2 variants. Antigen-binding molecules of the present disclosure are also demonstrated herein to inhibit interaction between ACE2 and sarbecovirus spike proteins with increased potency as compared to known sarbecovirus spike protein-binding antibodies.

The present disclosure also provides compositions comprising, and therapeutic/prophylactic intervention employing, novel combinations of antigen-binding molecules that provide for inhibition of infection of ACE2-expressing cells by sarbecoviruses with increased potency, and/or inhibition of infection of ACE2-expressing cells by a wider range of sarbecoviruses, as compared to known compositions/intervention.

### Sarbecoviruses, SARSr-CoV, SARS-CoV-2 and SARS-CoV-2 Variants

The present disclosure relates to sarbecoviruses. Sarbecoviruses are members of the subgenus Sarbecovirus of coronaviruses of the genus Betacoronavirus that infects humans, bats and certain other mammals. They are enveloped, positive-sense, single-stranded RNA viruses.

Based on their evolutionary relationship, sarbecoviruses can be divided into three main clades: clades 1, 2 and 3; see e.g. Xiang et al., Cell Rep. (2022) 39(13):111004 and Tortorici et al., Nature (2021) 597: 103-108.

Sarbecoviruses in clade 1 can be further grouped into clades 1a, 1b and 1c. Clade 1a sarbecoviruses include SARS-COV (also known as SARS-CoV-1), WIV-1, LYRa11, Rs4231, BtSY1, RsSHC014 and Rs9401. Clade 1b sarbecoviruses include SARS-CoV-2, SARS-CoV-2 variants, RaTG13, BANAL-20-51, BANAL-20-52, BANAL-20-236, BANAL-20-103, Rc-0319, RsSTT182, BtSY2, GX-P5L and GD-1. Clade 1c sarbecoviruses include RaTG15 and RpYN04. Sarbecoviruses in clade 2 include RmYN02, RacCS203, SL-ZX45, SL-ZXC21, BANAL-20-116, BANAL-20-247, PrC31, RpYN06, Rm1, Rf1, Rp3, HKU3-1, JTMC15, SX2013, HeB2013, Rs4237, 16BO133 and Anlong-103. Sarbecoviruses in clade 3 include BtKY72, BM48-31 and Khosta-2.

In some embodiments, a sarbecovirus according to the present disclosure is a sarbecovirus of clade 1, clade 2 or clade 3. In some embodiments, a sarbecovirus is a sarbecovirus of clade 1 or clade 3. In some embodiments, a sarbecovirus is a sarbecovirus of clade 1b or clade 3. In some embodiments, a sarbecovirus is not a sarbecovirus of clade 2. In some embodiments, a sarbecovirus is a sarbecovirus of clade 1. In some embodiments, a sarbecovirus is a sarbecovirus of clade 1a or 1b. In some embodiments, a sarbecovirus is a sarbecovirus of clade 1b.

In some embodiments, a sarbecovirus according to the present disclosure may be a sarbecovirus having a nucleotide sequence having at least 60% (e.g. one of ≥60%, ≥65%, ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the nucleotide sequence of GenBank: MN908947.3.

In some embodiments, a sarbecovirus according to the present disclosure is a severe acute respiratory syndrome-related coronavirus (SARSr-CoV). The virology of SARSr-CoV and epidemiology of disease associated with SARSr-CoV infection is reviewed, for example, in Cheng et al., Clin Microbiol Rev (2007) 20(4): 660-694 and de Wit et al., Nat Rev Microbiol (2016) 14: 523-534, both of which are hereby incorporated by reference in their entirety.

Two strains of SARSr-CoV have caused serious outbreaks of severe respiratory diseases in humans: SARS-COV, which caused an outbreak of severe acute respiratory syndrome (SARS) between 2002 and 2003, and SARS-CoV-2, which has caused the coronavirus disease 2019 (COVID-19) pandemic. There are hundreds of strains of SARSr-CoV known only to infect non-human species; bats are a major reservoir of many strains of SARS-related coronaviruses.

In some embodiments, a sarbecovirus according to the present disclosure is SARS-CoV-2 or a SARS-CoV-2 variant.

As used herein, ‘SARS-CoV-2’ refers to the SARSr-CoV having the nucleotide sequence of GenBank: MN908947.3 (‘Severe acute respiratory syndrome coronavirus 2 isolate Wuhan-Hu-1, complete genome’), reported in Wu et al., Nature (2020) 579: 265-269.

A large number of SARS-CoV-2 variants have been observed, and are described e.g. in Planas et al., Nat. Comm. (2023) 14: 824, Habib et al., Microbiol Resour Announc. (2023) 12(3): e00001-23, Katzmarzyk et al., Front Immunol. (2023) 14:1288794, Lasrado et al., Vaccine (2023) 41(47): 6904-6909 and Rahman et al. Microbiol Resour Announc. (2023) 12(10): e00562-23.

A ‘SARSr-CoV’ according to the present disclosure may refer to a sarbecovirus having a nucleotide sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the nucleotide sequence of GenBank: MN908947.3.

As used herein, a ‘SARS-CoV-2 variant’ refers to a SARSr-CoV having a nucleotide sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) sequence identity to the nucleotide sequence of GenBank: MN908947.3, wherein the nucleotide sequence is non-identical to the nucleotide sequence of GenBank: MN908947.3.

SARS-CoV-2 variants of particular interest in connection with the present disclosure include: BA.1 (also known as omicron; B.1.1.529; e.g. as represented by GISAID accession EPI_ISL_7358094.2); omicron subvariants such as BA.2 (e.g. as represented by GISAID accession EPI_ISL_6795834.2), BA.5 (GISAID accession EPI_ISL_12268495.2), BA.2.75 (e.g. as represented by GISAID accession EPI_ISL_13692860), BA.2.75.2 (e.g. as represented by GISAID accession EPI_ISL_15731524), BA.4.6.1 (e.g. as represented by GISAID accession EPI_ISL_13925521), BF.7 (e.g. as represented by GISAID accession EPI_ISL_13972569), BQ.1.1 (e.g. as represented by GISAID accession EPI_ISL_15731523), XBB (e.g. as represented by GISAID accession EPI_ISL_15503011) XBB.1 (e.g. as represented by GISAID accession EPI_ISL_15503005); XBB.1.16 (e.g. as represented by GISAID accession EPI_ISL_17646715); XBB.2.3 (e.g. as represented by GISAID accession EPI_ISL_17719186); EG.5 (e.g. as represented by EPI_ISL_17976635), EG.5.1 (e.g. as represented by GISAID accession EPI_ISL_18125149), B.1.1.7 (also known as alpha; GISAID accession EPI_ISL_674612); B.1.351 (also known as beta, and 501Y.V2; GISAID accession EPI_ISL_940877); B.1.617.2 (also known as delta; GISAID accession EPI_ISL_1921353); and P.1 (also known as gamma; GISAID accession EPI_ISL_2777382).

Accordingly, in some embodiments, a SARS-CoV-2 variant according to the present disclosure is selected from: BA.1, BA.2, BA.5, BA.2.75, BA.2.75.2, BA.4.6.1, BF.7, BQ.1.1, XBB, XBB.1, XBB.1.16, XBB.2.3, EG.5, EG.5.1, B.1.1.7, B.1.351, B.1.617.2 and P.1.

### SARS-CoV-2 Spike Protein and SARS-CoV-2 Variant Spike Proteins

The sarbecovirus genome encodes four major structural proteins: the spike(S) protein, the envelope (E) protein, the membrane (M) protein, and the nucleocapsid (N) protein. The present disclosure is particularly concerned with antigen-binding molecules that bind to the spike proteins of sarbecoviruses.

The canonical spike protein of SARS-CoV-2 (i.e. the spike protein encoded by the nucleotide sequence of GenBank: MN908947.3) has the amino acid sequence shown in SEQ ID NO:1. SARS-CoV-2 spike protein comprises S1 (SEQ ID NO:6) and S2 (SEQ ID NO:9) subunits. The S1 subunit comprises a minimal receptor-binding domain (RBD; SEQ ID NO:7) through which SARS-CoV-2 binds to ACE2 expressed by host cells. The RBD in turn comprises the receptor binding motif (RBM; SEQ ID NO:8), which is the region of the RBD that contacts ACE2.

In this specification, ‘SARS-CoV-2 spike protein’ refers to a polypeptide having the amino acid sequence of SEQ ID NO:1. The RBD of SARS-CoV-2 spike protein refers to the amino acid sequence of SEQ ID NO:7. The RBM of SARS-CoV-2 spike protein refers to the amino acid sequence of SEQ ID NO:8.

Many variants of SARS-CoV-2 spike protein (i.e. encoded by SARS-CoV-2 variants) have been reported, i.e. comprising one or more amino acid substitutions, deletions or insertions in the amino acid sequence of the spike protein. Such proteins may be referred to herein as SARS-CoV-2 variant spike proteins.

A ‘sarbecovirus spike protein’ according to the present disclosure refers to a polypeptide having an amino acid sequence having at least 60% (e.g. one of ≥60%, ≥65%, ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:1. A ‘SARSr-CoV spike protein’ according to the present disclosure refers to a polypeptide having an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:1.

A ‘SARS-CoV-2 variant spike protein’ according to the present disclosure refers to a polypeptide having an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:1, wherein the amino acid sequence is non-identical to SEQ ID NO:1.

In some embodiments, a SARS-CoV-2 variant spike protein comprises an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:7, wherein the amino acid sequence is non-identical to SEQ ID NO:7. In some embodiments, a SARS-CoV-2 variant spike protein comprises an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:8, wherein the amino acid sequence is non-identical to SEQ ID NO:8.

The following table summarises variations (i.e. amino acid substitutions and deletions (A) in the amino acid sequences of the spike proteins encoded by SARS-CoV-2 variants of particular interest. The variations of the SARS-CoV-2 variant spike proteins shown in the Table 1 are obtained from outbreak.info (Gangavarapu et al., Nature Methods (2023) 20:512-522). The numbering of positions of SARS-CoV-2 spike protein residues and variants can be determined relative to SEQ ID NO:1 of the present disclosure.

TABLE 1

| | | | | Column C |
|---|---|---|---|---|
| | SARS- | Column A | Column B | Spike protein |
| | CoV-2 | RBM | RBD variation | variation |
| | variant | variation | outside of RBM | outside of RBD |
| 1 | BA.1 | N440K, G446S, S477N, | G339D, S371L, S373P, | A67V, Δ69, Δ70, T95I, |
| | | T478K, E484A, Q493R, | S375F, K417N | G142D, Δ143, Δ144, Δ145, |
| | | G496S, Q498R, N501Y, | | N211I, Δ 212, 214:EPE, , |
| | | Y505H | | T547K, D614G, H655Y, |
| | | | | N679K, P681H, N764K, |
| | | | | D796Y, N856K, Q954H, |
| | | | | N969K, L981F |
| 2 | BA.2 | N440K, S477N, T478K, | G339D, S371F, S373P, | T19I, L24S, Δ25, Δ26, Δ27, |
| | | E484A, Q493R, Q498R, | S375F, T376A, D405N, | G142D, V213G, D614G, |
| | | N501Y, Y505H | R408S, K417N | H655Y, N679K, P681H, |
| | | | | N764K, D796Y, Q954H, |
| | | | | N969K |
| 3 | BA.5 | N440K, L452R, S477N, | G339D, S371F, S373P, | T19I, L24S, Δ25, Δ26, Δ27, |
| | | T478K, E484A, F486V, | S375F, T376A, D405N, | Δ69, Δ70, G142D, V213G, |
| | | Q498R, N501Y, Y505H | R408S, K417N | D614G, H655Y, N679K, |
| | | | | P681H, N764K, D796Y, |
| | | | | Q954H, N969K |
| 4 | BA.2.75 | N440K, G446S, N460K, | G339H, S371F, S373P, | T19I, L24S, Δ25, Δ26, Δ27, |
| | | S477N, T478K, E484A, | S375F, T376A, D405N, | G142D, K147E, W152R, |
| | | Q498R, N501Y, Y505H | R408S, K417N | F157L, I210V, V213G, |
| | | | | G257S, D614G, H655Y, |
| | | | | N679K, P681H, N764K, |
| | | | | D796Y, Q954H, N969K |
| 5 | BA.2.75.2 | N440K, G446S, N460K, | G339H, R346T, S371F, | T19I, L24S, Δ25, Δ26, Δ27, |
| | | S477N, T478K, E484A, | S373P, S375F, T376A, | G142D, K147E, W152R, |
| | | F486S, Q498R, N501Y, | D405N, R408S, K417N | F157L, I210V, V213G, |
| | | Y505H | | G257S, D614G, H655Y, |
| | | | | N679K, P681H, N764K, |
| | | | | D796Y, Q954H, N969K, |
| | | | | D1199N |
| 6 | BA.4.6.1 | N440K, L452R, S477N, | G339D, R346T, S371F, | T19I, L24S, Δ25, Δ26, Δ27, |
| | | T478K, E484A, F486V, | S373P, S375F, T376A, | Δ69, Δ70, G142D, W152L, |
| | | Q498R, N501Y, Y505H | D405N, R408S, K417N | V213G, D614G, H655Y, |
| | | | | N658S, N679K, P681H, |
| | | | | N764K, D796Y, Q954H, |
| | | | | N969K |
| 7 | BF.7 | N440K, L452R, S477N, | G339D, R346T, S371F, | T19I, L24S, Δ25, Δ26, Δ27, |
| | | T478K, E484A, F486V, | S373P, S375F, T376A, | Δ69, Δ70, G142D, V213G, |
| | | Q498R, N501Y, Y505H | D405N, R408S, K417N | D614G, H655Y, N679K, |
| | | | | P681H, N764K, D796Y, |
| | | | | Q954H, N969K |
| 8 | BQ.1.1 | N440K, K444T, L452R, | G339D, R346T, S371F, | T19I, L24S, Δ25, Δ26, Δ27, |
| | | N460K, S477N, T478K, | S373P, S375F, T376A, | Δ69, Δ70, G142D, V213G, |
| | | E484A, F486V, Q498R, | D405N, R408S, K417N | D614G, H655Y, N679K, |
| | | N501Y, Y505H | | P681H, N764K, D796Y, |
| | | | | Q954H, N969K |
| 9 | XBB.1 | N440K, V445P, G446S, | G339H, R346T, L368I, | T19I, L24S, Δ25, Δ26, Δ27, |
| | | N460K, S477N, T478K, | S371F, S373P, S375F, | V83A, G142D, Δ144, |
| | | E484A, F486S, F490S, | T376A, D405N, R408S, | H146Q, Q183E, V213E, |
| | | Q498R, N501Y, Y505H | K417N | G252V, D614G, H655Y, |
| | | | | N679K, P681H, N764K, |
| | | | | D796Y, Q954H, N969K |
| 10 | XBB.1.16 | N440K, V445P, G446S, | G339H, R346T, L368I, | T19I, L24S, Δ25, Δ26, Δ27, |
| | | N460K, S477N, T478R, | S371F, S373P, S375F, | V83A, G142D, Δ144, |
| | | E484A, F486P, F490S, | T376A, D405N, R408S, | H146Q, E180V, Q183E, |
| | | Q498R, N501Y, Y505H | K417N | V213E, G252V, D614G, |
| | | | | H655Y, N679K, P681H, |
| | | | | N764K, D796Y, Q954H, |
| | | | | N969K |
| 11 | XBB.2.3 | N440K, V445P, G446S, | G339H, R346T, L368I, | T19I, L24S, Δ25, Δ26, Δ27, |
| | | N460K, S477N, T478K, | S371F, S373P, S375F, | V83A, G142D, Δ144, |
| | | E484A, F486P, F490S, | T376A, D405N, R408S, | H146Q, Q183E, V213E, |
| | | Q498R, N501Y, Y505H | K417N | D253G, P521S, D614G, |
| | | | | H655Y, N679K, P681H, |
| | | | | N764K, D796Y, Q954H, |
| | | | | N969K |
| 12 | EG.5 | N440K, V445P, G446S, | G339H, R346T, L368I, | T19I, L24S, Δ25, Δ26, Δ27, |
| | | F456L, N460K, S477N | S371F, S373P, S375F, | V83A, G142D, Δ144, |
| | | T478K, E484A, F486P, | T376A, D405N, R408S, | H146Q, Q183E, V213E, |
| | | F490S, Q498R, N501Y, | K417N | G252V, D614G, H655Y, |
| | | Y505H | | N679K, P681H, N764K, |
| | | | | D796Y, Q954H, N969K |
| 13 | EG.5.1 | N440K, V445P, G446S, | G339H, R346T, L3681, | T19I, L24S, Δ25, Δ26, Δ27, |
| | | F456L, N460K, S477N, | S371F, S373P, S375F, | Q52H, V83A, G142D, |
| | | T478K, E484A, F486P, | T376A, D405N, R408S, | Δ144, H146Q, Q183E, |
| | | F490S, Q498R, N501Y, | K417N | V213E, G252V, D614G, |
| | | Y505H | | H655Y, N679K, P681H, |
| | | | | N764K, D796Y, Q954H, |
| | | | | N969K |
| 14 | B.1.1.7 | N501Y | | Δ69, Δ70, Δ144, A570D, |
| | | | | D614G, P681H, T716I, |
| | | | | S982A, D1118H |
| 15 | B.1.351 | E484K, N501Y | K417N | D80A, D215G, Δ241, Δ242, |
| | | | | Δ243, D614G, A701V |
| 16 | B.1.617.2 | L452R, T478K | | T19R, E156G, Δ157, Δ158, |
| | | | | D614G, P681R, D950N |
| 17 | P.1 | E484K, N501Y | K417T | L18F, T20N, P26S, D138Y, |
| | | | | R190S, D614G, H655Y, |
| | | | | T1027I, V1176F |
| 18 | XBB | N440K, V445P, G446S, | G339H, R346T, L368I, | T19I, L24S, Δ25, Δ26, Δ27, |
| | | N460K, S477N, T478K, | S371F, S373P, S375F, | V83A, G142D, Δ144, |
| | | E484A, F490S, Q498R, | T376A, D405N, R408S, | H146Q, Q183E, V213E, |
| | | N501Y, Y505H | K417N | D614G, H655Y, N679K, |
| | | | | P681H, N764K, D796Y, |
| | | | | Q954H, N969K |

In some embodiments, a SARS-CoV-2 variant spike protein according to the present disclosure has an amino acid sequence having at least 70% sequence identity to the amino acid sequence of SEQ ID NO:1, and comprises one or more of the variations shown in Table 1 above.

In some embodiments, a SARS-CoV-2 variant spike protein comprises, or consists of, an amino acid sequence having at least 70% sequence identity to the amino acid sequence of SEQ ID NO:1, and comprises the variation(s) shown in column A of row 1, 2, 3, 4, 5, 6, 7, 8, 9, 10, 11, 12, 13, 14, 15, 16, 17 or 18 of Table 1 above. By way of illustration, in some embodiments, a SARS-CoV-2 variant spike protein comprises, or consists of, an amino acid sequence having at least 70% sequence identity to the amino acid sequence of SEQ ID NO:1, and comprises N440K, S477N, T478K, E484A, Q493R, Q498R, N501Y, Y505H, G446S and G496S (i.e. the RBM variations of BA. 1, as shown in row 1).

In some embodiments, a SARS-CoV-2 variant spike protein comprises, or consists of, an amino acid sequence having at least 70% sequence identity to the amino acid sequence of SEQ ID NO:1, and comprises the variation(s) shown in column B of row 1, 2, 3, 4, 5, 6, 7, 8, 9, 10, 11, 12, 13, 14, 15, 16, 17 or 18 of Table 1 above. In some embodiments, a SARS-CoV-2 variant spike protein comprises, or consists of, an amino acid sequence having at least 70% sequence identity to the amino acid sequence of SEQ ID NO:1, and comprises the variation(s) shown in column C of row 1, 2, 3, 4, 5, 6, 7, 8, 9, 10, 11, 12, 13, 14, 15, 16 or 17 of Table 1 above.

In some embodiments, a SARS-CoV-2 variant spike protein comprises, or consists of, an amino acid sequence having at least 70% sequence identity to the amino acid sequence of SEQ ID NO:1, and comprises the variation(s) shown in columns A and B of row 1, 2, 3, 4, 5, 6, 7, 8, 9, 10, 11, 12, 13, 14, 15, 16, 17 or 18 of Table 1 above. By way of illustration, in some embodiments, a SARS-CoV-2 variant spike protein comprises, or consists of, an amino acid sequence having at least 70% sequence identity to the amino acid sequence of SEQ ID NO:1, and comprises N440K, S477N, T478K, E484A, Q493R, Q498R, N501Y, Y505H, G446S, G496S, G339D, S373P, S375F, K417N and S371L (i.e. the RBD variations of BA.1, as shown in row 1).

In some embodiments, a SARS-CoV-2 variant spike protein comprises, or consists of, an amino acid sequence having at least 70% sequence identity to the amino acid sequence of SEQ ID NO:1, and comprises the variation(s) shown in columns A, B and C of row 1, 2, 3, 4, 5, 6, 7, 8, 9, 10, 11, 12, 13, 14, 15, 16, 17 or 18 of Table 1 above. By way of illustration, in some embodiments, a SARS-CoV-2 variant spike protein comprises, or consists of, an amino acid sequence having at least 70% sequence identity to the amino acid sequence of SEQ ID NO:1, and comprises N440K, S477N, T478K, E484A, Q493R, Q498R, N501Y, Y505H, G446S, G496S, G339D, S373P, S375F, K417N, S371L, A67V, Δ69, Δ70, T951, G142D, Δ143, Δ144, Δ145, Δ211, L212I, +214EPE, T547K, D614G, H655Y, N679K, P681H, N764K, D796Y, N856K, Q954H, N969K and L981F (i.e. the spike protein variations of BA. 1, as shown in row 1).

In some embodiments, a SARS-CoV-2 variant spike protein comprises, or consists of, the amino acid sequence of the spike protein encoded by a SARS-CoV-2 variant selected from: BA.1, BA.2, BA.5, BA.2.75, BA.2.75.2, BA.4.6.1, BF.7, BQ.1.1, XBB, XBB.1, XBB.1.16, XBB.2.3, EG.5, EG.5.1, B.1.1.7, B.1.351, B.1.617.2 and P.1.

In some embodiments, a SARS-CoV-2 variant spike protein according to the present disclosure comprises an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:703, 704, 705, 706, 707, 708, 709, 710, 711, 712, 713, 714, 715, 716, 717, 718 or 719.

In some embodiments, a SARS-CoV-2 variant spike protein according to the present disclosure comprises an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:686, 687, 688, 689, 690, 691, 692, 693, 694, 695, 696, 697, 698, 699, 700, 701 or 702.

In some embodiments, a SARS-CoV-2 variant spike protein according to the present disclosure comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:11, 12, 13, 14, 15, 16, 17, 18, 19, 20, 21, 22, 23, 24, 25, 26 or 27.

In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:11, and comprises the variation(s) shown in (i) column A of row 1 of Table 1; (ii) columns A and B of row 1 of Table 1; or (iii) columns A, B and C of row 1 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:12, and comprises the variation(s) shown in (i) column A of row 2 of Table 1; (ii) columns A and B of row 2 of Table 1; or (iii) columns A, B and C of row 2 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:13, and comprises the variation(s) shown in (i) column A of row 3 of Table 1; (ii) columns A and B of row 3 of Table 1; or (iii) columns A, B and C of row 3 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:14, and comprises the variation(s) shown in (i) column A of row 4 of Table 1; (ii) columns A and B of row 4 of Table 1; or (iii) columns A, B and C of row 4 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:15, and comprises the variation(s) shown in (i) column A of row 5 of Table 1; (ii) columns A and B of row 5 of Table 1; or (iii) columns A, B and C of row 5 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:16, and comprises the variation(s) shown in (i) column A of row 6 of Table 1; (ii) columns A and B of row 6 of Table 1; or (iii) columns A, B and C of row 6 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:17, and comprises the variation(s) shown in (i) column A of row 7 of Table 1; (ii) columns A and B of row 7 of Table 1; or (iii) columns A, B and C of row 7 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:18, and comprises the variation(s) shown in (i) column A of row 8 of Table 1; (ii) columns A and B of row 8 of Table 1; or (iii) columns A, B and C of row 8 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:19, and comprises the variation(s) shown in (i) column A of row 9 of Table 1; (ii) columns A and B of row 9 of Table 1; or (iii) columns A, B and C of row 9 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:20, and comprises the variation(s) shown in (i) column A of row 10 of Table 1; (ii) columns A and B of row 10 of Table 1; or (iii) columns A, B and C of row 10 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:21, and comprises the variation(s) shown in (i) column A of row 11 of Table 1; (ii) columns A and B of row 11 of Table 1; or (iii) columns A, B and C of row 11 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:22, and comprises the variation(s) shown in (i) column A of row 12 of Table 1; (ii) columns A and B of row 12 of Table 1; or (iii) columns A, B and C of row 12 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:23, and comprises the variation(s) shown in (i) column A of row 13 of Table 1; (ii) columns A and B of row 13 of Table 1; or (iii) columns A, B and C of row 13 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:24, and comprises the variation(s) shown in (i) column A of row 14 of Table 1; (ii) columns A and B of row 14 of Table 1; or (iii) columns A, B and C of row 14 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:25, and comprises the variation(s) shown in (i) column A of row 15 of Table 1; (ii) columns A and B of row 15 of Table 1; or (iii) columns A, B and C of row 15 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:26, and comprises the variation(s) shown in (i) column A of row 16 of Table 1; (ii) columns A and B of row 15 of Table 1; or (iii) columns A, B and C of row 16 of Table 1. In some embodiments, a SARS-CoV-2 variant spike protein comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:27, and comprises the variation(s) shown in (i) column A of row 17 of Table 1; (ii) columns A and B of row 17 of Table 1; or (iii) columns A, B and C of row 15 of Table 1.

### ACE2

Angiotensin-converting enzyme 2 (ACE2) is the entry point into cells for SARSr-CoV, via interaction with the spike protein. SARSr-CoV spike proteins bind to the extracellular domain of ACE2 (Zhou et al., Nature (2020) 579: 270-273; Hoffmann et al., Cell (2020) 181: 271-280).

ACE2 is a single-pass type I transmembrane carboxypeptidase, which attaches to the cell membrane of cells of the outer surface tissues of lungs, arteries, heart, kidney, and intestines. The structure and function of ACE2 is described e.g. in Hamming et al., J Pathol (2004) 203(2): 631-637, which is hereby incorporated by reference in its entirety.

In this specification ‘ACE2’ refers to ACE2 from any species and includes ACE2 isoforms, fragments, variants or homologues from any species. In some embodiments, the ACE2 is ACE2 from a mammal (e.g. a therian, placental, epitherian, preptotheria, archontan, primate (rhesus, cynomolgous, non-human primate or human)). In some embodiments, the ACE2 is ACE2 from a human, bat, pangolin, civet or pig. Isoforms, fragments, variants or homologues of ACE2 may optionally be characterised as having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of an immature or mature ACE2 isoform from a given species, e.g. human.

Human ACE2 isoform 1 is shown in SEQ ID NO:28, and human ACE2 isoform 2 is shown in SEQ ID NO:35. The extracellular domain of human ACE2 is shown in SEQ ID NO:30.

Fragments of ACE2 may have a minimum length of one of 25, 50, 100, 200, 300, 400, 500, 600, 700 or 800 amino acids, and may have a maximum length of one of 50, 100, 200, 300, 400, 500, 600, 700 or 800 amino acids. Fragments of ACE2 may e.g. display association with a sarbecovirus spike protein (e.g. a SARSr-CoV spike protein; e.g. SARS-CoV-2 spike protein and/or one or more SARS-CoV-2 variant spike proteins).

In some embodiments, the ACE2 comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:28 or 35.

In some embodiments, a fragment of ACE2 comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:30.

### Antigen-Binding Molecules of the Disclosure

The present disclosure provides antigen-binding molecules capable of binding to sarbecovirus spike proteins (e.g. SARSr-CoV spike proteins; e.g. SARS-CoV-2 spike protein and/or one or more SARS-CoV-2 variant spike proteins). Such antigen-binding molecules may also be described as an antigen-binding molecules that bind to the relevant proteins.

An ‘antigen-binding molecule’ refers to a molecule that binds to a given target antigen. Antigen-binding molecules include antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH), etc. Antigen-binding fragments of antibodies include e.g. Fv, Fab, F(ab′)₂ and F(ab′) fragments. In some embodiments, an antigen-binding molecule may be an antibody or an antigen-binding fragment thereof.

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BIKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1) and Jayaraman et al., EBioMedicine (2020) 58:102931, both of which are hereby incorporated by reference in their entirety).

The antigen-binding molecule of the present disclosure comprises a moiety or moieties capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs). Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue): D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

In some embodiments, the antigen-binding molecule comprises the CDRs of an antigen-binding molecule that binds to a sarbecovirus spike protein (e.g. a SARSr-CoV spike protein; e.g. SARS-CoV-2 spike protein and/or one or more SARS-CoV-2 variant spike proteins). In some embodiments, the antigen-binding molecule comprises the FRs of an antigen-binding molecule that binds to a sarbecovirus spike protein (e.g. a SARSr-CoV spike protein; e.g. SARS-CoV-2 spike protein and/or one or more SARS-CoV-2 variant spike proteins). In some embodiments, the antigen-binding molecule comprises the CDRs and the FRs of an antigen-binding molecule that binds to a sarbecovirus spike protein (e.g. a SARSr-CoV spike protein; e.g. SARS-CoV-2 spike protein and/or one or more SARS-CoV-2 variant spike proteins). That is, in some embodiments, the antigen-binding molecule comprises the VH region and the VL region of an antigen-binding molecule that binds to a sarbecovirus spike protein (e.g. a SARSr-CoV spike protein; e.g. SARS-CoV-2 spike protein and/or one or more SARS-CoV-2 variant spike proteins).

In some embodiments, the antigen-binding molecule comprises the CDRs, FRs and/or the VH and/or VL regions of an antibody described herein (e.g. an antibody of Table C herein), or CDRs, FRs and/or VH and/or VL regions which are derived from those of antibody described herein (e.g. an antibody of Table C herein).

In some embodiments, the antigen-binding molecule comprises:
a VH region comprising HC-CDR1 (or a variant thereof in which 1 or 2 or 3 amino acids in HC-CDR1 are substituted with another amino acid), HC-CDR2 (or a variant thereof in which 1 or 2 or 3 amino acids in HC-CDR2 are substituted with another amino acid) and HC-CDR3 (or a variant thereof in which 1 or 2 or 3 amino acids in HC-CDR3 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A, wherein the HC-CDR1, HC-CDR2 and HC-CDR3 sequences of Column A are selected from the same row of Table A. By way of illustration, in some embodiments the antigen-binding molecule comprises a VH region comprising HC-CDR1 having the amino acid sequence of SEQ ID NO:37 (or a variant thereof in which 1 or 2 or 3 amino acids in SEQ ID NO:37 are substituted with another amino acid), HC-CDR2 having the amino acid sequence of SEQ ID NO:38 (or a variant thereof in which 1 or 2 or 3 amino acids in SEQ ID NO:38 are substituted with another amino acid) and HC-CDR3 having the amino acid sequence of SEQ ID NO:39 (or a variant thereof in which 1 or 2 or 3 amino acids in SEQ ID NO:39 are substituted with another amino acid). It will be appreciated that the HC-CDR1, HC-CDR2 and HC-CDR3 sequences of the preceding sentence are selected from Column A of the same row (row 1) of Table A.

In some embodiments, the antigen-binding molecule comprises:
a VH region comprising HC-FR1 (or a variant thereof in which 1 or 2 or 3 amino acids in HC-FR1 are substituted with another amino acid), HC-FR2 (or a variant thereof in which 1 or 2 or 3 amino acids in HC-FR2 are substituted with another amino acid), HC-FR3 (or a variant thereof in which 1 or 2 or 3 amino acids in HC-FR3 are substituted with another amino acid) and HC-FR4 (or a variant thereof in which 1 or 2 or 3 amino acids in HC-FR4 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B, wherein the HC-FR1, HC-FR2, HC-FR3 and HC-FR4 sequences of Column A are selected from the same row of Table B.

By way of illustration, in some embodiments, the antigen-binding molecule comprises a VH region comprising HC-FR1 having the amino acid sequence of SEQ ID NO:40 (or a variant thereof in which 1 or 2 or 3 amino acids in SEQ ID NO:40 are substituted with another amino acid), HC-FR2 having the amino acid sequence of SEQ ID NO:41 (or a variant thereof in which 1 or 2 or 3 amino acids in SEQ ID NO:41 are substituted with another amino acid), HC-FR3 having the amino acid sequence of SEQ ID NO:42 (or a variant thereof in which 1 or 2 or 3 amino acids in SEQ ID NO:42 are substituted with another amino acid) and HC-FR4 having the amino acid sequence of SEQ ID NO:43 (or a variant thereof in which 1 or 2 or 3 amino acids in SEQ ID NO:43 are substituted with another amino acid). It will be appreciated that the HC-FR1, HC-FR2, HC-FR3 and HC-FR4 sequences of the preceding sentence are selected from Column A of the same row (row 1) of Table B.

In some embodiments, the antigen-binding molecule comprises:
a VH region comprising:
   HC-CDR1 (or a variant thereof in which 1 or 2 or 3 amino acids in HC-CDR1 are substituted with another amino acid), HC-CDR2 (or a variant thereof in which 1 or 2 or 3 amino acids in HC-CDR2 are substituted with another amino acid) and HC-CDR3 (or a variant thereof in which 1 or 2 or 3 amino acids in HC-CDR3 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A, wherein the HC-CDR1, HC-CDR2 and HC-CDR3 sequences of Column A are selected from the same row of Table A; and
   HC-FR1 (or a variant thereof in which 1 or 2 or 3 amino acids in HC-FR1 are substituted with another amino acid), HC-FR2 (or a variant thereof in which 1 or 2 or 3 amino acids in HC-FR2 are substituted with another amino acid), HC-FR3 (or a variant thereof in which 1 or 2 or 3 amino acids in HC-FR3 are substituted with another amino acid) and HC-FR4 (or a variant thereof in which 1 or 2 or 3 amino acids in HC-FR4 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B, wherein the HC-FR1, HC-FR2, HC-FR3 and HC-FR4 sequences of Column A are selected from the same row of Table B.

In some embodiments, the antigen-binding molecule comprises:
a VH region comprising:
   HC-CDR1 (or a variant thereof in which 1 or 2 or 3 amino acids in HC-CDR1 are substituted with another amino acid), HC-CDR2 (or a variant thereof in which 1 or 2 or 3 amino acids in HC-CDR2 are substituted with another amino acid) and HC-CDR3 (or a variant thereof in which 1 or 2 or 3 amino acids in HC-CDR3 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A; and
   HC-FR1 (or a variant thereof in which 1 or 2 or 3 amino acids in HC-FR1 are substituted with another amino acid), HC-FR2 (or a variant thereof in which 1 or 2 or 3 amino acids in HC-FR2 are substituted with another amino acid), HC-FR3 (or a variant thereof in which 1 or 2 or 3 amino acids in HC-FR3 are substituted with another amino acid) and HC-FR4 (or a variant thereof in which 1 or 2 or 3 amino acids in HC-FR4 are substituted with another amino acid) as indicated in Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B;
   wherein the HC-CDR1, HC-CDR2, HC-CDR3 sequences of Column A of Table A and the HC-FR1, HC-FR2, HC-FR3 and HC-FR4 sequences of Column B of Table B are selected from rows having the same number.

By way of illustration, in some embodiments, the antigen-binding molecule comprises a VH region comprising: HC-CDR1 having the amino acid sequence of SEQ ID NO:37 (or a variant thereof in which 1 or 2 or 3 amino acids in SEQ ID NO:37 are substituted with another amino acid), HC-CDR2 having the amino acid sequence of SEQ ID NO:38 (or a variant thereof in which 1 or 2 or 3 amino acids in SEQ ID NO:38 are substituted with another amino acid) and HC-CDR3 having the amino acid sequence of SEQ ID NO:39 (or a variant thereof in which 1 or 2 or 3 amino acids in SEQ ID NO:39 are substituted with another amino acid), HC-FR1 having the amino acid sequence of SEQ ID NO:40 (or a variant thereof in which 1 or 2 or 3 amino acids in SEQ ID NO:40 are substituted with another amino acid), HC-FR2 having the amino acid sequence of SEQ ID NO:41 (or a variant thereof in which 1 or 2 or 3 amino acids in SEQ ID NO:41 are substituted with another amino acid), HC-FR3 having the amino acid sequence of SEQ ID NO:42 (or a variant thereof in which 1 or 2 or 3 amino acids in SEQ ID NO:42 are substituted with another amino acid) and HC-FR4 having the amino acid sequence of SEQ ID NO:43 (or a variant thereof in which 1 or 2 or 3 amino acids in SEQ ID NO:43 are substituted with another amino acid). It will be appreciated that the HC-CDR1, HC-CDR2 and HC-CDR3 sequences of the preceding sentence are selected from Column A of row 1 of Table A, and that the HC-FR1, HC-FR2, HC-FR3 and HC-FR4 sequences are selected from Column A of the row of Table B having the same number (row 1).

In some embodiments, the antigen-binding molecule comprises a VH region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of a VH region sequence selected from Column A of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table C.

In some embodiments, the antigen-binding molecule comprises:
a VL region comprising LC-CDR1 (or a variant thereof in which 1 or 2 or 3 amino acids in LC-CDR1 are substituted with another amino acid), LC-CDR2 (or a variant thereof in which 1 or 2 or 3 amino acids in LC-CDR2 are substituted with another amino acid) and LC-CDR3 (or a variant thereof in which 1 or 2 or 3 amino acids in LC-CDR3 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A, wherein the LC-CDR1, LC-CDR2 and LC-CDR3 sequences of Column B are selected from the same row of Table A.

In some embodiments, the antigen-binding molecule comprises:
a VL region comprising LC-FR1 (or a variant thereof in which 1 or 2 or 3 amino acids in LC-FR1 are substituted with another amino acid), LC-FR2 (or a variant thereof in which 1 or 2 or 3 amino acids in LC-FR2 are substituted with another amino acid), LC-FR3 (or a variant thereof in which 1 or 2 or 3 amino acids in LC-FR3 are substituted with another amino acid) and LC-FR4 (or a variant thereof in which 1 or 2 or 3 amino acids in LC-FR4 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B, wherein the LC-FR1, LC-FR2, LC-FR3 and LC-FR4 sequences of Column B are selected from the same row of Table B.

In some embodiments, the antigen-binding molecule comprises:
a VL region comprising:
   LC-CDR1 (or a variant thereof in which 1 or 2 or 3 amino acids in LC-CDR1 are substituted with another amino acid), LC-CDR2 (or a variant thereof in which 1 or 2 or 3 amino acids in LC-CDR2 are substituted with another amino acid) and LC-CDR3 (or a variant thereof in which 1 or 2 or 3 amino acids in LC-CDR3 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A, wherein the LC-CDR1, LC-CDR2 and LC-CDR3 sequences of Column B are selected from the same row of Table A; and
   LC-FR1 (or a variant thereof in which 1 or 2 or 3 amino acids in LC-FR1 are substituted with another amino acid), LC-FR2 (or a variant thereof in which 1 or 2 or 3 amino acids in LC-FR2 are substituted with another amino acid), LC-FR3 (or a variant thereof in which 1 or 2 or 3 amino acids in LC-FR3 are substituted with another amino acid) and LC-FR4 (or a variant thereof in which 1 or 2 or 3 amino acids in LC-FR4 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B, wherein the LC-FR1, LC-FR2, LC-FR3 and LC-FR4 sequences of Column B are selected from the same row of Table B.

In some embodiments, the antigen-binding molecule comprises:
a VL region comprising:
   LC-CDR1 (or a variant thereof in which 1 or 2 or 3 amino acids in LC-CDR1 are substituted with another amino acid), LC-CDR2 (or a variant thereof in which 1 or 2 or 3 amino acids in LC-CDR2 are substituted with another amino acid) and LC-CDR3 (or a variant thereof in which 1 or 2 or 3 amino acids in LC-CDR3 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table A; and
   LC-FR1 (or a variant thereof in which 1 or 2 or 3 amino acids in LC-FR1 are substituted with another amino acid), LC-FR2 (or a variant thereof in which 1 or 2 or 3 amino acids in LC-FR2 are substituted with another amino acid), LC-FR3 (or a variant thereof in which 1 or 2 or 3 amino acids in LC-FR3 are substituted with another amino acid) and LC-FR4 (or a variant thereof in which 1 or 2 or 3 amino acids in LC-FR4 are substituted with another amino acid) as indicated in Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table B;
   wherein the LC-CDR1, LC-CDR2, LC-CDR3 sequences of Column B of Table A and the LC-FR1, LC-FR2, LC-FR3 and LC-FR4 sequences of Column B of Table B are selected from rows having the same number.

In some embodiments, the antigen-binding molecule comprises a VL region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of a VL region sequence selected from Column B of row 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51 of Table C.

In some embodiments, the antigen-binding molecule comprises a VH region according to any one embodiment as described herein, and a VL region according to any one embodiment as described herein.

In embodiments in accordance with the present disclosure, one or more amino acids are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, |
| | | Trp, Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | Side-chain | Side-chain charge |
|---|---|---|
| Amino Acid | polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region that binds to a sarbecovirus spike protein (e.g. a SARSr-CoV spike protein; e.g. SARS-CoV-2 spike protein and/or one or more SARS-CoV-2 variant spike proteins). In some embodiments, the VH and VL regions of the Fv are provided as single polypeptide joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. Cκ or Cλ). In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, the antigen-binding molecule described herein comprises, or consists of, a whole antibody that binds to a sarbecovirus spike protein (e.g. a SARSr-CoV spike protein; e.g. SARS-CoV-2 spike protein and/or one or more SARS-CoV-2 variant spike proteins). As used herein, ‘whole antibody’ refers to an antibody having a structure which is substantially similar to the structure of an immunoglobulin (lg). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

Herein, a ‘CH1 domain’ refers to an amino acid sequence corresponding to the CH1 domain of an immunoglobulin (lg). The CH1 domain is the region of an Ig formed by positions 118 to 215 of the immunoglobulin constant domain, according to the EU numbering system (described in Edelman et al., Proc Natl Acad Sci USA (1969) 63(1): 78-85). A ‘hinge domain’ refers to an amino acid sequence corresponding to the hinge domain of an Ig. The hinge domain is the region of an Ig formed by positions 216 to 230 of the immunoglobulin constant domain, according to the EU numbering system. A ‘CH2 domain’ refers to an amino acid sequence corresponding to the CH2 domain of an Ig. The CH2 domain is the region of an Ig formed by positions 231 to 340 of the immunoglobulin constant domain, according to the EU numbering system. A ‘CH3 domain’ refers to an amino acid sequence corresponding to the CH3 domain of an immunoglobulin (lg). The CH3 domain is the region of an Ig formed by positions 341 to 447 of the immunoglobulin constant domain, according to the EU numbering system. A ‘CH2-CH3 region’ refers to an amino acid sequence corresponding to the CH2 and CH3 domains of an immunoglobulin (lg). The CH2-CH3 region is the region of an Ig formed by positions 231 to 447 of the immunoglobulin constant domain, according to the EU numbering system.

In some embodiments, the antigen-binding molecule described herein comprises, or consists of, an IgG (e.g. IgG1, IgG2, IgG3, IgG4), IgA (e.g. IgA1, IgA2), IgD, IgE, or IgM that binds to a sarbecovirus spike protein (e.g. a SARSr-CoV spike protein; e.g. SARS-CoV-2 spike protein and/or one or more SARS-CoV-2 variant spike proteins).

In some embodiments, the antigen-binding molecule of the present disclosure comprises one or more regions (e.g. CH1, CH2, CH3, etc.) of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments, the immunoglobulin heavy chain constant sequence is, or is derived from, the heavy chain constant sequence of a human IgG1 allotype (e.g. G1m1, G1m2, G1m3 or G1m17).

In some embodiments, the antigen-binding molecule comprises (e.g. comprises one or more polypeptides comprising) a CH1 region. In some embodiments, a CH1 region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:671 or 676.

In some embodiments, the antigen-binding molecule comprises (e.g. comprises one or more polypeptides comprising) a hinge region. In some embodiments, a hinge region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:672.

In some embodiments, the antigen-binding molecule comprises (e.g. comprises one or more polypeptides comprising) a CH2 region. In some embodiments, a CH2 region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:673.

In some embodiments, the antigen-binding molecule comprises (e.g. comprises one or more polypeptides comprising) a CH3 region. In some embodiments, a CH3 region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:674 or 677.

In some embodiments, the antigen-binding molecules of the present disclosure comprise an Fc region.

As used herein, an ‘Fc region’ refers to a polypeptide complex formed by interaction between two polypeptides, each polypeptide comprising the CH2-CH3 region of an immunoglobulin (lg) heavy chain constant sequence.

In some embodiments, a CH2 region, CH3 region and/or a CH2-CH3 region according to the present disclosure corresponds to the CH2 region/CH3 region/CH2-CH3 region of an IgG (e.g. IgG1, IgG2, IgG3, IgG4), IgA (e.g. IgA1, IgA2), IgD, IgE or IgM. In some embodiments, the CH2 region, CH3 region and/or a CH2-CH3 region corresponds to the CH2 region/CH3 region/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 region, CH3 region and/or a CH2-CH3 region corresponds to the CH2 region/CH3 region/CH2-CH3 region of a human IgG1 allotype (e.g. G1m1, G1m2, G1m3 or G1m17).

Fc regions provide for interaction with Fc receptors and other molecules of the immune system to bring about functional effects. Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73. In some embodiments, the antigen-binding molecule of the present disclosure comprises an Fc region comprising modification to increase or reduce an Fc-mediated function as compared to an antigen-binding molecule comprising the corresponding unmodified Fc region. Where an Fc region comprises a modification, the modification may be present in one or both of the polypeptide chains which together form the Fc region.

In some embodiments, the antigen-binding molecule comprises (e.g. comprises one or more polypeptides comprising) a CH2-CH3 region. In some embodiments, a CH2-CH3 region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:678 or 679.

In some embodiments, the antigen-binding molecule comprises (e.g. comprises one or more polypeptides comprising) a CH1-hinge-CH2-CH3 region. In some embodiments, a CH1-hinge-CH2-CH3 region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:670.

In some embodiments, the antigen-binding molecule of the present disclosure comprises one or more regions of an immunoglobulin light chain constant sequence. In some embodiments, the immunoglobulin light chain constant sequence is human immunoglobulin kappa constant (IGKC; Cκ). In some embodiments, the immunoglobulin light chain constant sequence is a human immunoglobulin lambda constant (IGLC; Cλ), e.g. IGLC1, IGLC2, IGLC3, IGLC6 or IGLC7.

In some embodiments, the antigen-binding molecule comprises (e.g. comprises one or more polypeptides comprising) a CL region. In some embodiments, a CL region comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) sequence identity to the amino acid sequence of SEQ ID NO:680, 681, 682, 683, 684 or 685.

In some embodiments, the antigen-binding molecule is or comprises a monoclonal antibody, or an antigen-binding fragment thereof.

In some embodiments, the antigen-binding molecule is or comprises a fully human antibody/antibody fragment. A fully human antibody/antibody fragment may be encoded by human nucleic acid sequence(s). A fully human antibody/antibody fragment may be devoid of non-human amino acid sequences.

Aspects of the present disclosure relate to multispecific antigen-binding molecules. By ‘multispecific’ it is meant that the antigen-binding molecule displays specific binding to more than one target. In some embodiments, the antigen-binding molecule is a bispecific antigen-binding molecule. In some embodiments, the antigen-binding molecule comprises at least two different antigen-binding domains (i.e. at least two antigen-binding domains, e.g. comprising non-identical VHs and VLs).

In some embodiments, the antigen-binding molecule binds to a sarbecovirus spike protein (e.g. a SARSr-CoV spike protein; e.g. SARS-CoV-2 spike protein and/or one or more SARS-CoV-2 variant spike proteins) and another target other than a sarbecovirus spike protein, and so is at least bispecific. The term ‘bispecific’ means that the antigen-binding molecule is able to bind specifically to at least two distinct antigenic determinants.

It will be appreciated that an antigen-binding molecule according to the present disclosure (e.g. a multispecific antigen-binding molecule) may comprise antigen-binding molecules capable of binding to the targets for which the antigen-binding molecule is specific. For example, an antigen-binding molecule that binds to a sarbecovirus spike protein (e.g. a SARSr-CoV spike protein; e.g. SARS-CoV-2 spike protein and/or one or more SARS-CoV-2 variant spike proteins) and another target other than a sarbecovirus spike protein may comprise: (i) an antigen-binding molecule that binds to SARS-CoV-2 spike protein and/or one or more SARS-CoV-2 variant spike proteins, and (ii) an antigen-binding molecule that binds to an antigen other than a sarbecovirus spike protein.

It will also be appreciated that an antigen-binding molecule according to the present disclosure (e.g. a multispecific antigen-binding molecule) may comprise antigen-binding polypeptides or antigen-binding polypeptide complexes capable of binding to the targets for which the antigen-binding molecule is specific.

In some embodiments, a component antigen-binding molecule of a larger antigen-binding molecule (e.g. a multispecific antigen-binding molecule) may be referred to e.g. as an ‘antigen-binding domain’ or ‘antigen-binding region’ of the larger antigen-binding molecule.

In some embodiments, the antigen-binding molecule is an immune cell engager. Immune cell engagers are reviewed e.g. in Goebeler and Bargou, Nat. Rev. Clin. Oncol. (2020) 17: 418-434 and Ellerman, Methods (2019) 154:102-117, both of which are hereby incorporated by reference in their entirety. Immune cell engager molecules comprise an antigen-binding region for a target antigen of interest, and an antigen-binding region for recruiting/engaging an immune cell of interest. Immune cell engagers recruit/engage immune cells through an antigen-binding region specific for an immune cell surface molecule.

The best studied immune cell engagers are bispecific T cell engagers (BiTEs), which comprise a target antigen binding domain, and a CD3 polypeptide (typically CD3ε)-binding domain, through which the BiTE recruits T cells. Binding of the BiTE to its target antigen and to the CD3 polypeptide expressed by the T cell results in activation of the T cell, and ultimately directs T cell effector activity against cells expressing the target antigen. Other kinds of immune cell engagers are well known in the art, and include natural killer cell engagers such as bispecific killer engagers (BiKEs), which recruit and activate NK cells.

In some embodiments, the immune cell engaged by the immune cell engager is a T cell or an NK cell. In some embodiments, the immune cell engager is a T cell-engager.

Multispecific antigen-binding molecules according to the present disclosure may be provided in any suitable format, such as those formats described in described in Brinkmann and Kontermann, MAbs (2017) 9(2): 182-212, which is hereby incorporated by reference in its entirety. Suitable formats include those shown in FIG. 2 of Brinkmann and Kontermann, MAbs (2017) 9(2): 182-212: antibody conjugates, e.g. IgG2, F(ab′) 2 or CovX-Body; IgG or IgG-like molecules, e.g. IgG, chimeric IgG, κA-body common HC; CH1/CL fusion proteins, e.g. scFv2-CH1/CL, VHH2-CH1/CL; ‘variable domain only’ bispecific antigen-binding molecules, e.g. tandem scFv (taFV), triplebodies, diabodies (Db), dsDb, Db(kih), DART, scDB, dsFv-dsFv, tandAbs, triple heads, tandem dAb/VHH, tertravalent dAb.VHH; Non-Ig fusion proteins, e.g. scFV₂-albumin, scDb-albumin, taFv-albumin, taFv-toxin, miniantibody, DNL-Fab₂, DNL-Fab₂-scFv, DNL-Fab₂-IgG-cytokine₂, ImmTAC (TCR-scFv); modified Fc and CH3 fusion proteins, e.g. scFv-Fc(kih), scFv-Fc(CH3 charge pairs), scFv-Fc (EW-RVT), scFv-fc (HA-TF), scFv-Fc (SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc (SEEDbody), DART-Fc, scFv-CH3(kih), TriFabs; Fc fusions, e.g. Di-diabody, scDb-Fc, taFv-Fc, scFv-Fc-scFv, HCAb-VHH, Fab-scFv-Fc, scFv4-Ig, scFv2-Fcab; CH3 fusions, e.g. Dia-diabody, scDb-CH3; IgE/lgM CH2 fusions, e.g. scFv-EHD2-scFv, scFvMHD2-scFv; Fab fusion proteins, e.g. Fab-scFv (bibody), Fab-scFv2 (tribody), Fab-Fv, Fab-dsFv, Fab-VHH, orthogonal Fab-Fab; non-Ig fusion proteins, e.g. DNL-Fab3, DNL-Fab2-scFv, DNL-Fab₂-IgG-cytokine₂; asymmetric IgG or IgG-like molecules, e.g. IgG(kih), IgG(kih) common LC, ZW1 IgG common LC, Biclonics common LC, CrossMab, CrossMab(kih), scFab-IgG(kih), Fab-scFab-IgG(kih), orthogonal Fab IgG(kih), DuetMab, CH3 charge pairs+CH1/CL charge pairs, hinge/CH3 charge pairs, SEED-body, Duobody, four-in-one-CrossMab (kih), LUZ-Y common LC; LUZ-Y scFab-IgG, FcFc*; appended and Fc-modified IgGs, e.g. IgG(kih)-Fv, IgG HA-TF-Fv, IgG(kih) scFab, scFab-Fc(kih)-scFv2, scFab-Fc(kih)-scFv, half DVD-Ig, DVI-Ig (four-in-one), CrossMab-Fab; modified Fc and CH3 fusion proteins, e.g. Fab-Fc(kih)-scFv, Fab-scFv-Fc(kih), Fab-scFv-Fc (BEAT), Fab-scFv-Fc-SEEDbody, TriFab; appended IgGs-HC fusions, e.g. IgG-HC, scFv, IgG-dAb, IgG-taFV, IgG-CrossFab, IgG-orthogonal Fab, IgG-(CαCβ) Fab, scFv-HC-IgG, tandem Fab-IgG (orthogonal Fab), Fab-IgG(CαCβ Fab), Fab-IgG(CR3), Fab-hinge-IgG(CR3); appended IgGs-LC fusions, e.g. IgG-scFv(LC), scFv(LC)-IgG, dAb-IgG; appended IgGs-HC and LC fusions, e.g. DVD-Ig, TVD-Ig, CODV-Ig, scFv₄-IgG, Zybody; Fc fusions, e.g. Fab-scFv-Fc, scFv₄-Ig; F(ab′)2 fusions, e.g. F(ab′)₂-scFv₂; CH1/CL fusion proteins e.g. scFV₂-CH1-hinge/CL; modified IgGs, e.g. DAF (two-in one-IgG), DutaMab, Mab²; and non-Ig fusions, e.g. DNL-Fab₄-IgG. The skilled person is readily able to design and produce multispecific antigen-binding molecules.

The present disclosure also provides Chimeric Antigen Receptors (CARs). CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The antigen-binding domain of a CAR according to the present disclosure comprises or consists of an antigen-binding molecule that binds to a sarbecovirus spike protein (e.g. a SARSr-CoV spike protein; e.g. SARS-CoV-2 spike protein and/or one or more SARS-CoV-2 variant spike proteins), as described herein. Accordingly, a CAR according to the present disclosure comprises an antigen-binding molecule as described herein.

It will be appreciated that an antigen-binding molecule according to the present disclosure forms, or is comprised in, the antigen-binding domain of the CAR. Accordingly, in some embodiments, the antigen-binding molecule of the present disclosure is comprised in a CAR.

It will also be appreciated that an antigen-binding molecule according to the present disclosure may be a CAR. A CAR having an antigen-binding domain comprising or consisting of an antigen-binding molecule of the present disclosure (e.g. a sarbecovirus spike protein (e.g. a SARS-COV spike protein; e.g. SARS-CoV-2 spike protein and/or one or more SARS-CoV-2 variant spike proteins)-binding Fv) is an antigen-binding molecule. The antigen-binding domain of the CAR of the present disclosure may be provided with any suitable format, e.g. scFv, scFab, etc.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-7, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases CARs are engineered to provide for co-stimulation of different intracellular signalling pathways. For example, signalling associated with CD28 costimulation preferentially activates the phosphatidylinositol 3-kinase (PI3K) pathway, whereas the 4-1BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1BB, ICOS and CD27.

An optional hinge region may provide separation between the antigen-binding domain and the transmembrane domain, and may act as a flexible linker. Hinge regions may be derived from IgG1 or IgG4. In some embodiments, the CAR of the present disclosure comprises a hinge region comprising or consisting of an amino acid sequence which comprises, consists of, or is derived from, the amino acid sequence of the hinge region of IgG1 or IgG4.

Also provided is a cell comprising a CAR according to the present disclosure. The CAR according to the present disclosure may be used to generate CAR-expressing immune cells, e.g. CAR-T or CAR-NK cells. Engineering of CARs into immune cells may be performed during culture, in vitro.

### Functional Properties of the Antigen-Binding Molecules of the Disclosure

The antigen-binding molecules described herein may be characterised by reference to certain functional properties. In some embodiments, the antigen-binding molecule described herein may possess one or more of the following properties:
binds to a sarbecovirus spike protein (e.g. a SARSr-CoV spike protein; e.g. SARS-CoV-2 spike protein and/or one or more SARS-CoV-2 variant spike proteins);
inhibits interaction between a sarbecovirus spike protein (e.g. a SARSr-CoV spike protein; e.g. SARS-CoV-2 spike protein and/or one or more SARS-CoV-2 variant spike proteins) and ACE2; and/or
inhibits infection of ACE2-expressing cells by a sarbecovirus (e.g. a SARSr-CoV; e.g. SARS-CoV-2 and/or one or more SARS-CoV-2 variants).

It will be appreciated that a given antigen-binding molecule may display more than one of the properties recited in the preceding paragraph. A given antigen-binding molecule may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject.

Where assays are cell-based assays, they may comprise treating cells with a given antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of a given antigen-binding molecule (e.g. a dilution series). It will be appreciated that the cells preferably express the target antigen for the antigen-binding molecule (i.e. a sarbecovirus spike protein (e.g. SARS-CoV-2 spike protein/a SARS-CoV-2 variant spike protein)).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’.

Depending on the property, the EC₅₀ may also be referred to as the ‘half-maximal inhibitory concentration’ or ‘IC₅₀’, this being the concentration of the agent at which 50% of the maximal level of inhibition of a given property is observed.

The antigen-binding molecules described herein bind to a sarbecovirus spike protein (e.g. a SARSr-CoV spike protein; e.g. SARS-CoV-2 spike protein and/or one or more SARS-CoV-2 variant spike proteins).

The ability of a given antigen-binding molecule to bind specifically to a given peptide/polypeptide can be determined by analysis according to methods known in the art, such as by ELISA, Surface Plasmon Resonance (SPR; see e.g. Hearty et al., Methods Mol Biol (2012) 907:411-442), Bio-Layer Interferometry (BLI; see e.g. Lad et al., (2015) J Biomol Screen 20(4): 498-507), flow cytometry, or by a radiolabelled antigen-binding assay (RIA) enzyme-linked immunosorbent assay. Through such analysis binding to a given molecule can be measured and quantified. In some embodiments, the binding may be the response detected in a given assay.

In some embodiments, an antigen-binding molecule according to the present disclosure binds to SARS-CoV-2 spike protein. In some embodiments, an antigen-binding molecule according to the present disclosure binds to a polypeptide comprising, or consisting of, the amino acid sequence of SEQ ID NO:1.

In some embodiments, an antigen-binding molecule according to the present disclosure binds to a SARS-CoV-2 variant spike protein as described herein. In some embodiments, an antigen-binding molecule according to the present disclosure binds to a polypeptide comprising, or consisting of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:1, wherein the amino acid sequence is non-identical to SEQ ID NO:1. In some embodiments, an antigen-binding molecule according to the present disclosure binds to a polypeptide comprising, or consisting of, SEQ ID NO:11, 12, 13, 14, 15, 16, 17, 18, 19, 20, 21, 22, 23, 24, 25, 26 or 27.

In some embodiments, an antigen-binding molecule binds to a polypeptide comprising, or consisting of, SEQ ID NO:18. In some embodiments, an antigen-binding molecule binds to a polypeptide comprising, or consisting of, SEQ ID NO:19. In some embodiments, an antigen-binding molecule binds to a polypeptide comprising, or consisting of, SEQ ID NO:20. In some embodiments, an antigen-binding molecule binds to a polypeptide comprising, or consisting of, SEQ ID NO:21. In some embodiments, an antigen-binding molecule binds to a polypeptide comprising, or consisting of, SEQ ID NO:26. In some embodiments, an antigen-binding molecule binds to a polypeptide comprising, or consisting of, SEQ ID NO:27.

In some embodiments, an antigen-binding molecule according to the present disclosure binds to a polypeptide comprising, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:7, wherein the amino acid sequence is non-identical to SEQ ID NO:7. In some embodiments, an antigen-binding molecule according to the present disclosure binds to a polypeptide comprising, or consisting of, SEQ ID NO:686, 687, 688, 689, 690, 691, 692, 693, 694, 695, 696, 697, 698, 699, 700, 701 or 702.

In some embodiments, an antigen-binding molecule binds to a polypeptide comprising SEQ ID NO:693. In some embodiments, an antigen-binding molecule binds to a polypeptide comprising SEQ ID NO:694.

In some embodiments, an antigen-binding molecule binds to a polypeptide comprising SEQ ID NO:695. In some embodiments, an antigen-binding molecule binds to a polypeptide comprising SEQ ID NO:696. In some embodiments, an antigen-binding molecule binds to a polypeptide comprising SEQ ID NO:697. In some embodiments, an antigen-binding molecule binds to a polypeptide comprising SEQ ID NO:698.

In some embodiments, an antigen-binding molecule according to the present disclosure binds to a polypeptide comprising, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:8, wherein the amino acid sequence is non-identical to SEQ ID NO:8. In some embodiments, an antigen-binding molecule according to the present disclosure binds to a polypeptide comprising, or consisting of, SEQ ID NO:703, 704, 705, 706, 707, 708, 709, 710, 711, 712, 713, 714, 715, 716, 717, 718 or 719.

In some embodiments, an antigen-binding molecule binds to a polypeptide comprising SEQ ID NO:710. In some embodiments, an antigen-binding molecule binds to a polypeptide comprising SEQ ID NO:711. In some embodiments, an antigen-binding molecule binds to a polypeptide comprising SEQ ID NO:712. In some embodiments, an antigen-binding molecule binds to a polypeptide comprising SEQ ID NO:713.

In some embodiments, an antigen-binding molecule binds to a polypeptide comprising SEQ ID NO:714. In some embodiments, an antigen-binding molecule binds to a polypeptide comprising SEQ ID NO:715.

In some embodiments, an antigen-binding molecule according to the present disclosure is capable of binding (independently) to two or more (e.g. 2, 3, 4, 5, 6, 7, 8, 9, 10, 11, 12, 13, 14, 15 or more) proteins selected from SARS-CoV-2 spike protein and SARS-CoV-2 variant spike proteins. That is, in some embodiments, an antigen-binding molecule that binds to a given (first) protein selected from SARS-CoV-2 spike protein and a SARS-CoV-2 variant spike protein also binds to one or more further (second, third, etc.) proteins selected from SARS-CoV-2 spike protein and a SARS-CoV-2 variant spike protein, wherein the one or more further proteins have an amino sequence which is different to the amino acid sequence of the first protein. Such antigen-binding molecules may be described as being ‘cross-reactive’ for the first and further proteins, or may be said to display ‘cross-reactivity’ or ‘cross-reactive binding’, or to ‘bind cross-reactively’ to the first and further proteins.

In some embodiments, an antigen-binding molecule according to the present disclosure binds cross-reactively to two or more (e.g. 2, 3, 4, 5, 6, 7, 8, 9, 10, 11, 12, 13, 14, 15, 16, 17 or all 18) polypeptides selected from: a polypeptide consisting of the amino acid sequence of SEQ ID NO:1, a polypeptide consisting of the amino acid sequence of SEQ ID NO:11, a polypeptide consisting of the amino acid sequence of SEQ ID NO:12, a polypeptide consisting of the amino acid sequence of SEQ ID NO:13, a polypeptide consisting of the amino acid sequence of SEQ ID NO:14, a polypeptide consisting of the amino acid sequence of SEQ ID NO:15, a polypeptide consisting of the amino acid sequence of SEQ ID NO:16, a polypeptide consisting of the amino acid sequence of SEQ ID NO:17, a polypeptide consisting of the amino acid sequence of SEQ ID NO:18, a polypeptide consisting of the amino acid sequence of SEQ ID NO:19, a polypeptide consisting of the amino acid sequence of SEQ ID NO:20, a polypeptide consisting of the amino acid sequence of SEQ ID NO:21, a polypeptide consisting of the amino acid sequence of SEQ ID NO:22, a polypeptide consisting of the amino acid sequence of SEQ ID NO:23, a polypeptide consisting of the amino acid sequence of SEQ ID NO:24, a polypeptide consisting of the amino acid sequence of SEQ ID NO:25, a polypeptide consisting of the amino acid sequence of SEQ ID NO:26, and a polypeptide consisting of the amino acid sequence of SEQ ID NO:27.

In some embodiments, an antigen-binding molecule according to the present disclosure binds cross-reactively to two or more (e.g. 2, 3, 4, 5, 6, 7, 8, 9, 10, 11, 12, 13, 14, 15, 16, 17 or all 18) polypeptides selected from: a polypeptide comprising the amino acid sequence of SEQ ID NO:7, a polypeptide comprising the amino acid sequence of SEQ ID NO:686, a polypeptide comprising the amino acid sequence of SEQ ID NO:687, a polypeptide comprising the amino acid sequence of SEQ ID NO:688, a polypeptide comprising the amino acid sequence of SEQ ID NO:689, a polypeptide comprising the amino acid sequence of SEQ ID NO:690, a polypeptide comprising the amino acid sequence of SEQ ID NO:691, a polypeptide comprising the amino acid sequence of SEQ ID NO:692, a polypeptide comprising the amino acid sequence of SEQ ID NO:693, a polypeptide comprising the amino acid sequence of SEQ ID NO:694, a polypeptide comprising the amino acid sequence of SEQ ID NO:695, a polypeptide comprising the amino acid sequence of SEQ ID NO:696, a polypeptide comprising the amino acid sequence of SEQ ID NO:697, a polypeptide comprising the amino acid sequence of SEQ ID NO:698, a polypeptide comprising the amino acid sequence of SEQ ID NO:699, a polypeptide comprising the amino acid sequence of SEQ ID NO:700, a polypeptide comprising the amino acid sequence of SEQ ID NO:701, and a polypeptide comprising the amino acid sequence of SEQ ID NO:702.

In some embodiments, an antigen-binding molecule according to the present disclosure binds cross-reactively to two or more (e.g. 2, 3, 4, 5, 6, 7, 8, 9, 10, 11, 12, 13, 14, 15, 16, 17 or all 18) polypeptides selected from: a polypeptide comprising the amino acid sequence of SEQ ID NO:8, a polypeptide comprising the amino acid sequence of SEQ ID NO:703, a polypeptide comprising the amino acid sequence of SEQ ID NO:704, a polypeptide comprising the amino acid sequence of SEQ ID NO:705, a polypeptide comprising the amino acid sequence of SEQ ID NO:706, a polypeptide comprising the amino acid sequence of SEQ ID NO:707, a polypeptide comprising the amino acid sequence of SEQ ID NO:708, a polypeptide comprising the amino acid sequence of SEQ ID NO:709, a polypeptide comprising the amino acid sequence of SEQ ID NO:710, a polypeptide comprising the amino acid sequence of SEQ ID NO:711, a polypeptide comprising the amino acid sequence of SEQ ID NO:712, a polypeptide comprising the amino acid sequence of SEQ ID NO:713, a polypeptide comprising the amino acid sequence of SEQ ID NO:714, a polypeptide comprising the amino acid sequence of SEQ ID NO:715, a polypeptide comprising the amino acid sequence of SEQ ID NO:716, a polypeptide comprising the amino acid sequence of SEQ ID NO:717, a polypeptide comprising the amino acid sequence of SEQ ID NO:718, and a polypeptide comprising the amino acid sequence of SEQ ID NO:719.

In preferred embodiments, an antigen-binding molecule according to the present disclosure binds (i.e. cross-reactively) to: a polypeptide comprising, or consisting of, the amino acid sequence of SEQ ID NO:1, a polypeptide comprising, or consisting of, the amino acid sequence of SEQ ID NO:11, a polypeptide comprising, or consisting of, the amino acid sequence of SEQ ID NO:12, a polypeptide comprising, or consisting of, the amino acid sequence of SEQ ID NO:13, a polypeptide comprising, or consisting of, the amino acid sequence of SEQ ID NO:14, a polypeptide comprising, or consisting of, the amino acid sequence of SEQ ID NO:15, a polypeptide comprising, or consisting of, the amino acid sequence of SEQ ID NO:16 and a polypeptide comprising, or consisting of, the amino acid sequence of SEQ ID NO:17. In preferred embodiments, an antigen-binding molecule according to the present disclosure binds (i.e. cross-reactively) to: a polypeptide comprising, or consisting of, the amino acid sequence of SEQ ID NO:1, a polypeptide comprising, or consisting of, the amino acid sequence of SEQ ID NO:11, a polypeptide comprising, or consisting of, the amino acid sequence of SEQ ID NO:12, a polypeptide comprising, or consisting of, the amino acid sequence of SEQ ID NO:13, a polypeptide comprising, or consisting of, the amino acid sequence of SEQ ID NO:14, a polypeptide comprising, or consisting of, the amino acid sequence of SEQ ID NO:15, a polypeptide comprising, or consisting of, the amino acid sequence of SEQ ID NO:16, a polypeptide comprising, or consisting of, the amino acid sequence of SEQ ID NO:17, a polypeptide comprising, or consisting of, the amino acid sequence of SEQ ID NO:18 and a polypeptide comprising, or consisting of, the amino acid sequence of SEQ ID NO:19. In preferred embodiments, an antigen-binding molecule according to the present disclosure binds (i.e. cross-reactively) to: a polypeptide comprising, or consisting of, the amino acid sequence of SEQ ID NO:1, a polypeptide comprising, or consisting of, the amino acid sequence of SEQ ID NO:11, a polypeptide comprising, or consisting of, the amino acid sequence of SEQ ID NO:12, a polypeptide comprising, or consisting of, the amino acid sequence of SEQ ID NO:13, a polypeptide comprising, or consisting of, the amino acid sequence of SEQ ID NO:14, a polypeptide comprising, or consisting of, the amino acid sequence of SEQ ID NO:15, a polypeptide comprising, or consisting of, the amino acid sequence of SEQ ID NO:16, a polypeptide comprising, or consisting of, the amino acid sequence of SEQ ID NO:17, a polypeptide comprising, or consisting of, the amino acid sequence of SEQ ID NO:18, a polypeptide comprising, or consisting of, the amino acid sequence of SEQ ID NO:19, a polypeptide comprising, or consisting of, the amino acid sequence of SEQ ID NO:20, a polypeptide comprising, or consisting of, the amino acid sequence of SEQ ID NO:21, a polypeptide comprising, or consisting of, the amino acid sequence of SEQ ID NO:26 and a polypeptide comprising, or consisting of, the amino acid sequence of SEQ ID NO:27.

In preferred embodiments, an antigen-binding molecule according to the present disclosure binds (i.e. cross-reactively) to: a polypeptide comprising the amino acid sequence of SEQ ID NO:7, a polypeptide comprising the amino acid sequence of SEQ ID NO:686, a polypeptide comprising the amino acid sequence of SEQ ID NO:687, a polypeptide comprising the amino acid sequence of SEQ ID NO:688, a polypeptide comprising the amino acid sequence of SEQ ID NO:689, a polypeptide comprising the amino acid sequence of SEQ ID NO:690, a polypeptide comprising the amino acid sequence of SEQ ID NO:691 and a polypeptide comprising the amino acid sequence of SEQ ID NO:692. In preferred embodiments, an antigen-binding molecule according to the present disclosure binds (i.e. cross-reactively) to: a polypeptide comprising the amino acid sequence of SEQ ID NO:7, a polypeptide comprising the amino acid sequence of SEQ ID NO:686, a polypeptide comprising the amino acid sequence of SEQ ID NO:687, a polypeptide comprising the amino acid sequence of SEQ ID NO:688, a polypeptide comprising the amino acid sequence of SEQ ID NO:689, a polypeptide comprising the amino acid sequence of SEQ ID NO:690, a polypeptide comprising the amino acid sequence of SEQ ID NO:691, a polypeptide comprising the amino acid sequence of SEQ ID NO:692, a polypeptide comprising the amino acid sequence of SEQ ID NO:693 and a polypeptide comprising the amino acid sequence of SEQ ID NO:694. In preferred embodiments, an antigen-binding molecule according to the present disclosure binds (i.e. cross-reactively) to: a polypeptide comprising the amino acid sequence of SEQ ID NO:7, a polypeptide comprising the amino acid sequence of SEQ ID NO:686, a polypeptide comprising the amino acid sequence of SEQ ID NO:687, a polypeptide comprising the amino acid sequence of SEQ ID NO:688, a polypeptide comprising the amino acid sequence of SEQ ID NO:689, a polypeptide comprising the amino acid sequence of SEQ ID NO:690, a polypeptide comprising the amino acid sequence of SEQ ID NO:691, a polypeptide comprising the amino acid sequence of SEQ ID NO:692, a polypeptide comprising the amino acid sequence of SEQ ID NO:693, a polypeptide comprising the amino acid sequence of SEQ ID NO:694, a polypeptide comprising the amino acid sequence of SEQ ID NO:695, a polypeptide comprising the amino acid sequence of SEQ ID NO:696, a polypeptide comprising the amino acid sequence of SEQ ID NO:697 and a polypeptide comprising the amino acid sequence of SEQ ID NO:698.

In preferred embodiments, an antigen-binding molecule according to the present disclosure binds (i.e. cross-reactively) to: a polypeptide comprising the amino acid sequence of SEQ ID NO:8, a polypeptide comprising the amino acid sequence of SEQ ID NO:703, a polypeptide comprising the amino acid sequence of SEQ ID NO:704, a polypeptide comprising the amino acid sequence of SEQ ID NO:705, a polypeptide comprising the amino acid sequence of SEQ ID NO:706, a polypeptide comprising the amino acid sequence of SEQ ID NO:707, a polypeptide comprising the amino acid sequence of SEQ ID NO:708 and a polypeptide comprising the amino acid sequence of SEQ ID NO:709. In preferred embodiments, an antigen-binding molecule according to the present disclosure binds (i.e. cross-reactively) to: a polypeptide comprising the amino acid sequence of SEQ ID NO:8, a polypeptide comprising the amino acid sequence of SEQ ID NO:703, a polypeptide comprising the amino acid sequence of SEQ ID NO:704, a polypeptide comprising the amino acid sequence of SEQ ID NO:705, a polypeptide comprising the amino acid sequence of SEQ ID NO:706, a polypeptide comprising the amino acid sequence of SEQ ID NO:707, a polypeptide comprising the amino acid sequence of SEQ ID NO:708, a polypeptide comprising the amino acid sequence of SEQ ID NO:709, a polypeptide comprising the amino acid sequence of SEQ ID NO:710 and a polypeptide comprising the amino acid sequence of SEQ ID NO:711. In preferred embodiments, an antigen-binding molecule according to the present disclosure binds (i.e. cross-reactively) to: a polypeptide comprising the amino acid sequence of SEQ ID NO:8, a polypeptide comprising the amino acid sequence of SEQ ID NO:703, a polypeptide comprising the amino acid sequence of SEQ ID NO:704, a polypeptide comprising the amino acid sequence of SEQ ID NO:705, a polypeptide comprising the amino acid sequence of SEQ ID NO:706, a polypeptide comprising the amino acid sequence of SEQ ID NO:707, a polypeptide comprising the amino acid sequence of SEQ ID NO:708, a polypeptide comprising the amino acid sequence of SEQ ID NO:709, a polypeptide comprising the amino acid sequence of SEQ ID NO:710, a polypeptide comprising the amino acid sequence of SEQ ID NO:711, a polypeptide comprising the amino acid sequence of SEQ ID NO:712, a polypeptide comprising the amino acid sequence of SEQ ID NO:713, a polypeptide comprising the amino acid sequence of SEQ ID NO:714 and a polypeptide comprising the amino acid sequence of SEQ ID NO:715.

The antigen-binding molecules and antigen-binding domains described herein preferably display specific binding to a sarbecovirus spike protein (e.g. a SARSr-CoV spike protein; e.g. SARS-CoV-2 spike protein and/or one or more SARS-CoV-2 variant spike proteins). As used herein, ‘specific binding’ refers to binding which is selective for the antigen, and which can be discriminated from non-specific binding to non-target antigen. An antigen-binding molecule/domain that specifically binds to a target molecule preferably binds the target with greater affinity, and/or with greater duration than it binds to other, non-target molecules.

In some embodiments, the extent of binding of the antigen-binding molecule to a non-target molecule is less than about 10% of the binding of the antibody to the target molecule as measured, e.g. by ELISA, SPR, BLI or by RIA. Alternatively, binding specificity may be reflected in terms of binding affinity where the antigen-binding molecule binds with a dissociation constant (K_{D}) that is at least 0.1 order of magnitude (i.e. 0.1×10ⁿ, where n is an integer representing the order of magnitude) greater than the K_{D} of the antigen-binding molecule towards a non-target molecule. This may optionally be one of at least 0.2, 0.3, 0.4, 0.5, 0.6, 0.7, 0.8, 0.9, 1.0, 1.5, or 2.0.

In some embodiments, the antigen-binding molecule described herein binds to a sarbecovirus spike protein (e.g. a SARSr-CoV spike protein; e.g. SARS-CoV-2 spike protein and/or one or more SARS-CoV-2 variant spike proteins) with an affinity in the micromolar range, i.e. K_{D}=9.9×10⁻⁴ to 1×10⁻⁶ M. In some embodiments, the antigen-binding molecule described herein binds to a sarbecovirus spike protein (e.g. a SARSr-CoV spike protein; e.g. SARS-CoV-2 spike protein and/or one or more SARS-CoV-2 variant spike proteins) with sub-micromolar affinity, i.e. K_{D}<1×10⁻⁶ M. In some embodiments, the antigen-binding molecule described herein binds to a sarbecovirus spike protein (e.g. a SARSr-CoV spike protein; e.g. SARS-CoV-2 spike protein and/or one or more SARS-CoV-2 variant spike proteins) with an affinity in the nanomolar range, i.e. K_{D}=9.9×10⁻⁷ to 1×10⁻⁹ M. In some embodiments, the antigen-binding molecule described herein binds to a given SARS-CoV-2 spike protein with sub-nanomolar affinity, i.e. K_{D}<1×10⁻⁹ M. In some embodiments, the antigen-binding molecule described herein binds to a sarbecovirus spike protein (e.g. a SARSr-CoV spike protein; e.g. SARS-CoV-2 spike protein and/or one or more SARS-CoV-2 variant spike proteins) with an affinity in the picomolar range, i.e. K_{D}=9.9×10⁻¹⁰ to 1×10⁻¹² M. In some embodiments, the antigen-binding molecule described herein binds to a sarbecovirus spike protein (e.g. a SARSr-CoV spike protein; e.g. SARS-CoV-2 spike protein and/or one or more SARS-CoV-2 variant spike proteins) with sub-picomolar affinity, i.e. K_{D}<1×10⁻¹² M.

The antigen-binding molecules of the present disclosure may bind to a particular region of interest of a sarbecovirus spike protein (e.g. a SARSr-CoV spike protein; e.g. SARS-CoV-2 spike protein and/or one or more SARS-CoV-2 variant spike proteins). Antigen-binding molecules according to the present disclosure may bind to linear epitope of a sarbecovirus spike protein (e.g. a SARSr-CoV spike protein; e.g. SARS-CoV-2 spike protein and/or one or more SARS-CoV-2 variant spike proteins), consisting of a contiguous sequence of amino acids (i.e. an amino acid primary sequence). In some embodiments, an antigen-binding molecules may bind to a conformational epitope of a sarbecovirus spike protein (e.g. a SARSr-CoV spike protein; e.g. SARS-CoV-2 spike protein and/or one or more SARS-CoV-2 variant spike proteins), consisting of a discontinuous sequence of amino acids of the amino acid sequence.

The region of a given target molecule to which an antigen-binding molecule binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, the antigen-binding molecule is capable of binding the same region, or an overlapping region, of a sarbecovirus spike protein (e.g. a SARSr-CoV spike protein; e.g. SARS-CoV-2 spike protein and/or one or more SARS-CoV-2 variant spike proteins), to the region bound by an antibody comprising the VH and VL regions of an antibody as indicated in Table C.

Whether a test antigen-binding molecule binds to the same or an overlapping region of a given target as a reference antigen-binding molecule can be evaluated, for example, by analysis of (i) interaction between the test antigen-binding molecule and the target in the absence of the reference binding molecule, and (ii) interaction between the test antigen-binding molecule in the presence of the reference antigen-binding molecule, or following incubation of the target with the reference antigen-binding molecule. Determination of a reduced level of interaction between the test antigen-binding molecule and the target following analysis according to (ii) as compared to (i) might support an inference that the test and reference antigen-binding molecule bind to the same or an overlapping region of the target. Suitable assays for such analysis include e.g. competition ELISA assays and epitope binning assays.

In some embodiments, the antigen-binding molecule binds to a sarbecovirus spike protein (e.g. a SARSr-CoV spike protein; e.g. SARS-CoV-2 spike protein and/or one or more SARS-CoV-2 variant spike proteins) in the region which is bound by an interaction partner for the protein, e.g. ACE2. In some embodiments, the antigen-binding molecule reduces/inhibits interaction between a sarbecovirus spike protein (e.g. a SARSr-CoV spike protein; e.g. SARS-CoV-2 spike protein and/or one or more SARS-CoV-2 variant spike proteins) and an interaction partner for the SARS-CoV-2 spike protein/SARS-CoV-2 variant spike protein(s) (e.g. ACE2). In some embodiments, the antigen-binding molecule is a competitive inhibitor of binding of an interaction partner for a sarbecovirus spike protein (e.g. a SARSr-CoV spike protein; e.g. SARS-CoV-2 spike protein and/or one or more SARS-CoV-2 variant spike proteins), e.g. ACE2, to the sarbecovirus spike protein(s). In some embodiments, the antigen-binding molecule binds to a sarbecovirus spike protein (e.g. a SARSr-CoV spike protein; e.g. SARS-CoV-2 spike protein and/or one or more SARS-CoV-2 variant spike proteins) in the region bound by a polypeptide comprising or consisting of the sequence shown in SEQ ID NO:30.

Antigen-binding molecules which inhibit interaction between ACE2 and a sarbecovirus spike protein (e.g. a SARSr-CoV spike protein; e.g. SARS-CoV-2 spike protein and/or one or more SARS-CoV-2 variant spike proteins) may be described as inhibitors/antagonists of such interaction, and may be referred to as neutralising antigen-binding molecules to a sarbecovirus (e.g. a SARSr-CoV, e.g. SARS-CoV-2 and/or one or more SARS-CoV-2 variants).

In some embodiments, an antigen-binding molecule according to the present disclosure inhibits interaction between ACE2 and SARS-CoV-2 spike protein. In some embodiments, an antigen-binding molecule according to the present disclosure inhibits interaction between ACE2 and a polypeptide comprising, or consisting of, the amino acid sequence of SEQ ID NO:1.

In some embodiments, an antigen-binding molecule inhibits interaction between ACE2 and a SARS-CoV-2 variant spike protein. In some embodiments, an antigen-binding molecule inhibits interaction between ACE2 and a polypeptide comprising, or consisting of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:1, wherein the amino acid sequence is non-identical to SEQ ID NO:1. In some embodiments, an antigen-binding molecule inhibits interaction between ACE2 and a polypeptide comprising, or consisting of, SEQ ID NO:11, 12, 13, 14, 15, 16, 17, 18, 19, 20, 21, 22, 23, 24, 25, 26 or 27.

In some embodiments, an antigen-binding molecule inhibits interaction between ACE2 and a polypeptide comprising, or consisting of, SEQ ID NO:18. In some embodiments, an antigen-binding molecule inhibits interaction between ACE2 and a polypeptide comprising, or consisting of, SEQ ID NO:19. In some embodiments, an antigen-binding molecule inhibits interaction between ACE2 and a polypeptide comprising, or consisting of, SEQ ID NO:20. In some embodiments, an antigen-binding molecule inhibits interaction between ACE2 and a polypeptide comprising, or consisting of, SEQ ID NO:21. In some embodiments, an antigen-binding molecule inhibits interaction between ACE2 and a polypeptide comprising, or consisting of, SEQ ID NO:26. In some embodiments, an antigen-binding molecule inhibits interaction between ACE2 and a polypeptide comprising, or consisting of, SEQ ID NO:27.

In some embodiments, an antigen-binding molecule inhibits interaction between ACE2 and a polypeptide comprising, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:7, wherein the amino acid sequence is non-identical to SEQ ID NO:7. In some embodiments, an antigen-binding molecule inhibits interaction between ACE2 and a polypeptide comprising, or consisting of, SEQ ID NO:686, 687, 688, 689, 690, 691, 692, 693, 694, 695, 696, 697, 698, 699, 700, 701 or 702.

In some embodiments, an antigen-binding molecule inhibits interaction between ACE2 and a polypeptide comprising SEQ ID NO:693. In some embodiments, an antigen-binding molecule inhibits interaction between ACE2 and a polypeptide comprising SEQ ID NO:694. In some embodiments, an antigen-binding molecule inhibits interaction between ACE2 and a polypeptide comprising SEQ ID NO:695. In some embodiments, an antigen-binding molecule inhibits interaction between ACE2 and a polypeptide comprising SEQ ID NO:696. In some embodiments, an antigen-binding molecule inhibits interaction between ACE2 and a polypeptide comprising SEQ ID NO:697. In some embodiments, an antigen-binding molecule inhibits interaction between ACE2 and a polypeptide comprising SEQ ID NO:698.

In some embodiments, an antigen-binding molecule inhibits interaction between ACE2 and a polypeptide comprising, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99%) amino acid sequence identity to SEQ ID NO:8, wherein the amino acid sequence is non-identical to SEQ ID NO:8. In some embodiments, an antigen-binding molecule inhibits interaction between ACE2 and a polypeptide comprising, or consisting of, SEQ ID NO:703, 704, 705, 706, 707, 708, 709, 710, 711, 712, 713, 714, 715, 716, 717, 718 or 719.

In some embodiments, an antigen-binding molecule inhibits interaction between ACE2 and a polypeptide comprising SEQ ID NO:710. In some embodiments, an antigen-binding molecule inhibits interaction between ACE2 and a polypeptide comprising SEQ ID NO:711. In some embodiments, an antigen-binding molecule inhibits interaction between ACE2 and a polypeptide comprising SEQ ID NO:712. In some embodiments, an antigen-binding molecule inhibits interaction between ACE2 and a polypeptide comprising SEQ ID NO:713. In some embodiments, an antigen-binding molecule inhibits interaction between ACE2 and a polypeptide comprising SEQ ID NO:714. In some embodiments, an antigen-binding molecule inhibits interaction between ACE2 and a polypeptide comprising SEQ ID NO:715.

In some embodiments, an antigen-binding molecule according to the present disclosure is capable of inhibiting interaction between ACE2 and two or more (e.g. 2, 3, 4, 5, 6, 7, 8, 9, 10, 11, 12, 13, 14, 15 or more) proteins (independently) selected from SARS-CoV-2 spike protein and SARS-CoV-2 variant spike proteins. That is, in some embodiments, an antigen-binding molecule that inhibits interaction between ACE2 and a given (first) protein selected from SARS-CoV-2 spike protein and a SARS-CoV-2 variant spike protein also inhibits interaction between ACE2 and one or more further (second, third, etc.) proteins selected from SARS-CoV-2 spike protein and a SARS-CoV-2 variant spike protein, wherein the one or more further proteins have an amino sequence which is different to the amino acid sequence of the first protein.

In some embodiments, an antigen-binding molecule according to the present disclosure inhibits interaction between ACE2 and two or more (e.g. 2, 3, 4, 5, 6, 7, 8, 9, 10, 11, 12, 13, 14, 15, 16, 17 or all 18) polypeptides selected from: a polypeptide consisting of the amino acid sequence of SEQ ID NO:1, a polypeptide consisting of the amino acid sequence of SEQ ID NO:11, a polypeptide consisting of the amino acid sequence of SEQ ID NO:12, a polypeptide consisting of the amino acid sequence of SEQ ID NO:13, a polypeptide consisting of the amino acid sequence of SEQ ID NO:14, a polypeptide consisting of the amino acid sequence of SEQ ID NO:15, a polypeptide consisting of the amino acid sequence of SEQ ID NO:16, a polypeptide consisting of the amino acid sequence of SEQ ID NO:17, a polypeptide consisting of the amino acid sequence of SEQ ID NO:18, a polypeptide consisting of the amino acid sequence of SEQ ID NO:19, a polypeptide consisting of the amino acid sequence of SEQ ID NO:20, a polypeptide consisting of the amino acid sequence of SEQ ID NO:21, a polypeptide consisting of the amino acid sequence of SEQ ID NO:22, a polypeptide consisting of the amino acid sequence of SEQ ID NO:23, a polypeptide consisting of the amino acid sequence of SEQ ID NO:24, a polypeptide consisting of the amino acid sequence of SEQ ID NO:25, a polypeptide consisting of the amino acid sequence of SEQ ID NO:26, and a polypeptide consisting of the amino acid sequence of SEQ ID NO:27.

In some embodiments, an antigen-binding molecule according to the present disclosure inhibits interaction between ACE2 and two or more (e.g. 2, 3, 4, 5, 6, 7, 8, 9, 10, 11, 12, 13, 14, 15, 16, 17 or all 18) polypeptides selected from: a polypeptide comprising the amino acid sequence of SEQ ID NO:7, a polypeptide comprising the amino acid sequence of SEQ ID NO:686, a polypeptide comprising the amino acid sequence of SEQ ID NO:687, a polypeptide comprising the amino acid sequence of SEQ ID NO:688, a polypeptide comprising the amino acid sequence of SEQ ID NO:689, a polypeptide comprising the amino acid sequence of SEQ ID NO:690, a polypeptide comprising the amino acid sequence of SEQ ID NO:691, a polypeptide comprising the amino acid sequence of SEQ ID NO:692, a polypeptide comprising the amino acid sequence of SEQ ID NO:693, a polypeptide comprising the amino acid sequence of SEQ ID NO:694, a polypeptide comprising the amino acid sequence of SEQ ID NO:695, a polypeptide comprising the amino acid sequence of SEQ ID NO:696, a polypeptide comprising the amino acid sequence of SEQ ID NO:697, a polypeptide comprising the amino acid sequence of SEQ ID NO:698, a polypeptide comprising the amino acid sequence of SEQ ID NO:699, a polypeptide comprising the amino acid sequence of SEQ ID NO:700, a polypeptide comprising the amino acid sequence of SEQ ID NO:701, and a polypeptide comprising the amino acid sequence of SEQ ID NO:702.

In some embodiments, an antigen-binding molecule according to the present disclosure inhibits interaction between ACE2 and two or more (e.g. 2, 3, 4, 5, 6, 7, 8, 9, 10, 11, 12, 13, 14, 15, 16, 17 or all 18) polypeptides selected from: a polypeptide comprising the amino acid sequence of SEQ ID NO:8, a polypeptide comprising the amino acid sequence of SEQ ID NO:703, a polypeptide comprising the amino acid sequence of SEQ ID NO:704, a polypeptide comprising the amino acid sequence of SEQ ID NO:705, a polypeptide comprising the amino acid sequence of SEQ ID NO:706, a polypeptide comprising the amino acid sequence of SEQ ID NO:707, a polypeptide comprising the amino acid sequence of SEQ ID NO:708, a polypeptide comprising the amino acid sequence of SEQ ID NO:709, a polypeptide comprising the amino acid sequence of SEQ ID NO:710, a polypeptide comprising the amino acid sequence of SEQ ID NO:711, a polypeptide comprising the amino acid sequence of SEQ ID NO:712, a polypeptide comprising the amino acid sequence of SEQ ID NO:713, a polypeptide comprising the amino acid sequence of SEQ ID NO:714, a polypeptide comprising the amino acid sequence of SEQ ID NO:715, a polypeptide comprising the amino acid sequence of SEQ ID NO:716, a polypeptide comprising the amino acid sequence of SEQ ID NO:717, a polypeptide comprising the amino acid sequence of SEQ ID NO:718, and a polypeptide comprising the amino acid sequence of SEQ ID NO:719.

In preferred embodiments, an antigen-binding molecule according to the present disclosure: inhibits interaction between ACE2 and a polypeptide comprising, or consisting of, the amino acid sequence of SEQ ID NO:1; and inhibits interaction between ACE2 and a polypeptide comprising, or consisting of, the amino acid sequence of SEQ ID NO:11; and inhibits interaction between ACE2 and a polypeptide comprising, or consisting of, the amino acid sequence of SEQ ID NO:12; and inhibits interaction between ACE2 and a polypeptide comprising, or consisting of, the amino acid sequence of SEQ ID NO:13; and inhibits interaction between ACE2 and a polypeptide comprising, or consisting of, the amino acid sequence of SEQ ID NO:14; and inhibits interaction between ACE2 and a polypeptide comprising, or consisting of, the amino acid sequence of SEQ ID NO:15; and inhibits interaction between ACE2 and a polypeptide comprising, or consisting of, the amino acid sequence of SEQ ID NO:16; and inhibits interaction between ACE2 and a polypeptide comprising, or consisting of, the amino acid sequence of SEQ ID NO:17. In preferred embodiments, an antigen-binding molecule according to the present disclosure: inhibits interaction between ACE2 and a polypeptide comprising, or consisting of, the amino acid sequence of SEQ ID NO:1; and inhibits interaction between ACE2 and a polypeptide comprising, or consisting of, the amino acid sequence of SEQ ID NO:11; and inhibits interaction between ACE2 and a polypeptide comprising, or consisting of, the amino acid sequence of SEQ ID NO:12; and inhibits interaction between ACE2 and a polypeptide comprising, or consisting of, the amino acid sequence of SEQ ID NO:13; and inhibits interaction between ACE2 and a polypeptide comprising, or consisting of, the amino acid sequence of SEQ ID NO:14; and inhibits interaction between ACE2 and a polypeptide comprising, or consisting of, the amino acid sequence of SEQ ID NO:15; and inhibits interaction between ACE2 and a polypeptide comprising, or consisting of, the amino acid sequence of SEQ ID NO:16; and inhibits interaction between ACE2 and a polypeptide comprising, or consisting of, the amino acid sequence of SEQ ID NO:17; and inhibits interaction between ACE2 and a polypeptide comprising, or consisting of, the amino acid sequence of SEQ ID NO:18; and inhibits interaction between ACE2 and a polypeptide comprising, or consisting of, the amino acid sequence of SEQ ID NO:19. In preferred embodiments, an antigen-binding molecule according to the present disclosure: inhibits interaction between ACE2 and a polypeptide comprising, or consisting of, the amino acid sequence of SEQ ID NO:1; and inhibits interaction between ACE2 and a polypeptide comprising, or consisting of, the amino acid sequence of SEQ ID NO:11; and inhibits interaction between ACE2 and a polypeptide comprising, or consisting of, the amino acid sequence of SEQ ID NO:12; and inhibits interaction between ACE2 and a polypeptide comprising, or consisting of, the amino acid sequence of SEQ ID NO:13; and inhibits interaction between ACE2 and a polypeptide comprising, or consisting of, the amino acid sequence of SEQ ID NO:14; and inhibits interaction between ACE2 and a polypeptide comprising, or consisting of, the amino acid sequence of SEQ ID NO:15; and inhibits interaction between ACE2 and a polypeptide comprising, or consisting of, the amino acid sequence of SEQ ID NO:16; and inhibits interaction between ACE2 and a polypeptide comprising, or consisting of, the amino acid sequence of SEQ ID NO:17; and inhibits interaction between ACE2 and a polypeptide comprising, or consisting of, the amino acid sequence of SEQ ID NO:18; and inhibits interaction between ACE2 and a polypeptide comprising, or consisting of, the amino acid sequence of SEQ ID NO:19; and inhibits interaction between ACE2 and a polypeptide comprising, or consisting of, the amino acid sequence of SEQ ID NO:20; and inhibits interaction between ACE2 and a polypeptide comprising, or consisting of, the amino acid sequence of SEQ ID NO:21; and inhibits interaction between ACE2 and a polypeptide comprising, or consisting of, the amino acid sequence of SEQ ID NO:26 and inhibits interaction between ACE2 and a polypeptide comprising, or consisting of, the amino acid sequence of SEQ ID NO:27.

In preferred embodiments, an antigen-binding molecule according to the present disclosure: inhibits interaction between ACE2 and a polypeptide comprising the amino acid sequence of SEQ ID NO:7; and inhibits interaction between ACE2 and a polypeptide comprising the amino acid sequence of SEQ ID NO:686; and inhibits interaction between ACE2 and a polypeptide comprising the amino acid sequence of SEQ ID NO:687; and inhibits interaction between ACE2 and a polypeptide comprising the amino acid sequence of SEQ ID NO:688; and inhibits interaction between ACE2 and a polypeptide comprising the amino acid sequence of SEQ ID NO:689; and inhibits interaction between ACE2 and a polypeptide comprising the amino acid sequence of SEQ ID NO:690; and inhibits interaction between ACE2 and a polypeptide comprising the amino acid sequence of SEQ ID NO:691; and inhibits interaction between ACE2 and a polypeptide comprising the amino acid sequence of SEQ ID NO:692. In preferred embodiments, an antigen-binding molecule according to the present disclosure: inhibits interaction between ACE2 and a polypeptide comprising the amino acid sequence of SEQ ID NO:7; and inhibits interaction between ACE2 and a polypeptide comprising the amino acid sequence of SEQ ID NO:686; and inhibits interaction between ACE2 and a polypeptide comprising the amino acid sequence of SEQ ID NO:687; and inhibits interaction between ACE2 and a polypeptide comprising the amino acid sequence of SEQ ID NO:688; and inhibits interaction between ACE2 and a polypeptide comprising the amino acid sequence of SEQ ID NO:689; and inhibits interaction between ACE2 and a polypeptide comprising the amino acid sequence of SEQ ID NO:690; and inhibits interaction between ACE2 and a polypeptide comprising the amino acid sequence of SEQ ID NO:691; and inhibits interaction between ACE2 and a polypeptide comprising the amino acid sequence of SEQ ID NO:692; and inhibits interaction between ACE2 and a polypeptide comprising the amino acid sequence of SEQ ID NO:693; and inhibits interaction between ACE2 and a polypeptide comprising the amino acid sequence of SEQ ID NO:694. In preferred embodiments, an antigen-binding molecule according to the present disclosure: inhibits interaction between ACE2 and a polypeptide comprising the amino acid sequence of SEQ ID NO:7; and inhibits interaction between ACE2 and a polypeptide comprising the amino acid sequence of SEQ ID NO:686; and inhibits interaction between ACE2 and a polypeptide comprising the amino acid sequence of SEQ ID NO:687; and inhibits interaction between ACE2 and a polypeptide comprising the amino acid sequence of SEQ ID NO:688; and inhibits interaction between ACE2 and a polypeptide comprising the amino acid sequence of SEQ ID NO:689; and inhibits interaction between ACE2 and a polypeptide comprising the amino acid sequence of SEQ ID NO:690; and inhibits interaction between ACE2 and a polypeptide comprising the amino acid sequence of SEQ ID NO:691; and inhibits interaction between ACE2 and a polypeptide comprising the amino acid sequence of SEQ ID NO:692; and inhibits interaction between ACE2 and a polypeptide comprising the amino acid sequence of SEQ ID NO:693; and inhibits interaction between ACE2 and a polypeptide comprising the amino acid sequence of SEQ ID NO:694; and inhibits interaction between ACE2 and a polypeptide comprising the amino acid sequence of SEQ ID NO:695; and inhibits interaction between ACE2 and a polypeptide comprising the amino acid sequence of SEQ ID NO:696; and inhibits interaction between ACE2 and a polypeptide comprising the amino acid sequence of SEQ ID NO:697 and a polypeptide comprising the amino acid sequence of SEQ ID NO:698.

In preferred embodiments, an antigen-binding molecule according to the present disclosure: inhibits interaction between ACE2 and a polypeptide comprising the amino acid sequence of SEQ ID NO:8; and inhibits interaction between ACE2 and a polypeptide comprising the amino acid sequence of SEQ ID NO:703; and inhibits interaction between ACE2 and a polypeptide comprising the amino acid sequence of SEQ ID NO:704; and inhibits interaction between ACE2 and a polypeptide comprising the amino acid sequence of SEQ ID NO:705; and inhibits interaction between ACE2 and a polypeptide comprising the amino acid sequence of SEQ ID NO:706; and inhibits interaction between ACE2 and a polypeptide comprising the amino acid sequence of SEQ ID NO:707; and inhibits interaction between ACE2 and a polypeptide comprising the amino acid sequence of SEQ ID NO:708; and inhibits interaction between ACE2 and a polypeptide comprising the amino acid sequence of SEQ ID NO:709. In preferred embodiments, an antigen-binding molecule according to the present disclosure: inhibits interaction between ACE2 and a polypeptide comprising the amino acid sequence of SEQ ID NO:8; and inhibits interaction between ACE2 and a polypeptide comprising the amino acid sequence of SEQ ID NO:703; and inhibits interaction between ACE2 and a polypeptide comprising the amino acid sequence of SEQ ID NO:704; and inhibits interaction between ACE2 and a polypeptide comprising the amino acid sequence of SEQ ID NO:705; and inhibits interaction between ACE2 and a polypeptide comprising the amino acid sequence of SEQ ID NO:706; and inhibits interaction between ACE2 and a polypeptide comprising the amino acid sequence of SEQ ID NO:707; and inhibits interaction between ACE2 and a polypeptide comprising the amino acid sequence of SEQ ID NO:708; and inhibits interaction between ACE2 and a polypeptide comprising the amino acid sequence of SEQ ID NO:709; and inhibits interaction between ACE2 and a polypeptide comprising the amino acid sequence of SEQ ID NO:710; and inhibits interaction between ACE2 and a polypeptide comprising the amino acid sequence of SEQ ID NO:711. In preferred embodiments, an antigen-binding molecule according to the present disclosure: inhibits interaction between ACE2 and a polypeptide comprising the amino acid sequence of SEQ ID NO:8; and inhibits interaction between ACE2 and a polypeptide comprising the amino acid sequence of SEQ ID NO:703; and inhibits interaction between ACE2 and a polypeptide comprising the amino acid sequence of SEQ ID NO:704; and inhibits interaction between ACE2 and a polypeptide comprising the amino acid sequence of SEQ ID NO:705; and inhibits interaction between ACE2 and a polypeptide comprising the amino acid sequence of SEQ ID NO:706; and inhibits interaction between ACE2 and a polypeptide comprising the amino acid sequence of SEQ ID NO:707; and inhibits interaction between ACE2 and a polypeptide comprising the amino acid sequence of SEQ ID NO:708; and inhibits interaction between ACE2 and a polypeptide comprising the amino acid sequence of SEQ ID NO:709; and inhibits interaction between ACE2 and a polypeptide comprising the amino acid sequence of SEQ ID NO:710; and inhibits interaction between ACE2 and a polypeptide comprising the amino acid sequence of SEQ ID NO:711; and inhibits interaction between ACE2 and a polypeptide comprising the amino acid sequence of SEQ ID NO:712; and inhibits interaction between ACE2 and a polypeptide comprising the amino acid sequence of SEQ ID NO:713; and inhibits interaction between ACE2 and a polypeptide comprising the amino acid sequence of SEQ ID NO:714 and a polypeptide comprising the amino acid sequence of SEQ ID NO:715.

The ability of a given antigen-binding molecule to inhibit interaction between SARS-CoV-2 spike protein and/or a given SARS-CoV-2 variant spike protein and ACE2 can be determined for example by analysis of interaction in the presence of, or following incubation of one or both of the interaction partners with, the antigen-binding molecule. An antigen-binding molecule which inhibits interaction between SARS-CoV-2 spike protein/a given SARS-CoV-2 variant spike protein and ACE2 is identified by the observation of a reduction/decrease in the level of interaction between the interaction partners in the presence of—or following incubation of the interaction partners with—the antigen-binding molecule, as compared to the level of interaction observed in the absence of the antigen-binding molecule (or in the presence of an appropriate control antigen-binding molecule known not to affect interaction between the SARS-CoV-2 spike protein/SARS-CoV-2 variant spike protein and ACE2). Suitable analysis can be performed in vitro, e.g. using recombinant interaction partners, or using cells expressing the interaction partners. Cells expressing the interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

The ability of a given antigen-binding molecule to inhibit interaction between ACE2 and a sarbecovirus spike protein (e.g. SARS-CoV-2 spike protein/a SARS-CoV-2 variant spike protein) can be evaluated in a pseudovirus neutralisation assay. Pseudovirus neutralisation assays employ e.g. vesicular stomatitis virus (VSV) or retrovirus (RV) vectors pseudotyped with SARS-CoV-2 spike protein or a SARS-CoV-2 variant spike protein. Pseudovirus neutralisation assays that may be employed to evaluate the ability of a given antigen-binding molecule to inhibit interaction between ACE2 and a sarbecovirus spike protein (e.g. SARS-CoV-2 spike protein/a SARS-CoV-2 variant spike protein) are described e.g. in Donofrio et al., Vaccines (Basel) (2021) 9(4): 389, Nie et al., Emerg. Microbes Infect. (2020) 9:680-686, Chia et al., Sci Adv. (2023) 9(30): eade3470 and Tan et al., Nature Biotechnology (2020) 38:1073-1078, all of which are hereby incorporated by reference in their entirety.

The ability of a given antigen-binding molecule to inhibit interaction between ACE2 and a sarbecovirus spike protein (e.g. SARS-CoV-2 spike protein/a SARS-CoV-2 variant spike protein) can also be evaluated in a surrogate virus neutralization test (sVNT). Surrogate virus neutralization tests investigate binding of SARS-CoV-2 spike protein/SARS-CoV-2 variant spike proteins (or a domain thereof, e.g. the RBD thereof) to ACE2, using labelled species in an ELISA-based assay, to infer inhibition of interaction. Surrogate virus neutralization tests that may be employed to evaluate the ability of a given antigen-binding molecule to inhibit interaction between ACE2 and a sarbecovirus spike protein (e.g. SARS-CoV-2 spike protein/a SARS-CoV-2 variant spike protein) are described e.g. in Chia et al., Sci Adv. (2023) 9(30):eade3470, Tan et al., Nature Biotechnology (2020) 38: 1073-1078 and Springer et al., Diagnostics (Basel). (2023) 13(13): 2278 (hereby incorporated by reference in its entirety.

In some embodiments, the ability of an antigen-binding molecule to inhibit interaction between ACE2 and a sarbecovirus spike protein (e.g. SARS-CoV-2 spike protein/a SARS-CoV-2 variant spike protein) is analysed essentially as described in Example 1.2 herein. In some embodiments, the ability of an antigen-binding molecule to inhibit interaction between ACE2 and a sarbecovirus spike protein (e.g. SARS-CoV-2 spike protein/a SARS-CoV-2 variant spike protein) is analysed essentially as described in Example 1.3 herein.

In some embodiments, an antigen-binding molecule according to the present disclosure reduces/inhibits interaction between SARS-CoV-2 spike protein/a given SARS-CoV-2 variant spike protein and ACE2 to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction between SARS-CoV-2 spike protein/the SARS-CoV-2 variant spike protein and ACE2 observed in the absence of the antigen-binding molecule (or in the presence of an appropriate control antigen-binding molecule known not to affect interaction between SARS-CoV-2 spike protein/the SARS-CoV-2 variant spike protein and ACE2).

In some embodiments, the antigen-binding molecule is capable of inhibiting interaction between SARS-CoV-2 spike protein/a given SARS-CoV-2 variant spike protein and ACE2 with an IC₅₀ of less than 1 μg/ml, preferably one of ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤10 ng/ml, ≤9 ng/ml, ≤8 ng/ml, ≤7 ng/ml, ≤6 ng/ml, ≤5 ng/ml, ≤4 ng/ml, ≤3 ng/ml, ≤2 ng/ml, ≤1 ng/ml, ≤900 pg/ml, ≤800 pg/ml, ≤700 pg/ml, ≤600 pg/ml, ≤500 pg/ml, ≤400 pg/ml, ≤300 pg/ml, ≤200 pg/ml or ≤100 μg/ml, e.g. as determined in a pseudovirus neutralisation assay performed as described in Example 1.2 herein. In some embodiments, the antigen-binding molecule is capable of inhibiting interaction between SARS-CoV-2 spike protein/a given SARS-CoV-2 variant spike protein and ACE2 with an IC₅₀ of less than 1 μg/ml, preferably one of ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤10 ng/ml, ≤9 ng/ml, ≤8 ng/ml, ≤7 ng/ml, ≤6 ng/ml, ≤5 ng/ml, ≤4 ng/ml, ≤3 ng/ml, ≤2 ng/ml, ≤1 ng/ml, ≤900 pg/ml, ≤800 pg/ml, ≤700 pg/ml, ≤600 pg/ml, ≤500 pg/ml, ≤400 pg/ml, ≤300 pg/ml, ≤200 pg/ml or ≤100 pg/ml, e.g. as determined in a surrogate virus neutralisation test performed as described in Example 1.3 herein.

In some embodiments, an antigen-binding molecule according to the present disclosure reduces/inhibits infection of ACE2-expressing cells by a sarbecovirus (e.g. a SARS-COV, e.g. SARS-CoV-2 and/or one or more SARS-CoV-2 variants). Such antigen-binding molecules may be described as inhibiting/antagonising infection of ACE2-expressing cells, or may be referred to as neutralising infection of such cells, by sarbecovirus(es).

In some embodiments, an antigen-binding molecule according to the present disclosure reduces/inhibits infection of ACE2-expressing cells by a SARS-CoV-2 variant selected from: BA.1, BA.2, BA.5, BA.2.75, BA.2.75.2, BA.4.6.1, BF.7, BQ.1.1, XBB, XBB.1, XBB.1.16, XBB.2.3, EG.5, EG.5.1, B.1.1.7, B.1.351, B.1.617.2 and P.1.

In some embodiments, an antigen-binding molecule according to the present disclosure is capable of (independently) inhibiting infection of ACE2-expressing cells by two or more (e.g. 2, 3, 4, 5, 6, 7, 8, 9, 10, 11, 12, 13, 14, 15 or more) SARSr-CoVs selected from SARS-CoV-2 and SARS-CoV-2 variants. That is, in some embodiments, an antigen-binding molecule that inhibits infection of ACE2-expressing cells by a given (first) SARSr-CoV selected from SARS-CoV-2 and SARS-CoV-2 variants also inhibits infection of ACE2-expressing cells by one or more further (second, third, etc.) SARSr-CoVs selected from SARS-CoV-2 and SARS-CoV-2 variants, wherein the one or more further SARSr-CoVs have a nucleotide sequence which is different to the nucleotide sequence of the first SARSr-CoV.

In some embodiments, an antigen-binding molecule according to the present disclosure inhibits infection of ACE2-expressing cells by two or more (e.g. 2, 3, 4, 5, 6, 7, 8, 9, 10, 11, 12, 13, 14, 15, 16, 17 or all 18) SARSr-CoVs selected from: SARS-CoV-2, BA.1, BA.2, BA.5, BA.2.75, BA.2.75.2, BA.4.6.1, BF.7, BQ.1.1, XBB, XBB.1, XBB.1.16, XBB.2.3, EG.5, EG.5.1, B.1.1.7, B.1.351, B.1.617.2 and P.1.

In preferred embodiments, an antigen-binding molecule according to the present disclosure inhibits infection of ACE2-expressing cells by: SARS-CoV-2, BA.1, BA.2, BA.5, BA.2.75, BA.2.75.2, BA.4.6.1 and BF.7. In preferred embodiments, an antigen-binding molecule according to the present disclosure inhibits infection of ACE2-expressing cells by: SARS-CoV-2, BA.1, BA.2, BA.5, BA.2.75, BA.2.75.2, BA.4.6.1, BF.7, BQ.1.1 and XBB.1. In preferred embodiments, an antigen-binding molecule according to the present disclosure inhibits infection of ACE2-expressing cells by: SARS-CoV-2, BA.1, BA.2, BA.5, BA.2.75, BA.2.75.2, BA.4.6.1, BF.7, BQ.1.1, XBB.1, XBB.1.16, XBB.2.3, EG.5 and EG.5.1.

The ability of a given antigen-binding molecule to inhibit infection of ACE2-expressing cells by SARS-CoV-2/a SARS-CoV-2 variant can be analysed by detecting/quantifying infection of ACE2-expressing cells by SARS-CoV-2/the SARS-CoV-2 variant (or a pseudovirus pseudotyped with the spike protein encoded by SARS-CoV-2/the SARS-CoV-2 variant) in the presence of the antigen-binding molecule, and comparing the level of infection to the level observed in the absence of the antigen-binding molecule (and/or the level of infection observed in presence of an appropriate control antigen-binding molecule known not to affect infection of ACE2-expressing cells by the relevant virus). Such methods may comprise determining the absolute number of, or the proportion of, cells infected with (e.g. comprising) the relevant virus.

The ability of a given antigen-binding molecule to inhibit infection of ACE2-expressing cells by SARS-CoV-2/a SARS-CoV-2 variant can be analysed in a pseudovirus neutralisation assay, e.g. as described in Chia et al., Sci Adv. (2023) 9(30):eade3470 or Tan et al., Nature Biotechnology (2020) 38: 1073-1078.

In some embodiments, an antigen-binding molecule according to the present disclosure reduces/inhibits infection of ACE2-expressing cells by SARS-CoV-2/a SARS-CoV-2 variant (or a pseudovirus pseudotyped with the spike protein encoded by SARS-CoV-2/the SARS-CoV-2 variant) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of infection of ACE2-expressing cells observed in the absence of the antigen-binding molecule (or in the presence of an appropriate control antigen-binding molecule known not to affect infection of ACE2-expressing cells by SARS-CoV-2/the SARS-CoV-2 variant).

In some embodiments, the antigen-binding molecule is capable of inhibiting infection of ACE2-expressing cells by SARS-CoV-2/a SARS-CoV-2 variant (or a pseudovirus pseudotyped with the spike protein encoded by SARS-CoV-2/the SARS-CoV-2 variant) with an IC₅₀ of less than 1 μg/ml, preferably one of ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤10 ng/ml, ≤9 ng/ml, ≤8 ng/ml, ≤7 ng/ml, ≤6 ng/ml, ≤5 ng/ml, ≤4 ng/ml, ≤3 ng/ml, ≤2 ng/ml, ≤1 ng/ml, ≤900 pg/ml, ≤800 pg/ml, ≤700 pg/ml, ≤600 pg/ml, ≤500 pg/ml, ≤400 pg/ml, ≤300 pg/ml, ≤200 pg/ml or ≤100 pg/ml, e.g. as determined in a pseudovirus neutralisation assay performed as described in Example 1.2 herein.

In some embodiments, an antigen-binding molecule according to the present disclosure possesses one or more novel, similar or improved functional properties as compared to a known antigen-binding molecule that binds to SARS-CoV-2 spike protein and/or one or more SARS-CoV-2 variant spike proteins.

In some embodiments, an antigen-binding molecule possesses one or more novel, similar or improved functional properties as compared to SS6V11-E7 (also referred to herein as ‘E7’) described e.g. in WO 2022/245288 A1. For the purposes of comparison of functional properties in the following paragraphs, ‘SS6V11-E7’ refers to the antigen-binding molecule formed by association between two polypeptides consisting of SEQ ID NO:837, and two polypeptides consisting of SEQ ID NO:838.

In some embodiments, an antigen-binding molecule possesses one or more novel, similar or improved functional properties as compared to LyCov-1404 (also known as bebtelovimab; DrugBank Accession No. DB16755). For the purposes of comparison of functional properties in the following paragraphs, ‘LyCov-1404’ refers to the antigen-binding molecule formed by association between two polypeptides consisting of SEQ ID NO:854, and two polypeptides consisting of SEQ ID NO:855.

In some embodiments, an antigen-binding molecule described herein may display one or more of the following:
binds to a SARS-CoV-2 variant spike protein that SS6V11-E7 and/or LyCov-1404 does not bind to;
inhibits interaction between ACE2 and a SARS-CoV-2 variant spike protein whose interaction with ACE2 is not inhibited by SS6V11-E7 and/or LyCov-1404;
inhibits infection of ACE2-expressing cells by a sarbecovirus (e.g. SARS-CoV-2 variant) whose infection of ACE2-expressing cells is not inhibited by SS6V11-E7 and/or LyCov-1404;
binds to a sarbecovirus spike protein (e.g. a SARSr-CoV spike protein; e.g. SARS-CoV-2 spike protein and/or one or more SARS-CoV-2 variant spike proteins) with similar or increased affinity (e.g. a similar or lower K_{D}), as compared to the affinity with which the relevant protein(s) is/are bound by SS6V11-E7 and/or LyCov-1404.
inhibits interaction between a sarbecovirus spike protein (e.g. a SARSr-CoV spike protein; e.g. SARS-CoV-2 spike protein and/or one or more SARS-CoV-2 variant spike proteins) and ACE2 with similar or increased potency (e.g. a similar or lower IC50) as compared to the potency with which such interaction is inhibited by SS6V11-E7 and/or LyCov-1404; and/or
inhibits infection of ACE2-expressing cells by a sarbecovirus (e.g. a SARSr-CoV, e.g. SARS-CoV-2 and/or one or more SARS-CoV-2 variants) with similar or increased potency (e.g. a similar or lower IC50) as compared to the potency with which such infection is inhibited by SS6V11-E7 and/or LyCov-1404.

In accordance with the preceding paragraph, a K_{D}/IC50 value which is ‘similar’ to a reference K_{D}/IC50 value may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the reference K_{D}/IC50 value. A K_{D}/IC50 value which is ‘lower’ relative to a reference K_{D}/IC50 value may be less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the K_{D}/IC50 value.

It will be appreciated that for the purposes of such evaluations, equivalent amounts/concentrations of the antigen-binding molecule and SS6V11-E7 and/or LyCov-1404 may be compared.

In some embodiments, the antigen-binding molecule of the present disclosure binds to SARS-CoV-2 spike protein and/or a given SARS-CoV-2 variant spike protein with a KD that is similar to or less than the KD with which SS6V11-E7 and/or LyCov-1404 binds to the relevant protein, as determined in the same assay. In some embodiments, the antigen-binding molecule of the present disclosure binds to SARS-CoV-2 spike protein and/or a given SARS-CoV-2 variant spike protein with a KD that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the KD with which SS6V11-E7 and/or LyCov-1404 binds to the relevant protein, as determined in the same assay. In some embodiments, the antigen-binding molecule of the present disclosure binds to SARS-CoV-2 spike protein and/or a given SARS-CoV-2 variant spike protein with a KD that is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the KD with which SS6V11-E7 and/or LyCov-1404 binds to the relevant protein, as determined in the same assay.

In some embodiments, the antigen-binding molecule of the present disclosure inhibits interaction between SARS-CoV-2 spike protein and/or a given SARS-CoV-2 variant spike protein and ACE2 with an IC₅₀ that is similar to or less than the IC₅₀ with which SS6V11-E7 and/or LyCov-1404 inhibits interaction between the relevant protein and ACE2, as determined in the same assay. In some embodiments, the antigen-binding molecule inhibits interaction between SARS-CoV-2 spike protein and/or a given SARS-CoV-2 variant spike protein and ACE2 with an IC₅₀ that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the IC₅₀ for inhibition of interaction between the relevant protein and ACE2 by SS6V11-E7 and/or LyCov-1404, as determined in the same assay. In some embodiments, the antigen-binding molecule inhibits interaction between SARS-CoV-2 spike protein and/or a given SARS-CoV-2 variant spike protein and ACE2 with an IC₅₀ that is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the IC₅₀ for inhibition of interaction between the relevant protein and ACE2 by SS6V11-E7 and/or LyCov-1404, as determined in the same assay.

In some embodiments, the antigen-binding molecule of the present disclosure inhibits infection of ACE2-expressing cells by SARS-CoV-2/a SARS-CoV-2 variant (or a pseudovirus pseudotyped with the spike protein encoded by SARS-CoV-2/the SARS-CoV-2 variant) with an IC₅₀ that is similar to or less than the IC₅₀ with which SS6V11-E7 and/or LyCov-1404 inhibits interaction between the relevant protein and ACE2, as determined in the same assay. In some embodiments, the antigen-binding molecule inhibits infection of ACE2-expressing cells by SARS-CoV-2/a SARS-CoV-2 variant (or a pseudovirus pseudotyped with the spike protein encoded by SARS-CoV-2/the SARS-CoV-2 variant) with an IC₅₀ that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the IC₅₀ for inhibition of infection of such cells by the relevant SARSr-CoV by SS6V11-E7 and/or LyCov-1404, as determined in the same assay. In some embodiments, the antigen-binding molecule inhibits infection of ACE2-expressing cells by SARS-CoV-2/a SARS-CoV-2 variant (or a pseudovirus pseudotyped with the spike protein encoded by SARS-CoV-2/the SARS-CoV-2 variant) with an IC₅₀ that is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the IC₅₀ for inhibition of infection of such cells by the relevant SARSr-CoV by SS6V11-E7 and/or LyCov-1404, as determined in the same assay.

### Particular Exemplary Antigen-Binding Molecules and Polypeptides

The present disclosure also provides polypeptide constituents of antigen-binding molecules. The polypeptides may be provided in isolated or substantially purified form.

The antigen-binding molecule of the present disclosure may be, or may comprise, a complex of polypeptides.

In the present specification where a polypeptide comprises more than one domain or region, it will be appreciated that the plural domains/regions are preferably present in the same polypeptide chain. That is, the polypeptide comprising more than one domain or region is a fusion polypeptide comprising the domains/regions.

In some embodiments a polypeptide according to the present disclosure comprises, or consists of, a VH as described herein. In some embodiments a polypeptide according to the present disclosure comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1, CH2 region and/or a CH3 region of an immunoglobulin (lg).

In some embodiments, the polypeptide comprises one or more regions of an immunoglobulin heavy chain constant sequence. In some embodiments, the polypeptide comprises a CH1 region as described herein. In some embodiments, the polypeptide comprises a hinge region as described herein. In some embodiments, the polypeptide comprises a CH2 region as described herein. In some embodiments, the polypeptide comprises a CH3 region as described herein. In some embodiments, the polypeptide comprises a CH2-CH3 region as described herein. In some embodiments, the polypeptide comprises a CH1-hinge-CH2-CH3 region as described herein.

In some embodiments, the polypeptide comprises one or more regions of an immunoglobulin light chain constant sequence. In some embodiments, the polypeptide comprises a CL region as described herein.

In some embodiments, the polypeptide according to the present disclosure comprises a structure from N- to C-terminus according to one of the following:

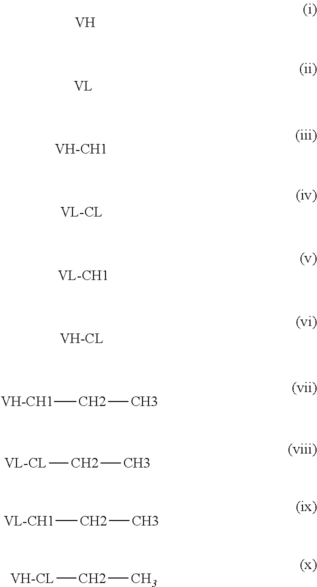

Also provided by the present disclosure are antigen-binding molecules composed of the polypeptides of the present disclosure. In some embodiments, the antigen-binding molecule of the present disclosure comprises one of the following combinations of polypeptides:

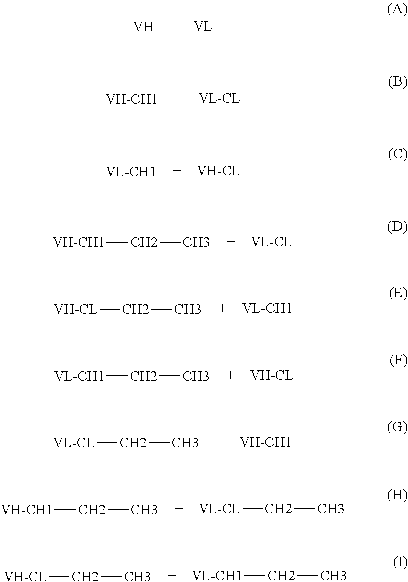

In some embodiments, the antigen-binding molecule comprises more than one of a polypeptide of the combinations shown in (A) to (I) above. By way of example, with reference to (D) above, In some embodiments, the antigen-binding molecule comprises two polypeptides comprising the structure VH-CH1-CH2-CH3, and two polypeptides comprising the structure VL-CL.

In accordance with (i) to (x) and (A) to (I) above, ‘VH’ refers to a VH region as described herein, ‘VL’ refers to a VL region as described herein.

In some embodiments, the antigen-binding molecule of the present disclosure comprises a polypeptide which comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:36, 52, 67, 83, 96, 105, 120, 136, 149, 164, 179, 193, 206, 220, 235, 249, 262, 274, 285, 299, 312, 325, 336, 350, 362, 368, 381, 393, 405, 416, 427, 436, 449, 453, 464, 475, 487, 496, 508, 522, 535, 547, 559, 572, 584, 592, 600, 614, 627, 643 or 656.

In some embodiments, the antigen-binding molecule of the present disclosure comprises a polypeptide which comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:44, 59, 75, 89, 102, 113, 128, 144, 157, 171, 187, 200, 213, 228, 242, 256, 270, 280, 291, 305, 318, 331, 343, 355, 366, 374, 388, 400, 411, 423, 432, 443, 451, 460, 471, 481, 491, 502, 515, 529, 542, 554, 567, 579, 587, 596, 607, 621, 635, 651, 663.

In some embodiments, the antigen-binding molecule of the present disclosure comprises a polypeptide which comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818 or 820.

In some embodiments, the antigen-binding molecule of the present disclosure comprises a polypeptide which comprises, or consists of, an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819 or 821.

In some embodiments, the antigen-binding molecule of the present disclosure comprises a polypeptide or polypeptides comprising a VH region comprising the heavy chain CDRs, and a VL region comprising the light chain CDRs, of an antibody selected from an antibody as shown in Table A herein. That is, in some embodiments, the antigen-binding molecule comprises a polypeptide or polypeptides comprising: (i) a VH region comprising HC-CDR1, HC-CDR2 and HC-CDR3 as indicated in column A of Table A, and (ii) a VL region comprising LC-CDR1, LC-CDR2 and LC-CDR3 as indicated in column B of Table A, wherein the sequences of Columns A and B are selected from the same row of Table A. In some embodiments, the antigen-binding molecule of the present disclosure comprises a polypeptide or polypeptides comprising a VH region comprising the heavy chain CDRs, and a VL region comprising the light chain CDRs, of an antibody as shown in Table A. In some embodiments, the antigen-binding molecule of the present disclosure comprises a polypeptide or polypeptides comprising a VH region comprising the heavy chain CDRs, and a VL region comprising the light chain CDRs, of an antibody as shown in Table A herein.

In some embodiments, the antigen-binding molecule of the present disclosure comprises a polypeptide or polypeptides comprising a VH region comprising the heavy chain FRs, and a VL region comprising the light chain FRs, of an antibody selected from an antibody as shown in Table B herein. That is, in some embodiments, the antigen-binding molecule comprises a polypeptide or polypeptides comprising: (i) a VH region comprising HC-FR1, HC-FR2, HC-FR3 and HC-FR4 as indicated in column A of Table B, and (ii) a VL region comprising LC-FR1, LC-FR2, LC-FR3, and LC-FR4 as indicated in column B of Table B, wherein the sequences of columns A and B are selected from the same row of Table B. In some embodiments, the antigen-binding molecule of the present disclosure comprises a polypeptide or polypeptides comprising a VH region comprising the heavy chain FRs, and a VL region comprising the light chain FRs, of an antibody as shown in Table B herein. In some embodiments, the antigen-binding molecule of the present disclosure comprises a polypeptide or polypeptides comprising a VH region comprising the heavy chain FRs, and a VL region comprising the light chain FRs, of an antibody as shown in Table B herein.

In some embodiments, the antigen-binding molecule of the present disclosure comprises a polypeptide or polypeptides comprising: (i) an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to an amino acid sequence indicated in column A of Table C, and (ii) an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to an amino acid sequence indicated in column B of Table C, wherein the sequences of columns A and B are selected from the same row of Table C.

In some embodiments, the antigen-binding molecule of the present disclosure comprises a polypeptide or polypeptides comprising a VH region and a VL region of an antibody clone selected from an antibody as shown in Table C herein. That is, in some embodiments, the antigen-binding molecule comprises a polypeptide or polypeptides comprising: (i) an amino acid sequence indicated in column A of Table C, and (ii) an amino acid sequence indicated in column B of Table C, wherein the sequences of columns A and B are selected from the same row of Table C. In some embodiments, the antigen-binding molecule of the present disclosure comprises a polypeptide or polypeptides comprising a VH region and a VL region of an antibody as shown in Table C herein. In some embodiments, the antigen-binding molecule of the present disclosure comprises a polypeptide or polypeptides comprising a VH region and a VL region of an antibody as shown in Table C herein.

In some embodiments, the antigen-binding molecule of the present disclosure comprises: (i) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to an amino acid sequence indicated in column A of Table D, and (ii) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to an amino acid sequence indicated in column B of Table D, wherein the sequences of columns A and B are selected from the same row of Table D.

In some embodiments, the antigen-binding molecule of the present disclosure comprises the polypeptides of an antigen-binding molecule according to Table D herein. That is, in some embodiments, the antigen-binding molecule comprises: (i) a polypeptide comprising or consisting of an amino acid sequence indicated in column A of Table D, and (ii) a polypeptide comprising or consisting of an amino acid sequence indicated in column B of Table D, wherein the sequences of columns A and B are selected from the same row of Table D.

In some embodiments, the antigen-binding molecule of the present disclosure comprises:
(1) (i) a polypeptide comprising or consisting of an amino acid sequence having at least 70% amino acid sequence identity to SEQ ID NO:722, and (ii) a polypeptide comprising or consisting of an amino acid sequence having at least 70% amino acid sequence identity to SEQ ID NO:723;
(2) (i) a polypeptide comprising or consisting of an amino acid sequence having at least 70% amino acid sequence identity to SEQ ID NO:720, and (ii) a polypeptide comprising or consisting of an amino acid sequence having at least 70% amino acid sequence identity to SEQ ID NO:721;
(3) (i) a polypeptide comprising or consisting of an amino acid sequence having at least 70% amino acid sequence identity to SEQ ID NO:724, and (ii) a polypeptide comprising or consisting of an amino acid sequence having at least 70% amino acid sequence identity to SEQ ID NO:725;
(4) (i) a polypeptide comprising or consisting of an amino acid sequence having at least 70% amino acid sequence identity to SEQ ID NO:726, and (ii) a polypeptide comprising or consisting of an amino acid sequence having at least 70% amino acid sequence identity to SEQ ID NO:727;
(5) (i) a polypeptide comprising or consisting of an amino acid sequence having at least 70% amino acid sequence identity to SEQ ID NO:794, and (ii) a polypeptide comprising or consisting of an amino acid sequence having at least 70% amino acid sequence identity to SEQ ID NO:795;
(6) (i) a polypeptide comprising or consisting of an amino acid sequence having at least 70% amino acid sequence identity to SEQ ID NO:752, and (ii) a polypeptide comprising or consisting of an amino acid sequence having at least 70% amino acid sequence identity to SEQ ID NO:753;
(7) (i) a polypeptide comprising or consisting of an amino acid sequence having at least 70% amino acid sequence identity to SEQ ID NO:748, and (ii) a polypeptide comprising or consisting of an amino acid sequence having at least 70% amino acid sequence identity to SEQ ID NO:749;
(8) (i) a polypeptide comprising or consisting of an amino acid sequence having at least 70% amino acid sequence identity to SEQ ID NO:740, and (ii) a polypeptide comprising or consisting of an amino acid sequence having at least 70% amino acid sequence identity to SEQ ID NO:741;
(9) (i) a polypeptide comprising or consisting of an amino acid sequence having at least 70% amino acid sequence identity to SEQ ID NO:754, and (ii) a polypeptide comprising or consisting of an amino acid sequence having at least 70% amino acid sequence identity to SEQ ID NO:754;
(10) (i) a polypeptide comprising or consisting of an amino acid sequence having at least 70% amino acid sequence identity to SEQ ID NO:766, and (ii) a polypeptide comprising or consisting of an amino acid sequence having at least 70% amino acid sequence identity to SEQ ID NO:767;
(11) (i) a polypeptide comprising or consisting of an amino acid sequence having at least 70% amino acid sequence identity to SEQ ID NO:774, and (ii) a polypeptide comprising or consisting of an amino acid sequence having at least 70% amino acid sequence identity to SEQ ID NO:775;
(12) (i) a polypeptide comprising or consisting of an amino acid sequence having at least 70% amino acid sequence identity to SEQ ID NO:776, and (ii) a polypeptide comprising or consisting of an amino acid sequence having at least 70% amino acid sequence identity to SEQ ID NO:777;
(13) (i) a polypeptide comprising or consisting of an amino acid sequence having at least 70% amino acid sequence identity to SEQ ID NO:778, and (ii) a polypeptide comprising or consisting of an amino acid sequence having at least 70% amino acid sequence identity to SEQ ID NO:779;
(14) (i) a polypeptide comprising or consisting of an amino acid sequence having at least 70% amino acid sequence identity to SEQ ID NO:780, and (ii) a polypeptide comprising or consisting of an amino acid sequence having at least 70% amino acid sequence identity to SEQ ID NO:781;
(15) (i) a polypeptide comprising or consisting of an amino acid sequence having at least 70% amino acid sequence identity to SEQ ID NO:784, and (ii) a polypeptide comprising or consisting of an amino acid sequence having at least 70% amino acid sequence identity to SEQ ID NO:785;
(16) (i) a polypeptide comprising or consisting of an amino acid sequence having at least 70% amino acid sequence identity to SEQ ID NO:786, and (ii) a polypeptide comprising or consisting of an amino acid sequence having at least 70% amino acid sequence identity to SEQ ID NO:787;
(17) (i) a polypeptide comprising or consisting of an amino acid sequence having at least 70% amino acid sequence identity to SEQ ID NO:728, and (ii) a polypeptide comprising or consisting of an amino acid sequence having at least 70% amino acid sequence identity to SEQ ID NO:729;
(18) (i) a polypeptide comprising or consisting of an amino acid sequence having at least 70% amino acid sequence identity to SEQ ID NO:790, and (ii) a polypeptide comprising or consisting of an amino acid sequence having at least 70% amino acid sequence identity to SEQ ID NO:791;
(19) (i) a polypeptide comprising or consisting of an amino acid sequence having at least 70% amino acid sequence identity to SEQ ID NO:806, and (ii) a polypeptide comprising or consisting of an amino acid sequence having at least 70% amino acid sequence identity to SEQ ID NO:807; or

### Known SARS-CoV-2 Spike Protein/SARS-CoV-2 Variant Spike Protein-Binding Antigen-Binding Molecules

Aspects and embodiments of the present disclosure also pertain to known SARS-CoV-2 spike protein/SARS-CoV-2 variant spike protein-binding antigen-binding molecules, and derivatives thereof. To be clear, where reference is made herein to ‘an antigen-binding molecule of the present disclosure’ reference to such known antigen-binding molecules is not intended.

In some embodiments, a known SARS-CoV-2 spike protein/SARS-CoV-2 variant spike protein-binding antigen-binding molecule is SS6V11-E7, or a derivative thereof. SS6V11-E7 (also referred to herein as ‘E7’) is described e.g. in WO 2022/245288 A1. E7 comprises the VH region shown in SEQ ID NO:824 and the VL region shown in SEQ ID NO:830. HC-CDR1, HC-CDR2 and HC-CDR3 of E7 are shown in SEQ ID NOs:825, 826 and 827 (respectively), and LC-CDR1, LC-CDR2 and LC-CDR3 of E7 are shown in SEQ ID NOs:831, 832 and 832 (respectively). HC-FR1, HC-FR2, HC-FR3 and HC-FR4 of E7 are shown in SEQ ID NOs:266, 828, 829 and 112 (respectively), and LC-FR1, LC-FR2, LC-FR3 and LC-FR4 of E7 are shown in SEQ ID NOs:834, 835, 826 and 486 (respectively). E7 in human IgG1 heavy chain, K light chain format is formed by association between two polypeptides having the sequence of SEQ ID NO:837, and two polypeptides having the sequence of SEQ ID NO:838.

In some embodiments, SS6V11-E7, or a derivative thereof, comprises: (i) a VH region comprising HC-CDR1=SEQ ID NO:825 (or a variant thereof in which 1 or 2 or 3 amino acids in HC-CDR1 are substituted with another amino acid), HC-CDR2=SEQ ID NO:826 (or a variant thereof in which 1 or 2 or 3 amino acids in HC-CDR2 are substituted with another amino acid) and HC-CDR3=SEQ ID NO:827 (or a variant thereof in which 1 or 2 or 3 amino acids in HC-CDR3 are substituted with another amino acid); and (ii) a VL region comprising LC-CDR1=SEQ ID NO:831 (or a variant thereof in which 1 or 2 or 3 amino acids in LC-CDR1 are substituted with another amino acid), LC-CDR2=SEQ ID NO:832 (or a variant thereof in which 1 or 2 or 3 amino acids in LC-CDR2 are substituted with another amino acid) and LC-CDR3=SEQ ID NO:833 (or a variant thereof in which 1 or 2 or 3 amino acids in HC-CDR3 are substituted with another amino acid). In some embodiments, SS6V11-E7, or a derivative thereof, comprises: (i) a VH region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:824; and (ii) a VL region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:830.

In some embodiments, a known SARS-CoV-2 spike protein/SARS-CoV-2 variant spike protein-binding antigen-binding molecule is LyCov-1404, or a derivative thereof. LyCov-1404 (also known as bebtelovimab; DrugBank Accession No. DB16755) comprises the VH region shown in SEQ ID NO:839 and the VL region shown in SEQ ID NO:846. HC-CDR1, HC-CDR2 and HC-CDR3 of LyCov-1404 are shown in SEQ ID NOs:840, 841 and 842 (respectively), and LC-CDR1, LC-CDR2 and LC-CDR3 of LyCov-1404 are shown in SEQ ID NOs:847, 848 and 849 (respectively). HC-FR1, HC-FR2, HC-FR3 and HC-FR4 of LyCov-1404 are shown in SEQ ID NOs:843, 844, 845 and 112 (respectively), and LC-FR1, LC-FR2, LC-FR3 and LC-FR4 of LyCov-1404 are shown in SEQ ID NOs:850, 851, 852 and 853 (respectively). LyCov-1404 in human IgG1 (G1m3) heavy chain, CACL2 light chain format is formed by association between two polypeptides having the sequence of SEQ ID NO:854, and two polypeptides having the sequence of SEQ ID NO:855.

In some embodiments, LyCov-1404, or a derivative thereof, comprises: (i) a VH region comprising HC-CDR1=SEQ ID NO:840 (or a variant thereof in which 1 or 2 or 3 amino acids in HC-CDR1 are substituted with another amino acid), HC-CDR2=SEQ ID NO:841 (or a variant thereof in which 1 or 2 or 3 amino acids in HC-CDR2 are substituted with another amino acid) and HC-CDR3=SEQ ID NO:842 (or a variant thereof in which 1 or 2 or 3 amino acids in HC-CDR3 are substituted with another amino acid); and (ii) a VL region comprising LC-CDR1=SEQ ID NO:847 (or a variant thereof in which 1 or 2 or 3 amino acids in LC-CDR1 are substituted with another amino acid), LC-CDR2=SEQ ID NO:848 (or a variant thereof in which 1 or 2 or 3 amino acids in LC-CDR2 are substituted with another amino acid) and LC-CDR3=SEQ ID NO:849 (or a variant thereof in which 1 or 2 or 3 amino acids in HC-CDR3 are substituted with another amino acid). In some embodiments, LyCov-1404, or a derivative thereof, comprises: (i) a VH region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:839; and (ii) a VL region comprising at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:846.

In some embodiments, a known SARS-CoV-2 spike protein/SARS-CoV-2 variant spike protein-binding antigen-binding molecule comprises:
(A) (i) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:824, and (ii) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:830;
(B) (i) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:839, and (ii) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:846;
(C) (i) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:837, and (ii) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:838; or
(D) (i) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:854, and (ii) a polypeptide comprising or consisting of an amino acid sequence having at least 70% (e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%) amino acid sequence identity to SEQ ID NO:855.

### Linkers and Additional Sequences

The antigen-binding molecules and polypeptides of the present disclosure may additionally comprise further amino acids or sequences of amino acids.

The antigen-binding molecules and polypeptides of the present disclosure may comprise one or more linker sequences between sequences of amino acids. For example, a linker sequence may be provided between a VH sequence and a VL sequence, providing linkage between the VH and VL (e.g. as in an scFv molecule).

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments, the linker sequence comprises or consists of glycine and serine residues. In some embodiments, the linker sequence has the structure: (GxS)n or (GxS)nGm; wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G₄S. In some embodiments, the linker sequence comprises or consists of (G₄S)₄ or (G₄S)₆. In some embodiments, the linker sequence has a length of 1-2, 1-3, 1-4, 1-5, 1-10, 1-15, 1-20, 1-25, or 1-30 amino acids.

The antigen-binding molecules and polypeptides of the present disclosure may comprise amino acid sequence(s) to facilitate expression, folding, trafficking, processing, purification or detection of the antigen-binding molecule/polypeptide. For example, antigen-binding molecules and polypeptides of the present disclosure may additionally comprise a sequence of amino acids forming a detectable moiety, e.g. as described hereinbelow.

The antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

The signal peptide may be present at the N-terminus of the antigen-binding molecule/polypeptide, and may be present in the newly synthesised antigen-binding molecule/polypeptide. The signal peptide provides for efficient trafficking of the antigen-binding molecule/polypeptide. Signal peptides are often removed by cleavage, and thus are not comprised in the mature antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

### Labels and Conjugates

In some embodiments, the antigen-binding molecule or polypeptide of the present disclosure comprises a detectable moiety.

In some embodiments, a detectable moiety is a fluorescent label, phosphorescent label, luminescent label, immuno-detectable label (e.g. an epitope tag), radiolabel, chemical, nucleic acid or enzymatic label. The antigen-binding molecule or polypeptide may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine ¹²³, Iodine¹²⁵, Iodine¹²⁶, Iodine¹³¹, Iodine¹³³, Bromine⁷⁷, Technetium^{99m}, Indium111, Indium 113m, Gallium67 Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thulium/¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the antigen-binding molecule/polypeptide comprises an epitope tag, e.g. a His, (e.g. 6×His), FLAG, c-Myc, StrepTag, haemagglutinin, E, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the antigen-binding molecule/polypeptide.

In some embodiments, the antigen-binding molecule/polypeptide comprises a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

In some embodiments, the antigen-binding molecule or polypeptide of the present disclosure comprises a chemical moiety. In some embodiments, the antigen-binding molecule/polypeptide of the present disclosure is conjugated to a chemical moiety.

The chemical moiety may be a moiety for providing a therapeutic effect, i.e. a drug moiety. A drug moiety may be a small molecule (e.g. a low molecular weight (<1000 daltons, typically between ~300-700 daltons) organic compound). Drug moieties are described e.g. in Parslow et al., Biomedicines. 2016 September; 4(3):14 (hereby incorporated by reference in its entirety). In some embodiments, a drug moiety may be or comprise a cytotoxic agent. In some embodiments, a drug moiety may be or comprise a chemotherapeutic agent. Drug moieties include e.g. calicheamicin, DM1, DM4, monomethylauristatin E (MMAE), monomethylauristatin F (MMAF), SN-38, doxorubicin, duocarmycin, D6.5 and PBD.

### Nucleic Acids and Vectors

The present disclosure provides a nucleic acid, or a plurality of nucleic acids, encoding an antigen-binding molecule or polypeptide according to the present disclosure. In some embodiments, the nucleic acid(s) comprise or consist of DNA and/or RNA.

An antigen-binding molecule or polypeptide according to the present disclosure may be produced within a cell by translation of RNA encoding the polypeptide(s). An antigen-binding molecule or polypeptide according to the present disclosure may be produced within a cell by transcription from nucleic acid encoding the polypeptide(s), and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a polypeptide according to the present disclosure to a cell. The vector may be an expression vector comprising elements required for expressing a polypeptide according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding an antigen-binding molecule or polypeptide according to the present disclosure. A vector may also include a termination codon (i.e. 3′ in the nucleotide sequence of the vector to the nucleotide sequence encoding the polypeptide(s)) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleic acid sequence(s) (e.g. a promoter and/or enhancers) are covalently linked in such a way as to place the expression of the nucleic acid encoding a polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule according to the present disclosure may be encoded by different nucleic acids of the plurality of nucleic acids, or by different vectors of the plurality of vectors.

### Producing the Antigen-Binding Molecules and Polypeptides

Antigen-binding molecules and polypeptides according to the present disclosure may be prepared according to methods for the production of polypeptides known to the skilled person.

Antigen-binding molecules and polypeptides may be prepared by chemical synthesis, e.g. liquid or solid phase synthesis. For example, peptides/polypeptides can be synthesised using the methods described in, for example, Chandrudu et al., Molecules (2013), 18: 4373-4388, which is hereby incorporated by reference in its entirety.

Alternatively, antigen-binding molecules and polypeptides may be produced by recombinant expression.

Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100:3451-3461, both of which are hereby incorporated by reference in their entirety.

In some cases, the antigen-binding molecules of the present disclosure are comprised of more than one polypeptide chain. In such cases, production of the antigen-binding molecule may comprise transcription and translation of more than one polypeptide, and subsequent association of the polypeptide chains to form the antigen-binding molecule.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Cells Comprising/Expressing the Antigen-Binding Molecules and Polypeptides

The present disclosure also provides a cell comprising or expressing an antigen-binding molecule or polypeptide according to the present disclosure. Also provided is a cell comprising or expressing a nucleic acid, a plurality of nucleic acids, a vector or a plurality of vectors according to the present disclosure.

It will be appreciated that where cells are referred to herein in the singular (i.e. ‘a/the cell’), pluralities/populations of such cells are also contemplated.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NSO and BHK cells. In preferred embodiments, the cell is, or is derived from, a CHO cell.

The present disclosure also provides a method for producing a cell comprising a nucleic acid(s) or vector(s) according to the present disclosure, comprising introducing a nucleic acid, a plurality of nucleic acids, a vector or a plurality of vectors according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an antigen-binding molecule or polypeptide according to the present disclosure, comprising introducing a nucleic acid, a plurality of nucleic acids, a vector or a plurality of vectors according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

Combination with Known SARS-CoV-2 Spike Protein/SARS-CoV-2 Variant Spike Protein-Binding Antigen-Binding Molecules

The present disclosure also provides a combination comprising (i) an antigen-binding molecule according to the present disclosure, and (ii) a known SARS-CoV-2 spike protein/SARS-CoV-2 variant spike protein-binding antigen-binding molecule. The present disclosure also provides a composition comprising (i) an antigen-binding molecule according to the present disclosure, and (ii) a known SARS-CoV-2 spike

It will be appreciated that the antigen-binding molecule of (i) according to the preceding paragraph may be an antigen-binding molecule according to any embodiment described in the section herein entitled ‘Antigen-binding molecules of the disclosure’. It will similarly be appreciated that the antigen-binding molecule of (ii) according to the preceding paragraph may be a known SARS-CoV-2 spike protein/SARS-CoV-2 variant spike protein-binding antigen-binding molecule according to any embodiment described in the section herein entitled ‘Known SARS-CoV-2 spike protein/SARS-CoV-2 variant spike protein-binding antigen-binding molecules’.

In some embodiments, the combination/composition of the present disclosure comprises (i) an antigen-binding molecule according to one of (1) to (19) in the section entitled ‘Particular exemplary antigen-binding molecules, and (ii) an antigen-binding molecule according to one of (A) to (D) in the section entitled ‘Known SARS-CoV-2 spike protein/SARS-CoV-2 variant spike protein-binding antigen-binding molecules’.

In some aspects and embodiments, the combination is a pharmaceutical combination. As used herein, a ‘pharmaceutical combination’ refers to a product that comprises plural (herein typically two) different active (i.e. therapeutic/prophylactic) agents, which are intended to be used in combination. The agents of a pharmaceutical combination may be formulated together or separately, but will typically be packaged together, typically with a package insert bearing instructions for the use of the agents in combination.

In some embodiments, the agents of a pharmaceutical combination are comprised in a single composition, e.g. a pharmaceutical composition comprising both agents. In some embodiments, the agents of a pharmaceutical combination are comprised in separate compositions; for example, the pharmaceutical combination may be provided as (i) a pharmaceutical composition comprising an antigen-binding molecule according to the present disclosure, and (ii) a pharmaceutical composition comprising a known SARS-CoV-2 spike protein/SARS-CoV-2 variant spike protein-binding antigen-binding molecule.

The present disclosure also provides compositions (e.g. pharmaceutical compositions and medicaments) comprising the agents described herein (i.e. (i) and (ii) above). Such compositions may comprise the relevant article in a formulation suitable for clinical use.

The present disclosure also provides combinations (and compositions comprising combinations) of antigen-binding molecules according to (A) or (B), with antigen-binding molecules according to (C) or (D) (i.e. as described in the section herein entitled ‘Known SARS-CoV-2 spike protein/SARS-CoV-2 variant spike protein-binding antigen-binding molecules’). In some embodiments, a combination comprises an antigen-binding molecule according to (A) and an antigen-binding molecule according to (B). In some embodiments, a combination comprises an antigen-binding molecule according to (C) and an antigen-binding molecule according to (D).

### Functional Properties of the Combinations of the Disclosure

The combinations described herein and compositions comprising such combinations (hereafter in this section, ‘combination(s)/composition(s)’) may be characterised by reference to certain functional properties. In some embodiments, a combination described herein may possess one or more of the following properties:
inhibits interaction between a sarbecovirus spike protein (e.g. a SARSr-CoV spike protein; e.g. SARS-CoV-2 spike protein and/or one or more SARS-CoV-2 variant spike proteins) and ACE2 with increased potency (e.g. a lower IC50) as compared to the potency with which such interaction is inhibited by a constituent agent of the combination/composition when used alone; and/or
inhibits infection of ACE2-expressing cells by a sarbecovirus (e.g. a SARSr-CoV, e.g. SARS-CoV-2 and/or one or more SARS-CoV-2 variants) with increased potency (e.g. a lower IC50) as compared to the potency with which such infection is inhibited by a constituent agent of the combination/composition when used alone.

In some embodiments, a combination/composition of the present disclosure inhibits interaction between SARS-CoV-2 spike protein and/or a given SARS-CoV-2 variant spike protein and ACE2 with an IC₅₀ that is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the IC₅₀ for inhibition of interaction between the relevant protein and ACE2 by a constituent agent of the combination/composition when used alone, as determined in the same assay.

In some embodiments, a combination/composition of the present disclosure inhibits infection of ACE2-expressing cells by a sarbecovirus (e.g. a SARSr-CoV, e.g. SARS-CoV-2 and/or one or more SARS-CoV-2 variants) with an IC₅₀ that is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the IC₅₀ for inhibition of infection of such cells by the relevant SARSr-CoV by a constituent agent of the combination/composition when used alone, as determined in the same assay.

In some embodiments, a combination/composition according to the present disclosure achieves a synergistic inhibition of interaction between SARS-CoV-2 spike protein and/or a given SARS-CoV-2 variant spike protein and ACE2. In some embodiments, a combination/composition according to the present disclosure achieves a synergistic inhibition of infection of ACE2-expressing cells by a sarbecovirus (e.g. a SARSr-CoV, e.g. SARS-CoV-2 and/or one or more SARS-CoV-2 variants). That is, in some embodiments, the combination/composition achieves a level of inhibition that is synergistic (i.e. super-additive), relative to what is observed when the antigen-binding molecule of the present disclosure is used alone, and/or relative to what is observed when the known SARS-CoV-2 spike protein/SARS-CoV-2 variant spike protein-binding antigen-binding molecule is used alone.

As used herein, a ‘synergistic’ or ‘super-additive’ level of a relevant effect (e.g. inhibition of interaction, inhibition of infection) for a given combination/composition refers to a level of the effect which is greater than the sum of the effects observed for the individual components of the combination/composition, when used alone.

Quantitative methods for assessing synergism are described e.g. in Tallarida, Genes Cancer. (2011) 2(11):1003-1008 and Chou, Cancer Res (2010) 70:440-446, both of which are hereby incorporated by reference in their entirety. Additive, synergistic and antagonistic effects may be evaluated in experiments in which a range of different doses of the combination/composition and the individual constituents thereof are evaluated for the relevant effect. Dose-response curves may be plotted, and evaluated in order to determine whether the combination/composition achieves a synergistic level of the relevant effect relative to the individual constituents of the combination/composition employed in isolation. In some embodiments, synergy may be evaluated using combination/composition index (CI) values calculated using the Chou-Talalay method described in Chou, Cancer Res (2010) 70:440-446. According to the Chou-Talalay method, for a given combination/composition CI=1 indicates an additive effect, CI<1 indicates synergism, and CI>1 indicates antagonism.

### Compositions

The present disclosure also provides compositions comprising the antigen-binding molecules, polypeptides, nucleic acids, expression vectors and/or cells described herein.

The antigen-binding molecules, polypeptides, nucleic acids, expression vectors and cells described herein may be formulated as pharmaceutical compositions or medicaments for clinical use and may comprise a pharmaceutically acceptable carrier, diluent, excipient or adjuvant. Thus, the present disclosure also provides a pharmaceutical composition/medicament comprising an antigen-binding molecule, polypeptide, nucleic acid/plurality, expression vector/plurality or cell described herein.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Compositions may be formulated for topical, parenteral, systemic, intracavitary, intravenous, intra-arterial, intramuscular, intrathecal, intraocular, intraconjunctival, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the relevant article in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel, tissue/organ of interest.

The present disclosure also provides methods for the production of pharmaceutically-useful compositions and medicaments. Such methods may comprise one or more steps selected from: producing an antigen-binding molecule, polypeptide, nucleic acid (or plurality thereof), expression vector (or plurality thereof) or cell described herein; isolating an antigen-binding molecule, polypeptide, nucleic acid (or plurality thereof), expression vector (or plurality thereof) or cell described herein; and/or mixing an antigen-binding molecule, polypeptide, nucleic acid (or plurality thereof), expression vector (or plurality thereof) or cell described herein with a pharmaceutically-acceptable carrier, adjuvant, excipient or diluent.

For example, a further aspect of the present disclosure relates to a method of formulating or producing a medicament or pharmaceutical composition for use in the treatment of a disease/condition (e.g. a disease/condition described herein), the method comprising formulating a pharmaceutical composition or medicament by mixing an antigen-binding molecule, polypeptide, nucleic acid (or plurality thereof), expression vector (or plurality thereof) or cell described herein with a pharmaceutically acceptable carrier, adjuvant, excipient or diluent.

### Therapeutic and Prophylactic Applications

The antigen-binding molecules, polypeptides, nucleic acids, expression vectors, cells, combinations and compositions described herein find use in therapeutic and prophylactic methods.

The present disclosure provides an antigen-binding molecule, polypeptide, nucleic acid (or plurality thereof), expression vector (or plurality thereof), cell, composition or combination described herein for use in a method of medical treatment or prophylaxis. Also provided is an antigen-binding molecule, polypeptide, nucleic acid (or plurality thereof), expression vector (or plurality thereof), cell, composition or combination described herein for use in a method of treating or preventing a disease or condition described herein. Also provided is the use of an antigen-binding molecule, polypeptide, nucleic acid (or plurality thereof), expression vector (or plurality thereof), cell, composition or combination described herein in the manufacture of a medicament for treating or preventing a disease or condition described herein. Also provided is a method of treating or preventing a disease or condition described herein, comprising administering to a subject a therapeutically or prophylactically effective amount of an antigen-binding molecule, polypeptide, nucleic acid (or plurality thereof), expression vector (or plurality thereof), cell, composition or combination described herein.

The present disclosure also provides an antigen-binding molecule according to the present disclosure for use in a method of treating or preventing a disease/condition described herein, wherein the method further comprises administering a known SARS-CoV-2 spike protein/SARS-CoV-2 variant spike protein-binding antigen-binding molecule. Also provided is a known SARS-CoV-2 spike protein/SARS-CoV-2 variant spike protein-binding antigen-binding molecule for use in a method of treating or preventing a disease/condition described herein, wherein the method further comprises administering an antigen-binding molecule according to the present disclosure.

Also provided is the use of an antigen-binding molecule according to the present disclosure in the manufacture of a medicament for use in a method of treating or preventing a disease/condition described herein, wherein the method further comprises administering a known SARS-CoV-2 spike protein/SARS-CoV-2 variant spike protein-binding antigen-binding molecule. Also provided is the use of a known SARS-CoV-2 spike protein/SARS-CoV-2 variant spike protein-binding antigen-binding molecule in the manufacture of a medicament for use in a method of treating or preventing a disease/condition described herein, wherein the method further comprises administering an antigen-binding molecule according to the present disclosure.

Further provided is a method of treating or preventing a disease/condition described herein, the method comprising administering a therapeutically- or prophylactically-effective amount of (i) an antigen-binding molecule according to the present disclosure and (ii) a known SARS-CoV-2 spike protein/SARS-CoV-2 variant spike protein-binding antigen-binding molecule to a subject in need of treatment.

The present disclosure also provides (i) an antigen-binding molecule according to the present disclosure and (ii) a known SARS-CoV-2 spike protein/SARS-CoV-2 variant spike protein-binding antigen-binding molecule for use in a method of treating or preventing a disease/condition described herein in a subject. Also provided is the use of (i) an antigen-binding molecule according to the present disclosure and (ii) a known SARS-CoV-2 spike protein/SARS-CoV-2 variant spike protein-binding antigen-binding molecule in the manufacture of a medicament for use in treating or preventing a disease/condition described herein in a subject. Also provided is a method of treating or preventing a disease/condition described herein in a subject, comprising administering to the subject a therapeutically- or prophylactically-effective amount of (i) an antigen-binding molecule according to the present disclosure and (ii) a known SARS-CoV-2 spike

In embodiments in accordance with aspects of the preceding paragraph, provision of (i) and (ii) may be as a combination therapy. In some embodiments, (i) and (ii) may be provided simultaneously or sequentially.

The articles, methods and uses of the present disclosure may be effective to reduce the development or progression of a disease/condition, alleviation of the symptoms of a disease/condition or reduction in the pathology of a disease/condition. The articles, methods and uses may be effective to prevent progression of the disease/condition, e.g. to prevent worsening of, or to slow the rate of development of, the disease/condition. In some embodiments, the articles, methods and uses may lead to an improvement in the disease/condition, e.g. a reduction in the symptoms of the disease/condition or reduction in some other correlate of the severity/activity of the disease/condition. In some embodiments, the articles, methods and uses may prevent development of the disease/condition a later stage (e.g. a chronic stage).

It will be appreciated that the articles of the present disclosure may be used for the treatment/prevention of any disease/condition that would derive therapeutic or prophylactic benefit from a reduction in the level of a sarbecovirus (e.g. a SARSr-CoV, e.g. SARS-CoV-2/a SARS-CoV-2 variant), or a reduction in the number of cells infected with a sarbecovirus (e.g. a SARS-COV, e.g. SARS-CoV-2/a SARS-CoV-2 variant). For example, the disease/condition may be a disease/condition in which infection with a sarbecovirus (e.g. a SARSr-CoV, e.g. SARS-CoV-2/a SARS-CoV-2 variant) is pathologically-implicated, e.g. a disease/condition for which infection with a sarbecovirus (e.g. a SARSr-CoV, e.g. SARS-CoV-2/a SARS-CoV-2 variant) is positively associated with the onset, development or progression of the disease/condition, and/or severity of one or more symptoms of the disease/condition, or for which infection with a sarbecovirus (e.g. a SARSr-CoV, e.g., e.g. SARS-CoV-2/a SARS-CoV-2 variant) is, is a risk factor for the onset, development or progression of the disease/condition.

In some embodiments, the disease/condition to be treated/prevented in accordance with the present disclosure is a disease/condition characterised by infection with a sarbecovirus (e.g. a SARSr-CoV, e.g. SARS-CoV-2/a SARS-CoV-2 variant), e.g. COVID-19. In some embodiments, the disease/condition is a disease/condition caused by infection with a sarbecovirus (e.g. a SARSr-CoV, e.g. SARS-CoV-2/a SARS-CoV-2 variant), e.g. COVID-19.

The clinical features of COVID-19 are described e.g. in Lechien et al., Journal of Internal Medicine (2020) 288(3): 335-344, International Severe Acute Respiratory and Emerging Infections Consortium (ISARIC). COVID-19 Report: 19 May 2020: ISARIC; 2020, Docherty et al., BMJ (2020) 369:m1985 and Bhardwaj et al., Int Rev Immunol. (2021) 2021:1-36, which are hereby incorporated by reference in their entirety. Common symptoms include cough, fever, headache, dyspnoea, anosmia, pharyngitis, nasal obstruction, rhinorrhoea, asthenia, myalgia, joint pain, gustatory dysfunction, abdominal pain, vomiting, and diarrhoea. The majority patients present with mild/moderate disease, however hospitalisation is sometimes required in particularly in elderly patients and/or patients having comorbidities such as diabetes and cardiovascular disease. A major complication in COVID-19 is progression to acute respiratory distress syndrome (ARDS), which presents as dyspnoea and acute respiratory failure, with patients requiring mechanical ventilation. A proportion of infected subjects are asymptomatic.

Treatment in accordance with the methods of the present disclosure may achieve one or more of: a reduction in the level or viral load of a sarbecovirus (e.g. a SARSr-CoV, e.g. SARS-CoV-2/a SARS-CoV-2 variant) in a subject or in a tissue/organ of the subject (e.g. the lungs), a reduction in the level of expression of a proinflammatory factor (e.g. IL-6, CCL2 and/or CXCL10) in a subject or in a tissue/organ of the subject (e.g. the lungs), an increase in the level of expression of IFNγ in the subject or in a tissue/organ of the subject (e.g. the lungs), a reduction in the number/proportion of cells infected with a sarbecovirus (e.g. a SARSr-CoV, e.g. SARS-CoV-2/a SARS-CoV-2 variant), inhibition of the development/progression of a disease/condition caused by infection with a sarbecovirus (e.g. a SARSr-CoV, e.g. SARS-CoV-2/a SARS-CoV-2 variant), e.g. COVID-19, in the subject, a reduction in the severity of symptoms of a disease/condition caused by infection with a sarbecovirus (e.g. a SARSr-CoV, e.g. SARS-CoV-2/a SARS-CoV-2 variant), e.g. COVID-19, in the subject, inhibition of the development/progression of acute respiratory distress syndrome (ARDS) in the subject, and an increase in survival of the subject.

In some embodiments, a subject may be selected for treatment described herein based on the determination of infection with a sarbecovirus (e.g. a SARS-COV, e.g. SARS-CoV-2/a SARS-CoV-2 variant), e.g. by detection of a sarbecovirus (e.g. a SARS-COV, e.g. SARS-CoV-2/a SARS-CoV-2 variant) in a sample obtained from the subject. In some embodiments, a subject may be selected for treatment described herein based on determination that the subject is at risk of having been infected with a sarbecovirus (e.g. a SARS-COV, e.g. SARS-CoV-2/a SARS-CoV-2 variant). For example, the subject might have been in close contact with a subject infected with a sarbecovirus (e.g. a SARSr-CoV, e.g. SARS-CoV-2/a SARS-CoV-2 variant).

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal, topical or transdermal. Administration may be by any suitable mode of nasal delivery, e.g. nasal drops, nasal spray, nebulizer, etc. Administration may be by injection or infusion.

Multiple doses of the antigen-binding molecule, polypeptide, nucleic acid (or plurality thereof), expression vector (or plurality thereof), cell, composition or combination described herein may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Administration of the articles of the present disclosure may be alone, or in combination with a further prophylactic/therapeutic agent, either simultaneously or sequentially dependent upon the disease/condition to be treated. The antigen-binding molecule, cell, composition or combination described herein and further prophylactic/therapeutic agent may be administered simultaneously or sequentially.

Simultaneous administration refers to administration of the antigen-binding molecule, polypeptide, nucleic acid (or plurality thereof), expression vector (or plurality thereof), cell, composition or combination of the present disclosure and the further prophylactic/therapeutic agent together, for example as a pharmaceutical composition containing both agents (combined preparation), or immediately after each other and optionally via the same route of administration, e.g. to the same artery, vein or other blood vessel. Sequential administration refers to administration of one of (i) the antigen-binding molecule, polypeptide, nucleic acid (or plurality thereof), expression vector (or plurality thereof), cell, composition or combination of the present disclosure, or (ii) the further prophylactic/therapeutic agent, followed after a given time interval by separate administration of the other of (i)/(ii). It is not required that (i) and (ii) are administered by the same route, although this is the case in some embodiments. The time interval may be any time interval.

The present disclosure further provides the use of an antigen-binding molecule/combination/composition according to the present disclosure to: inhibit interaction between a sarbecovirus spike protein (e.g. SARS-CoV-2 spike protein/a SARS-CoV-2 variant spike protein) and ACE2; and/or inhibit infection of ACE2-expressing cells by SARS-CoV-2/a SARS-CoV-2 variant. The present disclosure further provides methods for inhibiting interaction between a sarbecovirus spike protein (e.g. SARS-CoV-2 spike protein/a SARS-CoV-2 variant spike protein) and ACE2, and/or inhibiting infection of ACE2-expressing cells by SARS-CoV-2/a SARS-CoV-2 variant, using an antigen-binding molecule/combination/composition according to the present disclosure. Such uses/methods may be in vitro, or may be in vivo in a subject.

Accordingly, the present disclosure provides methods for inhibiting interaction between a sarbecovirus spike protein (e.g. SARS-CoV-2 spike protein/a SARS-CoV-2 variant spike protein) and ACE2, and/or inhibiting infection of ACE2-expressing cells by SARS-CoV-2/a SARS-CoV-2 variant, comprising administering to a subject an antigen-binding molecule/combination/composition according to the present disclosure.

### Methods of Detection

The present disclosure also provides the articles of the present disclosure for use in methods for detecting, localising or imaging a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), or cells comprising a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), e.g. as a consequence of infection with a sarbecovirus (e.g. a SARSr-CoV, e.g. SARS-CoV-2/a SARS-CoV-2 variant).

The antigen-binding molecules, combinations and compositions described herein may be used in methods that involve detecting binding of the antigen-binding molecule to a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein). Such methods may involve detection of the bound complex of an antigen-binding molecule and a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein). It will be appreciated that the sarbecovirus/sarbecovirus spike protein may be comprised in a cell, e.g. as a consequence of infection of the cell by the sarbecovirus.

As such, a method is provided, comprising contacting a sample containing, or suspected to contain, a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein) with an antigen-binding molecule/combination/composition according to the present disclosure, and detecting the formation of a complex of the antigen-binding molecule and a sarbecovirus/sarbecovirus spike protein. Also provided is a method comprising contacting a sample containing, or suspected to contain, a cell comprising a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein) with an antigen-binding molecule/combination/composition according to the present disclosure, and detecting the formation of a complex of the antigen-binding molecule and a sarbecovirus/sarbecovirus spike protein.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods comprising detecting a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), or cells comprising a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), include methods for diagnosing/prognosing a disease/condition described herein.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments, the method is performed in vivo.

Such methods may involve detecting or quantifying a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein) and/or cells comprising a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein), e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease/condition (e.g. COVID-19), predisposition to a disease/condition, or for providing a prognosis (prognosticating) for a disease/condition, e.g. a disease/condition described herein. The diagnosis or prognosis may relate to an existing (previously diagnosed) disease/condition.

A sample may be taken from any tissue or bodily fluid. The sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject.

A subject may be selected for diagnostic/prognostic evaluation based on the presence of symptoms indicative of a disease/condition described herein, or based on the subject being considered to be at risk of developing a disease/condition described herein.

The present disclosure also provides methods for selecting/stratifying a subject for treatment with a sarbecovirus (e.g. a SARSr-CoV, e.g. SARS-CoV-2/a SARS-CoV-2 variant)-targeted agent. In some embodiments a subject is selected for treatment/prevention in accordance with the methods of the present disclosure, or is identified as a subject which would benefit from such treatment/prevention, based on detection/quantification of a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), or cells comprising a sarbecovirus (e.g. a SARSr-CoV, e.g. SARS-CoV-2/a SARS-CoV-2 variant) and/or a sarbecovirus spike protein (e.g. SARS-CoV-2 spike protein/a SARS-CoV-2 variant spike protein), e.g. in a sample obtained from the individual.

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

In some embodiments, a subject may be selected for therapeutic or prophylactic intervention as described herein based on the detection of a sarbecovirus (e.g. a SARS-COV, e.g. SARS-CoV-2/a SARS-CoV-2 variant) and/or a sarbecovirus spike protein (e.g. SARS-CoV-2 spike protein/a SARS-CoV-2 variant spike protein), e.g. in a sample obtained from the subject.

### Kits

The present disclosure also provides kits of parts.

In some embodiments, the kit may have at least one container having a predetermined quantity of an antigen-binding molecule, polypeptide, nucleic acid (or plurality thereof), expression vector (or plurality thereof), cell, composition or combination described herein.

In some embodiments, the kit may comprise materials for producing an antigen-binding molecule, polypeptide, nucleic acid (or plurality thereof), expression vector (or plurality thereof), cell, composition or combination described herein. In some embodiments, the kit of parts may comprise materials for formulating an antigen-binding molecule, polypeptide, nucleic acid (or plurality thereof), expression vector (or plurality thereof), cell, composition or combination described herein to a pharmaceutical composition/medicament, e.g. in a composition further comprising a pharmaceutically-acceptable carrier, diluent, excipient or adjuvant.

The kit may provide the antigen-binding molecule, polypeptide, nucleic acid (or plurality thereof), expression vector (or plurality thereof), cell, composition or combination together with instructions for administration to a patient in order to treat a specified disease/condition (e.g. a disease/condition described herein).

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease/condition.

Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

### Sequences

| SEQ | | |
|---|---|---|
| ID | | |
| NO: | DESCRIPTION | SEQUENCE |
| 1 | SARS-CoV-2 spike | MFVFLVLLPLVSSQCVNLTTRTQLPPAYTNSFTRGVYYPDKVFRSSVLHSTQDLFLPFFS |
| | protein (UniProt: | NVTWFHAIHVSGTNGTKRFDNPVLPFNDGVYFASTEKSNIIRGWIFGTTLDSKTQSLLIV |
| | PODTC2-1, v1) | NNATNVVIKVCEFQFCNDPFLGVYYHKNNKSWMESEFRVYSSANNCTFEYVSQPFLMDLE |
| | | GKQGNFKNLREFVFKNIDGYFKIYSKHTPINLVRDLPQGFSALEPLVDLPIGINITRFQT |
| | | LLALHRSYLTPGDSSSGWTAGAAAYYVGYLQPRTFLLKYNENGTITDAVDCALDPLSETK |
| | | CTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGKIAD |
| | | YNYKLPDDFTGCVIAWNSNNLDSKVGGNYNYLYRLFRKSNLKPFERDISTEIYQAGSTPC |
| | | NGVEGFNCYFPLQSYGFQPTNGVGYQPYRVVVLSFELLHAPATVCGPKKSTNLVKNKCVN |
| | | FNFNGLTGTGVLTESNKKFLPFQQFGRDIADTTDAVRDPQTLEILDITPCSFGGVSVITP |
| | | GTNTSNQVAVLYQDVNCTEVPVAIHADQLTPTWRVYSTGSNVFQTRAGCLIGAEHVNNSY |
| | | ECDIPIGAGICASYQTQTNSPRRARSVASQSIIAYTMSLGAENSVAYSNNSIAIPTNFTI |
| | | SVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDC |
| | | LGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQDVVNQNAQALN |
| | | TLVKQLSSNFGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQEKNFTTAPA |
| | | ICHDGKAHFPREGVFVSNGTHWFVTQRNFYEPQIITTDNTFVSGNCDVVIGIVNNTVYDP |
| | | LQPELDSFKEELDKYFKNHTSPDVDLGDISGINASVVNIQKEIDRLNEVAKNLNESLIDL |
| | | QELGKYEQYIKWPWYIWLGFIAGLIAIVMVTIMLCCMTSCCSCLKGCCSCGSCCKFDEDD |
| | | SEPVLKGVKLHYT |
| 2 | SARS-CoV-2 spike | MFVFLVLLPLVS |
| | protein signal peptide | |
| | (PODTC2-1 positions 1- | |
| | 12) | |
| 3 | SARS-CoV-2 spike | SQCVNLTTRTQLPPAYTNSFTRGVYYPDKVFRSSVLHSTQDLFLPFFSNVTWFHAIHVSG |
| | protein extracellular | TNGTKRFDNPVLPFNDGVYFASTEKSNIIRGWIFGTTLDSKTQSLLIVNNATNVVIKVCE |
| | (PODTC2-1 positions | FQFCNDPFLGVYYHKNNKSWMESEFRVYSSANNCTFEYVSQPFLMDLEGKQGNFKNLREF |
| | 13-1213) | VFKNIDGYFKIYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSETKCTLKSFTVEKGI |
| | | YQTSNFRVQPTESIVRFPNITNLCPFGEVFNATRFASVYAWNRKRISNCVADYSVLYNSA |
| | | SFSTFKCYGVSPTKLNDLCFTNVYADSFVIRGDEVRQIAPGQTGKIADYNYKLPDDFTGC |
| | | VIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVL |
| | | TESNKKFLPFQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECDIPIGAGICA |
| | | SYQTQTNSPRRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQEVFAQVKQIYKTP |
| | | PIKDFGGFNFSQILPDPSKPSKRSFIEDLLFNKVTLADAGFIKQYGDCLGDIAARDLICA |
| | | QKFNGLTVLPPLLTDEMIAQYTSALLAGTITSGWTFGAGAALQIPFAMQMAYRFNGIGVT |
| | | QNVLYENQKLIANQFNSAIGKIQDSLSSTASALGKLQDVVNQNAQALNTLVKQLSSNFGA |
| | | ISSVLNDILSRLDKVEAEVQIDRLITGRLQSLQTYVTQQLIRAAEIRASANLAATKMSEC |
| | | VLGQSKRVDFCGKGYHLMSFPQSAPHGVVFLHVTYVPAQEKNFTTAPAICHDGKAHFPRE |
| | | GVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKW |
| | | P |
| 4 | SARS-CoV-2 spike | WYIWLGFIAGLIAIVMVTIML |
| | protein helical | |
| | (PODTC2-1 positions | |
| | 1214-1234) | |
| 5 | SARS-CoV-2 spike | CCMTSCCSCLKGCCSCGSCCKFDEDDSEPVLKGVKLHYT |
| | protein cytoplasmic | |
| | (PODTC2-1 positions | |
| | 1235-1273) | |
| 6 | SARS-CoV-2 spike | SQCVNLTTRTQLPPAYTNSFTRGVYYPDKVFRSSVLHSTQDLFLPFFSNVTWFHAIHVSG |
| | protein S1 (PODTC2-1 | TNGTKRFDNPVLPFNDGVYFASTEKSNIIRGWIFGTTLDSKTQSLLIVNNATNVVIKVCE |
| | positions 13-685) | FQFCNDPFLGVYYHKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSETKCTLKSFTVEKGI |
| | | YQTSNFRVQPTESIVRFPNITNLCPFGEVFNATRFASVYAWNRKRISNCVADYSVLYNSA |
| | | SFSTFKCYGVSPTKLNDLCFTNVYADSFVIRGDEVRQIAPGQTGKIADYNYKLPDDFTGC |
| | | VIAWNSNNLDSKVGGNYNYLYRLFRKSNLKPFERDISTEIYQAGSTPCNGVEGFNCYFPL |
| | | QSYGFQPTNGVGYQPYRVVVLSFELLHAPATVCGPKKSTNLVKNKCVNFNFNGLTGTGVL |
| | | TESNKKFLPFQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECDIPIGAGICA |
| | | SYQTQTNSPRRAR |
| 7 | SARS-CoV-2 spike | RVQPTESIVRFPNITNLCPFGEVFNATRFASVYAWNRKRISNCVADYSVLYNSASFSTFK |
| | protein RBD (PODTC2- | CYGVSPTKLNDLCFTNVYADSFVIRGDEVRQIAPGQTGKIADYNYKLPDDFTGCVIAWNS |
| | 1 positions 319-541) | NNLDSKVGGNYNYLYRLFRKSNLKPFERDISTEIYQAGSTPCNGVEGFNCYFPLQSYGFQ |
| | | PTNGVGYQPYRVVVLSFELLHAPATVCGPKKSTNLVKNKCVNF |
| 8 | SARS-CoV-2 spike | NSNNLDSKVGGNYNYLYRLFRKSNLKPFERDISTEIYQAGSTPCNGVEGFNCYFPLQSYG |
| | protein RBM (PODTC2- | FQPTNGVGYQPY |
| | 1 positions 437-508) | |
| 9 | SARS-CoV-2 spike | SVASQSIIAYTMSLGAENSVAYSNNSIAIPTNFTISVTTEILPVSMTKTSVDCTMYICGD |
| | protein S2 (PODTC2-1 | STECSNLLLQYGSFCTQLNRALTGIAVEQDKNTQEVFAQVKQIYKTPPIKDFGGFNFSQI |
| | positions 686-1273) | LPDPSKPSKRSFIEDLLFNKVTLADAGFIKQYGDCLGDIAARDLICAQKFNGLTVLPPLL |
| | | TDEMIAQYTSALLAGTITSGWTFGAGAALQIPFAMQMAYRFNGIGVTQNVLYENQKLIAN |
| | | QFNSAIGKIQDSLSSTASALGKLQDVVNQNAQALNTLVKQLSSNFGAISSVLNDILSRLD |
| | | KVEAEVQIDRLITGRLQSLQTYVTQQLIRAAEIRASANLAATKMSECVLGQSKRVDFCGK |
| | | GYHLMSFPQSAPHGVVFLHVTYVPAQEKNFTTAPAICHDGKAHFPREGVFVSNGTHWFVT |
| | | QRNFYEPQIITTDNTFVSGNCDVVIGIVNNTVYDPLQPELDSFKEELDKYFKNHTSPDVD |
| | | LGDISGINASVVNIQKEIDRLNEVAKNLNESLIDLQELGKYEQYIKWPWYIWLGFIAGLI |
| | | AIVMVTIMLCCMTSCCSCLKGCCSCGSCCKFDEDDSEPVLKGVKLHYT |
| 10 | SARS-CoV-2 spike | SFIEDLLFNKVTLADAGFIKQYGDCLGDIAARDLICAQKFNGLTVLPPLLTDEMIAQYTS |
| | protein S2' (PODTC2-1 | ALLAGTITSGWTFGAGAALQIPFAMQMAYRFNGIGVTQNVLYENQKLIANQFNSAIGKIQ |
| | positions 816-1273) | DSLSSTASALGKLQDVVNQNAQALNTLVKQLSSNFGAISSVLNDILSRLDKVEAEVQIDR |
| | | LITGRLQSLQTYVTQQLIRAAEIRASANLAATKMSECVLGQSKRVDFCGKGYHLMSFPQS |
| | | APHGVVFLHVTYVPAQEKNFTTAPAICHDGKAHFPREGVFVSNGTHWFVTQRNFYEPQII |
| | | TTDNTFVSGNCDVVIGIVNNTVYDPLQPELDSFKEELDKYFKNHTSPDVDLGDISGINAS |
| | | VVNIQKEIDRLNEVAKNLNESLIDLQELGKYEQYIKWPWYIWLGFIAGLIAIVMVTIMLC |
| | | CMTSCCSCLKGCCSCGSCCKFDEDDSEPVLKGVKLHYT |
| 11 | BA. 1 spike protein | MFVFLVLLPLVSSQCVNLTTRTQLPPAYTNSFTRGVYYPDKVFRSSVLHSTQDLFLPFFS |
| | | NVTWFHVISGTNGTKRFDNPVLPFNDGVYFASIEKSNIIRGWIFGTTLDSKTQSLLIVNN |
| | | ATNVVIKVCEFQFQNDPFLDHKNNKSWMESEFRVYSSANNCTFEYVSQPFLMDLEGKQGN |
| | | FKNLREFVFKNIDGYFKIYSKHTPIIVREPEDLPQGFSALEPLVDLPIGINITRFQTLLA |
| | | LHRSYLTPGDSSSGWTAGAAAYYVGYLQPRTFLLKYNENGTITDAVDCALDPLSETKCTL |
| | | KSFTVEKGIYQTSNFRVQPTESIVRFPNITNLCPFDEVFNATRFASVYAWNRKRISNCVA |
| | | DYSVLYNLAPFFTFKCYGVSPTKLNDLCFTNVYADSFVIRGDEVRQIAPGQTGNIADYNY |
| | | KLPDDFTGCVIAWNSNKLDSKVSGNYNYLYRLFRKSNLKPFERDISTEIYQAGNKPCNGV |
| | | AGFNCYFPLRSYSFRPTYGVGHQPYRVVVLSFELLHAPATVCGPKKSTNLVKNKCVNFNF |
| | | NGLKGTGVLTESNKKFLPFQQFGRDIADTTDAVRDPQTLEILDITPCSFGGVSVITPGTN |
| | | TSNQVAVLYQGVNCTEVPVAIHADQLTPTWRVYSTGSNVFQTRAGCLIGAEYVNNSYECD |
| | | IPIGAGICASYQTQTKSHRRARSVASQSIIAYTMSLGAENSVAYSNNSIAIPTNFTISVT |
| | | TEILPVSMTKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIKQYGDCLGD |
| | | IAARDLICAQKFKGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNHNAQALNTLV |
| | | KQLSSKFGAISSVLNDIFSRLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTDNTFVSGNCDVVIGIVNNTVYDPLQP |
| | | ELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 12 | BA.2 spike protein | MFVFLVLLPLVSSQCVNLITRTQSYTNSFTRGVYYPDKVFRSSVLHSTQDLFLPFFSNVT |
| | | WFHAIHVSGTNGTKRFDNPVLPFNDGVYFASTEKSNIIRGWIFGTTLDSKTQSLLIVNNA |
| | | TNVVIKVCEFQFCNDPFLDVYYHKNNKSWMESEFRVYSSANNCTFEYVSQPFLMDLEGKQ |
| | | GNFKNLREFVFKNIDGYFKIYSKHTPINLGRDLPQGFSALEPLVDLPIGINITRFQTLLA |
| | | LHRSYLTPGDSSSGWTAGAAAYYVGYLQPRTFLLKYNENGTITDAVDCALDPLSETKCTL |
| | | KSFTVEKGIYQTSNFRVQPTESIVRFPNITNLCPFDEVFNATRFASVYAWNRKRISNCVA |
| | | DYSVLYNFAPFFAFKCYGVSPTKLNDLCFTNVYADSFVIRGNEVSQIAPGQTGNIADYNY |
| | | KLPDDFTGCVIAWNSNKLDSKVGGNYNYLYRLFRKSNLKPFERDISTEIYQAGNKPCNGV |
| | | AGFNCYFPLRSYGFRPTYGVGHQPYRVVVLSFELLHAPATVCGPKKSTNLVKNKCVNFNF |
| | | NGLTGTGVLTESNKKFLPFQQFGRDIADTTDAVRDPQTLEILDITPCSFGGVSVITPGTN |
| | | TSNQVAVLYQGVNCTEVPVAIHADQLTPTWRVYSTGSNVFQTRAGCLIGAEYVNNSYECD |
| | | IPIGAGICASYQTQTKSHRRARSVASQSIIAYTMSLGAENSVAYSNNSIAIPTNFTISVT |
| | | TEILPVSMTKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIKQYGDCLGD |
| | | IAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNHNAQALNTLV |
| | | KQLSSKFGAISSVLNDILSRLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTDNTFVSGNCDVVIGIVNNTVYDPLQP |
| | | ELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 13 | BA.5 spike protein | MFVFLVLLPLVSSQCVNLITRTQSYTNSFTRGVYYPDKVFRSSVLHSTQDLFLPFFSNVT |
| | | WFHAISGTNGTKRFDNPVLPFNDGVYFASTEKSNIIRGWIFGTTLDSKTQSLLIVNNATN |
| | | VVIKVCEFQFQNDPFLDVYYHKNNKSWMESEFRVYSSANNCTFEYVSQPFLMDLEGKQGN |
| | | FKNLREFVFKNIDGYFKIYSKHTPINLGRDLPQGFSALEPLVDLPIGINITRFQTLLALH |
| | | RSYLTPGDSSSGWTAGAAAYYVGYLQPRTFLLKYNENGTITDAVDCALDPLSETKCTLKS |
| | | FTVEKGIYQTSNFRVQPTESIVRFPNITNLCPFDEVFNATRFASVYAWNRKRISNCVADY |
| | | SVLYNFAPFFAFKCYGVSPTKLNDLCFTNVYADSFVIRGNEVSQIAPGQTGNIADYNYKL |
| | | PDDFTGCVIAWNSNKLDSKVGGNYNYRYRLFRKSNLKPFERDISTEIYQAGNKPCNGVAG |
| | | VNCYFPLQSYGFRPTYGVGHQPYRVVVLSFELLHAPATVCGPKKSTNLVKNKCVNFNFNG |
| | | LTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIP |
| | | IGAGICASYQTQTKSHRRARSVASQSIIAYTMSLGAENSVAYSNNSIAIPTNFTISVTTE |
| | | ILPVSMTKTSVDCTMYICGDSTECSNLLLQYGSFCTQLKRALTGIAVEQDKNTQEVFAQV |
| | | KQIYKTPPIKYFGGFNFSQILPDPSKPSKRSFIEDLLFNKVTLADAGFIKQYGDCLGDIA |
| | | ARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNHNAQALNTLVKQ |
| | | LSSKFGAISSVLNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQEKNFTTAPAICHDG |
| | | KAHFPREGVFVSNGTHWFVTQRNFYEPQIITTDNTFVSGNCDVVIGIVNNTVYDPLQPEL |
| | | DSFKEELDKYFKNHTSPDVDLGDISGINASVVNIQKEIDRLNEVAKNLNESLIDLQELGK |
| | | YEQYIKWPWYIWLGFIAGLIAIVMVTIMLCCMTSCCSCLKGCCSCGSCCKFDEDDSEPVL |
| | | KGVKLHYT |
| 14 | BA.2.75 spike protein | MFVFLVLLPLVSSQCVNLITRTQSYTNSFTRGVYYPDKVFRSSVLHSTQDLFLPFFSNVT |
| | | WFHAIHVSGTNGTKRFDNPVLPFNDGVYFASTEKSNIIRGWIFGTTLDSKTQSLLIVNNA |
| | | TNVVIKVCEFQFCNDPFLDVYYHENNKSRMESELRVYSSANNCTFEYVSQPFLMDLEGKQ |
| | | GNFKNLREFVFKNIDGYFKIYSKHTPVNLGRDLPQGFSALEPLVDLPIGINITRFQTLLA |
| | | LHRSYLTPGDSSSSWTAGAAAYYVGYLQPRTFLLKYNENGTITDAVDCALDPLSETKCTL |
| | | KSFTVEKGIYQTSNFRVQPTESIVRFPNITNLCPFHEVFNATRFASVYAWNRKRISNCVA |
| | | DYSVLYNFAPFFAFKCYGVSPTKLNDLCFTNVYADSFVIRGNEVSQIAPGQTGNIADYNY |
| | | KLPDDFTGCVIAWNSNKLDSKVSGNYNYLYRLFRKSKLKPFERDISTEIYQAGNKPCNGV |
| | | AGFNCYFPLQSYGFRPTYGVGHQPYRVVVLSFELLHAPATVCGPKKSTNLVKNKCVNFNF |
| | | NGLTGTGVLTESNKKFLPFQQFGRDIADTTDAVRDPQTLEILDITPCSFGGVSVITPGTN |
| | | TSNQVAVLYQGVNCTEVPVAIHADQLTPTWRVYSTGSNVFQTRAGCLIGAEYVNNSYECD |
| | | IPIGAGICASYQTQTKSHRRARSVASQSIIAYTMSLGAENSVAYSNNSIAIPTNFTISVT |
| | | TEILPVSMTKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIKQYGDCLGD |
| | | IAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNHNAQALNTLV |
| | | KQLSSKFGAISSVLNDILSRLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTDNTFVSGNCDVVIGIVNNTVYDPLQP |
| | | ELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 15 | BA.2.75.2 spike protein | MFVFLVLLPLVSSQCVNLITRTQSYTNSFTRGVYYPDKVFRSSVLHSTQDLFLPFFSNVT |
| | | WFHAIHVSGTNGTKRFDNPVLPFNDGVYFASTEKSNIIRGWIFGTTLDSKTQSLLIVNNA |
| | | TNVVIKVCEFQFCNDPFLDVYYHENNKSRMESELRVYSSANNCTFEYVSQPFLMDLEGKQ |
| | | GNFKNLREFVFKNIDGYFKIYSKHTPVNLGRDLPQGFSALEPLVDLPIGINITRFQTLLA |
| | | LHRSYLTPGDSSSSWTAGAAAYYVGYLQPRTFLLKYNENGTITDAVDCALDPLSETKCTL |
| | | KSFTVEKGIYQTSNFRVQPTESIVRFPNITNLCPFHEVFNATTFASVYAWNRKRISNCVA |
| | | DYSVLYNFAPFFAFKCYGVSPTKLNDLCFTNVYADSFVIRGNEVSQIAPGQTGNIADYNY |
| | | KLPDDFTGCVIAWNSNKLDSKVSGNYNYLYRLFRKSKLKPFERDISTEIYQAGNKPCNGV |
| | | AGSNCYFPLQSYGFRPTYGVGHQPYRVVVLSFELLHAPATVCGPKKSTNLVKNKCVNFNF |
| | | NGLTGTGVLTESNKKFLPFQQFGRDIADTTDAVRDPQTLEILDITPCSFGGVSVITPGTN |
| | | TSNQVAVLYQGVNCTEVPVAIHADQLTPTWRVYSTGSNVFQTRAGCLIGAEYVNNSYECD |
| | | IPIGAGICASYQTQTKSHRRARSVASQSIIAYTMSLGAENSVAYSNNSIAIPTNFTISVT |
| | | TEILPVSMTKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIKQYGDCLGD |
| | | IAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNHNAQALNTLV |
| | | KQLSSKFGAISSVLNDILSRLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTDNTFVSGNCDVVIGIVNNTVYDPLQP |
| | | ELDSFKEELDKYFKNHTSPDVDLGDISGINASVVNIQKEIDRLNEVAKNLNESLINLQEL |
| | | GKYEQYIKWPWYIWLGFIAGLIAIVMVTIMLCCMTSCCSCLKGCCSCGSCCKFDEDDSEP |
| | | VLKGVKLHYT |
| 16 | BA.4.6.1 spike protein | MFVFLVLLPLVSSQCVNLITRTQSYTNSFTRGVYYPDKVFRSSVLHSTQDLFLPFFSNVT |
| | | WFHAISGTNGTKRFDNPVLPFNDGVYFASTEKSNIIRGWIFGTTLDSKTQSLLIVNNATN |
| | | VVIKVCEFQFQNDPFLDVYYHKNNKSLMESEFRVYSSANNCTFEYVSQPFLMDLEGKQGN |
| | | FKNLREFVFKNIDGYFKIYSKHTPINLGRDLPQGFSALEPLVDLPIGINITRFQTLLALH |
| | | RSYLTPGDSSSGWTAGAAAYYVGYLQPRTFLLKYNENGTITDAVDCALDPLSETKCTLKS |
| | | FTVEKGIYQTSNFRVQPTESIVRFPNITNLCPFDEVFNATTFASVYAWNRKRISNCVADY |
| | | SVLYNFAPFFAFKCYGVSPTKLNDLCFTNVYADSFVIRGNEVSQIAPGQTGNIADYNYKL |
| | | PDDFTGCVIAWNSNKLDSKVGGNYNYRYRLFRKSNLKPFERDISTEIYQAGNKPCNGVAG |
| | | VNCYFPLQSYGFRPTYGVGHQPYRVVVLSFELLHAPATVCGPKKSTNLVKNKCVNFNFNG |
| | | LTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNSSYECDIP |
| | | IGAGICASYQTQTKSHRRARSVASQSIIAYTMSLGAENSVAYSNNSIAIPTNFTISVTTE |
| | | ILPVSMTKTSVDCTMYICGDSTECSNLLLQYGSFCTQLKRALTGIAVEQDKNTQEVFAQV |
| | | KQIYKTPPIKYFGGFNFSQILPDPSKPSKRSFIEDLLFNKVTLADAGFIKQYGDCLGDIA |
| | | ARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNHNAQALNTLVKQ |
| | | LSSKFGAISSVLNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQEKNFTTAPAICHDG |
| | | KAHFPREGVFVSNGTHWFVTQRNFYEPQIITTDNTFVSGNCDVVIGIVNNTVYDPLQPEL |
| | | DSFKEELDKYFKNHTSPDVDLGDISGINASVVNIQKEIDRLNEVAKNLNESLIDLQELGK |
| | | YEQYIKWPWYIWLGFIAGLIAIVMVTIMLCCMTSCCSCLKGCCSCGSCCKFDEDDSEPVL |
| | | KGVKLHYT |
| 17 | BF.7 spike protein | MFVFLVLLPLVSSQCVNLITRTQSYTNSFTRGVYYPDKVFRSSVLHSTQDLFLPFFSNVT |
| | | WFHAISGTNGTKRFDNPVLPFNDGVYFASTEKSNIIRGWIFGTTLDSKTQSLLIVNNATN |
| | | VVIKVCEFQFQNDPFLDVYYHKNNKSWMESEFRVYSSANNCTFEYVSQPFLMDLEGKQGN |
| | | FKNLREFVFKNIDGYFKIYSKHTPINLGRDLPQGFSALEPLVDLPIGINITRFQTLLALH |
| | | RSYLTPGDSSSGWTAGAAAYYVGYLQPRTFLLKYNENGTITDAVDCALDPLSETKCTLKS |
| | | FTVEKGIYQTSNFRVQPTESIVRFPNITNLCPFDEVFNATTFASVYAWNRKRISNCVADY |
| | | SVLYNFAPFFAFKCYGVSPTKLNDLCFTNVYADSFVIRGNEVSQIAPGQTGNIADYNYKL |
| | | PDDFTGCVIAWNSNKLDSKVGGNYNYRYRLFRKSNLKPFERDISTEIYQAGNKPCNGVAG |
| | | VNCYFPLQSYGFRPTYGVGHQPYRVVVLSFELLHAPATVCGPKKSTNLVKNKCVNFNFNG |
| | | LTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIP |
| | | IGAGICASYQTQTKSHRRARSVASQSIIAYTMSLGAENSVAYSNNSIAIPTNFTISVTTE |
| | | ILPVSMTKTSVDCTMYICGDSTECSNLLLQYGSFCTQLKRALTGIAVEQDKNTQEVFAQV |
| | | KQIYKTPPIKYFGGFNFSQILPDPSKPSKRSFIEDLLFNKVTLADAGFIKQYGDCLGDIA |
| | | ARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNHNAQALNTLVKQ |
| | | LSSKFGAISSVLNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQEKNFTTAPAICHDG |
| | | KAHFPREGVFVSNGTHWFVTQRNFYEPQIITTDNTFVSGNCDVVIGIVNNTVYDPLQPEL |
| | | DSFKEELDKYFKNHTSPDVDLGDISGINASVVNIQKEIDRLNEVAKNLNESLIDLQELGK |
| | | YEQYIKWPWYIWLGFIAGLIAIVMVTIMLCCMTSCCSCLKGCCSCGSCCKFDEDDSEPVL |
| | | KGVKLHYT |
| 18 | BQ.1.1 spike protein | MFVFLVLLPLVSSQCVNLITRTQSYTNSFTRGVYYPDKVFRSSVLHSTQDLFLPFFSNVT |
| | | WFHAISGTNGTKRFDNPVLPFNDGVYFASTEKSNIIRGWIFGTTLDSKTQSLLIVNNATN |
| | | VVIKVCEFQFQNDPFLDVYYHKNNKSWMESEFRVYSSANNCTFEYVSQPFLMDLEGKQGN |
| | | FKNLREFVFKNIDGYFKIYSKHTPINLGRDLPQGFSALEPLVDLPIGINITRFQTLLALH |
| | | RSYLTPGDSSSGWTAGAAAYYVGYLQPRTFLLKYNENGTITDAVDCALDPLSETKCTLKS |
| | | FTVEKGIYQTSNFRVQPTESIVRFPNITNLCPFDEVFNATTFASVYAWNRKRISNCVADY |
| | | SVLYNFAPFFAFKCYGVSPTKLNDLCFTNVYADSFVIRGNEVSQIAPGQTGNIADYNYKL |
| | | PDDFTGCVIAWNSNKLDSTVGGNYNYRYRLFRKSKLKPFERDISTEIYQAGNKPCNGVAG |
| | | VNCYFPLQSYGFRPTYGVGHQPYRVVVLSFELLHAPATVCGPKKSTNLVKNKCVNFNFNG |
| | | LTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIP |
| | | IGAGICASYQTQTKSHRRARSVASQSIIAYTMSLGAENSVAYSNNSIAIPTNFTISVTTE |
| | | ILPVSMTKTSVDCTMYICGDSTECSNLLLQYGSFCTQLKRALTGIAVEQDKNTQEVFAQV |
| | | KQIYKTPPIKYFGGFNFSQILPDPSKPSKRSFIEDLLFNKVTLADAGFIKQYGDCLGDIA |
| | | ARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNHNAQALNTLVKQ |
| | | LSSKFGAISSVLNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQEKNFTTAPAICHDG |
| | | KAHFPREGVFVSNGTHWFVTQRNFYEPQIITTDNTFVSGNCDVVIGIVNNTVYDPLQPEL |
| | | DSFKEELDKYFKNHTSPDVDLGDISGINASVVNIQKEIDRLNEVAKNLNESLIDLQELGK |
| | | YEQYIKWPWYIWLGFIAGLIAIVMVTIMLCCMTSCCSCLKGCCSCGSCCKFDEDDSEPVL |
| | | KGVKLHYT |
| 19 | XBB.1 spike protein | MFVFLVLLPLVSSQCVNLITRTQSYTNSFTRGVYYPDKVFRSSVLHSTQDLFLPFFSNVT |
| | | WFHAIHVSGTNGTKRFDNPALPFNDGVYFASTEKSNIIRGWIFGTTLDSKTQSLLIVNNA |
| | | TNVVIKVCEFQFCNDPFLDVYQKNNKSWMESEFRVYSSANNCTFEYVSQPFLMDLEGKEG |
| | | NFKNLREFVFKNIDGYFKIYSKHTPINLERDLPQGFSALEPLVDLPIGINITRFQTLLAL |
| | | HRSYLTPVDSSSGWTAGAAAYYVGYLQPRTFLLKYNENGTITDAVDCALDPLSETKCTLK |
| | | SFTVEKGIYQTSNFRVQPTESIVRFPNITNLCPFHEVENATTFASVYAWNRKRISNCVAD |
| | | YSVIYNFAPFFAFKCYGVSPTKLNDLCFTNVYADSFVIRGNEVSQIAPGQTGNIADYNYK |
| | | LPDDFTGCVIAWNSNKLDSKPSGNYNYLYRLFRKSKLKPFERDISTEIYQAGNKPCNGVA |
| | | GSNCYSPLQSYGFRPTYGVGHQPYRVVVLSFELLHAPATVCGPKKSTNLVKNKCVNFNFN |
| | | GLTGTGVLTESNKKFLPFQQFGRDIADTTDAVRDPQTLEILDITPCSFGGVSVITPGTNT |
| | | SNQVAVLYQGVNCTEVPVAIHADQLTPTWRVYSTGSNVFQTRAGCLIGAEYVNNSYECDI |
| | | PIGAGICASYQTQTKSHRRARSVASQSIIAYTMSLGAENSVAYSNNSIAIPTNFTISVTT |
| | | EILPVSMTKTSVDCTMYICGDSTECSNLLLQYGSFCTQLKRALTGIAVEQDKNTQEVFAQ |
| | | VKQIYKTPPIKYFGGFNFSQILPDPSKPSKRSFIEDLLFNKVTLADAGFIKQYGDCLGDI |
| | | AARDLICAQKFNGLTVLPPLLTDEMIAQYTSALLAGTITSGWTFGAGAALQIPFAMQMAY |
| | | RFNGIGVTQNVLYENQKLIANQFNSAIGKIQDSLSSTASALGKLQDVVNHNAQALNTLVK |
| | | QLSSKFGAISSVLNDILSRLDKVEAEVQIDRLITGRLQSLQTYVTQQLIRAAEIRASANL |
| | | AATKMSECVLGQSKRVDFCGKGYHLMSFPQSAPHGVVFLHVTYVPAQEKNFTTAPAICHD |
| | | GKAHFPREGVFVSNGTHWFVTQRNFYEPQIITTDNTFVSGNCDVVIGIVNNTVYDPLQPE |
| | | LDSFKEELDKYFKNHTSPDVDLGDISGINASVVNIQKEIDRLNEVAKNLNESLIDLQELG |
| | | KYEQYIKWPWYIWLGFIAGLIAIVMVTIMLCCMTSCCSCLKGCCSCGSCCKFDEDDSEPV |
| | | LKGVKLHYT |
| 20 | XBB.1.16 spike protein | MFVFLVLLPLVSSQCVNLITRTQSYTNSFTRGVYYPDKVFRSSVLHSTQDLFLPFFSNVT |
| | | WFHAIHVSGTNGTKRFDNPALPFNDGVYFASTEKSNIIRGWIFGTTLDSKTQSLLIVNNA |
| | | TNVVIKVCEFQFCNDPFLDVYQKNNKSWMESEFRVYSSANNCTFEYVSQPFLMDLVGKEG |
| | | NFKNLREFVFKNIDGYFKIYSKHTPINLERDLPQGFSALEPLVDLPIGINITRFQTLLAL |
| | | HRSYLTPVDSSSGWTAGAAAYYVGYLQPRTFLLKYNENGTITDAVDCALDPLSETKCTLK |
| | | SFTVEKGIYQTSNFRVQPTESIVRFPNITNLCPFHEVFNATTFASVYAWNRKRISNCVAD |
| | | YSVIYNFAPFFAFKCYGVSPTKLNDLCFTNVYADSFVIRGNEVSQIAPGQTGNIADYNYK |
| | | LPDDFTGCVIAWNSNKLDSKPSGNYNYLYRLFRKSKLKPFERDISTEIYQAGNRPCNGVA |
| | | GPNCYSPLQSYGFRPTYGVGHQPYRVVVLSFELLHAPATVCGPKKSTNLVKNKCVNFNFN |
| | | GLTGTGVLTESNKKFLPFQQFGRDIADTTDAVRDPQTLEILDITPCSFGGVSVITPGTNT |
| | | SNQVAVLYQGVNCTEVPVAIHADQLTPTWRVYSTGSNVFQTRAGCLIGAEYVNNSYECDI |
| | | PIGAGICASYQTQTKSHRRARSVASQSIIAYTMSLGAENSVAYSNNSIAIPTNFTISVTT |
| | | EILPVSMTKTSVDCTMYICGDSTECSNLLLQYGSFCTQLKRALTGIAVEQDKNTQEVFAQ |
| | | VKQIYKTPPIKYFGGFNFSQILPDPSKPSKRSFIEDLLFNKVTLADAGFIKQYGDCLGDI |
| | | AARDLICAQKFNGLTVLPPLLTDEMIAQYTSALLAGTITSGWTFGAGAALQIPFAMQMAY |
| | | RFNGIGVTQNVLYENQKLIANQFNSAIGKIQDSLSSTASALGKLQDVVNHNAQALNTLVK |
| | | QLSSKFGAISSVLNDILSRLDKVEAEVQIDRLITGRLQSLQTYVTQQLIRAAEIRASANL |
| | | AATKMSECVLGQSKRVDFCGKGYHLMSFPQSAPHGVVFLHVTYVPAQEKNFTTAPAICHD |
| | | GKAHFPREGVFVSNGTHWFVTQRNFYEPQIITTDNTFVSGNCDVVIGIVNNTVYDPLQPE |
| | | LDSFKEELDKYFKNHTSPDVDLGDISGINASVVNIQKEIDRLNEVAKNLNESLIDLQELG |
| | | KYEQYIKWPWYIWLGFIAGLIAIVMVTIMLCCMTSCCSCLKGCCSCGSCCKFDEDDSEPV |
| | | LKGVKLHYT |
| 21 | XBB.2.3 spike protein | MFVFLVLLPLVSSQCVNLITRTQSYTNSFTRGVYYPDKVFRSSVLHSTQDLFLPFFSNVT |
| | | WFHAIHVSGTNGTKRFDNPALPFNDGVYFASTEKSNIIRGWIFGTTLDSKTQSLLIVNNA |
| | | TNVVIKVCEFQFCNDPFLDVYQKNNKSWMESEFRVYSSANNCTFEYVSQPFLMDLEGKEG |
| | | NFKNLREFVFKNIDGYFKIYSKHTPINLERDLPQGFSALEPLVDLPIGINITRFQTLLAL |
| | | HRSYLTPGGSSSGWTAGAAAYYVGYLQPRTFLLKYNENGTITDAVDCALDPLSETKCTLK |
| | | SFTVEKGIYQTSNFRVQPTESIVRFPNITNLCPFHEVFNATTFASVYAWNRKRISNCVAD |
| | | YSVIYNFAPFFAFKCYGVSPTKLNDLCFTNVYADSFVIRGNEVSQIAPGQTGNIADYNYK |
| | | LPDDFTGCVIAWNSNKLDSKPSGNYNYLYRLFRKSKLKPFERDISTEIYQAGNKPCNGVA |
| | | GPNCYSPLQSYGFRPTYGVGHQPYRVVVLSFELLHASATVCGPKKSTNLVKNKCVNFNFN |
| | | GLTGTGVLTESNKKFLPFQQFGRDIADTTDAVRDPQTLEILDITPCSFGGVSVITPGTNT |
| | | SNQVAVLYQGVNCTEVPVAIHADQLTPTWRVYSTGSNVFQTRAGCLIGAEYVNNSYECDI |
| | | PIGAGICASYQTQTKSHRRARSVASQSIIAYTMSLGAENSVAYSNNSIAIPTNFTISVTT |
| | | EILPVSMTKTSVDCTMYICGDSTECSNLLLQYGSFCTQLKRALTGIAVEQDKNTQEVFAQ |
| | | VKQIYKTPPIKYFGGFNFSQILPDPSKPSKRSFIEDLLFNKVTLADAGFIKQYGDCLGDI |
| | | AARDLICAQKFNGLTVLPPLLTDEMIAQYTSALLAGTITSGWTFGAGAALQIPFAMQMAY |
| | | RFNGIGVTQNVLYENQKLIANQFNSAIGKIQDSLSSTASALGKLQDVVNHNAQALNTLVK |
| | | QLSSKFGAISSVLNDILSRLDKVEAEVQIDRLITGRLQSLQTYVTQQLIRAAEIRASANL |
| | | AATKMSECVLGQSKRVDFCGKGYHLMSFPQSAPHGVVFLHVTYVPAQEKNFTTAPAICHD |
| | | GKAHFPREGVFVSNGTHWFVTQRNFYEPQIITTDNTFVSGNCDVVIGIVNNTVYDPLQPE |
| | | LDSFKEELDKYFKNHTSPDVDLGDISGINASVVNIQKEIDRLNEVAKNLNESLIDLQELG |
| | | KYEQYIKWPWYIWLGFIAGLIAIVMVTIMLCCMTSCCSCLKGCCSCGSCCKFDEDDSEPV |
| | | LKGVKLHYT |
| 22 | B.1.1.7 spike protein | MFVFLVLLPLVSSQCVNLTTRTQLPPAYTNSFTRGVYYPDKVFRSSVLHSTQDLFLPFFS |
| | | NVTWFHAISGTNGTKRFDNPVLPFNDGVYFASTEKSNIIRGWIFGTTLDSKTQSLLIVNN |
| | | ATNVVIKVCEFQFQNDPFLGVYHKNNKSWMESEFRVYSSANNCTFEYVSQPFLMDLEGKQ |
| | | GNFKNLREFVFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVDCALDPLSETKCTL |
| | | KSFTVEKGIYQTSNFRVQPTESIVRFPNITNLCPFGEVFNATRFASVYAWNRKRISNCVA |
| | | DYSVLYNSASFSTFKCYGVSPTKLNDLCFTNVYADSFVIRGDEVRQIAPGQTGKIADYNY |
| | | KLPDDFTGCVIAWNSNNLDSKVGGNYNYLYRLFRKSNLKPFERDISTEIYQAGSTPCNGV |
| | | EGFNCYFPLQSYGFQPTYGVGYQPYRVVVLSFELLHAPATVCGPKKSTNLVKNKCVNFNF |
| | | NGLTGTGVLTESNKKFLPFQQFGRDIDDTTDAVRDPQTLEILDITPCSFGGVSVITPGTN |
| | | TSNQVAVLYQGVNCTEVPVAIHADQLTPTWRVYSTGSNVFQTRAGCLIGAEHVNNSYECD |
| | | IPIGAGICASYQTQTNSHRRARSVASQSIIAYTMSLGAENSVAYSNNSIAIPINFTISVT |
| | | TEILPVSMTKTSVDCTMYICGDSTECSNLLLQYGSFCTQLNRALTGIAVEQDKNTQEVFA |
| | | QVKQIYKTPPIKDFGGFNFSQILPDPSKPSKRSFIEDLLFNKVTLADAGFIKQYGDCLGD |
| | | IAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNQNAQALNTLV |
| | | KQLSSNFGAISSVLNDILARLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTHNTFVSGNCDVVIGIVNNTVYDPLQP |
| | | ELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 23 | B.1.351 spike protein | MFVFLVLLPLVSSQCVNLTTRTQLPPAYTNSFTRGVYYPDKVFRSSVLHSTQDLFLPFFS |
| | | NVTWFHAIHVSGTNGTKRFANPVLPFNDGVYFASTEKSNIIRGWIFGTTLDSKTQSLLIV |
| | | NNATNVVIKVCEFQFCNDPFLGVYYHKNNKSWMESEFRVYSSANNCTFEYVSQPFLMDLE |
| | | GKQGNFKNLREFVFKNIDGYFKIYSKHTPINLVRGLPQGFSALEPLVDLPIGINITRFQT |
| | | LHRSYLTPGDSSSGWTAGAAAYYVGYLQPRTFLLKYNENGTITDAVDCALDPLSETKCTL |
| | | KSFTVEKGIYQTSNFRVQPTESIVRFPNITNLCPFGEVFNATRFASVYAWNRKRISNCVA |
| | | DYSVLYNSASFSTFKCYGVSPTKLNDLCFTNVYADSFVIRGDEVRQIAPGQTGNIADYNY |
| | | KLPDDFTGCVIAWNSNNLDSKVGGNYNYLYRLFRKSNLKPFERDISTEIYQAGSTPCNGV |
| | | KGFNCYFPLQSYGFQPTYGVGYQPYRVVVLSFELLHAPATVCGPKKSTNLVKNKCVNFNF |
| | | NGLTGTGVLTESNKKFLPFQQFGRDIADTTDAVRDPQTLEILDITPCSFGGVSVITPGTN |
| | | TSNQVAVLYQGVNCTEVPVAIHADQLTPTWRVYSTGSNVFQTRAGCLIGAEHVNNSYECD |
| | | IPIGAGICASYQTQTNSPRRARSVASQSIIAYTMSLGVENSVAYSNNSIAIPTNFTISVT |
| | | TEILPVSMTKTSVDCTMYICGDSTECSNLLLQYGSFCTQLNRALTGIAVEQDKNTQEVFA |
| | | QVKQIYKTPPIKDFGGFNFSQILPDPSKPSKRSFIEDLLFNKVTLADAGFIKQYGDCLGD |
| | | IAARDLICAQKFNGLTVLPPLLTDEMIAQYTSALLAGTITSGWTFGAGAALQIPFAMQMA |
| | | YRFNGIGVTQNVLYENQKLIANQFNSAIGKIQDSLSSTASALGKLQDVVNQNAQALNTLV |
| | | KQLSSNFGAISSVLNDILSRLDKVEAEVQIDRLITGRLQSLQTYVTQQLIRAAEIRASAN |
| | | LAATKMSECVLGQSKRVDFCGKGYHLMSFPQSAPHGVVFLHVTYVPAQEKNFTTAPAICH |
| | | DGKAHFPREGVFVSNGTHWFVTQRNFYEPQIITTDNTFVSGNCDVVIGIVNNTVYDPLQP |
| | | ELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 24 | B.1.617.2 spike protein | MFVFLVLLPLVSSQCVNLITRTQLPPAYTNSFTRGVYYPDKVFRSSVLHSTQDLFLPFFS |
| | | NVTWFHAIHVSGTNGTKRFDNPVLPFNDGVYFASTEKSNIIRGWIFGTTLDSKTQSLLIV |
| | | NNATNVVIKVCEFQFCNDPFLGVYYHKNNKSWMESEFRVYSSANNCTFEYVSQPFLMDLE |
| | | GKQGNFKNLREFVFKNIDGYFKIYSKHTPINLVRDLPQGFSALEPLVDLPIGINITRFQT |
| | | LLALHRSYLTPGDSSSGWTAGAAAYYVGYLQPRTFLLKYNENGTITDAVDCALDPLSETK |
| | | CTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGKIAD |
| | | YNYKLPDDFTGCVIAWNSNNLDSKVGGNYNYRYRLFRKSNLKPFERDISTEIYQAGSKPC |
| | | NGVEGFNCYFPLQSYGFQPTNGVGYQPYRVVVLSFELLHAPATVCGPKKSTNLVKNKCVN |
| | | FNFNGLTGTGVLTESNKKFLPFQQFGRDIADTTDAVRDPQTLEILDITPCSFGGVSVITP |
| | | GTNTSNQVAVLYQGVNCTEVPVAIHADQLTPTWRVYSTGSNVFQTRAGCLIGAEHVNNSY |
| | | ECDIPIGAGICASYQTQTNSRRRARSVASQSIIAYTMSLGAENSVAYSNNSIAIPTNFTI |
| | | SVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDC |
| | | LGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQNVVNQNAQALN |
| | | TLVKQLSSNFGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQEKNFTTAPA |
| | | ICHDGKAHFPREGVFVSNGTHWFVTQRNFYEPQIITTDNTFVSGNCDVVIGIVNNTVYDP |
| | | LQPELDSFKEELDKYFKNHTSPDVDLGDISGINASVVNIQKEIDRLNEVAKNLNESLIDL |
| | | QELGKYEQYIKWPWYIWLGFIAGLIAIVMVTIMLCCMTSCCSCLKGCCSCGSCCKFDEDD |
| | | SEPVLKGVKLHYT |
| 25 | P.1 spike protein | MFVFLVLLPLVSSQCVNFTNRTQLPSAYTNSFTRGVYYPDKVFRSSVLHSTQDLFLPFFS |
| | | NVTWFHAIHVSGTNGTKRFDNPVLPFNDGVYFASTEKSNIIRGWIFGTTLDSKTQSLLIV |
| | | NNATNVVIKVCEFQFCNYPFLGVYYHKNNKSWMESEFRVYSSANNCTFEYVSQPFLMDLE |
| | | GKQGNFKNLSEFVFKNIDGYFKIYSKHTPINLVRDLPQGFSALEPLVDLPIGINITRFQT |
| | | LLALHRSYLTPGDSSSGWTAGAAAYYVGYLQPRTFLLKYNENGTITDAVDCALDPLSETK |
| | | CTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGTIAD |
| | | YNYKLPDDFTGCVIAWNSNNLDSKVGGNYNYLYRLFRKSNLKPFERDISTEIYQAGSTPC |
| | | NGVKGFNCYFPLQSYGFQPTYGVGYQPYRVVVLSFELLHAPATVCGPKKSTNLVKNKCVN |
| | | FNFNGLTGTGVLTESNKKFLPFQQFGRDIADTTDAVRDPQTLEILDITPCSFGGVSVITP |
| | | GTNTSNQVAVLYQGVNCTEVPVAIHADQLTPTWRVYSTGSNVFQTRAGCLIGAEYVNNSY |
| | | ECDIPIGAGICASYQTQTNSPRRARSVASQSIIAYTMSLGAENSVAYSNNSIAIPTNFTI |
| | | SVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDC |
| | | LGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQDVVNQNAQALN |
| | | TLVKQLSSNFGAISSVLNDILSRLDKVEAEVQIDRLITGRLQSLQTYVTQQLIRAAEIRA |
| | | SANLAAIKMSECVLGQSKRVDFCGKGYHLMSFPQSAPHGVVFLHVTYVPAQEKNFTTAPA |
| | | ICHDGKAHFPREGVFVSNGTHWFVTQRNFYEPQIITTDNTFVSGNCDVVIGIVNNTVYDP |
| | | LQPELDSFKEELDKYFKNHTSPDVDLGDISGINASFVNIQKEIDRLNEVAKNLNESLIDL |
| | | QELGKYEQYIKWPWYIWLGFIAGLIAIVMVTIMLCCMTSCCSCLKGCCSCGSCCKFDEDD |
| | | SEPVLKGVKLHYT |
| 26 | EG.5 spike protein | MFVFLVLLPLVSSQCVNLITRTQSYTNSFTRGVYYPDKVFRSSVLHSTQDLFLPFFSNVT |
| | | WFHAIHVSGTNGTKRFDNPALPFNDGVYFASTEKSNIIRGWIFGTTLDSKTQSLLIVNNA |
| | | TNVVIKVCEFQFCNDPFLDVYQKNNKSWMESEFRVYSSANNCTFEYVSQPFLMDLEGKSG |
| | | NFKNLREFVFKNIDGYFKIYSKHTPINLERDLPQGFSALEPLVDLPIGINITRFQTLLAL |
| | | HRSYLTPVDSSSGWTAGAAAYYVGYLQPRTFLLKYNENGTITDAVDCALDPLSETKCTLK |
| | | SFTVEKGIYQTSNFRVQPTESIVRFPNITNLCPFHEVFNATTFASVYAWNRKRISNCVAD |
| | | YSVIYNFAPFFAFKCYGVSPTKLNDLCFTNVYADSFVIRGNEVSQIAPGQTGNIADYNYK |
| | | LPDDFTGCVIAWNSNKLDSKPSGNYNYLYRLLRKSKLKPFERDISTEIYQAGNKPCNGVA |
| | | GPNCYSPLQSYGFRPTYGVGHQPYRVVVLSFELLHAPATVCGPKKSTNLVKNKCVNFNFN |
| | | GLTGTGVLTESNKKFLPFQQFGRDIADTTDAVRDPQTLEILDITPCSFGGVSVITPGTNT |
| | | SNQVAVLYQGVNCTEVPVAIHADQLTPTWRVYSTGSNVFQTRAGCLIGAEYVNNSYECDI |
| | | PIGAGICASYQTQTKSHRRARSVASQSIIAYTMSLGAENSVAYSNNSIAIPTNFTISVTT |
| | | EILPVSMTKTSVDCTMYICGDSTECSNLLLQYGSFCTQLKRALTGIAVEQDKNTQEVFAQ |
| | | VKQIYKTPPIKYFGGFNFSQILPDPSKPSKRSFIEDLLFNKVTLADAGFIKQYGDCLGDI |
| | | AARDLICAQKFNGLTVLPPLLTDEMIAQYTSALLAGTITSGWTFGAGAALQIPFAMQMAY |
| | | RFNGIGVTQNVLYENQKLIANQFNSAIGKIQDSLSSTASALGKLQDVVNHNAQALNTLVK |
| | | QLSSKFGAISSVLNDILSRLDKVEAEVQIDRLITGRLQSLQTYVTQQLIRAAEIRASANL |
| | | AATKMSECVLGQSKRVDFCGKGYHLMSFPQSAPHGVVFLHVTYVPAQEKNFTTAPAICHD |
| | | GKAHFPREGVFVSNGTHWFVTQRNFYEPQIITTDNTFVSGNCDVVIGIVNNTVYDPLQPE |
| | | LDSFKEELDKYFKNHTSPDVDLGDISGINASVVNIQKEIDRLNEVAKNLNESLIDLQELG |
| | | KYEQYIKWPWYIWLGFIA |
| 27 | EG.5.1 spike protein | MFVFLVLLPLVSSQCVNLITRTQSYTNSFTRGVYYPDKVFRSSVLHSTHDLFLPFFSNVT |
| | | WFHAIHVSGTNGTKRFDNPALPFNDGVYFASTEKSNIIRGWIFGTTLDSKTQSLLIVNNA |
| | | TNVVIKVCEFQFCNDPFLDVYQKNNKSWMESEFRVYSSANNCTFEYVSQPFLMDLEGKSG |
| | | NFKNLREFVFKNIDGYFKIYSKHTPINLERDLPQGFSALEPLVDLPIGINITRFQTLLAL |
| | | HRSYLTPVDSSSGWTAGAAAYYVGYLQPRTFLLKYNENGTITDAVDCALDPLSETKCTLK |
| | | SFTVEKGIYQTSNFRVQPTESIVRFPNITNLCPFHEVFNATTFASVYAWNRKRISNCVAD |
| | | YSVIYNFAPFFAFKCYGVSPTKLNDLCFTNVYADSFVIRGNEVSQIAPGQTGNIADYNYK |
| | | LPDDFTGCVIAWNSNKLDSKPSGNYNYLYRLLRKSKLKPFERDISTEIYQAGNKPCNGVA |
| | | GPNCYSPLQSYGFRPTYGVGHQPYRVVVLSFELLHAPATVCGPKKSTNLVKNKCVNFNFN |
| | | GLTGTGVLTESNKKFLPFQQFGRDIADTTDAVRDPQTLEILDITPCSFGGVSVITPGTNT |
| | | SNQVAVLYQGVNCTEVPVAIHADQLTPTWRVYSTGSNVFQTRAGCLIGAEYVNNSYECDI |
| | | PIGAGICASYQTQTKSHRRARSVASQSIIAYTMSLGAENSVAYSNNSIAIPTNFTISVTT |
| | | EILPVSMTKTSVDCTMYICGDSTECSNLLLQYGSFCTQLKRALTGIAVEQDKNTQEVFAQ |
| | | VKQIYKTPPIKYFGGFNFSQILPDPSKPSKRSFIEDLLFNKVTLADAGFIKQYGDCLGDI |
| | | AARDLICAQKFNGLTVLPPLLTDEMIAQYTSALLAGTITSGWTFGAGAALQIPFAMQMAY |
| | | RFNGIGVTQNVLYENQKLIANQFNSAIGKIQDSLSSTASALGKLQDVVNHNAQALNTLVK |
| | | QLSSKFGAISSVLNDILSRLDKVEAEVQIDRLITGRLQSLQTYVTQQLIRAAEIRASANL |
| | | AATKMSECVLGQSKRVDFCGKGYHLMSFPQSAPHGVVFLHVTYVPAQEKNFTTAPAICHD |
| | | GKAHFPREGVFVSNGTHWFVTQRNFYEPQIITTDNTFVSGNCDVVIGIVNNTVYDPLQPE |
| | | LDSFKEELDKYFKNHTSPDVDLGDISGINASVVNIQKEIDRLNEVAKNLNESLIDLQELG |
| | | KYEQYIKWPWYIWLGFIAGLIAIVMVTIMLCCMTSCCSCLKGCCSCGSCCKFDEDDSEPV |
| | | LKGVKLHYT |
| 28 | Human ACE2 isoform 1 | MSSSSWLLLSLVAVTAAQSTIEEQAKTFLDKFNHEAEDLFYQSSLASWNYNTNITEENVQ |
| | (UniProt: Q9BYF1-1, | NMNNAGDKWSAFLKEQSTLAQMYPLQEIQNLTVKLQLQALQQNGSSVLSEDKSKRLNTIL |
| | v1) | NTMSTIYSTGKVCNPDNPQECLLLEPGLNEIMANSLDYNERLWAWESWRSEVGKQLRPLY |
| | | EEYVVLKNEMARANHYEDYGDYWRGDYEVNGVDGYDYSRGQLIEDVEHTFEEIKPLYEHL |
| | | HAYVRAKLMNAYPSYISPIGCLPAHLLGDMWGRFWTNLYSLTVPFGQKPNIDVTDAMVDQ |
| | | AWDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGTLPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLH |
| | | KCDISNSTEAGQKLFNMLRLGKSEPWTLALENVVGAKNMNVRPLLNYFEPLFTWLKDQNK |
| | | NSFVGWSTDWSPYADQSIKVRISLKSALGDKAYEWNDNEMYLFRSSVAYAMRQYFLKVKN |
| | | QMILFGEEDVRVANLKPRISFNFFVTAPKNVSDIIPRTEVEKAIRMSRSRINDAFRLNDN |
| | | SLEFLGIQPTLGPPNQPPVSIWLIVFGVVMGVIVVGIVILIFTGIRDRKKKNKARSGENP |
| | | YASIDISKGENNPGFQNTDDVQTSF |
| 29 | Human ACE2 signal | MSSSSWLLLSLVAVTAA |
| | peptide (Q9BYF1-1 | |
| | positions 1-17) | |
| 30 | Human ACE2 | QSTIEEQAKTFLDKFNHEAEDLFYQSSLASWNYNTNITEENVQNMNNAGDKWSAFLKEQS |
| | extracellular (Q9BYF1- | TLAQMYPLQEIQNLTVKLQLQALQQNGSSVLSEDKSKRLNTILNTMSTIYSTGKVCNPDN |
| | 1 positions 18-740) | PQECLLLEPGLNEIMANSLDYNERLWAWESWRSEVGKQLRPLYEEYVVLKNEMARANHYE |
| | | DYGDYWRGDYEVNGVDGYDYSRGQLIEDVEHTFEEIKPLYEHLHAYVRAKLMNAYPSYIS |
| | | PIGCLPAHLLGDMWGRFWTNLYSLTVPFGQKPNIDVTDAMVDQAWDAQRIFKEAEKFFVS |
| | | VGLPNMTQGFWENSMLTDPGNVQKAVCHPTAWDLGKGDFRILMCTKVTMDDFLTAHHEMG |
| | | HIQYDMAYAAQPFLLRNGANEGFHEAVGEIMSLSAATPKHLKSIGLLSPDFQEDNETEIN |
| | | FLLKQALTIVGTLPFTYMLEKWRWMVFKGEIPKDQWMKKWWEMKREIVGVVEPVPHDETY |
| | | CDPASLFHVSNDYSFIRYYTRTLYQFQFQEALCQAAKHEGPLHKCDISNSTEAGQKLFNM |
| | | LRLGKSEPWTLALENVVGAKNMNVRPLLNYFEPLFTWLKDQNKNSFVGWSTDWSPYADQS |
| | | IKVRISLKSALGDKAYEWNDNEMYLFRSSVAYAMRQYFLKVKNQMILFGEEDVRVANLKP |
| | | RISFNFFVTAPKNVSDIIPRTEVEKAIRMSRSRINDAFRLNDNSLEFLGIQPTLGPPNQP |
| | | PVS |
| 31 | Human ACE2 helical | IWLIVFGVVMGVIVVGIVILI |
| | (Q9BYF1-1 positions | |
| | 741-761) | |
| 32 | Human ACE2 | FTGIRDRKKKNKARSGENPYASIDISKGENNPGFQNTDDVQTSF |
| | cytoplasmic (Q9BYF1-1 | |
| | positions 762-805) | |
| 33 | Human ACE2 enzyme | QSTIEEQAKTFLDKFNHEAEDLFYQSSLASWNYNTNITEENVQNMNNAGDKWSAFLKEQS |
| | (Q9BYF1-1 positions | TLAQMYPLQEIQNLTVKLQLQALQQNGSSVLSEDKSKRLNTILNTMSTIYSTGKVCNPDN |
| | 18-805) | PQECLLLEPGLNEIMANSLDYNERLWAWESWRSEVGKQLRPLYEEYVVLKNEMARANHYE |
| | | DYGDYWRGDYEVNGVDGYDYSRGQLIEDVEHTFEEIKPLYEHLHAYVRAKLMNAYPSYIS |
| | | PIGCLPAHLLGDMWGRFWTNLYSLTVPFGQKPNIDVTDAMVDQAWDAQRIFKEAEKFFVS |
| | | VGLPNMTQGFWENSMLTDPGNVQKAVCHPTAWDLGKGDFRILMCTKVTMDDFLTAHHEMG |
| | | HIQYDMAYAAQPFLLRNGANEGFHEAVGEIMSLSAATPKHLKSIGLLSPDFQEDNETEIN |
| | | FLLKQALTIVGTLPFTYMLEKWRWMVFKGEIPKDQWMKKWWEMKREIVGVVEPVPHDETY |
| | | CDPASLFHVSNDYSFIRYYTRTLYQFQFQEALCQAAKHEGPLHKCDISNSTEAGQKLFNM |
| | | LRLGKSEPWTLALENVVGAKNMNVRPLLNYFEPLFTWLKDQNKNSFVGWSTDWSPYADQS |
| | | IKVRISLKSALGDKAYEWNDNEMYLFRSSVAYAMRQYFLKVKNQMILFGEEDVRVANLKP |
| | | RISFNFFVTAPKNVSDIIPRTEVEKAIRMSRSRINDAFRLNDNSLEFLGIQPTLGPPNQP |
| | | PVSIWLIVFGVVMGVIVVGIVILIFTGIRDRKKKNKARSGENPYASIDISKGENNPGFQN |
| | | TDDVQTSF |
| 34 | Human ACE2 | QSTIEEQAKTFLDKFNHEAEDLFYQSSLASWNYNTNITEENVQNMNNAGDKWSAFLKEQS |
| | processed enzyme | TLAQMYPLQEIQNLTVKLQLQALQQNGSSVLSEDKSKRLNTILNTMSTIYSTGKVCNPDN |
| | (Q9BYF1-1 positions | PQECLLLEPGLNEIMANSLDYNERLWAWESWRSEVGKQLRPLYEEYVVLKNEMARANHYE |
| | 18-708) | DYGDYWRGDYEVNGVDGYDYSRGQLIEDVEHTFEEIKPLYEHLHAYVRAKLMNAYPSYIS |
| | | PIGCLPAHLLGDMWGRFWTNLYSLTVPFGQKPNIDVTDAMVDQAWDAQRIFKEAEKFFVS |
| | | VGLPNMTQGFWENSMLTDPGNVQKAVCHPTAWDLGKGDFRILMCTKVTMDDFLTAHHEMG |
| | | HIQYDMAYAAQPFLLRNGANEGFHEAVGEIMSLSAATPKHLKSIGLLSPDFQEDNETEIN |
| | | FLLKQALTIVGTLPFTYMLEKWRWMVFKGEIPKDQWMKKWWEMKREIVGVVEPVPHDETY |
| | | CDPASLFHVSNDYSFIRYYTRTLYQFQFQEALCQAAKHEGPLHKCDISNSTEAGQKLFNM |
| | | LRLGKSEPWTLALENVVGAKNMNVRPLLNYFEPLFTWLKDQNKNSFVGWSTDWSPYADQS |
| | | IKVRISLKSALGDKAYEWNDNEMYLFRSSVAYAMRQYFLKVKNQMILFGEEDVRVANLKP |
| | | RISFNFFVTAPKNVSDIIPRTEVEKAIRMSR |
| 35 | Human ACE2 isoform 2 | MSSSSWLLLSLVAVTAAQSTIEEQAKTFLDKFNHEAEDLFYQSSLASWNYNTNITEENVQ |
| | (UniProt: Q9BYF1-2) | NMNNAGDKWSAFLKEQSTLAQMYPLQEIQNLTVKLQLQALQQNGSSVLSEDKSKRLNTIL |
| | | NTMSTIYSTGKVCNPDNPQECLLLEPGLNEIMANSLDYNERLWAWESWRSEVGKQLRPLY |
| | | EEYVVLKNEMARANHYEDYGDYWRGDYEVNGVDGYDYSRGQLIEDVEHTFEEIKPLYEHL |
| | | HAYVRAKLMNAYPSYISPIGCLPAHLLGDMWGRFWTNLYSLTVPFGQKPNIDVTDAMVDQ |
| | | AWDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGTLPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLH |
| | | KCDISNSTEAGQKLL |
| 36 | Ab1 (B11.2) VH | EVQLVESGGGLVQPGGSLRLSCAASEIIVSRNYMTWVRQAPGKGLEWLAVLYAGGSSFYA |
| | | DSVKGRFTISRDNSKNTLFLQMNSLRAEDTAVYYCARDLSLSGGFDYWGQGALVTVSS |
| 37 | Ab1, Ab2, HC-CDR1 | EIIVSRNY |
| 38 | Ab1 HC-CDR2 | LYAGGSS |
| 39 | Ab1 HC-CDR3 | ARDLSLSGGFDY |
| 40 | Ab1 HC-FR1 | EVQLVESGGGLVQPGGSLRLSCAAS |
| 41 | Ab1 HC-FR2 | MTWVRQAPGKGLEWLAV |
| 42 | Ab1 HC-FR3 | FYADSVKGRFTISRDNSKNTLFLQMNSLRAEDTAVYYC |
| 43 | Ab1, Ab12, Ab20 HC- | WGQGALVTVSS |
| | FR4 | |
| 44 | Ab1 (B11.2) VL | DIQLTQSPSFLSASVGDRVTITCRASQGISNYLAWYQQNPGKAPKLLIYAVSTLHSGVPS |
| | | RFSGSGSGTEFTLTISSLQPEDFATYYCQHLNTDSCTFGQGTKLEIK |
| 45 | Ab1, Ab47 LC-CDR1 | QGISNY |
| 46 | Ab1 LC-CDR2 | AVS |
| 47 | Ab1 LC-CDR3 | QHLNTDSCT |
| 48 | Ab1 LC-FR1 | DIQLTQSPSFLSASVGDRVTITCRAS |
| 49 | Ab1 LC-FR2 | LAWYQQNPGKAPKLLIY |
| 50 | Ab1 LC-FR3 | TLHSGVPSRFSGSGSGTEFTLTISSLQPEDFATYYC |
| 51 | Ab1, Ab16, Ab28, | FGQGTKLEIK |
| | Ab32, Ab43, Ab48 LC- | |
| | FR4 | |
| 52 | Ab2 (H12.2) VH | EFQLVESGGRLVRPGGSLRLSCVASEIIVSRNYMSWIRQAPGKGLEWVSILYAGGTTYYA |
| | | DSVKGRFTISRDNSKNTLYLQLNSLRVEDTAIYYCVRPIVGGRGGMDVWGQGTTVTVSS |
| 53 | Ab2 HC-CDR2 | LYAGGTT |
| 54 | Ab2 HC-CDR3 | VRPIVGGRGGMDV |
| 55 | Ab2 HC-FR1 | EFQLVESGGRLVRPGGSLRLSCVAS |
| 56 | Ab2, Ab5 HC-FR2 | MSWIRQAPGKGLEWVSI |
| 57 | Ab2 HC-FR3 | YYADSVKGRFTISRDNSKNTLYLQLNSLRVEDTAIYYC |
| 58 | Ab2, Ab4, Ab5, Ab21, | WGQGTTVTVSS |
| | Ab22, Ab23, Ab25, | |
| | Ab27, Ab33, Ab37, | |
| | Ab48 HC-FR4 | |
| 59 | Ab2 (H12.2) VL | DIQMTQSPSSLSASVGDRVTITCQASQDINKYLNWYQQKPGKAPKLLIYDASNLEPGVPS |
| | | RFSGSGSGTDFVFTITSLQPEDIATYYCHYYDDVPYTFGQGTQLEIK |
| 60 | Ab2 Ab5 LC-CDR1 | QDINKY |
| 61 | Ab2, Ab6, Ab26, Ab35, | DAS |
| | Ab43, Ab46 LC-CDR2 | |
| 62 | Ab2 LC-CDR3 | HYYDDVPYT |
| 63 | Ab2, Ab5 LC-FR1 | DIQMTQSPSSLSASVGDRVTITCQAS |
| 64 | Ab2, Ab5 LC-FR2 | LNWYQQKPGKAPKLLIY |
| 65 | Ab2, Ab5 LC-FR3 | NLEPGVPSRFSGSGSGTDFVFTITSLQPEDIATYYC |
| 66 | Ab2, Ab5 LC-FR4 | FGQGTQLEIK |
| 67 | Ab3 (C2.2) VH | QVQLVQSGAEVKKPGSSVRVSCEASGDTFSRYAISWVRQAPGQGLEWMGRIIPMFGMANS |
| | | AQKFQARVTISADKSTSTAYMEVSSLRSEDTAVYYCATDTFYPNDGVHRMEYWGQGALVI |
| | | VSS |
| 68 | Ab3, Ab24, Ab31, Ab47 | GDTFSRYA |
| | HC-CDR1 | |
| 69 | Ab3 HC-CDR2 | IIPMFGMA |
| 70 | Ab3 HC-CDR3 | ATDTFYPNDGVHRMEY |
| 71 | Ab3 HC-FR1 | QVQLVQSGAEVKKPGSSVRVSCEAS |
| 72 | Ab3, Ab4, Ab21, Ab22, | ISWVRQAPGQGLEWMGR |
| | Ab25, Ab31, Ab38 HC- | |
| | FR2 | |
| 73 | Ab3 HC-FR3 | NSAQKFQARVTISADKSTSTAYMEVSSLRSEDTAVYYC |
| 74 | Ab3 HC-FR4 | WGQGALVIVSS |
| 75 | Ab3 (C2.2) VL | DIQMTQSPSSLSASVGDRVTITCRASQTISNYLNWYQQKPGKAPNLLIYGTSILQSGVPS |
| | | RFSGSGSGTDFTLTISSLQPEDFATYYCQQSHSAPRTFGPGTKVDIK |
| 76 | Ab3 LC-CDR1 | QTISNY |
| 77 | Ab3, Ab38 LC-CDR2 | GTS |
| 78 | Ab3 LC-CDR3 | QQSHSAPRT |
| 79 | Ab3, Ab4, Ab12, Ab30, | DIQMTQSPSSLSASVGDRVTITCRAS |
| | Ab40, Ab42, Ab43, | |
| | Ab47 LC-FR1 | |
| 80 | Ab3, Ab31 LC-FR2 | LNWYQQKPGKAPNLLIY |
| 81 | Ab3 LC-FR3 | ILQSGVPSRFSGSGSGTDFTLTISSLQPEDFATYYC |
| 82 | Ab3, Ab9, Ab27, Ab33, | FGPGTKVDIK |
| | Ab46, Ab52 LC-FR4 | |
| 83 | Ab4 (G7.2) VH | QVQLVQSGAEVKKPGSSVKVSCEASGGTFSTYAISWVRQAPGQGLEWMGRIIPIFGIANY |
| | | AQKFQGRVTITADKSTSTAYMEVSSLRSEDTAVYYCATTFYDHSSTYRTHSMDVWGQGTT |
| | | VTVSS |
| 84 | Ab4 HC-CDR1 | GGTFSTYA |
| 85 | Ab4 HC-CDR2 | IIPIFGIA |
| 86 | Ab4 HC-CDR3 | ATTFYDHSSTYRTHSMDV |
| 87 | Ab4, Ab24, Ab47 HC- | QVQLVQSGAEVKKPGSSVKVSCEAS |
| | FR1 | |
| 88 | Ab4 HC-FR3 | NYAQKFQGRVTITADKSTSTAYMEVSSLRSEDTAVYYC |
| 89 | Ab4 (G7.2) VL | DIQMTQSPSSLSASVGDRVTITCRASQSISNYLNWYQQRPGKAPKLLIYAASGLQSGVPS |
| | | RFSGSGSGTNFTLTISTLHPEDFATYYCQQTHSTPRAFGGGTKVEIK |
| 90 | Ab4 LC-CDR1 | QSISNY |
| 91 | Ab4, Ab11, Ab13, | AAS |
| | Ab14, Ab27, Ab31, | |
| | Ab33, Ab34, Ab37, | |
| | Ab39, Ab42 LC-CDR2 | |
| 92 | Ab4, Ab31 LC-CDR3 | QQTHSTPRA |
| 93 | Ab4 LC-FR2 | LNWYQQRPGKAPKLLIY |
| 94 | Ab4 LC-FR3 | GLQSGVPSRFSGSGSGTNFTLTISTLHPEDFATYYC |
| 95 | Ab4, Ab11, Ab31, | FGGGTKVEIK |
| | Ab39, Ab49 LC-FR4 | |
| 96 | Ab5 (10x-L8N-c25) VH | DFQLVESGGGLVRPGGSLRLSCVASEITVSRNYMSWIRQAPGKGLEWVSIIYPGGTTYYA |
| | | DSVKGRFTISRDNSKNTMYLQLNSLRPEDTAIYYCVRPIVRGGGGMDVWGQGTTVTVSS |
| 97 | Ab5 HC-CDR1 | EITVSRNY |
| 98 | Ab5 HC-CDR2 | IYPGGTT |
| 99 | Ab5 HC-CDR3 | VRPIVRGGGGMDV |
| 100 | Ab5 HC-FR1 | DFQLVESGGGLVRPGGSLRLSCVAS |
| 101 | Ab5 HC-FR3 | YYADSVKGRFTISRDNSKNTMYLQLNSLRPEDTAIYYC |
| 102 | Ab5 (10x-L8N-c25) VL | DIQMTQSPSSLSASVGDRVTITCQASQDINKYLNWYQQKPGKAPKLLIYDATNLEPGVPS |
| | | RFSGSGSGTDFVFTITSLQPEDIATYYCQYYDDVPYTFGQGTQLEIK |
| 103 | Ab5 LC-CDR2 | DAT |
| 104 | Ab5 LC-CDR3 | QYYDDVPYT |
| 105 | Ab6 (K23) VH | EVQLVESGGGLVQPGGSLRLSCSASGFTFNNYVMHWVRQAPGKGLEYVSAINSNGGSTYY |
| | | AGSVKGRFTISRDNSNNTLYLQMSSLRAEDTAVYYCIKDAGYYSSLGVDSWGQGTLVTVS |
| | | S |
| 106 | Ab6 HC-CDR1 | GFTFNNYV |
| 107 | Ab6 HC-CDR2 | INSNGGST |
| 108 | Ab6 HC-CDR3 | IKDAGYYSSLGVDS |
| 109 | Ab6 HC-FR1 | EVQLVESGGGLVQPGGSLRLSCSAS |
| 110 | Ab6 HC-FR2 | MHWVRQAPGKGLEYVSA |
| 111 | Ab6 HC-FR3 | YYAGSVKGRFTISRDNSNNTLYLQMSSLRAEDTAVYYC |
| 112 | Ab6, Ab10, Ab13, | WGQGTLVTVSS |
| | Ab15, Ab18, Ab19, | |
| | Ab24, Ab26, Ab28, | |
| | Ab29, Ab38, Ab39, | |
| | Ab40, Ab41, Ab42, | |
| | Ab45, Ab46, Ab52, E7, | |
| | LyCov-1404 HC-FR4 | |
| 113 | Ab6 (K23) VL | EIVLTQSPATLSLSPGERATLSCRASQSVGSYLAWFQQKPGQAPRLLIYDASYRATGIPA |
| | | RFSGSGSGTDFTLTISSLEPEDVGIYYCQQRSNWPQTFGGGTKVDIK |
| 114 | Ab6, Ab35 LC-CDR1 | QSVGSY |
| 115 | Ab6 LC-CDR3 | QQRSNWPQT |
| 116 | Ab6, Ab35 LC-FR1 | EIVLTQSPATLSLSPGERATLSCRAS |
| 117 | Ab6 LC-FR2 | LAWFQQKPGQAPRLLIY |
| 118 | Ab6 LC-FR3 | YRATGIPARFSGSGSGTDFTLTISSLEPEDVGIYYC |
| 119 | Ab6 LC-FR4 | FGGGTKVDIK |
| 120 | Ab7 (G1) VH | EVQLVESGGGLVKPGGSLRLSCAASGFTVSTYIINWVRQAPGKGLEWVSSITSESDYMFD |
| | | ADSVRGRFTISRDNAKNLVYLQMNSLRAEDTAVYYCARDQGAYSGYDLSPGGDAFDVWGQ |
| | | GTMVTVSS |
| 121 | Ab7 HC-CDR1 | GFTVSTYI |
| 122 | Ab7 HC-CDR2 | ITSESDYM |
| 123 | Ab7 HC-CDR3 | ARDQGAYSGYDLSPGGDAFDV |
| 124 | Ab7, Ab19, Ab20, Ab43 | EVQLVESGGGLVKPGGSLRLSCAAS |
| | HC-FR1 | |
| 125 | Ab7 HC-FR2 | INWVRQAPGKGLEWVSS |
| 126 | Ab7 HC-FR3 | FDADSVRGRFTISRDNAKNLVYLQMNSLRAEDTAVYYC |
| 127 | Ab7, Ab16 HC-FR4 | WGQGTMVTVSS |
| 128 | Ab7 (G1) VL | DIQMTQSPSTLSASVGDRVTITCRASQSISTWLAWYQQKPGKAPKLLIYKASNLESGVPS |
| | | RFSGSGSGTEFTLTISRLQPDDSATYCCQHYNSYPYTFGQGTKVEIK |
| 129 | Ab7 LC-CDR1 | QSISTW |
| 130 | Ab7, Ab8, Ab32, Ab44 | KAS |
| | LC-CDR2 | |
| 131 | Ab7 LC-CDR3 | QHYNSYPYT |
| 132 | Ab7, Ab8, Ab16, Ab17, | DIQMTQSPSTLSASVGDRVTITCRAS |
| | Ab18, Ab44, Ab49 LC- | |
| | FR1 | |
| 133 | Ab7, Ab17, Ab27, | LAWYQQKPGKAPKLLIY |
| | Ab33, Ab34 LC-FR2 | |
| 134 | Ab7 LC-FR3 | NLESGVPSRFSGSGSGTEFTLTISRLQPDDSATYCC |
| 135 | Ab7, Ab8, Ab12, Ab17, | FGQGTKVEIK |
| | Ab18, Ab24, Ab29, | |
| | Ab44, Ab47 LC-FR4 | |
| 136 | Ab8 (B9) VH | EVQLVQSRAEVKKPGESLKISCKGSGYSFTTYWIGWVRQMPGKGLEWMGIIRPGDSDTRY |
| | | SPSFQGQVTISADKSITTAYLQWSSLKASDTAMYYCAMTYSGDQYDFWGQGTVVTVSS |
| 137 | Ab8 HC-CDR1 | GYSFTTYW |
| 138 | Ab8 HC-CDR2 | IRPGDSDT |
| 139 | Ab8 HC-CDR3 | AMTYSGDQYDF |
| 140 | Ab8 HC-FR1 | EVQLVQSRAEVKKPGESLKISCKGS |
| 141 | Ab8 HC-FR2 | IGWVRQMPGKGLEWMGI |
| 142 | Ab8 HC-FR3 | RYSPSFQGQVTISADKSITTAYLQWSSLKASDTAMYYC |
| 143 | Ab8 HC-FR4 | WGQGTVVTVSS |
| 144 | Ab8 (B9) VL | DIQMTQSPSTLSASVGDRVTITCRASQSIHTWLAWYQQTPGKAPKLLIYKASLLESGVPS |
| | | RFSGSGSGTEFTLTISSLQPGDFATYYCQHYNSYSHTFGQGTKVEIK |
| 145 | Ab8 LC-CDR1 | QSIHTW |
| 146 | Ab8 LC-CDR3 | QHYNSYSHT |
| 147 | Ab8 LC-FR2 | LAWYQQTPGKAPKLLIY |
| 148 | Ab8 LC-FR3 | LLESGVPSRFSGSGSGTEFTLTISSLQPGDFATYYC |
| 149 | Ab9 (C68) VH | QVQLVQSGAEVKKPGASVKVSCKISGYTLTDFSIHWVRQAPGKGLEWMAGFDPEHRETIF |
| | | AQKFQGRVAMTEATSTDTAYMELSSLRSDDTAVYYCATTGDFDSWRGYYLWGQGTLVTVS |
| | | A |
| 150 | Ab9 HC-CDR1 | GYTLTDFS |
| 151 | Ab9 HC-CDR2 | FDPEHRET |
| 152 | Ab9 HC-CDR3 | ATTGDFDSWRGYYL |
| 153 | Ab9 HC-FR1 | QVQLVQSGAEVKKPGASVKVSCKIS |
| 154 | Ab9 HC-FR2 | IHWVRQAPGKGLEWMAG |
| 155 | Ab9 HC-FR3 | IFAQKFQGRVAMTEATSTDTAYMELSSLRSDDTAVYYC |
| 156 | Ab9 HC-FR4 | WGQGTLVTVSA |
| 157 | Ab9 (C68) VL | DIQLTQSPSSVSASVGDSVTITCRASQGISRWLAWYQQKPGKAPRLLIYSASTLQSGVPS |
| | | RFSGSGSGTDFTLTISSLQPDDFATYYCQHTNNFPFSFGPGTKVDIK |
| 158 | Ab9 LC-CDR1 | QGISRW |
| 159 | Ab9, Ab20, Ab51 LC- | SAS |
| | CDR2 | |
| 160 | Ab9 LC-CDR3 | QHTNNFPFS |
| 161 | Ab9 LC-FR1 | DIQLTQSPSSVSASVGDSVTITCRAS |
| 162 | Ab9 LC-FR2 | LAWYQQKPGKAPRLLIY |
| 163 | Ab9 LC-FR3 | TLQSGVPSRFSGSGSGTDFTLTISSLQPDDFATYYC |
| 164 | Ab10 (C115) VH | QVQLVQSGAEVKKPGSSVKVSCRASGDTFSTYTITWVRQAPGQGLEWMGRIIPILDTADY |
| | | AQKFQGRVTITADKSTSTAYMELSSLRSEDTAMYYCAKNYPNGYSGYDYFSWDGFDPWGQ |
| | | GTLVTVSS |
| 165 | Ab10 HC-CDR1 | GDTFSTYT |
| 166 | Ab10 HC-CDR2 | IIPILDTA |
| 167 | Ab10 HC-CDR3 | AKNYPNGYSGYDYFSWDGFDP |
| 168 | Ab10 HC-FR1 | QVQLVQSGAEVKKPGSSVKVSCRAS |
| 169 | Ab10, Ab47 HC-FR2 | ITWVRQAPGQGLEWMGR |
| 170 | Ab10 HC-FR3 | DYAQKFQGRVTITADKSTSTAYMELSSLRSEDTAMYYC |
| 171 | Ab10 (C115) VL | QSVLTQPPSVSGAPGQRVTISCTGSSSNIGANYEVHWYQQLPGTAPKLLIFGHSNRPSAV |
| | | PDRFSGSKSGTSASLAITGLQTEDEADYYCQSYDSSLSGVVFGGGTKLTVL |
| 172 | Ab10 LC-CDR1 | SSNIGANYE |
| 173 | Ab10 LC-CDR2 | GHS |
| 174 | Ab10 LC-CDR3 | QSYDSSLSGVV |
| 175 | Ab10 LC-FR1 | QSVLTQPPSVSGAPGQRVTISCTGS |
| 176 | Ab10 LC-FR2 | VHWYQQLPGTAPKLLIF |
| 177 | Ab10 LC-FR3 | NRPSAVPDRFSGSKSGTSASLAITGLQTEDEADYYC |
| 178 | Ab10, Ab15, Ab21, | FGGGTKLTVL |
| | Ab22, Ab25 LC-FR4 | |
| 179 | Ab11 (D4) VH | QVQLQESGPRLVKPSGTLSLSCAVSGGPFSNTNWWSWIRQTPGKGLEWIGEINDSGNTVY |
| | | NPALKSRVTMSVDKSKKQFSLNLHSLTAADTAVYFCARVWGHFDYWGQGVRVTVSS |
| 180 | Ab11 HC-CDR1 | GGPFSNTNW |
| 181 | Ab11 HC-CDR2 | INDSGNT |
| 182 | Ab11 HC-CDR3 | ARVWGHFDY |
| 183 | Ab11 HC-FR1 | QVQLQESGPRLVKPSGTLSLSCAVS |
| 184 | Ab11 HC-FR2 | WSWIRQTPGKGLEWIGE |
| 185 | Ab11 HC-FR3 | VYNPALKSRVTMSVDKSKKQFSLNLHSLTAADTAVYFC |
| 186 | Ab11 HC-FR4 | WGQGVRVTVSS |
| 187 | Ab11 (D4) VL | DIQMTQSPSSLYASVADRVTITCRASQGISNSLAWYQQQPGKAPQLLLYAASTLESGVPS |
| | | RFSGSGSGTDFTLTISSLQPEDFATYYCQQYYSLRTFGGGTKVEIK |
| 188 | Ab11 LC-CDR1 | QGISNS |
| 189 | Ab11 LC-CDR3 | QQYYSLRT |
| 190 | Ab11 LC-FR1 | DIQMTQSPSSLYASVADRVTITCRAS |
| 191 | Ab11 LC-FR2 | LAWYQQQPGKAPQLLLY |
| 192 | Ab11 LC-FR3 | TLESGVPSRFSGSGSGTDFTLTISSLQPEDFATYYC |
| 193 | Ab12 (H5) VH | EVQLLESGGGLVQPGGSLRLSCAASGFTFSGSDMSWVRQAPGKGLEWVSVIGGSGTYAYY |
| | | SDSVKGRFTISRDNSKNMLFLQMNSLRAEDTAIYYCAKETGFLWFGELLDSWGQGALVTV |
| | | SS |
| 194 | Ab12 HC-CDR1 | GFTFSGSD |
| 195 | Ab12 HC-CDR2 | IGGSGTYA |
| 196 | Ab12 HC-CDR3 | AKETGFLWFGELLDS |
| 197 | Ab12 HC-FR1 | EVQLLESGGGLVQPGGSLRLSCAAS |
| 198 | Ab12, Ab34 HC-FR2 | MSWVRQAPGKGLEWVSV |
| 199 | Ab12 HC-FR3 | YYSDSVKGRFTISRDNSKNMLFLQMNSLRAEDTAIYYC |
| 200 | Ab12 (H5) VL | DIQMTQSPSSLSASVGDRVTITCRASEAISNSLAWYQQRPGKAPRLLLYAAATLESGVPP |
| | | RFSGSGSGTDFTLTISTLQPEDFATYYCQQYYSPPPRTFGQGTKVEIK |
| 201 | Ab12 LC-CDR1 | EAISNS |
| 202 | Ab12 LC-CDR2 | AAA |
| 203 | Ab12 LC-CDR3 | QQYYSPPPRT |
| 204 | Ab12 LC-FR2 | LAWYQQRPGKAPRLLLY |
| 205 | Ab12 LC-FR3 | TLESGVPPRFSGSGSGTDFTLTISTLQPEDFATYYC |
| 206 | Ab13 (D10) VH | EVQLVESGGGLVQPGRSLRLSCAASGFTFDEYAMHWVRQAPGKGLEWVSGISFNSGSVGY |
| | | AGAAKGRFTISRDNAKKSLYLEMNSLRDEDTAFYYCAKDRGEHWLVRLFDSWGQGTLVTV |
| | | SS |
| 207 | Ab13 HC-CDR1 | GFTFDEYA |
| 208 | Ab13 HC-CDR2 | ISFNSGSV |
| 209 | Ab13 HC-CDR3 | AKDRGEHWLVRLFDS |
| 210 | Ab13 HC-FR1 | EVQLVESGGGLVQPGRSLRLSCAAS |
| 211 | Ab13 HC-FR2 | MHWVRQAPGKGLEWVSG |
| 212 | Ab13 HC-FR3 | GYAGAAKGRFTISRDNAKKSLYLEMNSLRDEDTAFYYC |
| 213 | Ab13 (D10) VL | DIQMTQSPSTLSASVGDRVTITCRPSQSIDRWLAWYQQKPGKAPTLLISAASSLETGVPS |
| | | RFSGSGSGTEFTLTISSLQPDDFATYYCQQYNGYSMFGPGTKVEIK |
| 214 | Ab13 LC-CDR1 | QSIDRW |
| 215 | Ab13 LC-CDR3 | QQYNGYSM |
| 216 | Ab13 LC-FR1 | DIQMTQSPSTLSASVGDRVTITCRPS |
| 217 | Ab13 LC-FR2 | LAWYQQKPGKAPTLLIS |
| 218 | Ab13 LC-FR3 | SLETGVPSRFSGSGSGTEFTLTISSLQPDDFATYYC |
| 219 | Ab13, Ab34 LC-FR4 | FGPGTKVEIK |
| 220 | Ab14 (F9) VH | EVQLVESGGGLVQPGGSLRLSCATSGFTFDDFAMHWVRQGPGKGLEWVSGISWNSGKIAY |
| | | TDSVKGRFSISRDNAKNSLYLQMNSLRPEDTALYYCTKDHAPSAILGDILTGFDPWGQGT |
| | | LVT |
| 221 | Ab14 HC-CDR1 | GFTFDDFA |
| 222 | Ab14 HC-CDR2 | ISWNSGKI |
| 223 | Ab14 HC-CDR3 | TKDHAPSAILGDILTGFDP |
| 224 | Ab14 HC-FR1 | EVQLVESGGGLVQPGGSLRLSCATS |
| 225 | Ab14 HC-FR2 | MHWVRQGPGKGLEWVSG |
| 226 | Ab14 HC-FR3 | AYTDSVKGRFSISRDNAKNSLYLQMNSLRPEDTALYYC |
| 227 | Ab14 HC-FR4 | WGQGTLVT |
| 228 | Ab14 (F9) VL | DIQMTQSPVSLSASVGDRVTITCRASQSISVYLNWYQQKPGKAPKLLMYAASILQSGVPS |
| | | RFSGSGSATDFTLTITSLQPEDFATYFCQQSFTMPPTFGQGTNLEIK |
| 229 | Ab14 LC-CDR1 | QSISVY |
| 230 | Ab14 LC-CDR3 | QQSFTMPPT |
| 231 | Ab14 LC-FR1 | DIQMTQSPVSLSASVGDRVTITCRAS |
| 232 | Ab14 LC-FR2 | LNWYQQKPGKAPKLLMY |
| 233 | Ab14 LC-FR3 | ILQSGVPSRFSGSGSATDFTLTITSLQPEDFATYFC |
| 234 | Ab14 LC-FR4 | FGQGTNLEIK |
| 235 | Ab15 (E1.1) VH | QVQLVQSGAEVKKPGASVKVSCKASGYIFNNYAIQWVRQAPGQRLEWMAWIHTGNGDTKY |
| | | SQKFQGRVTITRDTSASTAYMELSSLRSEDTAIYYCARVRPNWNTLGWFDPWGQGTLVTV |
| | | SS |
| 236 | Ab15 HC-CDR1 | GYIFNNYA |
| 237 | Ab15 HC-CDR2 | IHTGNGDT |
| 238 | Ab15 HC-CDR3 | ARVRPNWNTLGWFDP |
| 239 | Ab15 HC-FR1 | QVQLVQSGAEVKKPGASVKVSCKAS |
| 240 | Ab15 HC-FR2 | IQWVRQAPGQRLEWMAW |
| 241 | Ab15 HC-FR3 | KYSQKFQGRVTITRDTSASTAYMELSSLRSEDTAIYYC |
| 242 | Ab15 (E1.1) VL | NFMLTQPHSVSESPGKTVTISCTGSGGSIATNYVQWYQQRPGSAPTTVIYEDNERPSGVP |
| | | DRFSGSIDSSSNSASLTISGLRTEDEADYYCQSYDISTHWVFGGGTKLTVL |
| 243 | Ab15 LC-CDR1 | GGSIATNY |
| 244 | Ab15 LC-CDR2 | EDN |
| 245 | Ab15 LC-CDR3 | QSYDISTHWV |
| 246 | Ab15 LC-FR1 | NFMLTQPHSVSESPGKTVTISCTGS |
| 247 | Ab15 LC-FR2 | VQWYQQRPGSAPTTVIY |
| 248 | Ab15 LC-FR3 | ERPSGVPDRFSGSIDSSSNSASLTISGLRTEDEADYYC |
| 249 | Ab16 (G1.1) VH | EVQLVESGGTSVRPGGSLRLSCSASGFSVRSNFMTWVRQAPGKGLECVSVIYSGSGGSTF |
| | | YADSVKGRFTISKDDSKNTLYLQMNSLRAEDTAIYYCAREVSHAFDLWGQGTMVTVSS |
| 250 | Ab16 HC-CDR1 | GFSVRSNFMT |
| 251 | Ab16 HC-CDR2 | SGSGGST |
| 252 | Ab16, Ab32 HC-CDR3 | AREVSHAFDL |
| 253 | Ab16 HC-FR1 | EVQLVESGGTSVRPGGSLRLSCSAS |
| 254 | Ab16 HC-FR2 | WVRQAPGKGLECVSVIY |
| 255 | Ab16 HC-FR3 | FYADSVKGRFTISKDDSKNTLYLQMNSLRAEDTAIYYC |
| 256 | Ab16 (G1.1) VL | DIQMTQSPSTLSASVGDRVTITCRASQTIGRWLAWYQQRPGKAPSLLIYMASILESGVPL |
| | | RFSGSGSGTEFTLTISGLQPDDFATYYCQQYNSDSPYSFGQGTKLEIK |
| 257 | Ab16 LC-CDR1 | QTIGRW |
| 258 | Ab16, Ab17, Ab18 LC- | MAS |
| | CDR2 | |
| 259 | Ab16 LC-CDR3 | QQYNSDSPYS |
| 260 | Ab16 LC-FR2 | LAWYQQRPGKAPSLLIY |
| 261 | Ab16 LC-FR3 | ILESGVPLRFSGSGSGTEFTLTISGLQPDDFATYYC |
| 262 | Ab17 (B5.1) VH | QVQLQESGPGLVKPSETLSLTCTVSGGSLTSYYWSWIRQPPGGGLEWIGHIYYTGITDHN |
| | | PSLKSRVTISLDTSRNQFSLKVRSVTAADTALYYCARAAGSSDYFDFWGQGTPVTVSS |
| 263 | Ab17 HC-CDR1 | GGSLTSYY |
| 264 | Ab17 HC-CDR2 | IYYTGIT |
| 265 | Ab17 HC-CDR3 | ARAAGSSDYFDF |
| 266 | Ab17, Ab18, Ab35, E7 | QVQLQESGPGLVKPSETLSLTCTVS |
| | HC-FR1 | |
| 267 | Ab17 HC-FR2 | WSWIRQPPGGGLEWIGH |
| 268 | Ab17 HC-FR3 | DHNPSLKSRVTISLDTSRNQFSLKVRSVTAADTALYYC |
| 269 | Ab17 HC-FR4 | WGQGTPVTVSS |
| 270 | Ab17 (B5.1) VL | DIQMTQSPSTLSASVGDRVTITCRASQSISRWLAWYQQKPGKAPKLLIYMASILESGVPS |
| | | RFSGGGSGTEFTLTISSLQPDDFATYYCQQYNSYRTFGQGTKVEIK |
| 271 | Ab17 LC-CDR1 | QSISRW |
| 272 | Ab17 LC-CDR3 | QQYNSYRT |
| 273 | Ab17 LC-FR3 | ILESGVPSRFSGGGSGTEFTLTISSLQPDDFATYYC |
| 274 | Ab18 (H9.1) VH | QVQLQESGPGLVKPSETLSLTCTVSGGSITSYYWSWIRQSPGKGLEWIGHIYYSGSTDYN |
| | | PSLKSRVTISVDTSKNQFSLKLTYVAAADTAVYFCARAGGSSDYFDYWGQGTLVTVSS |
| 275 | Ab18 HC-CDR1 | GGSITSYY |
| 276 | Ab18 HC-CDR2 | IYYSGST |
| 277 | Ab18 HC-CDR3 | ARAGGSSDYFDY |
| 278 | Ab18 HC-FR2 | WSWIRQSPGKGLEWIGH |
| 279 | Ab18 HC-FR3 | DYNPSLKSRVTISVDTSKNQFSLKLTYVAAADTAVYFC |
| 280 | Ab18 (H9.1) VL | DIQMTQSPSTLSASVGDRVTITCRASQSIGRWLAWYQQKPGKAPKFLIYMASILEDGVPS |
| | | RFSGSGSGTEFTLTITSLQPDDFATYYCQQYNDYRTFGQGTKVEIK |
| 281 | Ab18 LC-CDR1 | QSIGRW |
| 282 | Ab18 LC-CDR3 | QQYNDYRT |
| 283 | Ab18 LC-FR2 | LAWYQQKPGKAPKFLIY |
| 284 | Ab18 LC-FR3 | ILEDGVPSRFSGSGSGTEFTLTITSLQPDDFATYYC |
| 285 | Ab19 (F5.1) VH | EVQLVESGGGLVKPGGSLRLSCAASGFIFSRNGMHWVRQAPGKGLEWVSSIDNDGTYMYY |
| | | ADSVRGRFTVSRDNAKNSLFLQLNSLRAEDTAVYYCARDRFGYYDALTDSYNAGYFDSWG |
| | | QGTLVTVSS |
| 286 | Ab19 HC-CDR1 | GFIFSRNG |
| 287 | Ab19 HC-CDR2 | IDNDGTYM |
| 288 | Ab19 HC-CDR3 | ARDRFGYYDALTDSYNAGYFDS |
| 289 | Ab19 HC-FR2 | MHWVRQAPGKGLEWVSS |
| 290 | Ab19 HC-FR3 | YYADSVRGRFTVSRDNAKNSLFLQLNSLRAEDTAVYYC |
| 291 | Ab19 (F5.1) VL | DIQMTQSPSSLSASVGDSVTITCRASQDIYNYLAWFQQKPGKAPKSLIYTASKLESGVPS |
| | | KFSGSGSGTDFTLTISSLQPEDFATYYCQQYHSSPITFGQGTRVEIK |
| 292 | Ab19 LC-CDR1 | QDIYNY |
| 293 | Ab19 LC-CDR2 | TAS |
| 294 | Ab19 LC-CDR3 | QQYHSSPIT |
| 295 | Ab19 LC-FR1 | DIQMTQSPSSLSASVGDSVTITCRAS |
| 296 | Ab19, Ab51 LC-FR2 | LAWFQQKPGKAPKSLIY |
| 297 | Ab19 LC-FR3 | KLESGVPSKFSGSGSGTDFTLTISSLQPEDFATYYC |
| 298 | Ab19 LC-FR4 | FGQGTRVEIK |
| 299 | Ab20 (C6.1) VH | EVQLVESGGGLVKPGGSLRLSCAASGFAFSTNGMNWVRQVPGKGLEWVSSISSTSEYTYY |
| | | TESVKGRFTISRDNAKNSLFLQMNSLRAEDTAVYYCVRDRFGYYDVLASSYNVGFFQSWG |
| | | QGALVTVSS |
| 300 | Ab20 HC-CDR1 | GFAFSTNG |
| 301 | Ab20 HC-CDR2 | ISSTSEYT |
| 302 | Ab20 HC-CDR3 | VRDRFGYYDVLASSYNVGFFQS |
| 303 | Ab20 HC-FR2 | MNWVRQVPGKGLEWVSS |
| 304 | Ab20 HC-FR3 | YYTESVKGRFTISRDNAKNSLFLQMNSLRAEDTAVYYC |
| 305 | Ab20 (C6.1) VL | DIQMTQSPSSLSASVGDGVTITCRASQDISNNLAWFQQKPGKAPKCLIYSASSLQSGVPL |
| | | KFRGSGSGTDFTLSITSLDPEDFATYYCQQYISYPITFGQGTRLDIK |
| 306 | Ab20 LC-CDR1 | QDISNN |
| 307 | Ab20 LC-CDR3 | QQYISYPIT |
| 308 | Ab20 LC-FR1 | DIQMTQSPSSLSASVGDGVTITCRAS |
| 309 | Ab20 LC-FR2 | LAWFQQKPGKAPKCLIY |
| 310 | Ab20 LC-FR3 | SLQSGVPLKFRGSGSGTDFTLSITSLDPEDFATYYC |
| 311 | Ab20, Ab30 LC-FR4 | FGQGTRLDIK |
| 312 | Ab21 (E6.1) VH | QVQLVQSGAEVKKPGSSVEVSCKASGGTFSSYAISWVRQAPGQGLEWMGRIIPSLGITNS |
| | | AENFQGRVTITADKSTSTVYMELSSLRSEDTAVYYCARDFHPLYQSCSSTSCYDEWEPRM |
| | | DVWGQGTTVTVSS |
| 313 | Ab21 HC-CDR1 | GGTFSSYA |
| 314 | Ab21 HC-CDR2 | TIPSLGIT |
| 315 | Ab21 HC-CDR3 | ARDFHPLYQSCSSTSCYDEWEPRMDV |
| 316 | Ab21 HC-FR1 | QVQLVQSGAEVKKPGSSVEVSCKAS |
| 317 | Ab21 HC-FR3 | NSAENFQGRVTITADKSTSTVYMELSSLRSEDTAVYYC |
| 318 | Ab21 (E6.1) VL | QSVLTQPPSASGTPGQRVTISCHGSSSNIGSKTVNWYQQLPGTAPKLLIYSNDQRPSGVP |
| | | DRFSGSKSGTSASLAISGLQSEDEADYYCAAWDGSLDGPVFGGGTKLTVL |
| 319 | Ab21 LC-CDR1 | SSNIGSKT |
| 320 | Ab21, Ab22, Ab25 LC- | SND |
| | CDR2 | |
| 321 | Ab21 LC-CDR3 | AAWDGSLDGPV |
| 322 | Ab21 LC-FR1 | QSVLTQPPSASGTPGQRVTISCHGS |
| 323 | Ab21, Ab23 LC-FR2 | VNWYQQLPGTAPKLLIY |
| 324 | Ab21, Ab22, Ab25 LC- | QRPSGVPDRFSGSKSGTSASLAISGLQSEDEADYYC |
| | FR3 | |
| 325 | Ab22 (C9.1) VH | QVQLVQSGAEVKKPGSSVKVSCKASGGTFSSYGISWVRQAPGQGLEWMGRIIPSLSITNS |
| | | AEKFQGRVTITADKSTSTAYMELSRLRSEDTALYYCARDFHPRYEFCDSTSCYDEWEPRM |
| | | DVWGQGTTVTVSS |
| 326 | Ab22 HC-CDR1 | GGTFSSYG |
| 327 | Ab22, Ab25 HC-CDR2 | IIPSLSIT |
| 328 | Ab22 HC-CDR3 | ARDFHPRYEFCDSTSCYDEWEPRMDV |
| 329 | Ab22, Ab25, Ab26, | QVQLVQSGAEVKKPGSSVKVSCKAS |
| | Ab30, Ab31, Ab38, | |
| | Ab49 HC-FR1 | |
| 330 | Ab22 HC-FR3 | NSAEKFQGRVTITADKSTSTAYMELSRLRSEDTALYYC |
| 331 | Ab22 (C9.1) VL | QSVLTQSPSASGTPGQRVIISCSGSSSNIGRKTVNWYQQLPGTAPKLVIYSNDQRPSGVP |
| | | DRFSGSKSGTSASLAISGLQSEDEADYYCAAWDDSLDGPVFGGGTKLTVL |
| 332 | Ab22, Ab25 LC-CDR1 | SSNIGRKT |
| 333 | Ab22, Ab25 LC-CDR3 | AAWDDSLDGPV |
| 334 | Ab22, Ab25 LC-FR1 | QSVLTQSPSASGTPGQRVIISCSGS |
| 335 | Ab22 LC-FR2 | VNWYQQLPGTAPKLVIY |
| 336 | Ab23 (B10.1) VH | QVQLVQSGAEVKKPGSAVKVSCKASGGTFSSYPITWVRQAPGQGLEWVGRAIPILGITST |
| | | AQKFQGRVTIIADKSTSTAYMELSRLRSEDTAVYYCARDFHPRYQDCDSTSCYDQWEPRM |
| | | DVWGQGTTVTVSS |
| 337 | Ab23 HC-CDR1 | GGTFSSYP |
| 338 | Ab23 HC-CDR2 | AIPILGIT |
| 339 | Ab23 HC-CDR3 | ARDFHPRYQDCDSTSCYDQWEPRMDV |
| 340 | Ab23 HC-FR1 | QVQLVQSGAEVKKPGSAVKVSCKAS |
| 341 | Ab23 HC-FR2 | ITWVRQAPGQGLEWVGR |
| 342 | Ab23 HC-FR3 | STAQKFQGRVTIIADKSTSTAYMELSRLRSEDTAVYYC |
| 343 | Ab23 (B10.1) VL | QSVLTQPPSASGTPGQRVTISCSGSSSNIGSNTVNWYQQLPGTAPKLLIYTNNQRPSGVP |
| | | NRFSASKSGTSASLAISGLQSEDEADYYCAAWDDRLSGPVFGGGTKVTVL |
| 344 | Ab23 LC-CDR1 | SSNIGSNT |
| 345 | Ab23 LC-CDR2 | TNN |
| 346 | Ab23 LC-CDR3 | AAWDDRLSGPV |
| 347 | Ab23 LC-FR1 | QSVLTQPPSASGTPGQRVTISCSGS |
| 348 | Ab23 LC-FR3 | QRPSGVPNRFSASKSGTSASLAISGLQSEDEADYYC |
| 349 | Ab23 LC-FR4 | FGGGTKVTVL |
| 350 | Ab24 (G1.2) VH | QVQLVQSGAEVKKPGSSVKVSCEASGDTFSRYAINWVRQAPGQGLEWMGRIIPMFGNTNY |
| | | AQKFQGRFTITADKSRGTAYMEVIGLTSADTAVYYCATSPFYYSDGGYPFDFWGQGTLVT |
| | | VSS |
| 351 | Ab24 HC-CDR2 | IIPMFGNT |
| 352 | Ab24 HC-CDR3 | ATSPFYYSDGGYPFDF |
| 353 | Ab24, Ab29, Ab39 HC- | INWVRQAPGQGLEWMGR |
| | FR2 | |
| 354 | Ab24 HC-FR3 | NYAQKFQGRFTITADKSRGTAYMEVIGLTSADTAVYYC |
| 355 | Ab24 (G1.2) VL | EIVLTQSPGTLSLSPGERATLSCRASQSLNSAYLAWYQQRAGLAPRLLIYGASSRATGIP |
| | | DKFSGSGSGTDFTLTISRLEPEDFAVYYCQQYAYSPRTFGQGTKVEIK |
| 356 | Ab24 LC-CDR1 | QSLNSAY |
| 357 | Ab24, Ab28, Ab29, | GAS |
| | Ab30, Ab45, Ab47 LC- | |
| | CDR2 | |
| 358 | Ab24 LC-CDR3 | QQYAYSPRT |
| 359 | Ab24, Ab28, Ab29, | EIVLTQSPGTLSLSPGERATLSCRAS |
| | Ab38 LC-FR1 | |
| 360 | Ab24 LC-FR2 | LAWYQQRAGLAPRLLIY |
| 361 | Ab24 LC-FR3 | SRATGIPDKFSGSGSGTDFTLTISRLEPEDFAVYYC |
| 362 | Ab25 (H10.2) VH | QVQLVQSGAEVKKPGSSVKVSCKASRGTFSNYAISWVRQAPGQGLEWMGRIIPSLSITNS |
| | | AEKFQGRVTMTADKSTSTVYMELSRLRSEDTAVYYCARDFHPRYEYCSSTSCYDEWEPRM |
| | | DVWGQGTTVTVSS |
| 363 | Ab25 HC-CDR1 | RGTFSNYA |
| 364 | Ab25 HC-CDR3 | ARDFHPRYEYCSSTSCYDEWEPRMDV |
| 365 | Ab25 HC-FR3 | NSAEKFQGRVTMTADKSTSTVYMELSRLRSEDTAVYYC |
| 366 | Ab25 (H10.2) VL | QSVLTQSPSASGTPGQRVIISCSGSSSNIGRKTVNWYQQLPGTAPKLLMYSNDQRPSGVP |
| | | DRFSGSKSGTSASLAISGLQSEDEADYYCAAWDDSLDGPVFGGGTKLTVL |
| 367 | Ab25 LC-FR2 | VNWYQQLPGTAPKLLMY |
| 368 | Ab26 (G4.2) VH | QVQLVQSGAEVKKPGSSVKVSCKASGGTFSSHTVSWVRQAPGQGLEWMGRIIPPFGIVNY |
| | | AQKFQGRVTMTADESTSTAYMELSSLRSEDTAVYYCARSNVVVVTEAGWFDPWGQGTLVT |
| | | VSS |
| 369 | Ab26, Ab46 HC-CDR1 | GGTFSSHT |
| 370 | Ab26, Ab46 HC-CDR2 | IIPPFGIV |
| 371 | Ab26, Ab46 HC-CDR3 | ARSNVVVVTEAGWFDP |
| 372 | Ab26, Ab46 HC-FR2 | VSWVRQAPGQGLEWMGR |
| 373 | Ab26 HC-FR3 | NYAQKFQGRVTMTADESTSTAYMELSSLRSEDTAVYYC |
| 374 | Ab26 (G4.2) VL | DIQMTQSPSSVSASVGDRVSISCRASQDISSSLAWYQQKPGQAPKVLIYDASTLQSGVPS |
| | | RFSGSGSGTDFTLTISNLRPDDFATYFCQQAHSFPFTFGPGTKVDIR |
| 375 | Ab26 LC-CDR1 | QDISSS |
| 376 | Ab26, Ab46 LC-CDR3 | QQAHSFPFT |
| 377 | Ab26 LC-FR1 | DIQMTQSPSSVSASVGDRVSISCRAS |
| 378 | Ab26 LC-FR2 | LAWYQQKPGQAPKVLIY |
| 379 | Ab26 LC-FR3 | TLQSGVPSRFSGSGSGTDFTLTISNLRPDDFATYFC |
| 380 | Ab26 LC-FR4 | FGPGTKVDIR |
| 381 | Ab27 (E12.2) VH | EVQLVESGGGLVQPGGSLRLSCGASGITVSSNYMNWVRQAPGKGLEWVSTLYAGGSTFYA |
| | | DSVKGRFIISRDNSKNTLYLQMNSLRADDTAVYYCARDLVDYGMDVWGQGTTVTVSS |
| 382 | Ab27, Ab33 HC-CDR1 | GITVSSNY |
| 383 | Ab27, Ab33 HC-CDR2 | LYAGGST |
| 384 | Ab27, Ab33 HC-CDR3 | ARDLVDYGMDV |
| 385 | Ab27, Ab33 HC-FR1 | EVQLVESGGGLVQPGGSLRLSCGAS |
| 386 | Ab27, Ab33 HC-FR2 | MNWVRQAPGKGLEWVST |
| 387 | Ab27 HC-FR3 | FYADSVKGRFIISRDNSKNTLYLQMNSLRADDTAVYYC |
| 388 | Ab27 (E12.2) VL | DIQLTQSPSSLSASVGDRVTITCRASQGISSYLAWYQQKPGKAPKLLIYAASTLQSGVPS |
| | | RFSGSGSGTDFTLTISSLQPEDFATYYCQQLNSYSPFTFGPGTKVDIK |
| 389 | Ab27, Ab33, Ab34 LC- | QGISSY |
| | CDR1 | |
| 390 | Ab27, Ab33 LC-CDR3 | QQLNSYSPFT |
| 391 | Ab27, Ab45 LC-FR1 | DIQLTQSPSSLSASVGDRVTITCRAS |
| 392 | Ab27, Ab30, Ab33 LC- | TLQSGVPSRFSGSGSGTDFTLTISSLQPEDFATYYC |
| | FR3 | |
| 393 | Ab28 (10x-L8N-c1) VH | QFQLVQSGAEVKKPGSSVKVSCRASGGSFTSHAISWVRQAPGQGFEWMGRIIPMFGIANY |
| | | APKFQGRVTMSADKFKDIVYMEVNSLTSEDTALYYCARSQPMTSVTTLWFDPWGQGTLVT |
| | | VSS |
| 394 | Ab28 HC-CDR1 | GGSFTSHA |
| 395 | Ab28, Ab38 HC-CDR2 | IIPMFGIA |
| 396 | Ab28 HC-CDR3 | ARSQPMTSVTTLWFDP |
| 397 | Ab28 HC-FR1 | QFQLVQSGAEVKKPGSSVKVSCRAS |
| 398 | Ab28 HC-FR2 | ISWVRQAPGQGFEWMGR |
| 399 | Ab28 HC-FR3 | NYAPKFQGRVTMSADKFKDIVYMEVNSLTSEDTALYYC |
| 400 | Ab28 (10x-L8N-c1) VL | EIVLTQSPGTLSLSPGERATLSCRASEPVGGSYLAWYQQKPGQAPRLLIHGASSRATGIP |
| | | DRFSGSGSGTDFVLTISRLEPEDFAVYHCQQYASSPYTFGQGTKLEIK |
| 401 | Ab28 LC-CDR1 | EPVGGSY |
| 402 | Ab28 LC-CDR3 | QQYASSPYT |
| 403 | Ab28 LC-FR2 | LAWYQQKPGQAPRLLIH |
| 404 | Ab28 LC-FR3 | SRATGIPDRFSGSGSGTDFVLTISRLEPEDFAVYHC |
| 405 | Ab29 (10x-L8N-c5) VH | QVHLVQSGAEVKKPGSSVKVSCKTSGDTFTRYAINWVRQAPGQGLEWMGRIIPMFGIPNY |
| | | AQKFQGRVTMTADKSTDIAYMELSSLRSEDTAVYYCARSSFYSDSSGYYLDYWGQGTLVT |
| | | VSS |
| 406 | Ab29 HC-CDR1 | GDTFTRYA |
| 407 | Ab29 HC-CDR2 | IIPMFGIP |
| 408 | Ab29 HC-CDR3 | ARSSFYSDSSGYYLDY |
| 409 | Ab29 HC-FR1 | QVHLVQSGAEVKKPGSSVKVSCKTS |
| 410 | Ab29 HC-FR3 | NYAQKFQGRVTMTADKSTDIAYMELSSLRSEDTAVYYC |
| 411 | Ab29 (10x-L8N-c5) VL | EIVLTQSPGTLSLSPGERATLSCRASQNLDSNYLAWYQQKPGQAPRLLIYGASIRATGIP |
| | | DRFSGSGSGTDFTLTISRLEPEDFAVYHCQQYHNSPRTFGQGTKVEIK |
| 412 | Ab29 LC-CDR1 | QNLDSNY |
| 413 | Ab29 LC-CDR3 | QQYHNSPRT |
| 414 | Ab29, Ab35, Ab38 LC- | LAWYQQKPGQAPRLLIY |
| | FR2 | |
| 415 | Ab29 LC-FR3 | IRATGIPDRFSGSGSGTDFTLTISRLEPEDFAVYHC |
| 416 | Ab30 (10x-L8N-c9) VH | QVQLVQSGAEVKKPGSSVKVSCKASGDTFTKYAITWVRQAPGEGLEWMGRIIPRFGMANY |
| | | AQNFQGRVTMTADQSTSTAYMELTSLRSNDTAVYYCATTFYFDSSYYHAMDYWGQGSLVT |
| | | VSS |
| 417 | Ab30 HC-CDR1 | GDTFTKYA |
| 418 | Ab30 HC-CDR2 | IIPRFGMA |
| 419 | Ab30 HC-CDR3 | ATTFYFDSSYYHAMDY |
| 420 | Ab30 HC-FR2 | ITWVRQAPGEGLEWMGR |
| 421 | Ab30 HC-FR3 | NYAQNFQGRVTMTADQSTSTAYMELTSLRSNDTAVYYC |
| 422 | Ab30, Ab47 HC-FR4 | WGQGSLVTVSS |
| 423 | Ab30 (10x-L8N-c9) VL | DIQMTQSPSSLSASVGDRVTITCRASQNIDTYLIWYQQKPGKAPNLLVYGASTLQSGVPS |
| | | RFSGSGSGTDFTLTISSLQPEDFATYYCQQTYNAPRTFGQGTRLDIK |
| 424 | Ab30 LC-CDR1 | QNIDTY |
| 425 | Ab30 LC-CDR3 | QQTYNAPRT |
| 426 | Ab30 LC-FR2 | LIWYQQKPGKAPNLLVY |
| 427 | Ab31 (10x-L8N-c10) | QVQLVQSGAEVKKPGSSVKVSCKASGDTFSRYAISWVRQAPGQGLEWMGRIIPMFGTANY |
| | VH | AQNFQGRVTITADKSTSTAYMELTSLRSEDTAVYYCATTYFYDSDRDRTHSMDVWGPGSA |
| | | VTVSS |
| 428 | Ab31 HC-CDR2 | IIPMFGTA |
| 429 | Ab31 HC-CDR3 | ATTYFYDSDRDRTHSMDV |
| 430 | Ab31 HC-FR3 | NYAQNFQGRVTITADKSTSTAYMELTSLRSEDTAVYYC |
| 431 | Ab31 HC-FR4 | WGPGSAVTVSS |
| 432 | Ab31 (10x-L8N-c10) VL | DIQMTQSPSSLSASVGDRVTITCRTSQSVGNYLNWYQQKPGKAPNLLIYAASTLQNGVPS |
| | | RFSGSGSGTDFTLTISSLQPEDFATYYCQQTHSTPRAFGGGTKVEIK |
| 433 | Ab31 LC-CDR1 | QSVGNY |
| 434 | Ab31 LC-FR1 | DIQMTQSPSSLSASVGDRVTITCRTS |
| 435 | Ab31 LC-FR3 | TLQNGVPSRFSGSGSGTDFTLTISSLQPEDFATYYC |
| 436 | Ab32 (10x-L8N-c12) | EVQLVESGGGLVQPGGSLRVTCVASGFAVRTNFMTWVRQAPGKGLQCVSVIYGDGSTYYA |
| | VH | DSVKGRFSISRDNSKNTVYLQMNSLSAEDTAVYYCAREVSHAFDLWGPGTMVTVSS |
| 437 | Ab32 HC-CDR1 | GFAVRTNF |
| 438 | Ab32 HC-CDR2 | IYGDGST |
| 439 | Ab32 HC-FR1 | EVQLVESGGGLVQPGGSLRVTCVAS |
| 440 | Ab32 HC-FR2 | MTWVRQAPGKGLQCVSV |
| 441 | Ab32 HC-FR3 | YYADSVKGRFSISRDNSKNTVYLQMNSLSAEDTAVYYC |
| 442 | Ab32 HC-FR4 | WGPGTMVTVSS |
| 443 | Ab32 (10x-L8N-c12) VL | DIQLTQSPSTLSASLGDRVTITCRASQSISGWLAWYQQKPGRAPQLLIYKASLLETGVPS |
| | | RFSGSGSGTVFTLTISSLQPDDFATYYCQQYDTYSPYTFGQGTKLEIK |
| 444 | Ab32 LC-CDR1 | QSISGW |
| 445 | Ab32 LC-CDR3 | QQYDTYSPYT |
| 446 | Ab32 LC-FR1 | DIQLTQSPSTLSASLGDRVTITCRAS |
| 447 | Ab32 LC-FR2 | LAWYQQKPGRAPQLLIY |
| 448 | Ab32 LC-FR3 | LLETGVPSRFSGSGSGTVFTLTISSLQPDDFATYYC |
| 449 | Ab33 (10x-L8N-c18) | EVQLVESGGGLVQPGGSLRLSCGASGITVSSNYMNWVRQAPGKGLEWVSTLYAGGSTFYA |
| | VH | DSVKGRFIISRDNSKNTLYLQMNSLRAEDTAVYYCARDLVDYGMDVWGQGTTVTVSS |
| 450 | Ab33 HC-FR3 | FYADSVKGRFIISRDNSKNTLYLQMNSLRAEDTAVYYC |
| 451 | Ab33 (10x-L8N-c18) VL | AIQLTQSPSSLSASVGDRVTITCRASQGISSYLAWYQQKPGKAPKLLIYAASTLQSGVPS |
| | | RFSGSGSGTDFTLTISSLQPEDFATYYCQQLNSYSPFTFGPGTKVDIK |
| 452 | Ab33, Ab37 LC-FR1 | AIQLTQSPSSLSASVGDRVTITCRAS |
| 453 | Ab34 (10x-L8N-c19) | EVQLVESGGGLVQSGGSLRLSCAASGIIVSRNYMSWVRQAPGKGLEWVSVIYPGGSTFYA |
| | VH | DSVKGRFTISRDNSKNTLYLQMNSLRAEDTAVFYCARDRGEDIFDFWGQGTMVSVSS |
| 454 | Ab34, Ab37 HC-CDR1 | GIIVSRNY |
| 455 | Ab34 HC-CDR2 | IYPGGST |
| 456 | Ab34 HC-CDR3 | ARDRGEDIFDF |
| 457 | Ab34 HC-FR1 | EVQLVESGGGLVQSGGSLRLSCAAS |
| 458 | Ab34 HC-FR3 | FYADSVKGRFTISRDNSKNTLYLQMNSLRAEDTAVFYC |
| 459 | Ab34 HC-FR4 | WGQGTMVSVSS |
| 460 | Ab34 (10x-L8N-c19) VL | DIQLTQSPTFLSASVGDRVTITCRASQGISSYLAWYQQKPGKAPKLLIYAASTLQGGVPS |
| | | RFSGSGSGTEFTLTISSLQPEDFATYYCQQLNSSPGFTFGPGTKVEIK |
| 461 | Ab34 LC-CDR3 | QQLNSSPGFT |
| 462 | Ab34 LC-FR1 | DIQLTQSPTFLSASVGDRVTITCRAS |
| 463 | Ab34 LC-FR3 | TLQGGVPSRFSGSGSGTEFTLTISSLQPEDFATYYC |
| 464 | Ab35 (10x-L8N-c20) | QVQLQESGPGLVKPSETLSLTCTVSGDSIYSYSWTWIRQPPGKGLEWIGQLYYNGSTSYN |
| | VH | ASLKSRVSISLDTSKNQFSLKLRFVTAADTAMYYCARVEYYSNYFDPWGQGTQVTVSS |
| 465 | Ab35 HC-CDR1 | GDSIYSYS |
| 466 | Ab35 HC-CDR2 | LYYNGST |
| 467 | Ab35 HC-CDR3 | ARVEYYSNYFDP |
| 468 | Ab35 HC-FR2 | WTWIRQPPGKGLEWIGQ |
| 469 | Ab35 HC-FR3 | SYNASLKSRVSISLDTSKNQFSLKLRFVTAADTAMYYC |
| 470 | Ab35 HC-FR4 | WGQGTQVTVSS |
| 471 | Ab35 (10x-L8N-c20) VL | EIVLTQSPATLSLSPGERATLSCRASQSVGSYLAWYQQKPGQAPRLLIYDASNRVPGIPA |
| | | RFSGSGSGTDFTLTISSLPPEDFAVYYCQQRSDWPPTFGGGTKVEIR |
| 472 | Ab35 LC-CDR3 | QQRSDWPPT |
| 473 | Ab35 LC-FR3 | NRVPGIPARFSGSGSGTDFTLTISSLPPEDFAVYYC |
| 474 | Ab35, Ab41, Ab42 LC- | FGGGTKVEIR |
| | FR4 | |
| 475 | Ab37 (10x-L8N-c22) | EVQLVESGGGLVQPGGSLRVSCAASGIIVSRNYMTWVRQAPGKGLEWVSVIYAGGSTFYA |
| | VH | DSVKGRFTISRDNSKNTLYLQMNSLRVDDTAVYYCARDLEIAGGMDVWGQGTTVTVSS |
| 476 | Ab37 HC-CDR2 | IYAGGST |
| 477 | Ab37 HC-CDR3 | ARDLEIAGGMDV |
| 478 | Ab37 HC-FR1 | EVQLVESGGGLVQPGGSLRVSCAAS |
| 479 | Ab37 HC-FR2 | MTWVRQAPGKGLEWVSV |
| 480 | Ab37 HC-FR3 | FYADSVKGRFTISRDNSKNTLYLQMNSLRVDDTAVYYC |
| 481 | Ab37 (10x-L8N-c22) VL | AIQLTQSPSSLSASVGDRVTITCRASEGISNYLAWYQQKPGKAPKVLIYAASTLQSGAPP |
| | | RFSGSGSGTDFTLTISSLQPEDFATYYCQQLNSYPITFGQGTRLEIK |
| 482 | Ab37 LC-CDR1 | EGISNY |
| 483 | Ab37 LC-CDR3 | QQLNSYPIT |
| 484 | Ab37 LC-FR2 | LAWYQQKPGKAPKVLIY |
| 485 | Ab37 LC-FR3 | TLQSGAPPRFSGSGSGTDFTLTISSLQPEDFATYYC |
| 486 | Ab37, Ab45, Ab51, E7 | FGQGTRLEIK |
| | LC-FR4 | |
| 487 | Ab38 (G8.2) VH | QVQLVQSGAEVKKPGSSVKVSCKASGDTFSRDAISWVRQAPGQGLEWMGRIIPMFGIANY |
| | | AQNFQGRVTMTADKYTSTAYMELSSLRSEDTAVYYCARGGYQYESSGYHLDHWGQGTLVT |
| | | VSS |
| 488 | Ab38 HC-CDR1 | GDTFSRDA |
| 489 | Ab38 HC-CDR3 | ARGGYQYESSGYHLDH |
| 490 | Ab38 HC-FR3 | NYAQNFQGRVTMTADKYTSTAYMELSSLRSEDTAVYYC |
| 491 | Ab38 (G8.2) VL | EIVLTQSPGTLSLSPGERATLSCRASQSLSSSYLAWYQQKPGQAPRLLIYGTSSRATGIP |
| | | DRFSGSGSGTDFTLTISRLEPEDFAVYYCHQYSNSPRTFGQGTKVEVK |
| 492 | Ab38 LC-CDR1 | QSLSSSY |
| 493 | Ab38 LC-CDR3 | HQYSNSPRT |
| 494 | Ab38, Ab41 LC-FR3 | SRATGIPDRFSGSGSGTDFTLTISRLEPEDFAVYYC |
| 495 | Ab38 LC-FR4 | FGQGTKVEVK |
| 496 | Ab39 (10x-L8N-c30) | QVQLVQSGAEVKKPGSSVKISCKASGDTSSSYTINWVRQAPGQGLEWMGRIIPMFNRANY |
| | VH | ARKFQGRVTMTADRSTDTAYMEVSSLTSDDTAVYYCARTWIEPHNWFDPWGQGTLVTVSS |
| 497 | Ab39 HC-CDR1 | GDTSSSYT |
| 498 | Ab39 HC-CDR2 | IIPMFNRA |
| 499 | Ab39 HC-CDR3 | ARTWIEPHNWFDP |
| 500 | Ab39 HC-FR1 | QVQLVQSGAEVKKPGSSVKISCKAS |
| 501 | Ab39 HC-FR3 | NYARKFQGRVTMTADRSTDTAYMEVSSLTSDDTAVYYC |
| 502 | Ab39 (10x-L8N-c30) VL | EIVLRQSPGTLSLSPGERATLSCRASQSVSGNHLAWYQRKPGQAPRLLIYAASSRATGIP |
| | | DRFSGSGSGTDFTLTINRLEPEDFAVFYCQQYGTSPPTFGGGTKVEIK |
| 503 | Ab39 LC-CDR1 | QSVSGNH |
| 504 | Ab39 LC-CDR3 | QQYGTSPPT |
| 505 | Ab39 LC-FR1 | EIVLRQSPGTLSLSPGERATLSCRAS |
| 506 | Ab39 LC-FR2 | LAWYQRKPGQAPRLLIY |
| 507 | Ab39 LC-FR3 | SRATGIPDRFSGSGSGTDFTLTINRLEPEDFAVFYC |
| 508 | Ab40 (10x-L8N-c32) | QVQLQESGPRLVKPSETLSLTCTVSGGSISSYYWTWIRQPPGKGLEWIGYISYSGSTNYN |
| | VH | PSLRSRVTMSVDTSKNQFSLDLNSVTAADTAVYYCATDGGGFYPGYFPHWGQGTLVTVSS |
| 509 | Ab40 HC-CDR1 | GGSISSYY |
| 510 | Ab40 HC-CDR2 | ISYSGST |
| 511 | Ab40 HC-CDR3 | ATDGGGFYPGYFPH |
| 512 | Ab40 HC-FR1 | QVQLQESGPRLVKPSETLSLTCTVS |
| 513 | Ab40 HC-FR2 | WTWIRQPPGKGLEWIGY |
| 514 | Ab40 HC-FR3 | NYNPSLRSRVTMSVDTSKNQFSLDLNSVTAADTAVYYC |
| 515 | Ab40 (10x-L8N-c32) VL | DIQMTQSPSSLSASVGDRVTITCRASQSISDYVNWYQQKPGRAPRLLIYAPSTLQSGVPS |
| | | RFSGSGSGTDFTLTISNLQPEDFGTYFCQQSYSTPPYTFGQGTKVEMK |
| 516 | Ab40 LC-CDR1 | QSISDY |
| 517 | Ab40 LC-CDR2 | APS |
| 518 | Ab40 LC-CDR3 | QQSYSTPPYT |
| 519 | Ab40 LC-FR2 | VNWYQQKPGRAPRLLIY |
| 520 | Ab40 LC-FR3 | TLQSGVPSRFSGSGSGTDFTLTISNLQPEDFGTYFC |
| 521 | Ab40 LC-FR4 | FGQGTKVEMK |
| 522 | Ab41 (10x-L8N-c39) | QVQLVQSGAEVKKPGASVKVSCKGSGYTFTDYGISWVRQAPGQGLEWMGWIRAYNGNTDY |
| | VH | AQKFQGRVTMTRDTSTTTAYMELRSLRSDDTAVYFCALNLIAAAATPFDHWGQGTLVTVS |
| | | S |
| 523 | Ab41 HC-CDR1 | GYTFTDYG |
| 524 | Ab41 HC-CDR2 | IRAYNGNT |
| 525 | Ab41 HC-CDR3 | ALNLIAAAATPFDH |
| 526 | Ab41 HC-FR1 | QVQLVQSGAEVKKPGASVKVSCKGS |
| 527 | Ab41 HC-FR2 | ISWVRQAPGQGLEWMGW |
| 528 | Ab41 HC-FR3 | DYAQKFQGRVTMTRDTSTTTAYMELRSLRSDDTAVYFC |
| 529 | Ab41 (10x-L8N-c39) VL | EIVLTQSPGTLSLSRGERATLSCRASQSVSSSYLAWYQQKRGQAPRLLIYGATSRATGIP |
| | | DRFSGSGSGTDFTLTISRLEPEDFAVYYCQHYGSSPPPFGGGTKVEIR |
| 530 | Ab41 LC-CDR1 | QSVSSSY |
| 531 | Ab41 LC-CDR2 | GAT |
| 532 | Ab41 LC-CDR3 | QHYGSSPPP |
| 533 | Ab41 LC-FR1 | EIVLTQSPGTLSLSRGERATLSCRAS |
| 534 | Ab41 LC-FR2 | LAWYQQKRGQAPRLLIY |
| 535 | Ab42 (10x-L8N-c41) | EVQLLESGGGLVQPGGSLRLSCAGSGFTFSSYAMSWVRQAPGKGLEWVSSISSSGGLTFY |
| | VH | ADSVRGRSTISRDHSRNTLYLQMNSLRAEDTALYYCAREQGDSYDDYFDYWGQGTLVTVS |
| | | S |
| 536 | Ab42 HC-CDR1 | GFTFSSYA |
| 537 | Ab42 HC-CDR2 | ISSSGGLT |
| 538 | Ab42 HC-CDR3 | AREQGDSYDDYFDY |
| 539 | Ab42 HC-FR1 | EVQLLESGGGLVQPGGSLRLSCAGS |
| 540 | Ab42 HC-FR2 | MSWVRQAPGKGLEWVSS |
| 541 | Ab42 HC-FR3 | FYADSVRGRSTISRDHSRNTLYLQMNSLRAEDTALYYC |
| 542 | Ab42 (10x-L8N-c41) VL | DIQMTQSPSSLSASVGDRVTITCRASQGIRSSLAWYQQRPGKAPKLLLSAASILESGVPS |
| | | RFSGSGSGTDYTLTINGLQPDDFATYYCQQYYVTPGITFGGGTKVEIR |
| 543 | Ab42 LC-CDR1 | QGIRSS |
| 544 | Ab42 LC-CDR3 | QQYYVTPGIT |
| 545 | Ab42 LC-FR2 | LAWYQQRPGKAPKLLLS |
| 546 | Ab42 LC-FR3 | ILESGVPSRFSGSGSGTDYTLTINGLQPDDFATYYC |
| 547 | Ab43 (10x-L8N-c42) | EVQLVESGGGLVKPGGSLRLSCAASGFTFSRHTMNWVRQAPGKGLEWVSSIGGSTPLSFY |
| | VH | AESVKGRFSISRDNAKSSLFLQMSSLRADDTAVYYCVRDSIASVTTLFDLWGRGTVVTVS |
| | | S |
| 548 | Ab43 HC-CDR1 | GFTFSRHT |
| 549 | Ab43 HC-CDR2 | IGGSTPLS |
| 550 | Ab43 HC-CDR3 | VRDSIASVTTLFDL |
| 551 | Ab43 HC-FR2 | MNWVRQAPGKGLEWVSS |
| 552 | Ab43 HC-FR3 | FYAESVKGRFSISRDNAKSSLFLQMSSLRADDTAVYYC |
| 553 | Ab43 HC-FR4 | WGRGTVVTVSS |
| 554 | Ab43 (10x-L8N-c42) VL | DIQMTQSPSSLSASVGDRVTITCRASQTIRNYINWYQQRPGKAPRLLIYDASTLQSGVPS |
| | | RFSGGGSGTDFTLTISSLEPEDFATYYCQQSYVSPPEDSFGQGTKLEIK |
| 555 | Ab43 LC-CDR1 | QTIRNY |
| 556 | Ab43 LC-CDR3 | QQSYVSPPEDS |
| 557 | Ab43 LC-FR2 | INWYQQRPGKAPRLLIY |
| 558 | Ab43 LC-FR3 | TLQSGVPSRFSGGGSGTDFTLTISSLEPEDFATYYC |
| 559 | Ab44 (10x-L8N-c53) | QVQLQQSGAEVKKPGSSVKVSCESSGGTFTSHAINWVRQAPGQRLEWMGRIIPVLGVAAY |
| | VH | AQKFQGRVTLTADKFTSTAYMELSSLRSEDTAVYFCARSAYNYDSSGYHFDYWGQGTLVL |
| | | VSS |
| 560 | Ab44 HC-CDR1 | GGTFTSHA |
| 561 | Ab44 HC-CDR2 | IIPVLGVA |
| 562 | Ab44 HC-CDR3 | ARSAYNYDSSGYHFDY |
| 563 | Ab44 HC-FR1 | QVQLQQSGAEVKKPGSSVKVSCESS |
| 564 | Ab44 HC-FR2 | INWVRQAPGQRLEWMGR |
| 565 | Ab44 HC-FR3 | AYAQKFQGRVTLTADKFTSTAYMELSSLRSEDTAVYFC |
| 566 | Ab44 HC-FR4 | WGQGTLVLVSS |
| 567 | Ab44 (10x-L8N-c53) VL | DIQMTQSPSTLSASVGDRVTITCRASQSISNWLAWYQQKPGTAPKLLIFKASTLQSGVPS |
| | | RFSGSGSGTEFTLTISSLQPDDFATYHCQQYHSSRSFGQGTKVEIK |
| 568 | Ab44 LC-CDR1 | QSISNW |
| 569 | Ab44 LC-CDR3 | QQYHSSRS |
| 570 | Ab44 LC-FR2 | LAWYQQKPGTAPKLLIF |
| 571 | Ab44 LC-FR3 | TLQSGVPSRFSGSGSGTEFTLTISSLQPDDFATYHC |
| 572 | Ab45 (10x-L8N-c62) | QVQLVQSGAEVKKPGSSMTVSCEASGDTFSRYSINWVRQAPGQGFEWMGRIIPMFGLATY |
| | VH | AHKFQGRVTISADKSTTTAHMELNRLTSEDTAIYYCATAPFYYDNSTYPFDFWGQGTLVT |
| | | VSS |
| 573 | Ab45 HC-CDR1 | GDTFSRYS |
| 574 | Ab45 HC-CDR2 | IIPMFGLA |
| 575 | Ab45 HC-CDR3 | ATAPFYYDNSTYPFDF |
| 576 | Ab45 HC-FR1 | QVQLVQSGAEVKKPGSSMTVSCEAS |
| 577 | Ab45 HC-FR2 | INWVRQAPGQGFEWMGR |
| 578 | Ab45 HC-FR3 | TYAHKFQGRVTISADKSTTTAHMELNRLTSEDTAIYYC |
| 579 | Ab45 (10x-L8N-c62) VL | DIQLTQSPSSLSASVGDRVTITCRASQSINNYLNWYQHKPGKAPKLLIYGASTLQSGVPS |
| | | RFSGSGSGSDFTLTISSLQPEDFATYYCQQSYNAPRTFGQGTRLEIK |
| 580 | Ab45 LC-CDR1 | QSINNY |
| 581 | Ab45 LC-CDR3 | QQSYNAPRT |
| 582 | Ab45 LC-FR2 | LNWYQHKPGKAPKLLIY |
| 583 | Ab45 LC-FR3 | TLQSGVPSRFSGSGSGSDFTLTISSLQPEDFATYYC |
| 584 | Ab46 (10x-L8N-c63) | QVHLVQSGAEVKKPGSSVKVSCKASGGTFSSHTVSWVRQAPGQGLEWMGRIIPPFGIVNY |
| | VH | AQKFQGRVTITADESTSTAYMELSSLSSEDTAVYYCARSNVVVVTEAGWFDPWGQGTLVT |
| | | VSS |
| 585 | Ab46 HC-FR1 | QVHLVQSGAEVKKPGSSVKVSCKAS |
| 586 | Ab46 HC-FR3 | NYAQKFQGRVTITADESTSTAYMELSSLSSEDTAVYYC |
| 587 | Ab46 (10x-L8N-c63) VL | DIQMTQSPSSVSASVGDRVTITCRASQGISSSLAWYQQKPGKAPKVLIFDASTLQSGVPS |
| | | RFSGSGSGTDFTLTISNLRPEDFATYYCQQAHSFPFTFGPGTKVDIK |
| 588 | Ab46 LC-CDR1 | QGISSS |
| 589 | Ab46 LC-FR1 | DIQMTQSPSSVSASVGDRVTITCRAS |
| 590 | Ab46 LC-FR2 | LAWYQQKPGKAPKVLIF |
| 591 | Ab46 LC-FR3 | TLQSGVPSRFSGSGSGTDFTLTISNLRPEDFATYYC |
| 592 | Ab47 (10x-L8N-c64) | QVQLVQSGAEVKKPGSSVKVSCEASGDTFSRYAITWVRQAPGQGLEWMGRIPMFGSANYA |
| | VH | QKFQGRVSFTADKSTSTASMELSSLRSEDTAVYYCATSPYYSDSGGYYCDYWGQGSLVTV |
| | | SS |
| 593 | Ab47 HC-CDR2 | IIPMFGSA |
| 594 | Ab47 HC-CDR3 | ATSPYYSDSGGYYCDY |
| 595 | Ab47 HC-FR3 | NYAQKFQGRVSFTADKSTSTASMELSSLRSEDTAVYYC |
| 596 | Ab47 (10x-L8N-c64) VL | DIQMTQSPSSLSASVGDRVTITCRASQGISNYLAWYQQKPGKAPKLLLYGASRLQSGVPS |
| | | RFSGSGSGTDYTLTISSLQPEDFATFYCQQYYSAPRTFGQGTKVEIK |
| 597 | Ab47 LC-CDR3 | QQYYSAPRT |
| 598 | Ab47 LC-FR2 | LAWYQQKPGKAPKLLLY |
| 599 | Ab47 LC-FR3 | RLQSGVPSRFSGSGSGTDYTLTISSLQPEDFATFYC |
| 600 | Ab48 (10x-L8N-c72) | QVQLQQWGAGLLKPSETLSLTCTVYGGSISGYYWNWIRQPPGKGLEWIGEIDHSGNTNYN |
| | VH | SSLKSRITIALDTSKKEISLKVTSVTAADTAVYYCARLDYYHYSQDAWGQGTTVTVSS |
| 601 | Ab48 HC-CDR1 | GGSISGYY |
| 602 | Ab48 HC-CDR2 | IDHSGNT |
| 603 | Ab48 HC-CDR3 | ARLDYYHYSQDA |
| 604 | Ab48 HC-FR1 | QVQLQQWGAGLLKPSETLSLTCTVY |
| 605 | Ab48 HC-FR2 | WNWIRQPPGKGLEWIGE |
| 606 | Ab48 HC-FR3 | NYNSSLKSRITIALDTSKKEISLKVTSVTAADTAVYYC |
| 607 | Ab48 (10x-L8N-c72) VL | EIVLTQSPDFQSVTPKERVTITCRASQSIGASLHWYQLKPNQSPKLLIKYASQSISGVPS |
| | | RFSGSGSGTDFTLTISSLEAEDAATYYCLQSSNLYSFGQGTKLEIK |
| 608 | Ab48 LC-CDR1 | QSIGAS |
| 609 | Ab48 LC-CDR2 | YAS |
| 610 | Ab48 LC-CDR3 | LQSSNLYS |
| 611 | Ab48 LC-FR1 | EIVLTQSPDFQSVTPKERVTITCRAS |
| 612 | Ab48 LC-FR2 | LHWYQLKPNQSPKLLIK |
| 613 | Ab48 LC-FR3 | QSISGVPSRFSGSGSGTDFTLTISSLEAEDAATYYC |
| 614 | Ab49 (10x-L8N-c73) | QVQLVQSGAEVKKPGSSVKVSCKASGGTFTTYAIAWVRQAPGQGLEWMGRIIPTLDNPNY |
| | VH | AQKFQGRVSITADKSTGTSYMELRSLRSDDTAVYYCAHVSYDSSGPHLSWYFNLWGRGTL |
| | | ITVSS |
| 615 | Ab49 HC-CDR1 | GGTFTTYA |
| 616 | Ab49 HC-CDR2 | IIPTLDNP |
| 617 | Ab49 HC-CDR3 | AHVSYDSSGPHLSWYFNL |
| 618 | Ab49 HC-FR2 | AIAWVRQAPGQGLEWMGR |
| 619 | Ab49 HC-FR3 | NYAQKFQGRVSITADKSTGTSYMELRSLRSDDTAVYYC |
| 620 | Ab49 HC-FR4 | WGRGTLITVSS |
| 621 | Ab49 (10x-L8N-c73) VL | DIQMTQSPSTLSASVGDRVTITCRASQSIPSRLAWYQHKPGEAPKLLIYEASTLESGVPS |
| | | RFSGSGSGTEFTLTITSLQPDDFATYFCQGWNAFGGGTKVEIK |
| 622 | Ab49 LC-CDR1 | QSIPSR |
| 623 | Ab49 LC-CDR2 | EAS |
| 624 | Ab49 LC-CDR3 | QGWNA |
| 625 | Ab49 LC-FR2 | LAWYQHKPGEAPKLLIY |
| 626 | Ab49 LC-FR3 | TLESGVPSRFSGSGSGTEFTLTITSLQPDDFATYFC |
| 627 | Ab50 (10x-L8N-c84) | QVQLVQSGAAVKKPGSSVKVSCEASGGTFSRFAVNWVRQAPGQGLEWMGRIIPLFAIANY |
| | VH | PQRFQGRVIMSADRSTNITYMELSRLTSEDTAVYYCAREIDTVIDPAMDVFHGMEVWGQG |
| | | TTVIVSS |
| 628 | Ab50 HC-CDR1 | GGTFSRFA |
| 629 | Ab50 HC-CDR2 | IIPLFAIA |
| 630 | Ab50 HC-CDR3 | AREIDTVIDPAMDVFHGMEV |
| 631 | Ab50 HC-FR1 | QVQLVQSGAAVKKPGSSVKVSCEAS |
| 632 | Ab50 HC-FR2 | VNWVRQAPGQGLEWMGR |
| 633 | Ab50 HC-FR3 | NYPQRFQGRVIMSADRSTNITYMELSRLTSEDTAVYYC |
| 634 | Ab50 HC-FR4 | WGQGTTVIVSS |
| 635 | Ab50 (10x-L8N-c84) VL | QSVLTQPPSASGTPGQRVTISCSGRSSNIGSNYVHWYQQLPGTAPKLLIYRNNQRPSGVP |
| | | DRFSGSKSGTSASLAISGLRSEDEADYYCAGWDDSLSGAVFGGGTKLSVL |
| 636 | Ab50 LC-CDR1 | SSNIGSNY |
| 637 | Ab50 LC-CDR2 | RNN |
| 638 | Ab50 LC-CDR3 | AGWDDSLSGAV |
| 639 | Ab50 LC-FR1 | QSVLTQPPSASGTPGQRVTISCSGR |
| 640 | Ab50 LC-FR2 | VHWYQQLPGTAPKLLIY |
| 641 | Ab50 LC-FR3 | QRPSGVPDRFSGSKSGTSASLAISGLRSEDEADYYC |
| 642 | Ab50 LC-FR4 | FGGGTKLSVL |
| 643 | Ab51 (10x-L8N-c86) | EVQLVESGGGLVKPGESLRLSCAASGFTFSSSSLNWVRQAPGKGLEWVSSISISGDYTYY |
| | VH | ADSVKGRFTISRDNGKNSVYLQMDRLRVEDTAMYYCARDRFGFYDMLAHSYNVGFFQSWG |
| | | QGTLVAVSS |
| 644 | Ab51 HC-CDR1 | GFTFSSSS |
| 645 | Ab51 HC-CDR2 | ISISGDYT |
| 646 | Ab51 HC-CDR3 | ARDRFGFYDMLAHSYNVGFFQS |
| 647 | Ab51 HC-FR1 | EVQLVESGGGLVKPGESLRLSCAAS |
| 648 | Ab51 HC-FR2 | LNWVRQAPGKGLEWVSS |
| 649 | Ab51 HC-FR3 | YYADSVKGRFTISRDNGKNSVYLQMDRLRVEDTAMYYC |
| 650 | Ab51 HC-FR4 | WGQGTLVAVSS |
| 651 | Ab51 (10x-L8N-c86) VL | DIRMTQSPSSLSASVGDSVTITCRASQDITNYLAWFQQKPGKAPKSLIYSASILQSGVPS |
| | | KFSGSGSGTDFTLTISSLQPEDFATYYCQQYLTYPITFGQGTRLEIK |
| 652 | Ab51 LC-CDR1 | QDITNY |
| 653 | Ab51 LC-CDR3 | QQYLTYPIT |
| 654 | Ab51 LC-FR1 | DIRMTQSPSSLSASVGDSVTITCRAS |
| 655 | Ab51 LC-FR3 | ILQSGVPSKFSGSGSGTDFTLTISSLQPEDFATYYC |
| 656 | Ab52 (10x-L8N-c89) | QVQLVQSGSEVKKPGSSVKVSCRASGDTFSSHSLSWVRQAPGQGLEWMGRIVPMFGITNY |
| | VH | AQNFQGRVTFTADKSTSTAYMELSSLRSEDTAVYYCARAGTMITFGGLTDDGQLNWFDPW |
| | | GQGTLVTVSS |
| 657 | Ab52 HC-CDR1 | GDTFSSHS |
| 658 | Ab52 HC-CDR2 | IVPMFGIT |
| 659 | Ab52 HC-CDR3 | ARAGTMITFGGLTDDGQLNWFDP |
| 660 | Ab52 HC-FR1 | QVQLVQSGSEVKKPGSSVKVSCRAS |
| 661 | Ab52 HC-FR2 | LSWVRQAPGQGLEWMGR |
| 662 | Ab52 HC-FR3 | NYAQNFQGRVTFTADKSTSTAYMELSSLRSEDTAVYYC |
| 663 | Ab52 (10x-L8N-c89) VL | DVVLTQSPLSLSVTLGQPASISCRSSQSLEYSDGDTYLNWFHQRPGQSPRRLIYKVSNRD |
| | | SGVPDRFSGSGAGTDFTLKISKVEAEDVGVYYCMQGTNWPPNFGPGTKVDIK |
| 664 | Ab52 LC-CDR1 | QSLEYSDGDTY |
| 665 | Ab52 LC-CDR2 | KVS |
| 666 | Ab52 LC-CDR3 | MQGTNWPPN |
| 667 | Ab52 LC-FR1 | DVVLTQSPLSLSVTLGQPASISCRSS |
| 668 | Ab52 LC-FR2 | LNWFHQRPGQSPRRLIY |
| 669 | Ab52 LC-FR3 | NRDSGVPDRFSGSGAGTDFTLKISKVEAEDVGVYYC |
| 670 | Human IgG1 constant | ASTKGPSVFPLAPSSKSTSGGTAALGCLVKDYFPEPVTVSWNSGALTSGVHTFPAVLQSS |
| | region (IGHG1; | GLYSLSSWVTVPSSSLGTQTYICNVNHKPSNTKVDKKVEPKSCDKTHTCPPCPAPELLGG |
| | UniProt:P01857-1, v1) | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 671 | CH1 IgG1 (positions 1- | ASTKGPSVFPLAPSSKSTSGGTAALGCLVKDYFPEPVTVSWNSGALTSGVHTFPAVLQSS |
| | 98 of P01857-1, v1) | GLYSLSSVVTVPSSSLGTQTYICNVNHKPSNTKVDKKV |
| 672 | Hinge IgG1 (positions | EPKSCDKTHTCP |
| | 99-110 of P01857-1, | |
| | v1) | |
| 673 | CH2 IgG1 (positions | PCPAPELLGGPSVFLFPPKPKDTLMISRTPEVTCVVVDVSHEDPEVKFNWYVDGVEVHNA |
| | 111-223 of P01857-1, | KTKPREEQYNSTYRVVSVLTVLHQDWLNGKEYKCKVSNKALPAPIEKTISKAK |
| | v1) | |
| 674 | CH3 IgG1 (positions | GQPREPQVYTLPPSRDELTKNQVSLTCLVKGFYPSDIAVEWESNGQPENNYKTTPPVLDS |
| | 224-330 of P01857-1, | DGSFFLYSKLTVDKSRWQQGNVFSCSVMHEALHNHYTQKSLSLSPGK |
| | v1) | |
| 675 | Human IgG1 constant | ASTKGPSVFPLAPSSKSTSGGTAALGCLVKDYFPEPVTVSWNSGALTSGVHTFPAVLQSS |
| | region G1m3 allotype | GLYSLSSVVTVPSSSLGTQTYICNVNHKPSNTKVDKRVEPKSCDKTHTCPPCPAPELLGG |
| | (K214R, D356E and | PSVFLFPPKPKDTLMISRTPEVTCVVVDVSHEDPEVKFNWYVDGVEVHNAKTKPREEQYN |
| | L358M (EU numbering) | STYRVVSVLTVLHQDWLNGKEYKCKVSNKALPAPIEKTISKAKGQPREPQVYTLPPSREE |
| | relative to P01857-1) | MTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 676 | CH1 IgG1 G1m3 | ASTKGPSVFPLAPSSKSTSGGTAALGCLVKDYFPEPVTVSWNSGALTSGVHTFPAVLQSS |
| | allotype | GLYSLSSVVTVPSSSLGTQTYICNVNHKPSNTKVDKRV |
| 677 | CH3 IgG1 G1m3 | GQPREPQVYTLPPSREEMTKNQVSLTCLVKGFYPSDIAVEWESNGQPENNYKTTPPVLDS |
| | allotype | DGSFFLYSKLTVDKSRWQQGNVFSCSVMHEALHNHYTQKSLSLSPGK |
| 678 | Human IgG1 CH2-CH3 | PCPAPELLGGPSVFLFPPKPKDTLMISRTPEVTCVVVDVSHEDPEVKFNWYVDGVEVHNA |
| | region | KTKPREEQYNSTYRVVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 679 | Human IgG1 G1m3 | PCPAPELLGGPSVFLFPPKPKDTLMISRTPEVTCVVVDVSHEDPEVKFNWYVDGVEVHNA |
| | allotype CH2-CH3 | KTKPREEQYNSTYRVVSVLTVLHQDWLNGKEYKCKVSNKALPAPIEKTISKAKGQPREPQ |
| | region | VYTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 680 | Cκ CL (IGKC; UniProt: | RTVAAPSVFIFPPSDEQLKSGTASVVCLLNNFYPREAKVQWKVDNALQSGNSQESVTEQD |
| | P01834-1, v2) | SKDSTYSLSSTLTLSKADYEKHKVYACEVTHQGLSSPVTKSFNRGEC |
| 681 | Cλ CL1 (IGLC1; | GQPKANPTVTLFPPSSEELQANKATLVCLISDFYPGAVTVAWKADGSPVKAGVETTKPSK |
| | UniProt: POCG04-1, v1) | QSNNKYAASSYLSLTPEQWKSHRSYSCQVTHEGSTVEKTVAPTECS |
| 682 | Cλ CL2 (IGLC2; | GQPKAAPSVTLFPPSSEELQANKATLVCLISDFYPGAVTVAWKADSSPVKAGVETTTPSK |
| | UniProt: PODOY2-1, | QSNNKYAASSYLSLTPEQWKSHRSYSCQVTHEGSTVEKTVAPTECS |
| | v1) | |
| 683 | Cλ CL3 (IGLC3; | GQPKAAPSVTLFPPSSEELQANKATLVCLISDFYPGAVTVAWKADSSPVKAGVETTTPSK |
| | UniProt: PODOY3-1, | QSNNKYAASSYLSLTPEQWKSHKSYSCQVTHEGSTVEKTVAPTECS |
| | v1) | |
| 684 | Cλ CL6 (IGLC6; | GQPKAAPSVTLFPPSSEELQANKATLVCLISDFYPGAVKVAWKADGSPVNTGVETTTPSK |
| | UniProt: POCF74-1, v1) | QSNNKYAASSYLSLTPEQWKSHRSYSCQVTHEGSTVEKTVAPAECS |
| 685 | Cλ CL7 (IGLC7; | GQPKAAPSVTLFPPSSEELQANKATLVCLVSDFNPGAVTVAWKADGSPVKVGVETTKPSK |
| | UniProt: ADM8Q6-1, | QSNNKYAASSYLSLTPEQWKSHRSYSCRVTHEGSTVEKTVAPAECS |
| | v3) | |
| 686 | BA.1 RBD | RVQPTESIVRFPNITNLCPFDEVFNATRFASVYAWNRKRISNCVADYSVLYNLAPFFTFK |
| | | CYGVSPTKLNDLCFTNVYADSFVIRGDEVRQIAPGQTGNIADYNYKLPDDFTGCVIAWNS |
| | | NKLDSKVSGNYNYLYRLFRKSNLKPFERDISTEIYQAGNKPCNGVAGFNCYFPLRSYSFR |
| | | PTYGVGHQPYRVVVLSFELLHAPATVCGPKKSTNLVKNKCVNF |
| 687 | BA.2 RBD | RVQPTESIVRFPNITNLCPFDEVFNATRFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVGGNYNYLYRLFRKSNLKPFERDISTEIYQAGNKPCNGVAGFNCYFPLRSYGFR |
| | | PTYGVGHQPYRVVVLSFELLHAPATVCGPKKSTNLVKNKCVNF |
| 688 | BA.5 RBD | RVQPTESIVRFPNITNLCPFDEVFNATRFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNF |
| 689 | BA.2.75 RBD | RVQPTESIVRFPNITNLCPFHEVFNATRFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVSGNYNYLYRLFRKSKLKPFERDISTEIYQAGNKPCNGVAGFNCYFPLQSYGFR |
| | | PTYGVGHQPYRVVVLSFELLHAPATVCGPKKSTNLVKNKCVNF |
| 690 | BA.2.75.2 RBD | RVQPTESIVRFPNITNLCPFHEVFNATTFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVSGNYNYLYRLFRKSKLKPFERDISTEIYQAGNKPCNGVAGSNCYFPLQSYGFR |
| | | PTYGVGHQPYRVVVLSFELLHAPATVCGPKKSTNLVKNKCVNF |
| 691 | BA.4.6.1 RBD | RVQPTESIVRFPNITNLCPFDEVFNATTFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNF |
| 692 | BF.7 RBD | RVQPTESIVRFPNITNLCPFDEVFNATTFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSKVGGNYNYRYRLFRKSNLKPFERDISTEIYQAGNKPCNGVAGVNCYFPLQSYGFR |
| | | PTYGVGHQPYRVVVLSFELLHAPATVCGPKKSTNLVKNKCVNF |
| 693 | BQ.1.1 RBD | RVQPTESIVRFPNITNLCPFDEVFNATTFASVYAWNRKRISNCVADYSVLYNFAPFFAFK |
| | | CYGVSPTKLNDLCFTNVYADSFVIRGNEVSQIAPGQTGNIADYNYKLPDDFTGCVIAWNS |
| | | NKLDSTVGGNYNYRYRLFRKSKLKPFERDISTEIYQAGNKPCNGVAGVNCYFPLQSYGFR |
| | | PTYGVGHQPYRVVVLSFELLHAPATVCGPKKSTNLVKNKCVNF |
| 694 | XBB.1 RBD | RVQPTESIVRFPNITNLCPFHEVFNATTFASVYAWNRKRISNCVADYSVIYNFAPFFAFK |
| | | CYGVSPTKLNDLCFTNVYADSFVIRGNEVSQIAPGQTGNIADYNYKLPDDFTGCVIAWNS |
| | | NKLDSKPSGNYNYLYRLFRKSKLKPFERDISTEIYQAGNKPCNGVAGSNCYSPLQSYGFR |
| | | PTYGVGHQPYRVVVLSFELLHAPATVCGPKKSTNLVKNKCVNF |
| 695 | XBB.1.16 RBD | RVQPTESIVRFPNITNLCPFHEVFNATTFASVYAWNRKRISNCVADYSVIYNFAPFFAFK |
| | | CYGVSPTKLNDLCFTNVYADSFVIRGNEVSQIAPGQTGNIADYNYKLPDDFTGCVIAWNS |
| | | NKLDSKPSGNYNYLYRLFRKSKLKPFERDISTEIYQAGNRPCNGVAGPNCYSPLQSYGFR |
| | | PTYGVGHQPYRVVVLSFELLHAPATVCGPKKSTNLVKNKCVNF |
| 696 | XBB.2.3 RBD | RVQPTESIVRFPNITNLCPFHEVFNATTFASVYAWNRKRISNCVADYSVIYNFAPFFAFK |
| | | CYGVSPTKLNDLCFTNVYADSFVIRGNEVSQIAPGQTGNIADYNYKLPDDFTGCVIAWNS |
| | | NKLDSKPSGNYNYLYRLFRKSKLKPFERDISTEIYQAGNKPCNGVAGPNCYSPLQSYGFR |
| | | PTYGVGHQPYRVVVLSFELLHASATVCGPKKSTNLVKNKCVNF |
| 697 | EG.5 RBD | RVQPTESIVRFPNITNLCPFHEVFNATTFASVYAWNRKRISNCVADYSVIYNFAPFFAFK |
| | | CYGVSPTKLNDLCFTNVYADSFVIRGNEVSQIAPGQTGNIADYNYKLPDDFTGCVIAWNS |
| | | NKLDSKPSGNYNYLYRLLRKSKLKPFERDISTEIYQAGNKPCNGVAGPNCYSPLQSYGFR |
| | | PTYGVGHQPYRVVVLSFELLHAPATVCGPKKSTNLVKNKCVNF |
| 698 | EG.5.1 RBD | RVQPTESIVRFPNITNLCPFHEVFNATTFASVYAWNRKRISNCVADYSVIYNFAPFFAFK |
| | | CYGVSPTKLNDLCFTNVYADSFVIRGNEVSQIAPGQTGNIADYNYKLPDDFTGCVIAWNS |
| | | NKLDSKPSGNYNYLYRLLRKSKLKPFERDISTEIYQAGNKPCNGVAGPNCYSPLQSYGFR |
| | | PTYGVGHQPYRVVVLSFELLHAPATVCGPKKSTNLVKNKCVNF |
| 699 | B.1.1.7 RBD | RVQPTESIVRFPNITNLCPFGEVFNATRFASVYAWNRKRISNCVADYSVLYNSASFSTFK |
| | | CYGVSPTKLNDLCFTNVYADSFVIRGDEVRQIAPGQTGKIADYNYKLPDDFTGCVIAWNS |
| | | NNLDSKVGGNYNYLYRLFRKSNLKPFERDISTEIYQAGSTPCNGVEGFNCYFPLQSYGFQ |
| | | PTYGVGYQPYRVVVLSFELLHAPATVCGPKKSTNLVKNKCVNF |
| 700 | B.1.351 RBD | RVQPTESIVRFPNITNLCPFGEVFNATRFASVYAWNRKRISNCVADYSVLYNSASFSTFK |
| | | CYGVSPTKLNDLCFTNVYADSFVIRGDEVRQIAPGQTGNIADYNYKLPDDFTGCVIAWNS |
| | | NNLDSKVGGNYNYLYRLFRKSNLKPFERDISTEIYQAGSTPCNGVKGFNCYFPLQSYGFQ |
| | | PTYGVGYQPYRVVVLSFELLHAPATVCGPKKSTNLVKNKCVNF |
| 701 | B.1.617.2 RBD | RVQPTESIVRFPNITNLCPFGEVFNATRFASVYAWNRKRISNCVADYSVLYNSASFSTFK |
| | | CYGVSPTKLNDLCFTNVYADSFVIRGDEVRQIAPGQTGKIADYNYKLPDDFTGCVIAWNS |
| | | NNLDSKVGGNYNYRYRLFRKSNLKPFERDISTEIYQAGSKPCNGVEGFNCYFPLQSYGFQ |
| | | PTNGVGYQPYRVVVLSFELLHAPATVCGPKKSTNLVKNKCVNF |
| 702 | P.1 RBD | RVQPTESIVRFPNITNLCPFGEVFNATRFASVYAWNRKRISNCVADYSVLYNSASFSTFK |
| | | CYGVSPTKLNDLCFTNVYADSFVIRGDEVRQIAPGQTGTIADYNYKLPDDFTGCVIAWNS |
| | | NNLDSKVGGNYNYLYRLFRKSNLKPFERDISTEIYQAGSTPCNGVKGFNCYFPLQSYGFQ |
| | | PTYGVGYQPYRVVVLSFELLHAPATVCGPKKSTNLVKNKCVNF |
| 703 | BA. 1 RBM | NSNKLDSKVSGNYNYLYRLFRKSNLKPFERDISTEIYQAGNKPCNGVAGENCYFPLRSYS |
| | | FRPTYGVGHQPY |
| 704 | BA.2 RBM | NSNKLDSKVGGNYNYLYRLFRKSNLKPFERDISTEIYQAGNKPCNGVAGENCYFPLRSYG |
| | | FRPTYGVGHQPY |
| 705 | BA.5 RBM | NSNKLDSKVGGNYNYRYRLFRKSNLKPFERDISTEIYQAGNKPCNGVAGVNCYFPLQSYG |
| | | FRPTYGVGHQPY |
| 706 | BA.2.75 RBM | NSNKLDSKVSGNYNYLYRLFRKSKLKPFERDISTEIYQAGNKPCNGVAGFNCYFPLQSYG |
| | | FRPTYGVGHQPY |
| 707 | BA.2.75.2 RBM | NSNKLDSKVSGNYNYLYRLFRKSKLKPFERDISTEIYQAGNKPCNGVAGSNCYFPLQSYG |
| | | FRPTYGVGHQPY |
| 708 | BA.4.6.1 RBM | NSNKLDSKVGGNYNYRYRLFRKSNLKPFERDISTEIYQAGNKPCNGVAGVNCYFPLQSYG |
| | | FRPTYGVGHQPY |
| 709 | BF.7 RBM | NSNKLDSKVGGNYNYRYRLFRKSNLKPFERDISTEIYQAGNKPCNGVAGVNCYFPLQSYG |
| | | FRPTYGVGHQPY |
| 710 | BQ.1.1 RBM | NSNKLDSTVGGNYNYRYRLFRKSKLKPFERDISTEIYQAGNKPCNGVAGVNCYFPLQSYG |
| | | FRPTYGVGHQPY |
| 711 | XBB.1 RBM | NSNKLDSKPSGNYNYLYRLFRKSKLKPFERDISTEIYQAGNKPCNGVAGSNCYSPLQSYG |
| | | FRPTYGVGHQPY |
| 712 | XBB.1.16 RBM | NSNKLDSKPSGNYNYLYRLFRKSKLKPFERDISTEIYQAGNRPCNGVAGPNCYSPLQSYG |
| | | FRPTYGVGHQPY |
| 713 | XBB.2.3 RBM | NSNKLDSKPSGNYNYLYRLFRKSKLKPFERDISTEIYQAGNKPCNGVAGPNCYSPLQSYG |
| | | FRPTYGVGHQPY |
| 714 | EG.5 RBM | NSNKLDSKPSGNYNYLYRLLRKSKLKPFERDISTEIYQAGNKPCNGVAGPNCYSPLQSYG |
| | | FRPTYGVGHQPY |
| 715 | EG.5.1 RBM | NSNKLDSKPSGNYNYLYRLLRKSKLKPFERDISTEIYQAGNKPCNGVAGPNCYSPLQSYG |
| | | FRPTYGVGHQPY |
| 716 | B.1.1.7 RBM | NSNNLDSKVGGNYNYLYRLFRKSNLKPFERDISTEIYQAGSTPCNGVEGFNCYFPLQSYG |
| | | FQPTYGVGYQPY |
| 717 | B.1.351 RBM | NSNNLDSKVGGNYNYLYRLFRKSNLKPFERDISTEIYQAGSTPCNGVKGFNCYFPLQSYG |
| | | FQPTYGVGYQPY |
| 718 | B.1.617.2 RBM | NSNNLDSKVGGNYNYRYRLFRKSNLKPFERDISTEIYQAGSKPCNGVEGENCYFPLQSYG |
| | | FQPTNGVGYQPY |
| 719 | P.1 RBM | NSNNLDSKVGGNYNYLYRLFRKSNLKPFERDISTEIYQAGSTPCNGVKGFNCYFPLQSYG |
| | | FQPTYGVGYQPY |
| 720 | Ab1 (B11.2) hIgG1 HC | EVQLVESGGGLVQPGGSLRLSCAASEIIVSRNYMTWVRQAPGKGLEWLAVLYAGGSSFYA |
| | | DSVKGRFTISRDNSKNTLFLQMNSLRAEDTAVYYCARDLSLSGGFDYWGQGALVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 721 | Ab1 (B11.2) κ LC | DIQLTQSPSFLSASVGDRVTITCRASQGISNYLAWYQQNPGKAPKLLIYAVSTLHSGVPS |
| | | RFSGSGSGTEFTLTISSLQPEDFATYYCQHLNTDSCTFGQGTKLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 722 | Ab2 (H12.2) hIgG1 HC | EFQLVESGGRLVRPGGSLRLSCVASEIIVSRNYMSWIRQAPGKGLEWVSILYAGGTTYYA |
| | | DSVKGRFTISRDNSKNTLYLQLNSLRVEDTAIYYCVRPIVGGRGGMDVWGQGTTVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 723 | Ab2 (H12.2) κ LC | DIQMTQSPSSLSASVGDRVTITCQASQDINKYLNWYQQKPGKAPKLLIYDASNLEPGVPS |
| | | RFSGSGSGTDFVFTITSLQPEDIATYYCHYYDDVPYTFGQGTQLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 724 | Ab3 (C2.2) hIgG1 HC | QVQLVQSGAEVKKPGSSVRVSCEASGDTFSRYAISWVRQAPGQGLEWMGRIIPMFGMANS |
| | | AQKFQARVTISADKSTSTAYMEVSSLRSEDTAVYYCATDTFYPNDGVHRMEYWGQGALVI |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 725 | Ab3 (C2.2) κ LC | DIQMTQSPSSLSASVGDRVTITCRASQTISNYLNWYQQKPGKAPNLLIYGTSILQSGVPS |
| | | RFSGSGSGTDFTLTISSLQPEDFATYYCQQSHSAPRTFGPGTKVDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 726 | Ab4 (G7.2) hIgG1 HC | QVQLVQSGAEVKKPGSSVKVSCEASGGTFSTYAISWVRQAPGQGLEWMGRIIPIFGIANY |
| | | AQKFQGRVTITADKSTSTAYMEVSSLRSEDTAVYYCATTFYDHSSTYRTHSMDVWGQGTT |
| | | VTVSSASTKGPSVFPLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 727 | Ab4 (G7.2) κ LC | DIQMTQSPSSLSASVGDRVTITCRASQSISNYLNWYQQRPGKAPKLLIYAASGLQSGVPS |
| | | RFSGSGSGTNFTLTISTLHPEDFATYYCQQTHSTPRAFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 728 | Ab5 (10x-L8N-c25) | DFQLVESGGGLVRPGGSLRLSCVASEITVSRNYMSWIRQAPGKGLEWVSIIYPGGTTYYA |
| | hIgG1 HC | DSVKGRFTISRDNSKNTMYLQLNSLRPEDTAIYYCVRPIVRGGGGMDVWGQGTTVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 729 | Ab5 (10x-L8N-c25) κ | DIQMTQSPSSLSASVGDRVTITCQASQDINKYLNWYQQKPGKAPKLLIYDATNLEPGVPS |
| | LC | RFSGSGSGTDFVFTITSLQPEDIATYYCQYYDDVPYTFGQGTQLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 730 | Ab6 (K23) hIgG1 HC | EVQLVESGGGLVQPGGSLRLSCSASGFTFNNYVMHWVRQAPGKGLEYVSAINSNGGSTYY |
| | | AGSVKGRFTISRDNSNNTLYLQMSSLRAEDTAVYYCIKDAGYYSSLGVDSWGQGTLVTVS |
| | | SASTKGPSVF |
| | | PLAPSSKSTSGGTAALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGNVFSCS |
| | | VMHEALHNHYTQKSLSLSPGK |
| 731 | Ab6 (K23) κ LC | EIVLTQSPATLSLSPGERATLSCRASQSVGSYLAWFQQKPGQAPRLLIYDASYRATGIPA |
| | | RFSGSGSGTDFTLTISSLEPEDVGIYYCQQRSNWPQTFGGGTKVDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 732 | Ab7 (G1) hIgG1 HC | EVQLVESGGGLVKPGGSLRLSCAASGFTVSTYIINWVRQAPGKGLEWVSSITSESDYMFD |
| | | ADSVRGRFTISRDNAKNLVYLQMNSLRAEDTAVYYCARDQGAYSGYDLSPGGDAFDVWGQ |
| | | GTMVTVSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 733 | Ab7 (G1) κ LC | DIQMTQSPSTLSASVGDRVTITCRASQSISTWLAWYQQKPGKAPKLLIYKASNLESGVPS |
| | | RFSGSGSGTEFTLTISRLQPDDSATYCCQHYNSYPYTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 734 | Ab8 (B9) hIgG1 HC | EVQLVQSRAEVKKPGESLKISCKGSGYSFTTYWIGWVRQMPGKGLEWMGIIRPGDSDTRY |
| | | SPSFQGQVTISADKSITTAYLQWSSLKASDTAMYYCAMTYSGDQYDFWGQGTVVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 735 | Ab8 (B9) κ LC | DIQMTQSPSTLSASVGDRVTITCRASQSIHTWLAWYQQTPGKAPKLLIYKASLLESGVPS |
| | | RFSGSGSGTEFTLTISSLQPGDFATYYCQHYNSYSHTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 736 | Ab9 (C68) hIgG1 HC | QVQLVQSGAEVKKPGASVKVSCKISGYTLTDFSIHWVRQAPGKGLEWMAGFDPEHRETIF |
| | | AQKFQGRVAMTEATSTDTAYMELSSLRSDDTAVYYCATTGDFDSWRGYYLWGQGTLVTVS |
| | | AASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 737 | Ab9 (C68) κ LC | DIQLTQSPSSVSASVGDSVTITCRASQGISRWLAWYQQKPGKAPRLLIYSASTLQSGVPS |
| | | RFSGSGSGTDFTLTISSLQPDDFATYYCQHTNNFPFSFGPGTKVDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 738 | Ab10 (C115) | QVQLVQSGAEVKKPGSSVKVSCRASGDTFSTYTITWVRQAPGQGLEWMGRIIPILDTADY |
| | hIgG1 HC | AQKFQGRVTITADKSTSTAYMELSSLRSEDTAMYYCAKNYPNGYSGYDYFSWDGFDPWGQ |
| | | GTLVTVSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 739 | Ab10 (C115) κ LC | QSVLTQPPSVSGAPGQRVTISCTGSSSNIGANYEVHWYQQLPGTAPKLLIFGHSNRPSAV |
| | | PDRFSGSKSGTSASLAITGLQTEDEADYYCQSYDSSLSGVVFGGGTKLTVLRTVAAPSVF |
| | | IFPPSDEQLKSGTASVVCLLNNFYPREAKVQWKVDNALQSGNSQESVTEQDSKDSTYSLS |
| | | STLTLSKADYEKHKVYACEVTHQGLSSPVTKSFNRGEC |
| 740 | Ab11 (D4) hIgG1 HC | QVQLQESGPRLVKPSGTLSLSCAVSGGPFSNTNWWSWIRQTPGKGLEWIGEINDSGNTVY |
| | | NPALKSRVTMSVDKSKKQFSLNLHSLTAADTAVYFCARVWGHFDYWGQGVRVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPGK |
| 741 | Ab11 (D4) κ LC | DIQMTQSPSSLYASVADRVTITCRASQGISNSLAWYQQQPGKAPQLLLYAASTLESGVPS |
| | | RFSGSGSGTDFTLTISSLQPEDFATYYCQQYYSLRTFGGGTKVEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 742 | Ab12 (H5) hIgG1 HC | EVQLLESGGGLVQPGGSLRLSCAASGFTFSGSDMSWVRQAPGKGLEWVSVIGGSGTYAYY |
| | | SDSVKGRFTISRDNSKNMLFLQMNSLRAEDTAIYYCAKETGFLWFGELLDSWGQGALVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 743 | Ab12 (H5) κ LC | DIQMTQSPSSLSASVGDRVTITCRASEAISNSLAWYQQRPGKAPRLLLYAAATLESGVPP |
| | | RFSGSGSGTDFTLTISTLQPEDFATYYCQQYYSPPPRTFGQGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 744 | Ab13 (D10) hIgG1 HC | EVQLVESGGGLVQPGRSLRLSCAASGFTFDEYAMHWVRQAPGKGLEWVSGISFNSGSVGY |
| | | AGAAKGRFTISRDNAKKSLYLEMNSLRDEDTAFYYCAKDRGEHWLVRLFDSWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 745 | Ab13 (D10) κ LC | DIQMTQSPSTLSASVGDRVTITCRPSQSIDRWLAWYQQKPGKAPTLLISAASSLETGVPS |
| | | RFSGSGSGTEFTLTISSLQPDDFATYYCQQYNGYSMFGPGTKVEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 746 | Ab14 (F9) hIgG1 HC | EVQLVESGGGLVQPGGSLRLSCATSGFTFDDFAMHWVRQGPGKGLEWVSGISWNSGKIAY |
| | | TDSVKGRFSISRDNAKNSLYLQMNSLRPEDTALYYCTKDHAPSAILGDILTGFDPWGQGT |
| | | LVTASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 747 | Ab14 (F9) κ LC | DIQMTQSPVSLSASVGDRVTITCRASQSISVYLNWYQQKPGKAPKLLMYAASILQSGVPS |
| | | RFSGSGSATDFTLTITSLQPEDFATYFCQQSFTMPPTFGQGTNLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 748 | Ab15 (E1.1) hIgG1 HC | QVQLVQSGAEVKKPGASVKVSCKASGYIFNNYAIQWVRQAPGQRLEWMAWIHTGNGDTKY |
| | | SQKFQGRVTITRDTSASTAYMELSSLRSEDTAIYYCARVRPNWNTLGWFDPWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 749 | Ab15 (E1.1) κ LC | NFMLTQPHSVSESPGKTVTISCTGSGGSIATNYVQWYQQRPGSAPTTVIYEDNERPSGVP |
| | | DRFSGSIDSSSNSASLTISGLRTEDEADYYCQSYDISTHWVFGGGTKLTVLRTVAAPSVF |
| | | IFPPSDEQLKSGTASWVCLLNNFYPREAKVQWKVDNALQSGNSQESVTEQDSKDSTYSLS |
| | | STLTLSKADYEKHKVYACEVTHQGLSSPVTKSFNRGEC |
| 750 | Ab16 (G1.1) hIgG1 HC | EVQLVESGGTSVRPGGSLRLSCSASGFSVRSNFMTWVRQAPGKGLECVSVIYSGSGGSTF |
| | | YADSVKGRFTISKDDSKNTLYLQMNSLRAEDTAIYYCAREVSHAFDLWGQGTMVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 751 | Ab16 (G1.1) κ LC | DIQMTQSPSTLSASVGDRVTITCRASQTIGRWLAWYQQRPGKAPSLLIYMASILESGVPL |
| | | RFSGSGSGTEFTLTISGLQPDDFATYYCQQYNSDSPYSFGQGTKLEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 752 | Ab17 (B5.1) hIgG1 HC | QVQLQESGPGLVKPSETLSLTCTVSGGSLTSYYWSWIRQPPGGGLEWIGHIYYTGITDHN |
| | | PSLKSRVTISLDTSRNQFSLKVRSVTAADTALYYCARAAGSSDYFDFWGQGTPVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 753 | Ab17 (B5.1) κ LC | DIQMTQSPSTLSASVGDRVTITCRASQSISRWLAWYQQKPGKAPKLLIYMASILESGVPS |
| | | RFSGGGSGTEFTLTISSLQPDDFATYYCQQYNSYRTFGQGTKVEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 754 | Ab18 (H9.1) hIgG1 HC | QVQLQESGPGLVKPSETLSLTCTVSGGSITSYYWSWIRQSPGKGLEWIGHIYYSGSTDYN |
| | | PSLKSRVTISVDTSKNQFSLKLTYVAAADTAVYFCARAGGSSDYFDYWGQGTLVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 755 | Ab18 (H9.1) κ LC | DIQMTQSPSTLSASVGDRVTITCRASQSIGRWLAWYQQKPGKAPKFLIYMASILEDGVPS |
| | | RFSGSGSGTEFTLTITSLQPDDFATYYCQQYNDYRTFGQGTKVEIK |
| 756 | Ab19 (F5.1) hIgG1 HC | EVQLVESGGGLVKPGGSLRLSCAASGFIFSRNGMHWVRQAPGKGLEWVSSIDNDGTYMYY |
| | | ADSVRGRFTVSRDNAKNSLFLQLNSLRAEDTAVYYCARDRFGYYDALTDSYNAGYFDSWG |
| | | QGTLVTVSSASTKGPSVFPLAPSSKSTSGGTAALGCLVKDYFPEPVTVSWNSGALTSGVH |
| | | TFPAVLQSSGLYSLSSWVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 757 | Ab19 (F5.1) κ LC | DIQMTQSPSSLSASVGDSVTITCRASQDIYNYLAWFQQKPGKAPKSLIYTASKLESGVPS |
| | | KFSGSGSGTDFTLTISSLQPEDFATYYCQQYHSSPITFGQGTRVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 758 | Ab20 (C6.1) hIgG1 HC | EVQLVESGGGLVKPGGSLRLSCAASGFAFSTNGMNWVRQVPGKGLEWVSSISSTSEYTYY |
| | | TESVKGRFTISRDNAKNSLFLQMNSLRAEDTAVYYCVRDRFGYYDVLASSYNVGFFQSWG |
| | | QGALVTVSSASTKGPSVFPLAPSSKSTSGGTAALGCLVKDYFPEPVTVSWNSGALTSGVH |
| | | TFPAVLQSSGLYSLSSWVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 759 | Ab20 (C6.1) κ LC | DIQMTQSPSSLSASVGDGVTITCRASQDISNNLAWFQQKPGKAPKCLIYSASSLQSGVPL |
| | | KFRGSGSGTDFTLSITSLDPEDFATYYCQQYISYPITFGQGTRLDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 760 | Ab21 (E6.1) hIgG1 HC | QVQLVQSGAEVKKPGSSVEVSCKASGGTFSSYAISWVRQAPGQGLEWMGRIIPSLGITNS |
| | | AENFQGRVTITADKSTSTVYMELSSLRSEDTAVYYCARDFHPLYQSCSSTSCYDEWEPRM |
| | | DVWGQGTTVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 761 | Ab21 (E6.1) κ LC | QSVLTQPPSASGTPGQRVTISCHGSSSNIGSKTVNWYQQLPGTAPKLLIYSNDQRPSGVP |
| | | DRFSGSKSGTSASLAISGLQSEDEADYYCAAWDGSLDGPVFGGGTKLTVLRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 762 | Ab22 (C9.1) hIgG1 HC | QVQLVQSGAEVKKPGSSVKVSCKASGGTFSSYGISWVRQAPGQGLEWMGRIIPSLSITNS |
| | | AEKFQGRVTITADKSTSTAYMELSRLRSEDTALYYCARDFHPRYEFCDSTSCYDEWEPRM |
| | | DVWGQGTTVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 763 | Ab22 (C9.1) κ LC | QSVLTQSPSASGTPGQRVIISCSGSSSNIGRKTVNWYQQLPGTAPKLVIYSNDQRPSGVP |
| | | DRFSGSKSGTSASLAISGLQSEDEADYYCAAWDDSLDGPVFGGGTKLTVLRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 764 | Ab23 (B10.1) hIgG1 | QVQLVQSGAEVKKPGSAVKVSCKASGGTFSSYPITWVRQAPGQGLEWVGRAIPILGITST |
| | HC | AQKFQGRVTIIADKSTSTAYMELSRLRSEDTAVYYCARDFHPRYQDCDSTSCYDQWEPRM |
| | | DVWGQGTTVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 765 | Ab23 (B10.1) κ LC | QSVLTQPPSASGTPGQRVTISCSGSSSNIGSNTVNWYQQLPGTAPKLLIYTNNQRPSGVP |
| | | NRFSASKSGTSASLAISGLQSEDEADYYCAAWDDRLSGPVFGGGTKVTVLRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 766 | Ab24 (G1.2) hIgG1 HC | QVQLVQSGAEVKKPGSSVKVSCEASGDTFSRYAINWVRQAPGQGLEWMGRIPMFGNTNYA |
| | | QKFQGRFTITADKSRGTAYMEVIGLTSADTAVYYCATSPFYYSDGGYPFDFWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 767 | Ab24 (G1.2) κ LC | EIVLTQSPGTLSLSPGERATLSCRASQSLNSAYLAWYQQRAGLAPRLLIYGASSRATGIP |
| | | DKFSGSGSGTDFTLTISRLEPEDFAVYYCQQYAYSPRTFGQGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 768 | Ab25 (H10.2) hIgG1 | QVQLVQSGAEVKKPGSSVKVSCKASRGTFSNYAISWVRQAPGQGLEWMGRIIPSLSITNS |
| | HC | AEKFQGRVTMTADKSTSTVYMELSRLRSEDTAVYYCARDFHPRYEYCSSTSCYDEWEPRM |
| | | DVWGQGTTVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 769 | Ab25 (H10.2) κ LC | QSVLTQSPSASGTPGQRVIISCSGSSSNIGRKTVNWYQQLPGTAPKLLMYSNDQRPSGVP |
| | | DRFSGSKSGTSASLAISGLQSEDEADYYCAAWDDSLDGPVFGGGTKLTVLRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 770 | Ab26 (G4.2) hIgG1 HC | QVQLVQSGAEVKKPGSSVKVSCKASGGTFSSHTVSWVRQAPGQGLEWMGRIIPPFGIVNY |
| | | AQKFQGRVTMTADESTSTAYMELSSLRSEDTAVYYCARSNVVVVTEAGWFDPWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 771 | Ab26 (G4.2) κ LC | DIQMTQSPSSVSASVGDRVSISCRASQDISSSLAWYQQKPGQAPKVLIYDASTLQSGVPS |
| | | RFSGSGSGTDFTLTISNLRPDDFATYFCQQAHSFPFTFGPGTKVDIRRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 772 | Ab27 (E12.2) hIgG1 | EVQLVESGGGLVQPGGSLRLSCGASGITVSSNYMNWVRQAPGKGLEWVSTLYAGGSTFYA |
| | HC | DSVKGRFIISRDNSKNTLYLQMNSLRADDTAVYYCARDLVDYGMDVWGQGTTVTVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 773 | Ab27 (E12.2) κ LC | DIQLTQSPSSLSASVGDRVTITCRASQGISSYLAWYQQKPGKAPKLLIYAASTLQSGVPS |
| | | RFSGSGSGTDFTLTISSLQPEDFATYYCQQLNSYSPFTFGPGTKVDIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 774 | Ab28 (10x-L8N-c1) | QFQLVQSGAEVKKPGSSVKVSCRASGGSFTSHAISWVRQAPGQGFEWMGRIIPMFGIANY |
| | hIgG1 HC | APKFQGRVTMSADKFKDIVYMEVNSLTSEDTALYYCARSQPMTSVTTLWFDPWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 775 | Ab28 (10x-L8N-c1) κ | EIVLTQSPGTLSLSPGERATLSCRASEPVGGSYLAWYQQKPGQAPRLLIHGASSRATGIP |
| | LC | DRFSGSGSGTDFVLTISRLEPEDFAVYHCQQYASSPYTFGQGTKLEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 776 | Ab29 (10x-L8N-c5) | QVHLVQSGAEVKKPGSSVKVSCKTSGDTFTRYAINWVRQAPGQGLEWMGRIIPMFGIPNY |
| | hIgG1 HC | AQKFQGRVTMTADKSTDIAYMELSSLRSEDTAVYYCARSSFYSDSSGYYLDYWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 777 | Ab29 (10x-L8N-c5) κ | EIVLTQSPGTLSLSPGERATLSCRASQNLDSNYLAWYQQKPGQAPRLLIYGASIRATGIP |
| | LC | DRFSGSGSGTDFTLTISRLEPEDFAVYHCQQYHNSPRTFGQGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 778 | Ab30 (10x-L8N-c9) | QVQLVQSGAEVKKPGSSVKVSCKASGDTFTKYAITWVRQAPGEGLEWMGRIIPRFGMANY |
| | hIgG1 HC | AQNFQGRVTMTADQSTSTAYMELTSLRSNDTAVYYCATTFYFDSSYYHAMDYWGQGSLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 779 | Ab30 (10x-L8N-c9) κ | DIQMTQSPSSLSASVGDRVTITCRASQNIDTYLIWYQQKPGKAPNLLVYGASTLQSGVPS |
| | LC | RFSGSGSGTDFTLTISSLQPEDFATYYCQQTYNAPRTFGQGTRLDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 780 | Ab31 (10x-L8N-c10) | QVQLVQSGAEVKKPGSSVKVSCKASGDTFSRYAISWVRQAPGQGLEWMGRIIPMFGTANY |
| | | AQNF |
| | hIgG1 HC | QGRVTITADKSTSTAYMELTSLRSEDTAVYYCATTYFYDSDRDRTHSMDVWGPGSAVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 781 | Ab31 (10x-L8N-c10) κ | DIQMTQSPSSLSASVGDRVTITCRTSQSVGNYLNWYQQKPGKAPNLLIYAASTLQNGVPS |
| | LC | RFSGSGSGTDFTLTISSLQPEDFATYYCQQTHSTPRAFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 782 | Ab32 (10x-L8N-c12) | EVQLVESGGGLVQPGGSLRVTCVASGFAVRTNFMTWVRQAPGKGLQCVSVIYGDGSTYYA |
| | hIgG1 HC | DSVKGRFSISRDNSKNTVYLQMNSLSAEDTAVYYCAREVSHAFDLWGPGTMVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPGK |
| 783 | Ab32 (10x-L8N-c12) κ | DIQLTQSPSTLSASLGDRVTITCRASQSISGWLAWYQQKPGRAPQLLIYKASLLETGVPS |
| | LC | RFSGSGSGTVFTLTISSLQPDDFATYYCQQYDTYSPYTFGQGTKLEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 784 | Ab33 (10x-L8N-c18) | EVQLVESGGGLVQPGGSLRLSCGASGITVSSNYMNWVRQAPGKGLEWVSTLYAGGSTFYA |
| | hIgG1 HC | DSVKGRFIISRDNSKNTLYLQMNSLRAEDTAVYYCARDLVDYGMDVWGQGTTVTVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 785 | Ab33 (10x-L8N-c18) κ | AIQLTQSPSSLSASVGDRVTITCRASQGISSYLAWYQQKPGKAPKLLIYAASTLQSGVPS |
| | LC | RFSGSGSGTDFTLTISSLQPEDFATYYCQQLNSYSPFTFGPGTKVDIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 786 | Ab34 (10x-L8N-c19) | EVQLVESGGGLVQSGGSLRLSCAASGIIVSRNYMSWVRQAPGKGLEWVSVIYPGGSTFYA |
| | hIgG1 HC | DSVKGRFTISRDNSKNTLYLQMNSLRAEDTAVFYCARDRGEDIFDFWGQGTMVSVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 787 | Ab34 (10x-L8N-c19) κ | DIQLTQSPTFLSASVGDRVTITCRASQGISSYLAWYQQKPGKAPKLLIYAASTLQGGVPS |
| | LC | RFSGSGSGTEFTLTISSLQPEDFATYYCQQLNSSPGFTFGPGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 788 | Ab35 (10x-L8N-c20) | QVQLQESGPGLVKPSETLSLTCTVSGDSIYSYSWTWIRQPPGKGLEWIGQLYYNGSTSYN |
| | hIgG1 HC | ASLKSRVSISLDTSKNQFSLKLRFVTAADTAMYYCARVEYYSNYFDPWGQGTQVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 789 | Ab35 (10x-L8N-c20) κ | EIVLTQSPATLSLSPGERATLSCRASQSVGSYLAWYQQKPGQAPRLLIYDASNRVPGIPA |
| | LC | RFSGSGSGTDFTLTISSLPPEDFAVYYCQQRSDWPPTFGGGTKVEIRRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 790 | Ab37 (10x-L8N-c22) | EVQLVESGGGLVQPGGSLRVSCAASGIIVSRNYMTWVRQAPGKGLEWVSVIYAGGSTFYA |
| | hIgG1 HC | DSVKGRFTISRDNSKNTLYLQMNSLRVDDTAVYYCARDLEIAGGMDVWGQGTTVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 791 | Ab37 (10x-L8N-c22) κ | AIQLTQSPSSLSASVGDRVTITCRASEGISNYLAWYQQKPGKAPKVLIYAASTLQSGAPP |
| | LC | RFSGSGSGTDFTLTISSLQPEDFATYYCQQLNSYPITFGQGTRLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 792 | Ab38 (G8.2) hIgG1 HC | QVQLVQSGAEVKKPGSSVKVSCKASGDTFSRDAISWVRQAPGQGLEWMGRIIPMFGIANY |
| | | AQNFQGRVTMTADKYTSTAYMELSSLRSEDTAVYYCARGGYQYESSGYHLDHWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 793 | Ab38 (G8.2) κ LC | EIVLTQSPGTLSLSPGERATLSCRASQSLSSSYLAWYQQKPGQAPRLLIYGTSSRATGIP |
| | | DRFSGSGSGTDFTLTISRLEPEDFAVYYCHQYSNSPRTFGQGTKVEVKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 794 | Ab39 (10x-L8N-c30) | QVQLVQSGAEVKKPGSSVKISCKASGDTSSSYTINWVRQAPGQGLEWMGRIIPMFNRANY |
| | hIgG1 HC | ARKFQGRVTMTADRSTDTAYMEVSSLTSDDTAVYYCARTWIEPHNWFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 795 | Ab39 (10x-L8N-c30) κ | EIVLRQSPGTLSLSPGERATLSCRASQSVSGNHLAWYQRKPGQAPRLLIYAASSRATGIP |
| | LC | DRFSGSGSGTDFTLTINRLEPEDFAVFYCQQYGTSPPTFGGGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 796 | Ab40 (10x-L8N-c32) | QVQLQESGPRLVKPSETLSLTCTVSGGSISSYYWTWIRQPPGKGLEWIGYISYSGSTNYN |
| | hIgG1 HC | PSLRSRVTMSVDTSKNQFSLDLNSVTAADTAVYYCATDGGGFYPGYFPHWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCWWVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 797 | Ab40 (10x-L8N-c32) κ | DIQMTQSPSSLSASVGDRVTITCRASQSISDYVNWYQQKPGRAPRLLIYAPSTLQSGVPS |
| | LC | RFSGSGSGTDFTLTISNLQPEDFGTYFCQQSYSTPPYTFGQGTKVEMKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 798 | Ab41 (10x-L8N-c39) | QVQLVQSGAEVKKPGASVKVSCKGSGYTFTDYGISWVRQAPGQGLEWMGWIRAYNGNTDY |
| | hIgG1 HC | AQKFQGRVTMTRDTSTTTAYMELRSLRSDDTAVYFCALNLIAAAATPFDHWGQGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 799 | Ab41 (10x-L8N-c39) κ | EIVLTQSPGTLSLSRGERATLSCRASQSVSSSYLAWYQQKRGQAPRLLIYGATSRATGIP |
| | LC | DRFSGSGSGTDFTLTISRLEPEDFAVYYCQHYGSSPPPFGGGTKVEIRRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 800 | Ab42 (10x-L8N-c41) | EVQLLESGGGLVQPGGSLRLSCAGSGFTFSSYAMSWVRQAPGKGLEWVSSISSSGGLTFY |
| | hIgG1 HC | ADSVRGRSTISRDHSRNTLYLQMNSLRAEDTALYYCAREQGDSYDDYFDYWGQGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 801 | Ab42 (10x-L8N-c41) κ | DIQMTQSPSSLSASVGDRVTITCRASQGIRSSLAWYQQRPGKAPKLLLSAASILESGVPS |
| | LC | RFSGSGSGTDYTLTINGLQPDDFATYYCQQYYVTPGITFGGGTKVEIRRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSFNRGEC |
| 802 | Ab43 (10x-L8N-c42) | EVQLVESGGGLVKPGGSLRLSCAASGFTFSRHTMNWVRQAPGKGLEWVSSIGGSTPLSFY |
| | hIgG1 HC | AESVKGRFSISRDNAKSSLFLQMSSLRADDTAVYYCVRDSIASVTTLFDLWGRGTVVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCWWVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 803 | Ab43 (10x-L8N-c42) κ | DIQMTQSPSSLSASVGDRVTITCRASQTIRNYINWYQQRPGKAPRLLIYDASTLQSGVPS |
| | LC | RFSGGGSGTDFTLTISSLEPEDFATYYCQQSYVSPPEDSFGQGTKLEIKRTVAAPSVFIF |
| | | PPSDEQLKSGTASVVCLLNNFYPREAKVQWKVDNALQSGNSQESVTEQDSKDSTYSLSST |
| | | LTLSKADYEKHKVYACEVTHQGLSSPVTKSFNRGEC |
| 804 | Ab44 (10x-L8N-c53) | QVQLQQSGAEVKKPGSSVKVSCESSGGTFTSHAINWVRQAPGQRLEWMGRIIPVLGVAAY |
| | hIgG1 HC | AQKFQGRVTLTADKFTSTAYMELSSLRSEDTAVYFCARSAYNYDSSGYHFDYWGQGTLVL |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 805 | Ab44 (10x-L8N-c53) κ | DIQMTQSPSTLSASVGDRVTITCRASQSISNWLAWYQQKPGTAPKLLIFKASTLQSGVPS |
| | LC | RFSGSGSGTEFTLTISSLQPDDFATYHCQQYHSSRSFGQGTKVEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 806 | Ab45 (10x-L8N-c62) | QVQLVQSGAEVKKPGSSMTVSCEASGDTFSRYSINWVRQAPGQGFEWMGRIIPMFGLATY |
| | hIgG1 HC | AHKFQGRVTISADKSTTTAHMELNRLTSEDTAIYYCATAPFYYDNSTYPFDFWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 807 | Ab45 (10x-L8N-c62) κ | DIQLTQSPSSLSASVGDRVTITCRASQSINNYLNWYQHKPGKAPKLLIYGASTLQSGVPS |
| | LC | RFSGSGSGSDFTLTISSLQPEDFATYYCQQSYNAPRTFGQGTRLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 808 | Ab46 (10x-L8N-c63) | QVHLVQSGAEVKKPGSSVKVSCKASGGTFSSHTVSWVRQAPGQGLEWMGRIIPPFGIVNY |
| | hIgG1 HC | AQKFQGRVTITADESTSTAYMELSSLSSEDTAVYYCARSNVVVVTEAGWFDPWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 809 | Ab46 (10x-L8N-c63) κ | DIQMTQSPSSVSASVGDRVTITCRASQGISSSLAWYQQKPGKAPKVLIFDASTLQSGVPS |
| | LC | RFSGSGSGTDFTLTISNLRPEDFATYYCQQAHSFPFTFGPGTKVDIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 810 | Ab47 (10x-L8N-c64) | QVQLVQSGAEVKKPGSSVKVSCEASGDTFSRYAITWVRQAPGQGLEWMGRIIPMFGSANY |
| | hIgG1 HC | AQKFQGRVSFTADKSTSTASMELSSLRSEDTAVYYCATSPYYSDSGGYYCDYWGQGSLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 811 | Ab47 (10x-L8N-c64) κ | DIQMTQSPSSLSASVGDRVTITCRASQGISNYLAWYQQKPGKAPKLLLYGASRLQSGVPS |
| | LC | RFSGSGSGTDYTLTISSLQPEDFATFYCQQYYSAPRTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 812 | Ab48 (10x-L8N-c72) | QVQLQQWGAGLLKPSETLSLTCTVYGGSISGYYWNWIRQPPGKGLEWIGEIDHSGNTNYN |
| | hIgG1 HC | SSLKSRITIALDTSKKEISLKVTSVTAADTAVYYCARLDYYHYSQDAWGQGTTVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 813 | Ab48 (10x-L8N-c72) κ | EIVLTQSPDFQSVTPKERVTITCRASQSIGASLHWYQLKPNQSPKLLIKYASQSISGVPS |
| | LC | RFSGSGSGTDFTLTISSLEAEDAATYYCLQSSNLYSFGQGTKLEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 814 | Ab49 (10x-L8N-c73) | QVQLVQSGAEVKKPGSSVKVSCKASGGTFTTYAIAWVRQAPGQGLEWMGRIIPTLDNPNY |
| | hIgG1 HC | AQKFQGRVSITADKSTGTSYMELRSLRSDDTAVYYCAHVSYDSSGPHLSWYFNLWGRGTL |
| | | ITVSSASTKGPSVFPLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 815 | Ab49 (10x-L8N-c73) κ | DIQMTQSPSTLSASVGDRVTITCRASQSIPSRLAWYQHKPGEAPKLLIYEASTLESGVPS |
| | LC | RFSGSGSGTEFTLTITSLQPDDFATYFCQGWNAFGGGTKVEIKRTVAAPSVFIFPPSDEQ |
| | | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| 816 | Ab50 (10x-L8N-c84) | QVQLVQSGAAVKKPGSSVKVSCEASGGTFSRFAVNWVRQAPGQGLEWMGRIIPLFAIANY |
| | hIgG1 HC | PQRFQGRVIMSADRSTNITYMELSRLTSEDTAVYYCAREIDTVIDPAMDVFHGMEVWGQG |
| | | TTVIVSSASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 817 | Ab50 (10x-L8N-c84) κ | QSVLTQPPSASGTPGQRVTISCSGRSSNIGSNYVHWYQQLPGTAPKLLIYRNNQRPSGVP |
| | LC | DRFSGSKSGTSASLAISGLRSEDEADYYCAGWDDSLSGAVFGGGTKLSVLRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 818 | Ab51 (10x-L8N-c86) | EVQLVESGGGLVKPGESLRLSCAASGFTFSSSSLNWVRQAPGKGLEWVSSISISGDYTYY |
| | hIgG1 HC | ADSVKGRFTISRDNGKNSVYLQMDRLRVEDTAMYYCARDRFGFYDMLAHSYNVGFFQSWG |
| | | QGTLVAVSSASTKGPSVFPLAPSSKSTSGGTAALGCLVKDYFPEPVTVSWNSGALTSGVH |
| | | TFPAVLQSSGLYSLSSVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 819 | Ab51 (10x-L8N-c86) κ | DIRMTQSPSSLSASVGDSVTITCRASQDITNYLAWFQQKPGKAPKSLIYSASILQSGVPS |
| | LC | KFSGSGSGTDFTLTISSLQPEDFATYYCQQYLTYPITFGQGTRLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 820 | Ab52 (10x-L8N-c89) | QVQLVQSGSEVKKPGSSVKVSCRASGDTFSSHSLSWVRQAPGQGLEWMGRIVPMFGITNY |
| | hIgG1 HC | AQNFQGRVTFTADKSTSTAYMELSSLRSEDTAVYYCARAGTMITFGGLTDDGQLNWFDPW |
| | | GQGTLVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 821 | Ab52 (10x-L8N-c89) κ | DVVLTQSPLSLSVTLGQPASISCRSSQSLEYSDGDTYLNWFHQRPGQSPRRLIYKVSNRD |
| | LC | SGVPDRFSGSGAGTDFTLKISKVEAEDVGVYYCMQGTNWPPNFGPGTKVDIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| 822 | Ref mAb heavy chain | EVQLVESGGXLVXPGGSLRLSCXASEIIVSRNYMXWXRQAPGKGLEWXXXLYAGGXXXYA |
| | (CDRs bold and | DSVKGRFTISRDNSKNTLXLQXNSLRXEDTAXYYCXRXXXXXGGXDXWGQGXXVTVSS |
| | underlined) | |
| 823 | Ref mAb light chain | DIQXTQSPSXLSASVGDRVTITCXASQXIXXYLXWYQQXPGKAPKLLIYXXSXLXXGVPS |
| | (CDRs bold and | RFSGSGSGTXFXXTIXSLQPEDXATYYCXXXXXXXXTFGQGTXLEIK |
| | underlined) | |
| 824 | E7 VH | QVQLQESGPGLVKPSETLSLTCTVSGGFIGPHYWSWVRQPPGKGLEWIGYIYISGSTNYN |
| | | PSLKSRLTISVDMSKSQFSLTLSSATAADTAVYYCARGGGYLETGPFEYWGQGTLVTVSS |
| 825 | E7 HC-CDR1 | GGFIGPHY |
| 826 | E7 HC-CDR2 | IYISGST |
| 827 | E7 HC-CDR3 | ARGGGYLETGPFEY |
| 828 | E7 HC-FR2 | WSWVRQPPGKGLEWIGY |
| 829 | E7 HC-FR3 | NYNPSLKSRLTISVDMSKSQFSLTLSSATAADTAVYYC |
| 830 | E7 VL | DIVMTQSPLSLPVTPGEPASISCRSSQSLLQNNGYNYLAWYLQKPGQSPQLLIYLSSTRA |
| | | SGVPDRFSGSGSGTDFTLKISRVEAEDVGVYYCMQSLQIPGTFGQGTRLEIK |
| 831 | E7 LC-CDR1 | QSLLQNNGYNY |
| 832 | E7 LC-CDR2 | LSS |
| 833 | E7 LC-CDR3 | MQSLQIPGT |
| 834 | E7 LC-FR1 | DIVMTQSPLSLPVTPGEPASISCRSS |
| 835 | E7 LC-FR2 | LAWYLQKPGQSPQLLIY |
| 836 | E7 LC-FR3 | TRASGVPDRFSGSGSGTDFTLKISRVEAEDVGVYYCM |
| 837 | E7 hIgG1 HC | QVQLQESGPGLVKPSETLSLTCTVSGGFIGPHYWSWVRQPPGKGLEWIGYIYISGSTNYN |
| | | PSLKSRLTISVDMSKSQFSLTLSSATAADTAVYYCARGGGYLETGPFEYWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 838 | E7 κ LC | DIVMTQSPLSLPVTPGEPASISCRSSQSLLQNNGYNYLAWYLQKPGQSPQLLIYLSSTRA |
| | | SGVPDRFSGSGSGTDFTLKISRVEAEDVGVYYCMQSLQIPGTFGQGTRLEIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| 839 | LyCov-1404 VH | QITLKESGPTLVKPTQTLTLTCTFSGFSLSISGVGVGWLRQPPGKALEWLALIYWDDDKR |
| | | YSPSLKSRLTISKDTSKNQVVLKMTNIDPVDTATYYCAHHSISTIFDHWGQGTLVTVSS |
| 840 | LyCov-1404 HC-CDR1 | GFSLSISGVG |
| 841 | LyCov-1404 HC-CDR2 | IYWDDDK |
| 842 | LyCov-1404 HC-CDR3 | AHHSISTIFDH |
| 843 | LyCov-1404 HC-FR1 | QITLKESGPTLVKPTQTLTLTCTFS |
| 844 | LyCov-1404 HC-FR2 | WLRQPPGKALEWLAL |
| 845 | LyCov-1404 HC-FR3 | RYSPSLKSRLTISKDTSKNQVVLKMTNIDPVDTATYYC |
| 846 | LyCov-1404 VL | QSALTQPASVSGSPGQSITISCTATSSDVGDYNYVSWYQQHPGKAPKLMIFEVSDRPSGI |
| | | SNRFSGSKSGNTASLTISGLQAEDEADYYCSSYTTSSAVFGGGTKLTVL |
| 847 | LyCov-1404 LC-CDR1 | SSDVGDYNY |
| 848 | LyCov-1404 LC-CDR2 | EVS |
| 849 | LyCov-1404 LC-CDR3 | SSYTTSSAV |
| 850 | LyCov-1404 LC-FR1 | QSALTQPASVSGSPGQSITISCTAT |
| 851 | LyCov-1404 LC-FR2 | VSWYQQHPGKAPKLMIF |
| 852 | LyCov-1404 LC-FR3 | DRPSGISNRFSGSKSGNTASLTISGLQAEDEADYYC |
| 853 | LyCov-1404 LC-FR4 | AVFGGGTKLTVL |
| 854 | LyCov-1404 hIgG1 | QITLKESGPTLVKPTQTLTLTCTFSGFSLSISGVGVGWLRQPPGKALEWLALIYWDDDKR |
| | (G1m3) HC | YSPSLKSRLTISKDTSKNQVVLKMTNIDPVDTATYYCAHHSISTIFDHWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKRVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 855 | LyCov-1404 CλCL2 LC | QSALTQPASVSGSPGQSITISCTATSSDVGDYNYVSWYQQHPGKAPKLMIFEVSDRPSGI |
| | | SNRFSGSKSGNTASLTISGLQAEDEADYYCSSYTTSSAVFGGGTKLTVLGQPKAAPSVTL |
| | | FPPSSEELQANKATLVCLISDFYPGAVTVAWKADSSPVKAGVETTTPSKQSNNKYAASSY |
| | | LSLTPEQWKSHRSYSCQVTHEGSTVEKTVAPTECS |

TABLE A

| | | Column A | | | Column B | | |
|---|---|---|---|---|---|---|---|
| | | VH | | | VL | | |
| Row | Antibody | HC-CDR1 | HC-CDR2 | HC-CDR3 | LC-CDR1 | LC-CDR2 | LC-CDR3 |
| 1 | Ab1 (B11.2) | SEQ ID NO: 37 | SEQ ID NO: 38 | SEQ ID NO: 39 | SEQ ID NO: 45 | SEQ ID NO: 46 | SEQ ID NO: 47 |
| 2 | Ab2 (H12.2) | SEQ ID NO: 37 | SEQ ID NO: 53 | SEQ ID NO: 54 | SEQ ID NO: 60 | SEQ ID NO: 61 | SEQ ID NO: 62 |
| 3 | Ab3 (C2.2) | SEQ ID NO: 68 | SEQ ID NO: 69 | SEQ ID NO: 70 | SEQ ID NO: 76 | SEQ ID NO: 77 | SEQ ID NO: 78 |
| 4 | Ab4 (G7.2) | SEQ ID NO: 84 | SEQ ID NO: 85 | SEQ ID NO: 86 | SEQ ID NO: 90 | SEQ ID NO: 91 | SEQ ID NO: 92 |
| 5 | Ab5 (10x- | SEQ ID NO: 97 | SEQ ID NO: 98 | SEQ ID NO: 99 | SEQ ID NO: 60 | SEQ ID NO: 103 | SEQ ID NO: 104 |
| | L8N-c25) | | | | | | |
| 6 | Ab6 (K23) | SEQ ID NO: 106 | SEQ ID NO: 107 | SEQ ID NO: 108 | SEQ ID NO: 114 | SEQ ID NO: 61 | SEQ ID NO: 115 |
| 7 | Ab7 (G1) | SEQ ID NO: 121 | SEQ ID NO: 122 | SEQ ID NO: 123 | SEQ ID NO: 129 | SEQ ID NO: 130 | SEQ ID NO: 131 |
| 8 | Ab8 (B9) | SEQ ID NO: 137 | SEQ ID NO: 138 | SEQ ID NO: 139 | SEQ ID NO: 145 | SEQ ID NO: 130 | SEQ ID NO: 146 |
| 9 | Ab9 (C68) | SEQ ID NO: 150 | SEQ ID NO: 151 | SEQ ID NO: 152 | SEQ ID NO: 158 | SEQ ID NO: 159 | SEQ ID NO: 160 |
| 10 | Ab10 (C115) | SEQ ID NO: 165 | SEQ ID NO: 166 | SEQ ID NO: 167 | SEQ ID NO: 172 | SEQ ID NO: 173 | SEQ ID NO: 174 |
| 11 | Ab11 (D4) | SEQ ID NO: 180 | SEQ ID NO: 181 | SEQ ID NO: 182 | SEQ ID NO: 188 | SEQ ID NO: 91 | SEQ ID NO: 189 |
| 12 | Ab12 (H5) | SEQ ID NO: 194 | SEQ ID NO: 195 | SEQ ID NO: 196 | SEQ ID NO: 201 | SEQ ID NO: 202 | SEQ ID NO: 203 |
| 13 | Ab13 (D10) | SEQ ID NO: 207 | SEQ ID NO: 208 | SEQ ID NO: 209 | SEQ ID NO: 214 | SEQ ID NO: 91 | SEQ ID NO: 215 |
| 14 | Ab14 (F9) | SEQ ID NO: 221 | SEQ ID NO: 222 | SEQ ID NO: 223 | SEQ ID NO: 229 | SEQ ID NO: 91 | SEQ ID NO: 230 |
| 15 | Ab15 (E1.1) | SEQ ID NO: 236 | SEQ ID NO: 237 | SEQ ID NO: 238 | SEQ ID NO: 243 | SEQ ID NO: 244 | SEQ ID NO: 245 |
| 16 | Ab16 (G1.1) | SEQ ID NO: 250 | SEQ ID NO: 251 | SEQ ID NO: 252 | SEQ ID NO: 257 | SEQ ID NO: 258 | SEQ ID NO: 259 |
| 17 | Ab17 (B5.1) | SEQ ID NO: 263 | SEQ ID NO: 264 | SEQ ID NO: 265 | SEQ ID NO: 271 | SEQ ID NO: 258 | SEQ ID NO: 272 |
| 18 | Ab18 (H9.1) | SEQ ID NO: 275 | SEQ ID NO: 276 | SEQ ID NO: 277 | SEQ ID NO: 281 | SEQ ID NO: 258 | SEQ ID NO: 282 |
| 19 | Ab19 (F5.1) | SEQ ID NO: 286 | SEQ ID NO: 287 | SEQ ID NO: 288 | SEQ ID NO: 292 | SEQ ID NO: 293 | SEQ ID NO: 294 |
| 20 | Ab20 (C6.1) | SEQ ID NO: 300 | SEQ ID NO: 301 | SEQ ID NO: 302 | SEQ ID NO: 306 | SEQ ID NO: 159 | SEQ ID NO: 307 |
| 21 | Ab21 (E6.1) | SEQ ID NO: 313 | SEQ ID NO: 314 | SEQ ID NO: 315 | SEQ ID NO: 319 | SEQ ID NO: 320 | SEQ ID NO: 321 |
| 22 | Ab22 (C9.1) | SEQ ID NO: 326 | SEQ ID NO: 327 | SEQ ID NO: 328 | SEQ ID NO: 332 | SEQ ID NO: 320 | SEQ ID NO: 333 |
| 23 | Ab23 (B10.1) | SEQ ID NO: 337 | SEQ ID NO: 338 | SEQ ID NO: 339 | SEQ ID NO: 344 | SEQ ID NO: 345 | SEQ ID NO: 346 |
| 24 | Ab24 (G1.2) | SEQ ID NO: 68 | SEQ ID NO: 351 | SEQ ID NO: 352 | SEQ ID NO: 356 | SEQ ID NO: 357 | SEQ ID NO: 358 |
| 25 | Ab25 (H10.2) | SEQ ID NO: 363 | SEQ ID NO: 327 | SEQ ID NO: 364 | SEQ ID NO: 332 | SEQ ID NO: 320 | SEQ ID NO: 333 |
| 26 | Ab26 (G4.2) | SEQ ID NO: 369 | SEQ ID NO: 370 | SEQ ID NO: 371 | SEQ ID NO: 375 | SEQ ID NO: 61 | SEQ ID NO: 376 |
| 27 | Ab27 (E12.2) | SEQ ID NO: 382 | SEQ ID NO: 383 | SEQ ID NO: 384 | SEQ ID NO: 389 | SEQ ID NO: 91 | SEQ ID NO: 390 |
| 28 | Ab28 (10x- | SEQ ID NO: 394 | SEQ ID NO: 395 | SEQ ID NO: 396 | SEQ ID NO: 401 | SEQ ID NO: 357 | SEQ ID NO: 402 |
| | L8N-c1) | | | | | | |
| 29 | Ab29 (10x- | SEQ ID NO: 406 | SEQ ID NO: 407 | SEQ ID NO: 408 | SEQ ID NO: 412 | SEQ ID NO: 357 | SEQ ID NO: 413 |
| | L8N-c5) | | | | | | |
| 30 | Ab30 (10x- | SEQ ID NO: 417 | SEQ ID NO: 418 | SEQ ID NO: 419 | SEQ ID NO: 424 | SEQ ID NO: 357 | SEQ ID NO: 425 |
| | L8N-c9) | | | | | | |
| 31 | Ab31 (10x- | SEQ ID NO: 68 | SEQ ID NO: 428 | SEQ ID NO: 429 | SEQ ID NO: 433 | SEQ ID NO: 91 | SEQ ID NO: 92 |
| | L8N-c10) | | | | | | |
| 32 | Ab32 (10x- | SEQ ID NO: 437 | SEQ ID NO: 438 | SEQ ID NO: 252 | SEQ ID NO: 444 | SEQ ID NO: 130 | SEQ ID NO: 445 |
| | L8N-c12) | | | | | | |
| 33 | Ab33 (10x- | SEQ ID NO: 382 | SEQ ID NO: 383 | SEQ ID NO: 384 | SEQ ID NO: 389 | SEQ ID NO: 91 | SEQ ID NO: 390 |
| | L8N-c18) | | | | | | |
| 34 | Ab34 (10x- | SEQ ID NO: 454 | SEQ ID NO: 455 | SEQ ID NO: 456 | SEQ ID NO: 389 | SEQ ID NO: 91 | SEQ ID NO: 461 |
| | L8N-c19) | | | | | | |
| 35 | Ab35 (10x- | SEQ ID NO: 465 | SEQ ID NO: 466 | SEQ ID NO: 467 | SEQ ID NO: 114 | SEQ ID NO: 61 | SEQ ID NO: 472 |
| | L8N-c20) | | | | | | |
| 36 | Ab37 (10x- | SEQ ID NO: 454 | SEQ ID NO: 476 | SEQ ID NO: 477 | SEQ ID NO: 482 | SEQ ID NO: 91 | SEQ ID NO: 483 |
| | L8N-c22) | | | | | | |
| 37 | Ab38 (G8.2) | SEQ ID NO: 488 | SEQ ID NO: 395 | SEQ ID NO: 489 | SEQ ID NO: 492 | SEQ ID NO: 77 | SEQ ID NO: 493 |
| 38 | Ab39 (10x- | SEQ ID NO: 497 | SEQ ID NO: 498 | SEQ ID NO: 499 | SEQ ID NO: 503 | SEQ ID NO: 91 | SEQ ID NO: 504 |
| | L8N-c30) | | | | | | |
| 39 | Ab40 (10x- | SEQ ID NO: 509 | SEQ ID NO: 510 | SEQ ID NO: 511 | SEQ ID NO: 516 | SEQ ID NO: 517 | SEQ ID NO: 518 |
| | L8N-c32) | | | | | | |
| 40 | Ab41 (10x- | SEQ ID NO: 523 | SEQ ID NO: 524 | SEQ ID NO: 525 | SEQ ID NO: 530 | SEQ ID NO: 531 | SEQ ID NO: 532 |
| | L8N-c39) | | | | | | |
| 41 | Ab42 (10x- | SEQ ID NO: 536 | SEQ ID NO: 537 | SEQ ID NO: 538 | SEQ ID NO: 543 | SEQ ID NO: 91 | SEQ ID NO: 544 |
| | L8N-c41) | | | | | | |
| 42 | Ab43 (10x- | SEQ ID NO: 548 | SEQ ID NO: 549 | SEQ ID NO: 550 | SEQ ID NO: 555 | SEQ ID NO: 61 | SEQ ID NO: 556 |
| | L8N-c42) | | | | | | |
| 43 | Ab44 (10x- | SEQ ID NO: 560 | SEQ ID NO: 561 | SEQ ID NO: 562 | SEQ ID NO: 568 | SEQ ID NO: 130 | SEQ ID NO: 569 |
| | L8N-c53) | | | | | | |
| 44 | Ab45 (10x- | SEQ ID NO: 573 | SEQ ID NO: 574 | SEQ ID NO: 575 | SEQ ID NO: 580 | SEQ ID NO: 357 | SEQ ID NO: 581 |
| | L8N-c62) | | | | | | |
| 45 | Ab46 (10x- | SEQ ID NO: 369 | SEQ ID NO: 370 | SEQ ID NO: 371 | SEQ ID NO: 588 | SEQ ID NO: 61 | SEQ ID NO: 376 |
| | L8N-c63) | | | | | | |
| 46 | Ab47 (10x- | SEQ ID NO: 68 | SEQ ID NO: 593 | SEQ ID NO: 594 | SEQ ID NO: 45 | SEQ ID NO: 357 | SEQ ID NO: 597 |
| | L8N-c64) | | | | | | |
| 47 | Ab48 (10x- | SEQ ID NO: 601 | SEQ ID NO: 602 | SEQ ID NO: 603 | SEQ ID NO: 608 | SEQ ID NO: 609 | SEQ ID NO: 610 |
| | L8N-c72) | | | | | | |
| 48 | Ab49 (10x- | SEQ ID NO: 615 | SEQ ID NO: 616 | SEQ ID NO: 617 | SEQ ID NO: 622 | SEQ ID NO: 623 | SEQ ID NO: 624 |
| | L8N-c73) | | | | | | |
| 49 | Ab50 (10x- | SEQ ID NO: 628 | SEQ ID NO: 629 | SEQ ID NO: 630 | SEQ ID NO: 636 | SEQ ID NO: 637 | SEQ ID NO: 638 |
| | L8N-c84) | | | | | | |
| 50 | Ab51 (10x- | SEQ ID NO: 644 | SEQ ID NO: 645 | SEQ ID NO: 646 | SEQ ID NO: 652 | SEQ ID NO: 159 | SEQ ID NO: 653 |
| | L8N-c86) | | | | | | |
| 51 | Ab52 (10x- | SEQ ID NO: 657 | SEQ ID NO: 658 | SEQ ID NO: 659 | SEQ ID NO: 664 | SEQ ID NO: 665 | SEQ ID NO: 666 |
| | L8N-c89) | | | | | | |

TABLE B

| | | Column A | | | | Column B | | | |
|---|---|---|---|---|---|---|---|---|---|
| | | VH | | | | VL | | | |
| Row | Antibody | HC-FR1 | HC-FR2 | HC-FR3 | HC-FR4 | LC-FR1 | LC-FR2 | LC-FR3 | LC-FR4 |
| 1 | Ab1 (B11.2) | SEQ ID | SEQ ID | SEQ ID | SEQ ID | SEQ ID | SEQ ID | SEQ ID | SEQ ID |
| | | NO: 40 | NO: 41 | NO: 42 | NO: 43 | NO: 48 | NO: 49 | NO: 50 | NO: 51 |
| 2 | Ab2 (H12.2) | SEQ ID | SEQ ID | SEQ ID | SEQ ID | SEQ ID | SEQ ID | SEQ ID | SEQ ID |
| | | NO: 55 | NO: 56 | NO: 57 | NO: 58 | NO: 63 | NO: 64 | NO: 65 | NO: 66 |
| 3 | Ab3 (C2.2) | SEQ ID | SEQ ID | SEQ ID | SEQ ID | SEQ ID | SEQ ID | SEQ ID | SEQ ID |
| | | NO: 71 | NO: 72 | NO: 73 | NO: 74 | NO: 79 | NO: 80 | NO: 81 | NO: 82 |
| 4 | Ab4 (G7.2) | SEQ ID | SEQ ID | SEQ ID | SEQ ID | SEQ ID | SEQ ID | SEQ ID | SEQ ID |
| | | NO: 87 | NO: 72 | NO: 88 | NO: 58 | NO: 79 | NO: 93 | NO: 94 | NO: 95 |
| 5 | Ab5 (10x- | SEQ ID | SEQ ID | SEQ ID | SEQ ID | SEQ ID | SEQ ID | SEQ ID | SEQ ID |
| | L8N-c25) | NO: 100 | NO: 56 | NO: 101 | NO: 58 | NO: 63 | NO: 64 | NO: 65 | NO: 66 |
| 6 | Ab6 (K23) | SEQ ID | SEQ ID | SEQ ID | SEQ ID | SEQ ID | SEQ ID | SEQ ID | SEQ ID |
| | | NO: 109 | NO: 110 | NO: 111 | NO: 112 | NO: 116 | NO: 117 | NO: 118 | NO: 119 |
| 7 | Ab7 (G1) | SEQ ID | SEQ ID | SEQ ID | SEQ ID | SEQ ID | SEQ ID | SEQ ID | SEQ ID |
| | | NO: 124 | NO: 125 | NO: 126 | NO: 127 | NO: 132 | NO: 133 | NO: 134 | NO: 135 |
| 8 | Ab8 (B9) | SEQ ID | SEQ ID | SEQ ID | SEQ ID | SEQ ID | SEQ ID | SEQ ID | SEQ ID |
| | | NO: 140 | NO: 141 | NO: 142 | NO: 143 | NO: 132 | NO: 147 | NO: 148 | NO: 135 |
| 9 | Ab9 (C68) | SEQ ID | SEQ ID | SEQ ID | SEQ ID | SEQ ID | SEQ ID | SEQ ID | SEQ ID |
| | | NO: 153 | NO: 154 | NO: 155 | NO: 156 | NO: 161 | NO: 162 | NO: 163 | NO: 82 |
| 10 | Ab10 (C115) | SEQ ID | SEQ ID | SEQ ID | SEQ ID | SEQ ID | SEQ ID | SEQ ID | SEQ ID |
| | | NO: 168 | NO: 169 | NO: 170 | NO: 112 | NO: 175 | NO: 176 | NO: 177 | NO: 178 |
| 11 | Ab11 (D4) | SEQ ID | SEQ ID | SEQ ID | SEQ ID | SEQ ID | SEQ ID | SEQ ID | SEQ ID |
| | | NO: 183 | NO: 184 | NO: 185 | NO: 186 | NO: 190 | NO: 191 | NO: 192 | NO: 95 |
| 12 | Ab12 (H5) | SEQ ID | SEQ ID | SEQ ID | SEQ ID | SEQ ID | SEQ ID | SEQ ID | SEQ ID |
| | | NO: 197 | NO: 198 | NO: 199 | NO: 43 | NO: 79 | NO: 204 | NO: 205 | NO: 135 |
| 13 | Ab13 (D10) | SEQ ID | SEQ ID | SEQ ID | SEQ ID | SEQ ID | SEQ ID | SEQ ID | SEQ ID |
| | | NO: 210 | NO: 211 | NO: 212 | NO: 112 | NO: 216 | NO: 217 | NO: 218 | NO: 219 |
| 14 | Ab14 (F9) | SEQ ID | SEQ ID | SEQ ID | SEQ ID | SEQ ID | SEQ ID | SEQ ID | SEQ ID |
| | | NO: 224 | NO: 225 | NO: 226 | NO: 227 | NO: 231 | NO: 232 | NO: 233 | NO: 234 |
| 15 | Ab15 (E1.1) | SEQ ID | SEQ ID | SEQ ID | SEQ ID | SEQ ID | SEQ ID | SEQ ID | SEQ ID |
| | | NO: 239 | NO: 240 | NO: 241 | NO: 112 | NO: 246 | NO: 247 | NO: 248 | NO: 178 |
| 16 | Ab16 (G1.1) | SEQ ID | SEQ ID | SEQ ID | SEQ ID | SEQ ID | SEQ ID | SEQ ID | SEQ ID |
| | | NO: 253 | NO: 254 | NO: 255 | NO: 127 | NO: 132 | NO: 260 | NO: 261 | NO: 51 |
| 17 | Ab17 (B5.1) | SEQ ID | SEQ ID | SEQ ID | SEQ ID | SEQ ID | SEQ ID | SEQ ID | SEQ ID |
| | | NO: 266 | NO: 267 | NO: 268 | NO: 269 | NO: 132 | NO: 133 | NO: 273 | NO: 135 |
| 18 | Ab18 (H9.1) | SEQ ID | SEQ ID | SEQ ID | SEQ ID | SEQ ID | SEQ ID | SEQ ID | SEQ ID |
| | | NO: 266 | NO: 278 | NO: 279 | NO: 112 | NO: 132 | NO: 283 | NO: 284 | NO: 135 |
| 19 | Ab19 (F5.1) | SEQ ID | SEQ ID | SEQ ID | SEQ ID | SEQ ID | SEQ ID | SEQ ID | SEQ ID |
| | | NO: 124 | NO: 289 | NO: 290 | NO: 112 | NO: 295 | NO: 296 | NO: 297 | NO: 298 |
| 20 | Ab20 (C6.1) | SEQ ID | SEQ ID | SEQ ID | SEQ ID | SEQ ID | SEQ ID | SEQ ID | SEQ ID |
| | | NO: 124 | NO: 303 | NO: 304 | NO: 43 | NO: 308 | NO: 309 | NO: 310 | NO: 311 |
| 21 | Ab21 (E6.1) | SEQ ID | SEQ ID | SEQ ID | SEQ ID | SEQ ID | SEQ ID | SEQ ID | SEQ ID |
| | | NO: 316 | NO: 72 | NO: 317 | NO: 58 | NO: 322 | NO: 323 | NO: 324 | NO: 178 |
| 22 | Ab22 (C9.1) | SEQ ID | SEQ ID | SEQ ID | SEQ ID | SEQ ID | SEQ ID | SEQ ID | SEQ ID |
| | | NO: 329 | NO: 72 | NO: 330 | NO: 58 | NO: 334 | NO: 335 | NO: 324 | NO: 178 |
| 23 | Ab23 (B10.1) | SEQ ID | SEQ ID | SEQ ID | SEQ ID | SEQ ID | SEQ ID | SEQ ID | SEQ ID |
| | | NO: 340 | NO: 341 | NO: 342 | NO: 58 | NO: 347 | NO: 323 | NO: 348 | NO: 349 |
| 24 | Ab24 (G1.2) | SEQ ID | SEQ ID | SEQ ID | SEQ ID | SEQ ID | SEQ ID | SEQ ID | SEQ ID |
| | | NO: 87 | NO: 353 | NO: 354 | NO: 112 | NO: 359 | NO: 360 | NO: 361 | NO: 135 |
| 25 | Ab25 (H10.2) | SEQ ID | SEQ ID | SEQ ID | SEQ ID | SEQ ID | SEQ ID | SEQ ID | SEQ ID |
| | | NO: 329 | NO: 72 | NO: 365 | NO: 58 | NO: 334 | NO: 367 | NO: 324 | NO: 178 |
| 26 | Ab26 (G4.2) | SEQ ID | SEQ ID | SEQ ID | SEQ ID | SEQ ID | SEQ ID | SEQ ID | SEQ ID |
| | | NO: 329 | NO: 372 | NO: 373 | NO: 112 | NO: 377 | NO: 378 | NO: 379 | NO: 380 |
| 27 | Ab27 (E12.2) | SEQ ID | SEQ ID | SEQ ID | SEQ ID | SEQ ID | SEQ ID | SEQ ID | SEQ ID |
| | | NO: 385 | NO: 386 | NO: 387 | NO: 58 | NO: 391 | NO: 133 | NO: 392 | NO: 82 |
| 28 | Ab28 (10x- | SEQ ID | SEQ ID | SEQ ID | SEQ ID | SEQ ID | SEQ ID | SEQ ID | SEQ ID |
| | L8N-c1) | NO: 397 | NO: 398 | NO: 399 | NO: 112 | NO: 359 | NO: 403 | NO: 404 | NO: 51 |
| 29 | Ab29 (10x- | SEQ ID | SEQ ID | SEQ ID | SEQ ID | SEQ ID | SEQ ID | SEQ ID | SEQ ID |
| | L8N-c5) | NO: 409 | NO: 353 | NO: 410 | NO: 112 | NO: 359 | NO: 414 | NO: 415 | NO: 135 |
| 30 | Ab30 (10x- | SEQ ID | SEQ ID | SEQ ID | SEQ ID | SEQ ID | SEQ ID | SEQ ID | SEQ ID |
| | L8N-c9) | NO: 329 | NO: 420 | NO: 421 | NO: 422 | NO: 79 | NO: 426 | NO: 392 | NO: 311 |
| 31 | Ab31 (10x- | SEQ ID | SEQ ID | SEQ ID | SEQ ID | SEQ ID | SEQ ID | SEQ ID | SEQ ID |
| | L8N-c10) | NO: 329 | NO: 72 | NO: 430 | NO: 431 | NO: 434 | NO: 80 | NO: 435 | NO: 95 |
| 32 | Ab32 (10x- | SEQ ID | SEQ ID | SEQ ID | SEQ ID | SEQ ID | SEQ ID | SEQ ID | SEQ ID |
| | L8N-c12) | NO: 439 | NO: 440 | NO: 441 | NO: 442 | NO: 446 | NO: 447 | NO: 448 | NO: 51 |
| 33 | Ab33 (10x- | SEQ ID | SEQ ID | SEQ ID | SEQ ID | SEQ ID | SEQ ID | SEQ ID | SEQ ID |
| | L8N-c18) | NO: 385 | NO: 386 | NO: 450 | NO: 58 | NO: 452 | NO: 133 | NO: 392 | NO: 82 |
| 34 | Ab34 (10x- | SEQ ID | SEQ ID | SEQ ID | SEQ ID | SEQ ID | SEQ ID | SEQ ID | SEQ ID |
| | L8N-c19) | NO: 457 | NO: 198 | NO: 458 | NO: 459 | NO: 462 | NO: 133 | NO: 463 | NO: 219 |
| 35 | Ab35 (10x- | SEQ ID | SEQ ID | SEQ ID | SEQ ID | SEQ ID | SEQ ID | SEQ ID | SEQ ID |
| | L8N-c20) | NO: 266 | NO: 468 | NO: 469 | NO: 470 | NO: 116 | NO: 414 | NO: 473 | NO: 474 |
| 36 | Ab37 (10x- | SEQ ID | SEQ ID | SEQ ID | SEQ ID | SEQ ID | SEQ ID | SEQ ID | SEQ ID |
| | L8N-c22) | NO: 478 | NO: 479 | NO: 480 | NO: 58 | NO: 452 | NO: 484 | NO: 485 | NO: 486 |
| 37 | Ab38 (G8.2) | SEQ ID | SEQ ID | SEQ ID | SEQ ID | SEQ ID | SEQ ID | SEQ ID | SEQ ID |
| | | NO: 329 | NO: 72 | NO: 490 | NO: 112 | NO: 359 | NO: 414 | NO: 494 | NO: 495 |
| 38 | Ab39 (10x- | SEQ ID | SEQ ID | SEQ ID | SEQ ID | SEQ ID | SEQ ID | SEQ ID | SEQ ID |
| | L8N-c30) | NO: 500 | NO: 353 | NO: 501 | NO: 112 | NO: 505 | NO: 506 | NO: 507 | NO: 95 |
| 39 | Ab40 (10x- | SEQ ID | SEQ ID | SEQ ID | SEQ ID | SEQ ID | SEQ ID | SEQ ID | SEQ ID |
| | L8N-c32) | NO: 512 | NO: 513 | NO: 514 | NO: 112 | NO: 79 | NO: 519 | NO: 520 | NO: 521 |
| 40 | Ab41 (10x- | SEQ ID | SEQ ID | SEQ ID | SEQ ID | SEQ ID | SEQ ID | SEQ ID | SEQ ID |
| | L8N-c39) | NO: 526 | NO: 527 | NO: 528 | NO: 112 | NO: 533 | NO: 534 | NO: 494 | NO: 474 |
| 41 | Ab42 (10x- | SEQ ID | SEQ ID | SEQ ID | SEQ ID | SEQ ID | SEQ ID | SEQ ID | SEQ ID |
| | L8N-c41) | NO: 539 | NO: 540 | NO: 541 | NO: 112 | NO: 79 | NO: 545 | NO: 546 | NO: 474 |
| 42 | Ab43 (10x- | SEQ ID | SEQ ID | SEQ ID | SEQ ID | SEQ ID | SEQ ID | SEQ ID | SEQ ID |
| | L8N-c42) | NO: 124 | NO: 551 | NO: 552 | NO: 553 | NO: 79 | NO: 557 | NO: 558 | NO: 51 |
| 43 | Ab44 (10x- | SEQ ID | SEQ ID | SEQ ID | SEQ ID | SEQ ID | SEQ ID | SEQ ID | SEQ ID |
| | L8N-c53) | NO: 563 | NO: 564 | NO: 565 | NO: 566 | NO: 132 | NO: 570 | NO: 571 | NO: 135 |
| 44 | Ab45 (10x- | SEQ ID | SEQ ID | SEQ ID | SEQ ID | SEQ ID | SEQ ID | SEQ ID | SEQ ID |
| | L8N-c62) | NO: 576 | NO: 577 | NO: 578 | NO: 112 | NO: 391 | NO: 582 | NO: 583 | NO: 486 |
| 45 | Ab46 (10x- | SEQ ID | SEQ ID | SEQ ID | SEQ ID | SEQ ID | SEQ ID | SEQ ID | SEQ ID |
| | L8N-c63) | NO: 585 | NO: 372 | NO: 586 | NO: 112 | NO: 589 | NO: 590 | NO: 591 | NO: 82 |
| 46 | Ab47 (10x- | SEQ ID | SEQ ID | SEQ ID | SEQ ID | SEQ ID | SEQ ID | SEQ ID | SEQ ID |
| | L8N-c64) | NO: 87 | NO: 169 | NO: 595 | NO: 422 | NO: 79 | NO: 598 | NO: 599 | NO: 135 |
| 47 | Ab48 (10x- | SEQ ID | SEQ ID | SEQ ID | SEQ ID | SEQ ID | SEQ ID | SEQ ID | SEQ ID |
| | L8N-c72) | NO: 604 | NO: 605 | NO: 606 | NO: 58 | NO: 611 | NO: 612 | NO: 613 | NO: 51 |
| 48 | Ab49 (10x- | SEQ ID | SEQ ID | SEQ ID | SEQ ID | SEQ ID | SEQ ID | SEQ ID | SEQ ID |
| | L8N-c73) | NO: 329 | NO: 618 | NO: 619 | NO: 620 | NO: 132 | NO: 625 | NO: 626 | NO: 95 |
| 49 | Ab50 (10x- | SEQ ID | SEQ ID | SEQ ID | SEQ ID | SEQ ID | SEQ ID | SEQ ID | SEQ ID |
| | L8N-c84) | NO: 631 | NO: 632 | NO: 633 | NO: 634 | NO: 639 | NO: 640 | NO: 641 | NO: 642 |
| 50 | Ab51 (10x- | SEQ ID | SEQ ID | SEQ ID | SEQ ID | SEQ ID | SEQ ID | SEQ ID | SEQ ID |
| | L8N-c86) | NO: 647 | NO: 648 | NO: 649 | NO: 650 | NO: 654 | NO: 296 | NO: 655 | NO: 486 |
| 51 | Ab52 (10x- | SEQ ID | SEQ ID | SEQ ID | SEQ ID | SEQ ID | SEQ ID | SEQ ID | SEQ ID |
| | L8N-c89) | NO: 660 | NO: 661 | NO: 662 | NO: 112 | NO: 667 | NO: 668 | NO: 669 | NO: 82 |

TABLE C

| | | Column A | Column B |
|---|---|---|---|
| Row | Antibody | VH | VL |
| 1 | Ab1 (B11.2) | SEQ ID NO: 36 | SEQ ID NO: 44 |
| 2 | Ab2 (H12.2) | SEQ ID NO: 52 | SEQ ID NO: 59 |
| 3 | Ab3 (C2.2) | SEQ ID NO: 67 | SEQ ID NO: 75 |
| 4 | Ab4 (G7.2) | SEQ ID NO: 83 | SEQ ID NO: 89 |
| 5 | Ab5 (10x-L8N-c25) | SEQ ID NO: 96 | SEQ ID NO: 102 |
| 6 | Ab6 (K23) | SEQ ID NO: 105 | SEQ ID NO: 113 |
| 7 | Ab7 (G1) | SEQ ID NO: 120 | SEQ ID NO: 128 |
| 8 | Ab8 (B9) | SEQ ID NO: 136 | SEQ ID NO: 144 |
| 9 | Ab9 (C68) | SEQ ID NO: 149 | SEQ ID NO: 157 |
| 10 | Ab10 (C115) | SEQ ID NO: 164 | SEQ ID NO: 171 |
| 11 | Ab11 (D4) | SEQ ID NO: 179 | SEQ ID NO: 187 |
| 12 | Ab12 (H5) | SEQ ID NO: 193 | SEQ ID NO: 200 |
| 13 | Ab13 (D10) | SEQ ID NO: 206 | SEQ ID NO: 213 |
| 14 | Ab14 (F9) | SEQ ID NO: 220 | SEQ ID NO: 228 |
| 15 | Ab15 (E1.1) | SEQ ID NO: 235 | SEQ ID NO: 242 |
| 16 | Ab16 (G1.1) | SEQ ID NO: 249 | SEQ ID NO: 256 |
| 17 | Ab17 (B5.1) | SEQ ID NO: 262 | SEQ ID NO: 270 |
| 18 | Ab18 (H9.1) | SEQ ID NO: 274 | SEQ ID NO: 280 |
| 19 | Ab19 (F5.1) | SEQ ID NO: 285 | SEQ ID NO: 291 |
| 20 | Ab20 (C6.1) | SEQ ID NO: 299 | SEQ ID NO: 305 |
| 21 | Ab21 (E6.1) | SEQ ID NO: 312 | SEQ ID NO: 318 |
| 22 | Ab22 (C9.1) | SEQ ID NO: 325 | SEQ ID NO: 331 |
| 23 | Ab23 (B10.1) | SEQ ID NO: 336 | SEQ ID NO: 343 |
| 24 | Ab24 (G1.2) | SEQ ID NO: 350 | SEQ ID NO: 355 |
| 25 | Ab25 (H10.2) | SEQ ID NO: 362 | SEQ ID NO: 366 |
| 26 | Ab26 (G4.2) | SEQ ID NO: 368 | SEQ ID NO: 374 |
| 27 | Ab27 (E12.2) | SEQ ID NO: 381 | SEQ ID NO: 388 |
| 28 | Ab28 (10x-L8N-c1) | SEQ ID NO: 393 | SEQ ID NO: 400 |
| 29 | Ab29 (10x-L8N-c5) | SEQ ID NO: 405 | SEQ ID NO: 411 |
| 30 | Ab30 (10x-L8N-c9) | SEQ ID NO: 416 | SEQ ID NO: 423 |
| 31 | Ab31 (10x-L8N-c10) | SEQ ID NO: 427 | SEQ ID NO: 432 |
| 32 | Ab32 (10x-L8N-c12) | SEQ ID NO: 436 | SEQ ID NO: 443 |
| 33 | Ab33 (10x-L8N-c18) | SEQ ID NO: 449 | SEQ ID NO: 451 |
| 34 | Ab34 (10x-L8N-c19) | SEQ ID NO: 453 | SEQ ID NO: 460 |
| 35 | Ab35 (10x-L8N-c20) | SEQ ID NO: 464 | SEQ ID NO: 471 |
| 36 | Ab37 (10x-L8N-c22) | SEQ ID NO: 475 | SEQ ID NO: 481 |
| 37 | Ab38 (G8.2) | SEQ ID NO: 487 | SEQ ID NO: 491 |
| 38 | Ab39 (10x-L8N-c30) | SEQ ID NO: 496 | SEQ ID NO: 502 |
| 39 | Ab40 (10x-L8N-c32) | SEQ ID NO: 508 | SEQ ID NO: 515 |
| 40 | Ab41 (10x-L8N-c39) | SEQ ID NO: 522 | SEQ ID NO: 529 |
| 41 | Ab42 (10x-L8N-c41) | SEQ ID NO: 535 | SEQ ID NO: 542 |
| 42 | Ab43 (10x-L8N-c42) | SEQ ID NO: 547 | SEQ ID NO: 554 |
| 43 | Ab44 (10x-L8N-c53) | SEQ ID NO: 559 | SEQ ID NO: 567 |
| 44 | Ab45 (10x-L8N-c62) | SEQ ID NO: 572 | SEQ ID NO: 579 |
| 45 | Ab46 (10x-L8N-c63) | SEQ ID NO: 584 | SEQ ID NO: 587 |
| 46 | Ab47 (10x-L8N-c64) | SEQ ID NO: 592 | SEQ ID NO: 596 |
| 47 | Ab48 (10x-L8N-c72) | SEQ ID NO: 600 | SEQ ID NO: 607 |
| 48 | Ab49 (10x-L8N-c73) | SEQ ID NO: 614 | SEQ ID NO: 621 |
| 49 | Ab50 (10x-L8N-c84) | SEQ ID NO: 627 | SEQ ID NO: 635 |
| 50 | Ab51 (10x-L8N-c86) | SEQ ID NO: 643 | SEQ ID NO: 651 |
| 51 | Ab52 (10x-L8N-c89) | SEQ ID NO: 656 | SEQ ID NO: 663 |

TABLE D

| | | Column A | Column B |
|---|---|---|---|
| Row | Antibody | Heavy Chain | Light Chain |
| 1 | Ab1 (B11.2) | SEQ ID NO: 720 | SEQ ID NO: 721 |
| 2 | Ab2 (H12.2) | SEQ ID NO: 722 | SEQ ID NO: 723 |
| 3 | Ab3 (C2.2) | SEQ ID NO: 724 | SEQ ID NO: 725 |
| 4 | Ab4 (G7.2) | SEQ ID NO: 726 | SEQ ID NO: 727 |
| 5 | Ab5 (10x-L8N-c25) | SEQ ID NO: 728 | SEQ ID NO: 729 |
| 6 | Ab6 (K23) | SEQ ID NO: 730 | SEQ ID NO: 731 |
| 7 | Ab7 (G1) | SEQ ID NO: 732 | SEQ ID NO: 733 |
| 8 | Ab8 (B9) | SEQ ID NO: 734 | SEQ ID NO: 735 |
| 9 | Ab9 (C68) | SEQ ID NO: 736 | SEQ ID NO: 737 |
| 10 | Ab10 (C115) | SEQ ID NO: 738 | SEQ ID NO: 739 |
| 11 | Ab11 (D4) | SEQ ID NO: 740 | SEQ ID NO: 741 |
| 12 | Ab12 (H5) | SEQ ID NO: 742 | SEQ ID NO: 743 |
| 13 | Ab13 (D10) | SEQ ID NO: 744 | SEQ ID NO: 745 |
| 14 | Ab14 (F9) | SEQ ID NO: 746 | SEQ ID NO: 747 |
| 15 | Ab15 (E1.1) | SEQ ID NO: 748 | SEQ ID NO: 749 |
| 16 | Ab16 (G1.1) | SEQ ID NO: 750 | SEQ ID NO: 751 |
| 17 | Ab17 (B5.1) | SEQ ID NO: 752 | SEQ ID NO: 753 |
| 18 | Ab18 (H9.1) | SEQ ID NO: 754 | SEQ ID NO: 755 |
| 19 | Ab19 (F5.1) | SEQ ID NO: 756 | SEQ ID NO: 757 |
| 20 | Ab20 (C6.1) | SEQ ID NO: 758 | SEQ ID NO: 759 |
| 21 | Ab21 (E6.1) | SEQ ID NO: 760 | SEQ ID NO: 761 |
| 22 | Ab22 (C9.1) | SEQ ID NO: 762 | SEQ ID NO: 763 |
| 23 | Ab23 (B10.1) | SEQ ID NO: 764 | SEQ ID NO: 765 |
| 24 | Ab24 (G1.2) | SEQ ID NO: 766 | SEQ ID NO: 767 |
| 25 | Ab25 (H10.2) | SEQ ID NO: 768 | SEQ ID NO: 769 |
| 26 | Ab26 (G4.2) | SEQ ID NO: 770 | SEQ ID NO: 771 |
| 27 | Ab27 (E12.2) | SEQ ID NO: 772 | SEQ ID NO: 773 |
| 28 | Ab28 (10x-L8N-c1) | SEQ ID NO: 774 | SEQ ID NO: 775 |
| 29 | Ab29 (10x-L8N-c5) | SEQ ID NO: 776 | SEQ ID NO: 777 |
| 30 | Ab30 (10x-L8N-c9) | SEQ ID NO: 778 | SEQ ID NO: 779 |
| 31 | Ab31 (10x-L8N-c10) | SEQ ID NO: 780 | SEQ ID NO: 781 |
| 32 | Ab32 (10x-L8N-c12) | SEQ ID NO: 782 | SEQ ID NO: 783 |
| 33 | Ab33 (10x-L8N-c18) | SEQ ID NO: 784 | SEQ ID NO: 785 |
| 34 | Ab34 (10x-L8N-c19) | SEQ ID NO: 786 | SEQ ID NO: 787 |
| 35 | Ab35 (10x-L8N-c20) | SEQ ID NO: 788 | SEQ ID NO: 789 |
| 36 | Ab37 (10x-L8N-c22) | SEQ ID NO: 790 | SEQ ID NO: 791 |
| 37 | Ab38 (G8.2) | SEQ ID NO: 792 | SEQ ID NO: 793 |
| 38 | Ab39 (10x-L8N-c30) | SEQ ID NO: 794 | SEQ ID NO: 795 |
| 39 | Ab40 (10x-L8N-c32) | SEQ ID NO: 796 | SEQ ID NO: 797 |
| 40 | Ab41 (10x-L8N-c39) | SEQ ID NO: 798 | SEQ ID NO: 799 |
| 41 | Ab42 (10x-L8N-c41) | SEQ ID NO: 800 | SEQ ID NO: 801 |
| 42 | Ab43 (10x-L8N-c42) | SEQ ID NO: 802 | SEQ ID NO: 803 |
| 43 | Ab44 (10x-L8N-c53) | SEQ ID NO: 804 | SEQ ID NO: 805 |
| 44 | Ab45 (10x-L8N-c62) | SEQ ID NO: 806 | SEQ ID NO: 807 |
| 45 | Ab46 (10x-L8N-c63) | SEQ ID NO: 808 | SEQ ID NO: 809 |
| 46 | Ab47 (10x-L8N-c64) | SEQ ID NO: 810 | SEQ ID NO: 811 |
| 47 | Ab48 (10x-L8N-c72) | SEQ ID NO: 812 | SEQ ID NO: 813 |
| 48 | Ab49 (10x-L8N-c73) | SEQ ID NO: 814 | SEQ ID NO: 815 |
| 49 | Ab50 (10x-L8N-c84) | SEQ ID NO: 816 | SEQ ID NO: 817 |
| 50 | Ab51 (10x-L8N-c86) | SEQ ID NO: 818 | SEQ ID NO: 819 |
| 51 | Ab52 (10x-L8N-c89) | SEQ ID NO: 820 | SEQ ID NO: 821 |

### Further Aspects and Embodiments of the Present Disclosure

The following section describes further aspects, embodiments and technical features in accordance with the present disclosure.

Broad range protein antigen-binding molecules such as neutralizing antibodies suitable for use in treatment or prevention of coronaviral infection particularly SARS-CoV-2 variants is envisaged.

Accordingly, an aspect of the disclosure refers to an antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises: (i) a heavy chain having the amino acid having at least 95% sequence identity to SEQ ID NO:822 or SEQ ID NO:36 or SEQ ID NO:52; and (ii) a light chain having the amino acid having at least 95% sequence identity to SEQ ID NO:823 or SEQ ID NO:44 or SEQ ID NO:59.

According to another aspect there is an antigen-binding molecule which binds to and neutralizes SARS-CoV-2 variant BQ.1.1 at a concentration less than the concentration of E7 required to bind to and neutralize SARS-CoV-2 variant BQ.1.1; at least one other SARS-CoV-2 variant and another sarbecovirus wherein the antigen-binding molecule comprises: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:179, SEQ ID NO:193, SEQ ID NO:206, SEQ ID NO:220, SEQ ID NO:285, SEQ ID NO:299, SEQ ID NO:312, SEQ ID NO:325, SEQ ID NO:336, SEQ ID NO:362, SEQ ID NO:368, SEQ ID NO:381, SEQ ID NO:393, SEQ ID NO:436, SEQ ID NO:464, SEQ ID NO:496, SEQ ID NO:508, SEQ ID NO:522, SEQ ID NO:535, SEQ ID NO:547, SEQ ID NO:559, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:187, SEQ ID NO:200, SEQ ID NO:213, SEQ ID NO:228, SEQ ID NO:291, SEQ ID NO:305, SEQ ID NO:318, SEQ ID NO:331, SEQ ID NO:343, SEQ ID NO:366, SEQ ID NO:374, SEQ ID NO:388, SEQ ID NO:400, SEQ ID NO:443, SEQ ID NO:471, SEQ ID NO:502, SEQ ID NO:515, SEQ ID NO:529, SEQ ID NO:542, SEQ ID NO:554, SEQ ID NO:567, SEQ ID NO:587, SEQ ID NO:596, SEQ ID NO:607, SEQ ID NO:621, SEQ ID NO:635, SEQ ID NO:651, and SEQ ID NO:663.

According to another aspect there is a composition of the antigen-binding molecule described herein above and any one of Bebtelovimab LY-CoV1404, and E7.

According to another aspect there is a method of treating a sarbecovirus infection comprising, administering a therapeutically effective amount of the antigen-binding molecule or composition described herein above to a patient in need.

According to another aspect there is a therapeutically effective amount of the antigen-binding molecule or composition described herein above for use in treating a sarbecovirus infection.

According to various embodiments there is an antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises: (i) a heavy chain having the amino acid having at least 95% sequence identity to SEQ ID NO:822 or SEQ ID NO:36 or SEQ ID NO:52, or SEQ ID NO:96; and (ii) a light chain having the amino acid having at least 95% sequence identity to SEQ ID NO:823 or SEQ ID NO:44 or SEQ ID NO:59 or SEQ ID NO:102.

In various embodiments: (i) the VH region incorporates the following CDRs: HC-CDR1 having the amino acid having at least 85% sequence identity to SEQ ID NO:37; HC-CDR2 having the amino acid having at least 85% sequence identity to SEQ ID NO:38 or SEQ ID NO:53; HC-CDR3 having the amino acid having at least 85% sequence identity to SEQ ID NO:39 or SEQ ID NO:54; and (ii) the VL region incorporates the following CDRs: LC-CDR1 having the amino acid having at least 85% sequence identity to SEQ ID NO:45 or SEQ ID NO:60; LC-CDR2 having the amino acid having at least 85% sequence identity to SEQ ID NO:46 or SEQ ID NO:61; LC-CDR3 having the amino acid having at least 85% sequence identity to SEQ ID NO:47 or SEQ ID NO:62.

This has the advantage of being able to neutralize the variants BQ.1.1 and XBB and potentially be used to treat those in need of treatment. Antibody 1, antibody 2 and antibody 5 were all able to effectively neutralize both SARS-CoV-2 variants BQ.1.1 and XBB at an IC50 concentration of 200 or less see [FIG. 1E], [FIG. 1F], [FIG. 2A] and [FIG. 3]. This demonstrates great improvement over antibody E7 which requires a much higher concentration to inhibit the variants BQ. 1.1 and XBB and is currently greatly needed as the Bebtelovimab (LY-CoV1404) antibody does not bind these variants. In various embodiments, a concentration less than the concentration of E7 required to bind to and neutralize SARS-CoV-2 variants BQ.1.1 and XBB refers to a concentration at least 2 times less than the concentration of E7 or at least 3, 4, 5, 6, 7, 8, 9, 10, 11, 15, 20, 29, 30, 34, 35, or 40 times less than the concentration of E7.

In various embodiments, antibody 1 comprises a VH region incorporating the following CDRs: HC-CDR1 having the amino acid of SEQ ID NO:37; HC-CDR2 having the amino acid of SEQ ID NO:38; and HC-CDR3 having the amino acid of SEQ ID NO:39; and a VL region incorporating the following CDRs: LC-CDR1 having the amino acid of SEQ ID NO:45; LC-CDR2 having the amino acid of SEQ ID NO:46; and LC-CDR3 having the amino acid of SEQ ID NO:47. In various embodiments, antibody 1 comprises a heavy chain having the amino acid of SEQ ID NO:36; and a light chain having the amino acid of SEQ ID NO:44. Antibody 1 required only 16.5 ng/ml to effectively neutralize SARS-CoV-2 variants BQ. 1.1 which is about 34 to 35 times less than the concentration of E7 required to bind to and neutralize SARS-CoV-2 variants BQ.1.1. Antibody 1 required only 11.6 ng/ml to effectively neutralize SARS-CoV-2 variants XBB which is about 29 to 30 times less than the concentration of E7 required to bind to and neutralize SARS-CoV-2 variants XBB.

In various embodiments, antibody 2 comprises a VH region incorporating the following CDRs: HC-CDR1 having the amino acid of SEQ ID NO:37; HC-CDR2 having the amino acid of SEQ ID NO:53; and HC-CDR3 having the amino acid of SEQ ID NO:54; and a VL region incorporating the following CDRs: LC-CDR1 having the amino acid of SEQ ID NO:60; LC-CDR2 having the amino acid of SEQ ID NO:61; and LC-CDR3 having the amino acid of SEQ ID NO:62. In various embodiments, antibody 2 comprises a heavy chain having the amino acid of SEQ ID NO:52; and a light chain having the amino acid of SEQ ID NO:59. Antibody 2 required only 16.6 ng/ml to effectively neutralize SARS-CoV-2 variants BQ. 1.1 which is about 34 to 35 times less than the concentration of E7 required to bind to and neutralize SARS-CoV-2 variants BQ.1.1. Antibody 2 required only 8.5 ng/ml to effectively neutralize SARS-CoV-2 variants XBB which is about 40 times less than the concentration of E7 required to bind to and neutralize SARS-CoV-2 variants XBB.

In various embodiments, antibody 5 comprises a heavy chain having the amino acid of SEQ ID NO:96; and a light chain having the amino acid of SEQ ID NO:102. Antibody 5 required only 57.5 ng/ml to effectively neutralize SARS-CoV-2 variants BQ.1.1 which is about 10 times less than the concentration of E7 required to bind to and neutralize SARS-CoV-2 variants BQ.1.1. Antibody 5 required only 40.8 ng/ml to effectively neutralize SARS-CoV-2 variants XBB which is about 8 times less than the concentration of E7 required to bind to and neutralize SARS-CoV-2 variants XBB.

The amino acid of SEQ ID NO:822 comprises a sequence that embraces the heavy chain amino acid of both SEQ ID NO:36 and SEQ ID NO:52 and the CDR's of SEQ ID Nos 107, 108, 109, 113, 114, and 115. The amino acid of SEQ ID NO:823 comprises a sequence that embraces the light chain amino acid of both SEQ ID NO:44 and SEQ ID NO:59 and the CDR's of SEQ ID Nos 110, 111, 112, 116, 117, and 118. In various embodiments, the E7 antibody includes a heavy chain having an amino acid sequence of SEQ ID NO:824, and a light chain having an amino acid sequence of SEQ ID NO:830. Any inhibition assay known in the art for determining inhibition of the variant from binding to ACE2, such as 50% inhibitory concentration (IC50; ng/ml) of monoclonal antibodies in blocking cell entry using pseudovirus neutralizing test format, may be used to determine antibodies that can be used at concentrations less than the concentration of E7 required to bind to and neutralize SARS-CoV-2 variants BQ.1.1 and XBB.

The higher neutralization potencies of the disclosed antibodies will enable lower dosages of the antigen-binding molecule to be used clinically as individual antigen-binding molecule or mixed in a cocktail of two or more antigen-binding molecule or an antigen-binding molecule with two or more different antigen-binding domains.

In various embodiments, antibody 1 comprises the following VH regions: HC-FR1—SEQ ID NO:40; HC-CDR1—SEQ ID NO:37; HC-FR2—SEQ ID NO:41; HC-CDR2—SEQ ID NO:38; HC-FR3—SEQ ID NO:42; HC-CDR3—SEQ ID NO:39; HC-FR4—SEQ ID NO:43; and the following VL regions: LC-FR1—SEQ ID NO:48; LC-CDR1—SEQ ID NO:45; LC-FR2—SEQ ID NO:49; LC-CDR2—SEQ ID NO:46; LC-FR3—SEQ ID NO:50; LC-CDR3—SEQ ID NO:47; LC-FR4—SEQ ID NO:51.

In various embodiments, antibody 2 comprises the following VH regions: HC-FR1—SEQ ID NO:55; HC-CDR1—SEQ ID NO:37; HC-FR2—SEQ ID NO:56; HC-CDR2—SEQ ID NO:53; HC-FR3—SEQ ID NO:57; HC-CDR3—SEQ ID NO:54; HC-FR4—SEQ ID NO:58; and the following VL regions: LC-FR1—SEQ ID NO:63; LC-CDR1—SEQ ID NO:60; LC-FR2—SEQ ID NO:64; LC-CDR2—SEQ ID NO:61; LC-FR3—SEQ ID NO:65; LC-CDR3—SEQ ID NO:62; LC-FR4—SEQ ID NO:66.

In various embodiments, the term binds to and neutralizes may comprise inhibition or neutralization of 50% or more binding between the sarbecovirus spike protein and ACE2. In various embodiments, inhibition or neutralization of 50% or more binding between the sarbecovirus spike protein and ACE2 may be selected from one of at least 50%, 55%, 60%, 61%, 62%, 63%, 64%, 65%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 95%, 99% or greater inhibition or neutralisation.

In various embodiments the heavy chain has at least 96%, or 97%, or 98% or 99% or 100% sequence identity to an amino acid to SEQ ID NO:822 or SEQ ID NO:36 or SEQ ID NO:52 or SEQ ID NO:96; and the light chain having the amino acid has at least 96%, or 97%, or 98% or 99% or 100% sequence identity to SEQ ID NO:823 or SEQ ID NO:44 or SEQ ID NO:59 or SEQ ID NO:102.

According to various embodiments there is an antigen-binding molecule which binds to and neutralizes SARS-CoV-2 variant BQ.1.1 at a concentration less than the concentration of E7 required to bind to and neutralize SARS-CoV-2 variant BQ.1.1; at least one other SARS-CoV-2 variant and another sarbecovirus other than SARS-CoV-2, wherein the antigen-binding molecule comprises: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:179, SEQ ID NO:193, SEQ ID NO:206, SEQ ID NO:220, SEQ ID NO:285, SEQ ID NO:299, SEQ ID NO:312, SEQ ID NO:325, SEQ ID NO:336, SEQ ID NO:362, SEQ ID NO:368, SEQ ID NO:381, SEQ ID NO:393, SEQ ID NO:436, SEQ ID NO:464, SEQ ID NO:496, SEQ ID NO:508, SEQ ID NO:522, SEQ ID NO:535, SEQ ID NO:547, SEQ ID NO:559, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:187, SEQ ID NO:200, SEQ ID NO:213, SEQ ID NO:228, SEQ ID NO:291, SEQ ID NO:305, SEQ ID NO:318, SEQ ID NO:331, SEQ ID NO:343, SEQ ID NO:366, SEQ ID NO:374, SEQ ID NO:388, SEQ ID NO:400, SSEQ ID NO:443, SEQ ID NO:471, SEQ ID NO:502, SEQ ID NO:515, SEQ ID NO:529, SEQ ID NO:542, SEQ ID NO:554, SEQ ID NO:567, SEQ ID NO:587, SEQ ID NO:596, SEQ ID NO:607, SEQ ID NO:621, SEQ ID NO:635, SEQ ID NO:651, and SEQ ID NO:663.

This has the advantage of being able to neutralize a variant, BQ.1.1, in the ongoing pandemic and be used at a suitable concentration to treat those in need (see for example [FIG. 1E], [FIG. 2A] [FIG. 3]). All antibodies 1 to 5 including antibody 1, antibody 2, antibody 3, antibody 4 and antibody 5 were all able to effectively neutralize SARS-CoV-2 variants BQ.1.1 at an IC50 concentration of 300 ng/ml or less see [FIG. 3]. This demonstrates great improvement over antibody E7 which requires a much higher concentration to inhibit the variant and is currently greatly needed as the Bebtelovimab (LY-CoV1404) antibody does not bind this variant BQ. 1.1. However, it also has the advantage of potentially helping to manage a future outbreak of another zoonotic sarbecovirus infection such as those currently observed in bats or Pangolins (See [FIG. 3]).

In various embodiments, a concentration less than the concentration of E7 required to bind to and neutralize SARS-CoV-2 variant BQ.1.1 refers to a concentration at least 1.1 times less than the concentration of E7 or at least 1.1, 1.5, 2, 3, 4, 5, 6, 7, 8, 9, 10, 11, 15, 20, 29, 30, 34, 35, or 40 times less than the concentration of E7. Antibody 1 required only 16.5 ng/ml to effectively neutralize SARS-CoV-2 variant BQ.1.1 which is about 34 to 35 times less than the concentration of E7 required to bind to and neutralize SARS-CoV-2 variant BQ.1.1. Antibody 2 required only 16.6 ng/ml to effectively neutralize SARS-CoV-2 variant BQ.1.1 which is about 34 to 35 times less than the concentration of E7 required to bind to and neutralize SARS-CoV-2 variant BQ.1.1. Antibody 3 required only 270.6 ng/ml to effectively neutralize SARS-CoV-2 variant BQ.1.1 which is about 2 times less than the concentration of E7 required to bind to and neutralize SARS-CoV-2 variants BQ.1.1. Antibody 4 required only 48.4 ng/ml to effectively neutralize SARS-CoV-2 variant BQ.1.1 which is about 11 times less than the concentration of E7 required to bind to and neutralize SARS-CoV-2 variant BQ.1.1. Antibody 5 required only 57.5 ng/ml to effectively neutralize SARS-CoV-2 variant BQ.1.1 which is about 10 times less than the concentration of E7 required to bind to and neutralize SARS-CoV-2 variant BQ.1.1. Antibodies 6-9, 11-14, 19-23, 25-28, 32, 35, 39-44 and 46-52 were also all able to effectively neutralize SARS-CoV-2 variants BQ.1.1 at an IC50 concentration of 550 ng/ml or less.

In various embodiments the above sequences refer to antibodies 1-5, 6-9, 11-14, 19-23, 25-28, 32, 35, 39-44 and 46-52 listed Table C herein. While contrastingly, antibodies 10, 15-18, 24, 29-31, 33, 34, 36-38, and 45 were in some cases able to bind and neutralize the SARS-CoV-2 variant BQ.1.1, however, each was less effective than the E7 antibody and hence required a higher concentration than E7 to bind to and neutralize SARS-CoV-2 variant BQ.1.1 (See [FIG. 3]).

In various embodiments, the at least one other SARS-CoV-2 variant comprises any one of Alpha COVID-19 variant SARS-CoV-2 B.1.1.7; the Beta COVID-19 variant SARS-CoV-2 B.1.351 also known as 20H/501Y.V2, or 501Y.V2 variant; the Gamma variant P.1, the Delta SARS-CoV-2 B.1.617.2; and the Omicron variants SARS-CoV-2 B.1.1.529 BA.1, BA.2, BA.5, BA.2.75, BA.2.75.2, BA.4.6.1, BF.7, BQ.1.1, and XBB. In various embodiments, the at least one other SARS-CoV-2 variant may comprises 2 or more, 3 or more, 4 or more, 5 or more, 6 or more, or 7 or more of any one of Alpha COVID-19 variant SARS-CoV-2 B.1.1.7; the Beta COVID-19 variant SARS-CoV-2 B.1.351 also known as 20H/501Y.V2, or 501Y.V2 variant; the Gamma variant P.1, the Delta SARS-CoV-2 B.1.617.2; and the Omicron variants SARS-CoV-2 B.1.1.529 BA.1, BA.2, BA.5, BA.2.75, BA.2.75.2, BA.4.6.1, BF.7, BQ.1.1, and XBB. As used herein, the term ‘another sarbecovirus’ refers to a sarbecovirus that is not SARS-CoV-2. In various embodiments, another sarbecovirus comprises SARS-COV, BANAL-52, WIV-1, SC2r-COV RaTG13, SC2r-COV GX-P5L, SC2r-CoV GD-1, SC2r-CoVRmYN02, RacCS203 or future unknown sarbecoviruses. In various embodiments, another sarbecovirus comprises any beta coronavirus that uses ACE2 receptor as entry into cells that is not SARS-CoV-2 or is a sarbecovirus other than SARS-CoV-2. A broad-spectrum antigen-binding molecule has the advantage of being able to block most sarbecoviruses effectively assisting in preventing infection of both known and unknown sarbecoviruses. In various embodiments the antigen-binding molecule comprises a monoclonal antibody (mAb). A mAb may be one of the most efficient and powerful tools for rapid development and deployment in fighting future emerging zoonotic viruses, and sarbecoviruses in particular.

According to various embodiments there is a composition of the antigen-binding molecule described herein above and any one of Bebtelovimab LY-CoV1404, and E7. In various embodiments, the Bebtelovimab LY-CoV1404 antibody comprises that described in WO/2021/183359. In various embodiments, such a composition or cocktail has the advantage of increasing the range of the antigen-binding molecule to bind and neutralize a broad range of SARS-CoV-2 variants and other sarbecovirus (see for example [FIG. 3]). In various embodiments, the composition comprises:
a first antigen-binding molecule comprising: (i) a VH region incorporating the following CDRs: HC-CDR1 comprising an amino acid having at least 85% sequence identity to SEQ ID NO:37; HC-CDR2 comprising an amino acid having at least 85% sequence identity to SEQ ID NO:38 or SEQ ID NO:53; HC-CDR3 comprising an amino acid having at least 85% sequence identity to SEQ ID NO:39 or SEQ ID NO:54; and (ii) a VL region incorporating the following CDRs: LC-CDR1 comprising an amino acid having at least 85% sequence identity to SEQ ID NO:45 or SEQ ID NO:60; LC-CDR2 comprising an amino acid having at least 85% sequence identity to SEQ ID NO:46 or SEQ ID NO:61; LC-CDR3 comprising an amino acid having at least 85% sequence identity to SEQ ID NO:47 or SEQ ID NO:62; and
a second antigen-binding molecule comprising: (iii) a heavy chain comprising an amino acid having at least 95% sequence identity to sequence of SEQ ID NO:824; and (iv) a light chain comprising an amino acid having at least 95% sequence identity to sequence of SEQ ID NO:830.

In various embodiments, the composition comprises an antigen-binding molecule selected from any one of antibodies 1-52 in combination with the antibody Bebtelovimab LY-CoV1404, or the antibody E7.

According to various embodiments there is a method of treating a sarbecovirus infection comprising, administering a therapeutically effective amount of the antigen-binding molecule or composition described herein above to a patient in need. In various embodiments a therapeutically effective amount of the antigen-binding molecule or composition described comprises an amount capable of neutralizing or inhibiting at least enough of the sarbecovirus infection to stop, minimise or reduce the symptoms of the sarbecovirus infection. According to various embodiments there is a method of treating a sarbecovirus infection comprising, administering to a patient in need a therapeutically effective amount of the antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises: (i) a VH region incorporating the following CDRs: HC-CDR1 having the amino acid having at least 85% sequence identity to SEQ ID NO:37; HC-CDR2 having the amino acid having at least 85% sequence identity to SEQ ID NO:38 or SEQ ID NO:53; HC-CDR3 having the amino acid having at least 85% sequence identity to SEQ ID NO:39 or SEQ ID NO:54; and (ii) a VL region incorporating the following CDRs: LC-CDR1 having the amino acid having at least 85% sequence identity to SEQ ID NO:45 or SEQ ID NO:60; LC-CDR2 having the amino acid having at least 85% sequence identity to SEQ ID NO:46 or SEQ ID NO:61; LC-CDR3 having the amino acid having at least 85% sequence identity to SEQ ID NO:47 or SEQ ID NO:62.

According to various embodiments there is a method of treating a sarbecovirus infection comprising, administering to a patient in need a therapeutically effective amount of the antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises: (i) a heavy chain having the amino acid having at least 95% sequence identity to SEQ ID NO:822 or SEQ ID NO:36 or SEQ ID NO:52, or SEQ ID NO:96; and (ii) a light chain having the amino acid having at least 95% sequence identity to SEQ ID NO:823 or SEQ ID NO:44 or SEQ ID NO:59 or SEQ ID NO:102.

According to various embodiments there is a method of treating a sarbecovirus infection comprising, administering to a patient in need a therapeutically effective amount of an antigen-binding molecule which binds to and neutralizes SARS-CoV-2 variant BQ.1.1 at a concentration less than the concentration of E7 required to bind to and neutralize SARS-CoV-2 variant BQ.1.1; at least one other SARS-CoV-2 variant and another sarbecovirus wherein the antigen-binding molecule comprises: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:179, SEQ ID NO:193, SEQ ID NO:206, SEQ ID NO:220, SEQ ID NO:285, SEQ ID NO:299, SEQ ID NO:312, SEQ ID NO:325, SEQ ID NO:336, SEQ ID NO:362, SEQ ID NO:368, SEQ ID NO:381, SEQ ID NO:393, SEQ ID NO:436, SEQ ID NO:464, SEQ ID NO:496, SEQ ID NO:508, SEQ ID NO:522, SEQ ID NO:535, SEQ ID NO:547, SEQ ID NO:559, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:187, SEQ ID NO:200, SEQ ID NO:213, SEQ ID NO:228, SEQ ID NO:291, SEQ ID NO:305, SEQ ID NO:318, SEQ ID NO:331, SEQ ID NO:343, SEQ ID NO:366, SEQ ID NO:374, SEQ ID NO:388, SEQ ID NO:400, SEQ ID NO:443, SEQ ID NO:471, SEQ ID NO:502, SEQ ID NO:515, SEQ ID NO:529, SEQ ID NO:542, SEQ ID NO:554, SEQ ID NO:567, SEQ ID NO:587, SEQ ID NO:596, SEQ ID NO:607, SEQ ID NO:621, SEQ ID NO:635, SEQ ID NO:651, and SEQ ID NO:663.

According to various embodiments there is a method of treating a sarbecovirus infection comprising, administering to a patient in need a therapeutically effective amount of a composition comprising the antigen-binding molecule described herein above, and any one of Bebtelovimab LY-CoV1404, and E7. A composition comprising a combination of antibody 1 and antibody E7 was able to increase the inhibition capabilities and resulted in effective inhibition of all SARS-CoV-2 variants tested and all sarbecovirus tested. Similarly, a composition comprising a combination of antibody 2 and antibody E7 was able to increase the inhibition capabilities and resulted in effective inhibition of all SARS-CoV-2 variants tested and all sarbecovirus tested.

In various embodiments, the patient in need may be an individual that have been diagnosed with a sarbecovirus infection. In various embodiments, the patient in need may be an individual that have been diagnosed with an infection caused by a SARS-CoV-2 variant. In various embodiments, the method comprises determining an infection is caused by a sarbecovirus such as a SARS-CoV-2 variant. In various embodiments, the patient in need may be an individual that have been diagnosed with COVID-19 caused by a sarbecovirus such as a SARS-CoV-2 variant. In various embodiments, the patient in need may be an individual that has been diagnosed with COVID-19 caused by a SARS-CoV-2 variant selected from BQ.1.1 and XBB.

According to various embodiments there is a therapeutically effective amount of the antigen-binding molecule or composition as described herein above or composition as described herein above for use in treating a sarbecovirus infection. In various embodiments, the sarbecovirus infection may be caused by a non-SARS-CoV-2 such as sarbecovirus earlier known to infect bats or pangolins or any currently unknown sarbecovirus. In various embodiments, the sarbecovirus infection may be caused by a SARS-CoV-2 variant. In various embodiments, the sarbecovirus infection may be caused by a SARS-CoV-2 variant selected from BQ.1.1 and XBB. In various embodiments, the antigen-binding molecule as discussed herein above, is suitable for use in treatment of individuals that have been diagnosed with a sarbecovirus infection.

The following numbered paragraphs (paras) describe particular aspects and embodiments of the present disclosure:

1. An antigen-binding molecule which binds to and neutralizes SARS-CoV-2 variants BQ. 1.1 and XBB at a concentration less than the concentration of E7 required to bind to and neutralize SARS-CoV-2 variants BQ.1.1 and XBB wherein the antigen-binding molecule comprises: a heavy chain having the amino acid having at least 95% sequence identity to SEQ ID NO:822 or SEQ ID NO:36 or SEQ ID NO:52 or SEQ ID NO:96; and a light chain having the amino acid having at least 95% sequence identity to SEQ ID NO:823 or SEQ ID NO:44 or SEQ ID NO:59, or SEQ ID NO:102.

2. The antigen-binding molecule according to para 1, wherein (i) the heavy chain variable (VH) region incorporates the following CDRs: HC-CDR1 having the amino acid having at least 85% sequence identity to SEQ ID NO:37; HC-CDR2 having the amino acid having at least 85% sequence identity to SEQ ID NO:38 or SEQ ID NO:53; HC-CDR3 having the amino acid having at least 85% sequence identity to SEQ ID NO:39 or SEQ ID NO:54; and (ii) the light chain variable (VL) region incorporates the following CDRs: LC-CDR1 having the amino acid having at least 85% sequence identity to SEQ ID NO:45 or SEQ ID NO:60; LC-CDR2 having the amino acid having at least 85% sequence identity to SEQ ID NO:46 or SEQ ID NO:61; LC-CDR3 having the amino acid having at least 85% sequence identity to SEQ ID NO:47 or SEQ ID NO:62.

3. An antigen-binding molecule which binds to and neutralizes SARS-CoV-2 variant BQ. 1.1 at a concentration less than the concentration of E7 required to bind to and neutralize SARS-CoV-2 variant BQ.1.1; at least one other SARS-CoV-2 variant and another sarbecovirus wherein the antigen-binding molecule comprises: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:179, SEQ ID NO:193, SEQ ID NO:206, SEQ ID NO:220, SEQ ID NO:285, SEQ ID NO:299, SEQ ID NO:312, SEQ ID NO:325, SEQ ID NO:336, SEQ ID NO:362, SEQ ID NO:368, SEQ ID NO:381, SEQ ID NO:393, SEQ ID NO:436, SEQ ID NO:464, SEQ ID NO:496, SEQ ID NO:508, SEQ ID NO:522, SEQ ID NO:535, SEQ ID NO:547, SEQ ID NO:559, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:187, SEQ ID NO:200, SEQ ID NO:213, SEQ ID NO:228, SEQ ID NO:291, SEQ ID NO:305, SEQ ID NO:318, SEQ ID NO:331, SEQ ID NO:343, SEQ ID NO:366, SEQ ID NO:374, SEQ ID NO:388, SEQ ID NO:400, SEQ ID NO:443, SEQ ID NO:471, SEQ ID NO:502, SEQ ID NO:515, SEQ ID NO:529, SEQ ID NO:542, SEQ ID NO:554, SEQ ID NO:567, SEQ ID NO:587, SEQ ID NO:596, SEQ ID NO:607, SEQ ID NO:621, SEQ ID NO:635, SEQ ID NO:651, and SEQ ID NO:663.

4. A composition comprising the antigen-binding molecule of any one of paras 1 to 3, and any one of Bebtelovimab LY-CoV1404, and E7.

5. The composition according to para 4 wherein antigen-binding molecule comprises: (i) the heavy chain variable (VH) region incorporates the following CDRs: HC-CDR1 comprising an amino acid having at least 85% sequence identity to SEQ ID NO:37; HC-CDR2 comprising an amino acid having at least 85% sequence identity to SEQ ID NO:38 or SEQ ID NO:53; HC-CDR3 comprising an amino acid having at least 85% sequence identity to SEQ ID NO:39 or SEQ ID NO:54; and (ii) the light chain variable (VL) region incorporates the following CDRs: LC-CDR1 comprising an amino acid having at least 85% sequence identity to SEQ ID NO:45 or SEQ ID NO:60; LC-CDR2 comprising an amino acid having at least 85% sequence identity to SEQ ID NO:46 or SEQ ID NO:61; LC-CDR3 comprising an amino acid having at least 85% sequence identity to SEQ ID NO:47 or SEQ ID NO:62; and wherein E7 comprises a second antigen-binding molecule comprising: (iii) a heavy chain comprising an amino acid having at least 95% sequence identity to sequence set out in SEQ ID NO:824; and (iv) a light chain comprising an amino acid having at least 95% sequence identity to sequence set out in SEQ ID NO:830:

6. A composition which binds to and neutralizes SARS-CoV-2 variant BQ.1.1, at least three other SARS-CoV-2 variants and another sarbecovirus wherein the composition comprises antigen-binding molecule comprising: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:164, SEQ ID NO:179, SEQ ID NO:193, SEQ ID NO:206, SEQ ID NO:220, SEQ ID NO:235, SEQ ID NO:249, SEQ ID NO:262, SEQ ID NO:274, SEQ ID NO:285, SEQ ID NO:299, SEQ ID NO:312, SEQ ID NO:325, SEQ ID NO:336, SEQ ID NO:350, SEQ ID NO:362, SEQ ID NO:368, SEQ ID NO:381, SEQ ID NO:393, SEQ ID NO:405, SEQ ID NO:416, SEQ ID NO:427, SEQ ID NO:436, SEQ ID NO:449, SEQ ID NO:453, SEQ ID NO:464, SEQ ID NO:475, SEQ ID NO:487, SEQ ID NO:496, SEQ ID NO:508, SEQ ID NO:522, SEQ ID NO:535, SEQ ID NO:547, SEQ ID NO:559, SEQ ID NO:572, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:171, SEQ ID NO:187, SEQ ID NO:200, SEQ ID NO:213, SEQ ID NO:228, SEQ ID NO:242, SEQ ID NO:256, SEQ ID NO:270, SEQ ID NO:280, SEQ ID NO:291, SEQ ID NO:305, SEQ ID NO:318, SEQ ID NO:331, SEQ ID NO:343, SEQ ID NO:355, SEQ ID NO:366, SEQ ID NO:374, SEQ ID NO:388, SEQ ID NO:400, SEQ ID NO:411, SEQ ID NO:423, SEQ ID NO:432, SEQ ID NO:443, SEQ ID NO:451, SEQ ID NO:460, SEQ ID NO:471, SEQ ID NO:481, SEQ ID NO:491, SEQ ID NO:502, SEQ ID NO:515, SEQ ID NO:529, SEQ ID NO:542, SEQ ID NO:554, SEQ ID NO:567, SEQ ID NO:579, SEQ ID NO:587, SEQ ID NO:596, SEQ ID NO:607, SEQ ID NO:621, SEQ ID NO:635, SEQ ID NO:651, and SEQ ID NO:663; and a second antigen-binding molecule comprising any one of Bebtelovimab LY-CoV1404, and E7.

6. A method of treating a sarbecovirus infection comprising, administering to a patient in need a therapeutically effective amount of an antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises:
(i) a heavy chain variable (VH) region incorporating the following CDRs: HC-CDR1 having the amino acid having at least 85% sequence identity to SEQ ID NO:37; HC-CDR2 having the amino acid having at least 85% sequence identity to SEQ ID NO:38 or SEQ ID NO:53; HC-CDR3 having the amino acid having at least 85% sequence identity to SEQ ID NO:39 or SEQ ID NO:54; and
(ii) a light chain variable (VL) region incorporating the following CDRs: LC-CDR1 having the amino acid having at least 85% sequence identity to SEQ ID NO:45 or SEQ ID NO:60; LC-CDR2 having the amino acid having at least 85% sequence identity to SEQ ID NO:46 or SEQ ID NO:61; LC-CDR3 having the amino acid having at least 85% sequence identity to SEQ ID NO:47 or SEQ ID NO:62.

7. A method of treating a sarbecovirus infection comprising, administering to a patient in need a therapeutically effective amount of an antigen-binding molecule which binds to and neutralizes SARS-CoV-2 variants BQ.1.1 and XBB at a concentration less than the concentration of E7 required to bind to and neutralize SARS-CoV-2 variants BQ.1.1 and XBB wherein the antigen-binding molecule comprises: (i) a heavy chain having the amino acid having at least 95% sequence identity to SEQ ID NO:822 or SEQ ID NO:36 or SEQ ID NO:52 or SEQ ID NO:96; and (ii) a light chain having the amino acid having at least 95% sequence identity to SEQ ID NO:823 or SEQ ID NO:44 or SEQ ID NO:59 or SEQ ID NO:102.

8. A method of treating a sarbecovirus infection comprising, administering to a patient in need a therapeutically effective amount of an antigen-binding molecule which binds to and neutralizes SARS-CoV-2 variant BQ.1.1 at a concentration less than the concentration of E7 required to bind to and neutralize SARS-CoV-2 variant BQ.1.1; at least one other SARS-CoV-2 variant and another sarbecovirus wherein the antigen-binding molecule comprises: (i) a heavy chain having at least 95% sequence identity to an amino acid selected from any one of SEQ ID NO:822, SEQ ID NO:36, SEQ ID NO:52, SEQ ID NO:67, SEQ ID NO:83, SEQ ID NO:96, SEQ ID NO:105, SEQ ID NO:120, SEQ ID NO:136, SEQ ID NO:149, SEQ ID NO:179, SEQ ID NO:193, SEQ ID NO:206, SEQ ID NO:220, SEQ ID NO:285, SEQ ID NO:299, SEQ ID NO:312, SEQ ID NO:325, SEQ ID NO:336, SEQ ID NO:362, SEQ ID NO:368, SEQ ID NO:381, SEQ ID NO:393, SEQ ID NO:436, SEQ ID NO:464, SEQ ID NO:496, SEQ ID NO:508, SEQ ID NO:522, SEQ ID NO:535, SEQ ID NO:547, SEQ ID NO:559, SEQ ID NO:584, SEQ ID NO:592, SEQ ID NO:600, SEQ ID NO:614, SEQ ID NO:627, SEQ ID NO:643, and SEQ ID NO:656; and (ii) a light chain having at least 95% sequence identity to an amino acid selected from any one of SEQ ID NO:823, SEQ ID NO:44, SEQ ID NO:59, SEQ ID NO:75, SEQ ID NO:89, SEQ ID NO:102, SEQ ID NO:113, SEQ ID NO:128, SEQ ID NO:144, SEQ ID NO:157, SEQ ID NO:187, SEQ ID NO:200, SEQ ID NO:213, SEQ ID NO:228, SEQ ID NO:291, SEQ ID NO:305, SEQ ID NO:318, SEQ ID NO:331, SEQ ID NO:343, SEQ ID NO:366, SEQ ID NO:374, SEQ ID NO:388, SEQ ID NO:400, SEQ ID NO:443, SEQ ID NO:471, SEQ ID NO:502, SEQ ID NO:515, SEQ ID NO:529, SEQ ID NO:542, SEQ ID NO:554, SEQ ID NO:567, SEQ ID NO:587, SEQ ID NO:596, SEQ ID NO:607, SEQ ID NO:621, SEQ ID NO:635, SEQ ID NO:651, and SEQ ID NO:663.

9. A method of treating a sarbecovirus infection comprising, administering to a patient in need a therapeutically effective amount of a composition comprising the antigen-binding molecule of para 1 or 3, and any one of Bebtelovimab LY-CoV1404, and E7.

10. A therapeutically effective amount of the antigen-binding molecule according to any one of paras 1 to 3 or composition according to para 4 or 5 for use in treating a sarbecovirus infection.

11. The antigen-binding molecule or composition for use in para 10, wherein the sarbecovirus infection is caused by a SARS-CoV-2 variant comprising BQ.1.1.

12. The therapeutically effective amount of the antigen-binding molecule or composition for use according to para 10 wherein the sarbecovirus infection is caused by a SARS-CoV-2 variant comprising BQ.1.1 or XBB.

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, an amino acid sequence, or a region of a polypeptide which ‘corresponds’ to a specified reference amino acid sequence or region of a polypeptide has at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Söding, J. 2005, Bioinformatics 21, 951-960).

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

FIGS. 1A to 1F. Graphs % inhibition of infection of ACE2-expressing cells by pseudoviruses expressing the spike proteins of (1A) SARS-CoV-2 (1B) BA.2.75.2, (1C) BF.7, (1D) BA.4.6.1, (1E) BQ.1.1 and (1F) XBB.1 for the indicated antibodies, as determined by pseudovirus neutralisation tests (pVNT).

FIGS. 2A to 2C. Tables summarising the IC50 values for inhibition of infection of ACE2-expressing cells by pseudoviruses expressing the spike proteins of the indicated sarbecoviruses by the indicated antibodies/combinations of antibodies, as determined by pVNT.

FIG. 3. Bar chart showing the IC50 values for inhibition of infection of ACE2-expressing cells by pseudoviruses expressing the spike proteins of the indicated sarbecoviruses by the indicated antibodies, as determined by pVNT.

FIG. 4. Bar chart showing the IC50 values for inhibition of infection of ACE2-expressing cells by pseudoviruses expressing the spike proteins of the indicated sarbecoviruses by the indicated antibodies/combinations of antibodies, as determined by pVNT, performed in the presence of human serum.

FIG. 5. Graphs showing the IC50 values for inhibition of interaction between human ACE2 and the RBD of the spike proteins of the indicated sarbecoviruses by the indicated antibodies/combinations of antibodies, as determined by surrogate virus neutralisation test (sVNT).

### EXAMPLES

### Example 1—Materials and Methods

### 1.1 Pseudovirus Production

Vesicular stomatitis virus (VSV) pseudotyped with full-length SARS-CoV-2 Wuhan-hu-1 (ancestral), Omicron BA.1, Omicron BA.2, Omicron BA.5, Omicron BA.2.75, Omicron BA.2.75.2, Omicron BA.4.6.1, Omicron BF.7, Omicron BQ.1.1, Omicron XBB.1, Omicron XBB.1.16, Omicron XBB.2.3, Omicron EG.5, Omicron EG.5.1, BANAL-52, GD-1, GX-P5L, WIV-1 or SARS-CoV-1 spike proteins were produced and packaged as described in Tan et al., Nat. Biotechnol. (2020) 38:1073-1078, with minor modifications. Briefly, 5 million HEK293T cells were transfected with 20 μg of pCAGGS plasmid encoding the relevant spike protein using FuGENE6 (Promega). At 24 h post transfection, cells were incubated with VSVAG luc seed virus (at MOI of 5) for 2 h. Following two phosphate-buffered saline (PBS) washes, infected cells were replenished with complete growth media supplemented with 1:5000 diluted anti-VSV-G mAb (Clone 8GF11, Kerafast). At 24 h post infection, pseudoviruses were harvested by centrifugation at 2,000×g for 5 min.

1.2 Pseudovirus Neutralisation Test (pVNT)

For the pVNTs, 3×10⁶ RLU of pseudoviruses were pre-incubated with serially diluted monoclonal antibodies in PBS buffer. For serum-spiked pVNTs, the buffer was supplemented with human serum at a dilution of 1:20 to emulate ex vivo conditions. The starting concentration of monoclonal antibodies was 20 μg/ml serially diluted four-fold into a final volume of 50 μL for 1 h at 37° C., followed by infection of the pseudovirus-mAbs mixture onto A549 cells stably expressing human ACE2. At 20-24 h post-infection, an equal volume of ONE-Glo luciferase substrate (Promega) was added and the luminescence signal was measured using the Cytation 5 microplate reader (BioTek) with Gen5 software version 3.10.

1.3 Multiplex Surrogate Virus Neutralising Test (sVNT)

Multiplex surrogate Virus Neutralizing Tests (sVNTs) were performed essentially as described in Tan et al., Nat. Biotechnol. (2020) 38:1073-1078, using receptor binding domain (RBD) proteins from eleven different sarbecoviruses: SARS-CoV-2; SARS-CoV-2 B.1.351 (beta); SARS-CoV-2 B.1.617.2 (delta); SARS-CoV-2 B.1.1.529.1 (BA.1); SARS-CoV-2 B.1.1.529.5 (BA.5); SARS-CoV-2 XBB.1; SC1r-COV Rs2018B; SC1r-CoV RsSHC014; SARS-COV and Bat CoV Khosta-2.

Briefly, AviTag-biotinylated RBDs from different sarbecoviruses were coated on a MagPlex Avidin microsphere (Luminex) at 5 μg/1 million beads. RBD-coated microspheres (600 beads/antigen) were pre-incubated with test monoclonal antibodies at a starting concentration of 10 μg/mL serially diluted four-fold for 15 min at 37° C. with 250 rpm agitation. After 15 min incubation, 50 μL of 2 μg/mL phycoerythrin (PE)-conjugated hACE2 (GenScript) were added to the wells and incubated for 15 min at 37° C. with agitation, followed by two PBS-1% bovine serum albumin washes. The data were acquired using MAGPIX (Luminex) system.

### Example 2—Analysis of Ability of Antibodies to Neutralise Infection of ACE2-Expressing Cells by Pseudo-Sarbecoviruses Expressing Sarbecovirus Spike Proteins

Antibodies capable of binding to SARS-CoV-2 spike protein were obtained, and their sequence features are summarised in Tables A to C.

The ability of the different antibodies to neutralize infection of human ACE2-expressing cells by pseudovirus expressing the spike protein of SARS-CoV-2 and six SARS-CoV-2 variants (BA.2.75.2, BA.4.6.1, BF.7, BQ.1.1, and XBB) was analysed using a pseudovirus neutralising test (pVNT). The 51 different antibodies were shown to be able to bind to and neutralize infection by BQ.1.1 and/or other SARS-CoV-2 variants.

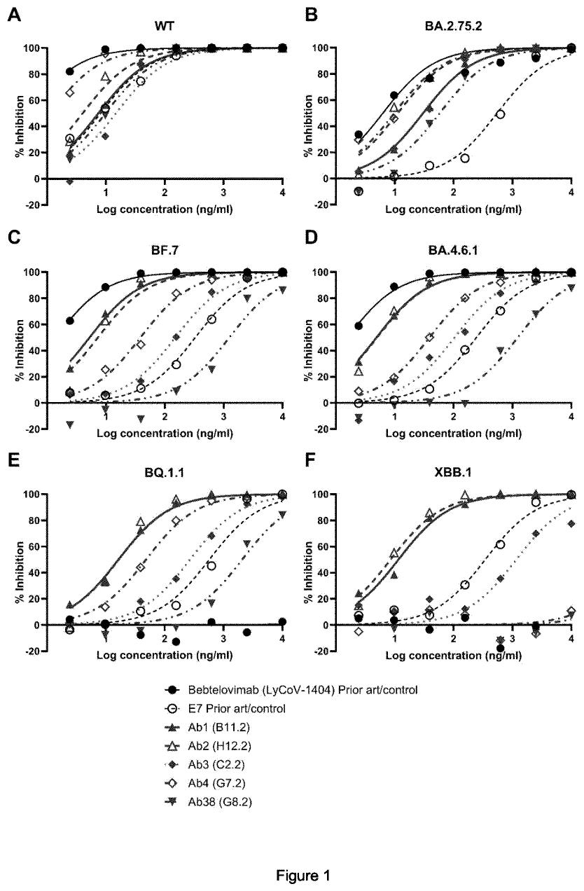

FIG. 1A to 1F shows the results obtained in the pVNT for two antibodies known antibodies to SARS-CoV-2 spike protein (LyCoV-1404 and E7), and Ab1, Ab2, Ab3, Ab4 and Ab38. Ab1, Ab2, Ab3 and Ab4 were found to inhibit infection of ACE2-expressing cells by pseudoviruses expressing the spike proteins of SARS-CoV-2 and all of the SARS-CoV-2 variants tested.

Ab1, Ab2, Ab3, Ab4 and Ab38 neutralised infection of ACE2-expressing cells by pseudovirus expressing the BQ.1.1 spike protein, whose infection of ACE2-expressing cells is not inhibited by LyCoV-1404 (FIG. 1E). Ab1, Ab2, Ab3 and Ab4 were moreover more potent at inhibiting infection of ACE2-expressing cells by pseudovirus expressing the BQ.1.1 spike protein than E7.

Ab1, Ab2 and Ab3 neutralised infection of ACE2-expressing cells by pseudovirus expressing the XBB.1 spike protein, whose infection of ACE2-expressing cells is not inhibited by LyCoV-1404 (FIG. 1F). Ab1 and Ab2 were moreover more potent at inhibiting infection of ACE2-expressing cells by pseudovirus expressing the XBB.1 spike protein than E7.

Ab1 and Ab2 moreover inhibited infection of ACE2-expressing cells by pseudoviruses expressing SARS-CoV-2, BA2.75.2, BF.7 or BA.4.6.1 spike proteins with similar potency to LyCoV-1404, and with improved potency as compared to E7.

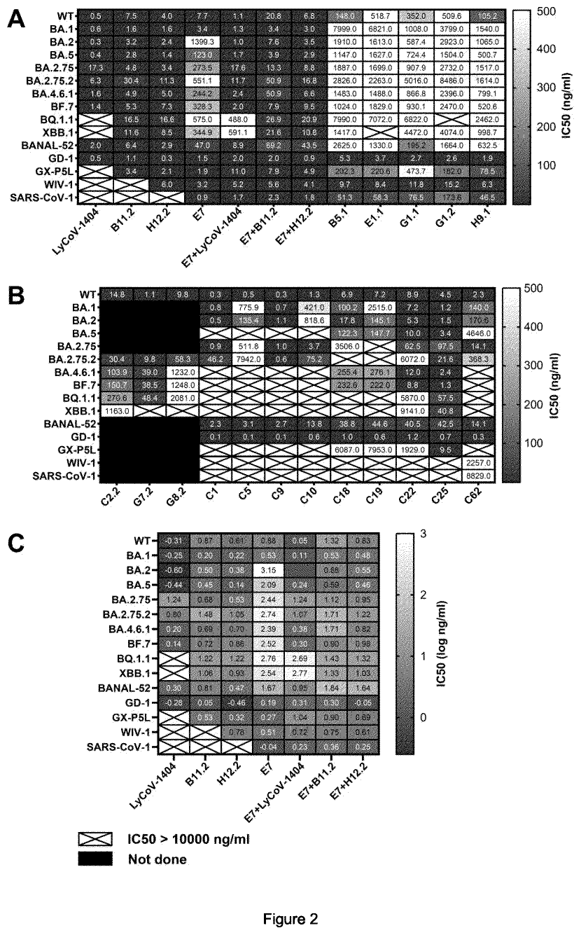

The antibodies were then evaluated in a pVNT against pseudoviruses expressing spike proteins derived from a wider range of sarbecoviruses, including SARS-CoV-2, the SARS-CoV-2 variants BA.1, BA.2, BA.5, BA.2.75, BA.2.75.2, BA.4.6.1, BF.7, BQ.1.1 and XBB, the clade 1b sarbecoviruses BANAL-20-52 (BANAL-52), GD-1, GX-P5L, and the clade 1a sarbecoviruses WIV-1 and SARS-CoV-1. The inventors also investigated the performance of certain combinations of antibodies (E7+LyCoV-1404, E7+Ab1, E7+H12.2), at a 1:1 ratio. IC₅₀ values derived from the % inhibition response curves fitted to the neutralisation data are shown in FIGS. 2A to 2C.

Many of the antibodies were determined to neutralised infection of ACE2-expressing cells by pseudoviruses expressing spike proteins derived from sarbecoviruses whose infection of ACE2-expressing cells was not inhibited by LyCoV-1404. Several of the antibodies inhibited infection of ACE2-expressing cells by pseudoviruses expressing spike proteins derived from sarbecoviruses with greater potency than E7.

Ab1 (B11.2) and Ab2 (H12.2) exhibited ultrapotent ability to neutralise 13 out of 15, and 14 out of 15, of the tested pseudo-sarbecoviruses respectively (that is, pseudoviruses expressing spike proteins derived from all of the clade 1b sarbecoviruses investigated), with IC50 values ranging from 1.1 to 30.4 ng/ml for Ab 1, and 0.3 to 16.6 ng/ml for Ab 2 (shown in FIG. 2A). Preparations comprising E7+Ab1 or E7+Ab2 were able to neutralise all of the tested pseudo-sarbecoviruses (that is, pseudoviruses expressing spike proteins derived from all of the clade 1a and 1b sarbecoviruses investigated), with IC50 values ranging from 0.9-69.2 ng/ml.

Control antibody LyCoV-1404 showed potent activity against 10 out of the 15 tested sarbecoviruses, but was inactive against the remaining 6, including SARS-CoV-2 variants BQ.1.1 or XBB.

Control antibody E7 demonstrated neutralising ability against all of the tested variants, although showed considerably lower potency compared to Ab 1 and Ab 2 against most of the tested SARS-CoV-2 variants, including BQ.1.1 and XBB.

These data demonstrate the broad-range, and highly-potent ability of newly developed antibodies Ab 1 and Ab 2, including their ability to neutralise clade 1 sarbecoviruses, including SARS-CoV-2 variants BQ.1.1 and XBB.1, which are not neutralised with high potency by known antibodies E7 and LyCoV-1404.

### Example 3—Analysis of Ability of Antibodies to Neutralise Infection of ACE2-Expressing Cells by Pseudo-Sarbecoviruses Expressing SARS-CoV-2 Variant XBB.1.16, XBB.2.3, EG.5 or EG5.1 Spike Proteins

The inventors next investigated the ability of Ab2 (H12.2) to inhibit the infection of ACE2-expressing cells by pseudoviruses expressing SARS-CoV-2 variant XBB.1.16, XBB.2.3, EG.5 or EG5.1 spike proteins in further pVNTs. The performance of E7 and LyCoV1404 against these pseudo-sarbecoviruses was also investigated.

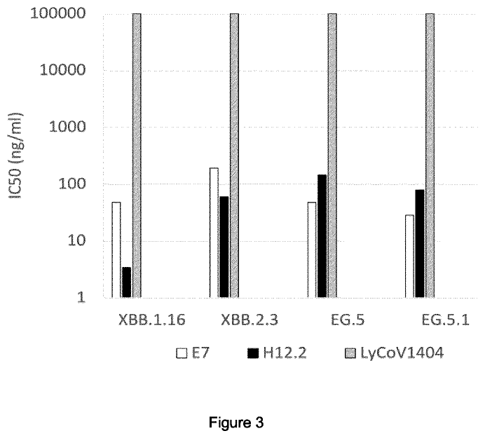

The results are shown in FIG. 3. LyCoV1404 was unable to neutralise pseudoviruses corresponding to these SARS-CoV-2 variants. Ab2 displayed similar or improved ability to neutralise pseudoviruses corresponding to these SARS-CoV-2 variants as compared to E7. In particular, Ab2 was extremely potent at inhibiting infection of ACE2-expressing cells by pseudoviruses expressing SARS-CoV-2 variant XBB.1.16 spike protein.

### Example 4—Serum-Spiked pVNTs

The inventors next investigated the ability of Ab2 (H12.2), E7, and the combination of Ab2+E7 (at a 1:1 ratio) to neutralise infection of ACE2-expressing cells by pseudoviruses expressing the spike proteins of different sarbecoviruses in a modified pVNT, in which human serum is included in the reaction buffer (see Example 1.2).

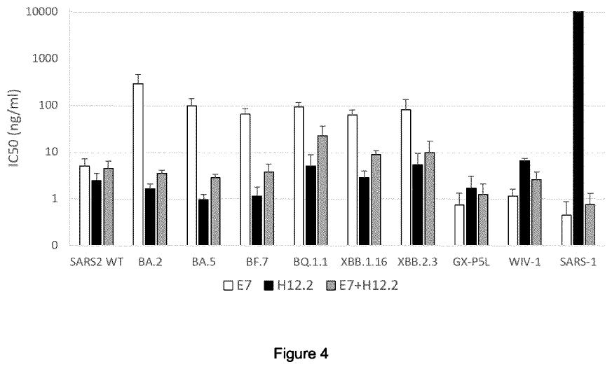

The results are shown in FIG. 4, and are consistent with the results described in Examples 2 and 3. Ab2 displayed similar or improved ability to neutralise infection of ACE2-expressing cells by all pseudo-sarbecoviruses expressing spike proteins from clade 1b sarbecoviruses, as compared to E7. Ab2 also demonstrated similar ability to neutralise infection of ACE2-expressing cells by pseudo-sarbecovirus expressing spike proteins from clade 1a sarbecovirus WIV-1, as compared to E7. The combination of Ab2+E7 potently neutralised all infection of ACE2-expressing cells by all pseudo-sarbecoviruses evaluated, include those expressing spike proteins from clade 1a sarbecoviruses (WIV-1 and SARS-CoV-1).

### Example 5—Ab 2 Demonstrates Neutralisation Against Clade 3 Sarbecovirus

The inventors next investigated the ability of Ab2 and E7 to inhibit interaction between ACE2 and the polypeptides consisting essentially of the RBD of the spike proteins of various different sarbecoviruses, in a multiplex surrogate virus neutralization test (sVNT; see Example 1.3).

The experiment investigated the ability of the antibodies to inhibit interaction between the RBD of Khosta-2 and ACE2. Khosta-2 is a clade 3 sarbecovirus which demonstrates binding to human ACE2, and resistance to current SARS-CoV-2 vaccines, therefore representing a potential threat for future human infection (see e.g. Seifert et al. PLOS Pathog. (2022) 18(9):e1010828).

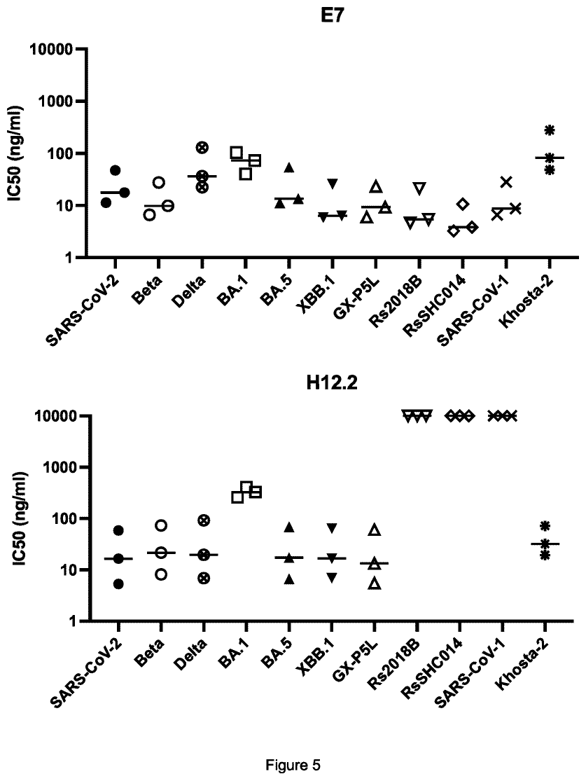

The results are shown in FIG. 5. Both Ab 2 and E7 inhibited interaction between Khosta-2 RBD and human ACE2. E7 inhibited interaction between ACE2 and the RBDs of all sarbecoviruses tested, while Ab2 inhibited interaction between ACE2 and the RBDs of all clade 1b and clade 3 sarbecoviruses analysed.

1. An antigen-binding molecule, optionally isolated, that binds to a sarbecovirus spike protein, wherein the antigen-binding molecule comprises:
(i) a heavy chain variable (VH) region incorporating the following CDRs:
HC-CDR1 having the amino acid sequence of SEQ ID NO:37
HC-CDR2 having the amino acid sequence of SEQ ID NO:53
HC-CDR3 having the amino acid sequence of SEQ ID NO:54; and
(ii) a light chain variable (VL) region incorporating the following CDRs:
LC-CDR1 having the amino acid sequence of SEQ ID NO:60
LC-CDR2 having the amino acid sequence of SEQ ID NO:61
LC-CDR3 having the amino acid sequence of SEQ ID NO:62.

2. The antigen-binding molecule according to claim 1, wherein the antigen-binding molecule comprises:
a VH region having an amino acid sequence having at least 70% amino acid sequence identity to SEQ ID NO:52; and
a VL region having an amino acid sequence having at least 70% amino acid sequence identity to SEQ ID NO:59.

3. The antigen-binding molecule according to claim 1 or claim 2, wherein the antigen-binding molecule is a multispecific antigen-binding molecule, and wherein the antigen-binding molecule further comprises an antigen-binding domain which binds to an antigen other than a sarbecovirus spike protein.

4. A chimeric antigen receptor (CAR) comprising an antigen-binding molecule according to any one of claims 1 to 3.

5. A nucleic acid, or a plurality of nucleic acids, optionally isolated, encoding an antigen-binding molecule according to any one of claims 1 to 3, or a CAR according to claim 4.

6. An expression vector, or a plurality of expression vectors, comprising a nucleic acid or a plurality of nucleic acids according to claim 5.

7. A cell comprising an antigen-binding molecule according to any one of claims 1 to 3, a CAR according to claim 4, a nucleic acid or a plurality of nucleic acids according to claim 5, or an expression vector or a plurality of expression vectors according to claim 6.

8. A method comprising culturing a cell according to claim 7 under conditions suitable for expression of an antigen-binding molecule or CAR by the cell.

9. A composition comprising an antigen-binding molecule according to any one of claims 1 to 3, a CAR according to claim 4, a nucleic acid or a plurality of nucleic acids according to claim 5, an expression vector or a plurality of expression vectors according to claim 6, or a cell according to claim 7, and a pharmaceutically acceptable carrier, diluent, excipient or adjuvant.

10. The composition according to claim 9, wherein the composition further comprises: an antigen-binding molecule that binds to a sarbecovirus spike protein, comprising a VH region having an amino acid sequence having at least 70% amino acid sequence identity to SEQ ID NO:824, and a VL region having an amino acid sequence having at least 70% amino acid sequence identity to SEQ ID NO:830.

11. A combination comprising: (i) an antigen-binding molecule according to any one of claims 1 to 3, and (ii) an antigen-binding molecule that binds to a sarbecovirus spike protein, comprising a VH region having an amino acid sequence having at least 70% amino acid sequence identity to SEQ ID NO:824, and a VL region having an amino acid sequence having at least 70% amino acid sequence identity to SEQ ID NO:830.

12. An antigen-binding molecule according to any one of claims 1 to 3, a CAR according to claim 4, a nucleic acid or a plurality of nucleic acids according to claim 5, an expression vector or a plurality of expression vectors according to claim 6, a cell according to claim 7, a composition according to claim 9 or claim 10, or a combination according to claim 11, for use in a method of medical treatment or prophylaxis.

13. An antigen-binding molecule according to any one of claims 1 to 3, a CAR according to claim 4, a nucleic acid or a plurality of nucleic acids according to claim 5, an expression vector or a plurality of expression vectors according to claim 6, a cell according to claim 7, a composition according to claim 9 or claim 10, or a combination according to claim 11, for use in treating or preventing a disease or condition characterised by infection with a sarbecovirus, optionally wherein the disease or condition characterised by infection with a sarbecovirus is COVID-19.

14. Use of an antigen-binding molecule according to any one of claims 1 to 3, a CAR according to claim 4, a nucleic acid or a plurality of nucleic acids according to claim 5, an expression vector or a plurality of expression vectors according to claim 6, a cell according to claim 7, a composition according to claim 9 or claim 10, or a combination according to claim 11 in the manufacture of a medicament for treating or preventing a disease or condition characterised by infection with a sarbecovirus, optionally wherein the disease or condition characterised by infection with a sarbecovirus is COVID-19.

15. A method of treating or preventing a disease or condition characterised by infection with a sarbecovirus in a subject, comprising administering to a subject a therapeutically- or prophylactically-effective amount of an antigen-binding molecule according to any one of claims 1 to 3, a CAR according to claim 4, a nucleic acid or a plurality of nucleic acids according to claim 5, an expression vector or a plurality of expression vectors according to claim 6, a cell according to claim 7, a composition according to claim 9 or claim 10, or a combination according to claim 11, optionally wherein the disease or condition characterised by infection with a sarbecovirus is COVID-19.

16. An in vitro complex, optionally isolated, comprising an antigen-binding molecule according to any one of claims 1 to 3 bound to a sarbecovirus or a sarbecovirus spike protein.

17. A method for detecting a sarbecovirus or a sarbecovirus spike protein in a sample, comprising contacting a sample containing, or suspected to contain, a sarbecovirus or a sarbecovirus spike protein with an antigen-binding molecule according to any one of claims 1 to 3, and detecting the formation of a complex of the antigen-binding molecule with a sarbecovirus or a sarbecovirus spike protein.

18. A method of selecting or stratifying a subject for treatment with a sarbecovirus-targeted agent, the method comprising contacting, in vitro, a sample from the subject with an antigen-binding molecule according to any one of claims 1 to 3, and detecting the formation of a complex of the antigen-binding molecule with a sarbecovirus or a sarbecovirus spike protein.

19. Use of an antigen-binding molecule according to any one of claims 1 to 3 as an in vitro or in vivo diagnostic or prognostic agent.
